SEQUENCE LISTING (129 sequences)

SEQ ID NO: 1 - protein, 5 aa, synthetic construct
  REGION 1..5: note = VH-CDR1
NYYIG

SEQ ID NO: 2 - protein, 17 aa, synthetic construct
  VARIANT 1: note = X is selected from Asp (D), Ile (I) and Arg (R)
  REGION 1..17: note = VH-CDR2
  VARIANT 4: note = X is selected from Pro (P) and Ser (S)
  VARIANT 7: note = X is selected from Asp (D), Ser (S) and Gly (G)
  VARIANT 9: note = X is selected from Ala (A) and Thr (T)
  VARIANT 11: note = X is selected from Ser (S) and Tyr (Y)
  VARIANT 12: note = X is selected from Asn (N), Ala (A) and Ser (S)
  VARIANT 13: note = X is selected from Glu (E), Asp (D), Pro (P) and Gln (Q)
  VARIANT 14: note = X is selected from Lys (K) and Ser (S)
  VARIANT 15: note = X is selected from Phe (F) and Val (V)
  VARIANT 16: note = X is selected from Lys (K) and Gln (Q)
XIFXGGXYXNXXXXXXG

SEQ ID NO: 3 - protein, 5 aa, synthetic construct
  REGION 1..5: note = VH-CDR3
RNFDY

SEQ ID NO: 4 - protein, 17 aa, synthetic construct
  REGION 1..17: note = VH-CDR2
DIFPGGDYANSNEKFKG

SEQ ID NO: 5 - protein, 17 aa, synthetic construct
  REGION 1..17: note = VH-CDR2
DIFPGGDYANSNEKVKG

SEQ ID NO: 6 - protein, 17 aa, synthetic construct
  REGION 1..17: note = VH-CDR2
DIFPGGGYTNYAEKFQG

SEQ ID NO: 7 - protein, 17 aa, synthetic construct
  REGION 1..17: note = VH-CDR2
DIFPGGSYTNYSESFQG

SEQ ID NO: 8 - protein, 17 aa, synthetic construct
  REGION 1..17: note = VH-CDR2
DIFPGGSYTNYADSVKG

SEQ ID NO: 9 - protein, 17 aa, synthetic construct
  REGION 1..17: note = VH-CDR2
RIFPGGGYTNYAQKFQG

SEQ ID NO: 10 - protein, 17 aa, synthetic construct
  REGION 1..17: note = VH-CDR2
IIFPGGSYTNYSPSFQG

SEQ ID NO: 11 - protein, 17 aa, synthetic construct
  REGION 1..17: note = VH-CDR2
DIFSGGSYTNYADSVKG

SEQ ID NO: 12 - protein, 11 aa, synthetic construct
  VARIANT 1: note = X is selected from Ser (S) and Arg (R)
  REGION 1..11: note = VL-CDR1
  VARIANT 8: note = X is absent, or is selected from Asn (N), Ser (S) and Gly (G)
XASSSVSXYMH

SEQ ID NO: 13 - skipped

SEQ ID NO: 14 - protein, 10 aa, synthetic construct
  VARIANT 1: note = X is selected from Gln (Q) and His (H)
  REGION 1..10: note = VL-CDR3
XQRSSYPLTF

SEQ ID NO: 15 - protein, 11 aa, synthetic construct
  REGION 1..11: note = VL-CDR1
  VARIANT 8: note = X is absent or is selected from Asn (N), Ser (S) and Gly (G)
SASSSVSXYMH

SEQ ID NO: 16 - protein, 7 aa, synthetic construct
  REGION 1..7: note = VL-CDR2
NTSNLPS

SEQ ID NO: 17 - protein, 10 aa, synthetic construct
  REGION 1..10: note = VL-CDR3
QQRSSYPLTF

SEQ ID NO: 18 - protein, 11 aa, synthetic construct
  REGION 1..11: note = VL-CDR1
  VARIANT 8: note = X is absent or is selected from Asn (N), Ser (S) and Gly (G)
RASSSVSXYMH

SEQ ID NO: 19 - protein, 7 aa, synthetic construct
  REGION 1..7: note = VL-CDR2
NTSNSPS

SEQ ID NO: 20 - protein, 7 aa, synthetic construct
  REGION 1..7: note = VL-CDR2
ATSNLQS

SEQ ID NO: 21 - protein, 10 aa, synthetic construct
  REGION 1..10: note = VL-CDR3
HQRSSYPLTF

SEQ ID NO: 22 - protein, 7 aa, synthetic construct
  REGION 1..7: note = VL-CDR2
NTSNRAT

SEQ ID NO: 23 - protein, 48 aa, Homo sapiens
  REGION 1..48: note = C determinant of hCD45RC
DVPGERSTASTFPTDPVSPLTTTLSLAHHSSAALPARTSNTTITANTS

SEQ ID NO: 24 - DNA, 144 bp, Homo sapiens
  misc_feature 1..144: note = Exon 6 of hCD45RC
  source 1..144: mol_type = other DNA
gatgtcccaggagagaggagtacagccagcacctttcctacagacccagtttccccattgacaaccaccctcagccttgcacaccacagctctgctgccttacctgcacgcacctccaacaccaccatcacagcgaacacctca

SEQ ID NO: 25 - protein, 30 aa, synthetic construct
  REGION 1..30: note = VH-FR1
QVQLQQSGAELVRPGTSVKMSCKAAGYTFT

SEQ ID NO: 26 - protein, 14 aa, synthetic construct
  REGION 1..14: note = VH-FR2
WVKQRPGHGLEWIG

SEQ ID NO: 27 - protein, 32 aa, synthetic construct
  REGION 1..32: note = VH-FR3
KATLTADTSSSTAYMQLSSLTSEDSAIYYCVR

SEQ ID NO: 28 - protein, 11 aa, synthetic construct
  REGION 1..11: note = VH-FR4
WGQGTTLTVSS

SEQ ID NO: 29 - protein, 30 aa, synthetic construct
  REGION 1..30: note = VH-FR1
QVQLVQSGAEVKKPGASVKVSCKASGYTFT

SEQ ID NO: 30 - protein, 14 aa, synthetic construct
  REGION 1..14: note = VH-FR2
WVRQAPGQGLEWIG

SEQ ID NO: 31 - protein, 32 aa, synthetic construct
  REGION 1..32: note = VH-FR3
RVTLTADTSISTAYMELSRLRSDDTVVYYCVR

SEQ ID NO: 32 - protein, 11 aa, synthetic construct
  REGION 1..11: note = VH-FR4
WGQGTLVTVSS

SEQ ID NO: 33 - protein, 30 aa, synthetic construct
  REGION 1..30: note = VH-FR1
EVQLVQSGAEVKKPGESLKISCKASGYTFT

SEQ ID NO: 34 - protein, 14 aa, synthetic construct
  REGION 1..14: note = VH-FR2
WVRQMPGKGLEWIG

SEQ ID NO: 35 - protein, 32 aa, synthetic construct
  REGION 1..32: note = VH-FR3
QVTLSADKSISTAYLQLSSLKASDTAMYYCVR

SEQ ID NO: 36 - protein, 30 aa, synthetic construct
  REGION 1..30: note = VH-FR1
QVQLVESGGGLVKPGGSLRLSCAASGYTFT

SEQ ID NO: 37 - protein, 14 aa, synthetic construct
  REGION 1..14: note = VH-FR2
WIRQAPGKGLEWIG

SEQ ID NO: 38 - protein, 32 aa, synthetic construct
  REGION 1..32: note = VH-FR3
RFTLSADTAKNSAYLQMNSLRAEDTAVYYCVR

SEQ ID NO: 39 - protein, 30 aa, synthetic construct
  REGION 1..30: note = VH-FR1
EVQLVQSGAEVKKPGESLKISCKGSGYTFT

SEQ ID NO: 40 - protein, 30 aa, synthetic construct
  REGION 1..30: note = VH-FR1
QVQLVESGGGLVKPGGSLRLSCAASGFTFS

SEQ ID NO: 41 - protein, 32 aa, synthetic construct
  REGION 1..32: note = VH-FR3
RFTLSADTAKNSLYLQMNSLRAEDTAVYYCVR

SEQ ID NO: 42 - protein, 30 aa, synthetic construct
  REGION 1..30: note = VH-FR1
EVQLVQSGAEVKKPGESLKISCKGSGYSFT

SEQ ID NO: 43 - protein, 14 aa, synthetic construct
  REGION 1..14: note = VH-FR2
WIRQAPGKGLEWVG

SEQ ID NO: 44 - protein, 23 aa, synthetic construct
  REGION 1..23: note = VL-FR1
QIVLTQSPTIMSASPGEKVTITC

SEQ ID NO: 45 - protein, 15 aa, synthetic construct
  REGION 1..15: note = VL-FR2
WFQQKTGTSPRLWIY

SEQ ID NO: 46 - protein, 32 aa, synthetic construct
  REGION 1..32: note = VL-FR3
  VARIANT 15: note = X is selected from Tyr (Y) and Phe (F)
GVPARFSGSGSGTSXSLTISRMEAEDAATYYC

SEQ ID NO: 47 - protein, 9 aa, synthetic construct
  REGION 1..9: note = VL-FR4
GAGTKLELK

SEQ ID NO: 48 - protein, 23 aa, synthetic construct
  REGION 1..23: note = VL-FR1
DIQLTQSPSFLSASVGDRVTITC

SEQ ID NO: 49 - protein, 15 aa, synthetic construct
  REGION 1..15: note = VL-FR2
WFQQKPGKAPKLWIY

SEQ ID NO: 50 - protein, 32 aa, synthetic construct
  REGION 1..32: note = VL-FR3
  VARIANT 15: note = X is selected from Tyr (Y) and Phe (F)
GVPSRFSGSGSGTEXTLTISSLQPEDFATYYC

SEQ ID NO: 51 - protein, 9 aa, synthetic construct
  REGION 1..9: note = VL-FR4
GGGTKVEIK

SEQ ID NO: 52 - protein, 23 aa, synthetic construct
  REGION 1..23: note = VL-FR1
EIVLTQSPDFQSVTPKEKVTITC

SEQ ID NO: 53 - protein, 15 aa, synthetic construct
  REGION 1..15: note = VL-FR2
WFQQKPDQSPKLWIY

SEQ ID NO: 54 - protein, 32 aa, synthetic construct
  REGION 1..32: note = VL-FR3
  VARIANT 15: note = X is selected from Tyr (Y) and Phe (F)
GVPSRFSGSGSGTDXTLTINSLEAEDAATYYC

SEQ ID NO: 55 - protein, 23 aa, synthetic construct
  REGION 1..23: note = VL-FR1
EIVLTQSPATLSLSPGERATLSC

SEQ ID NO: 56 - protein, 15 aa, synthetic construct
  REGION 1..15: note = VL-FR2
WFQQKPGQAPRLWIY

SEQ ID NO: 57 - protein, 32 aa, synthetic construct
  REGION 1..32: note = VL-FR3
  VARIANT 15: note = X is selected from Tyr (Y) and Phe (F)
GIPARFSGSGSGTDXTLTISSLEPEDFAVYYC

SEQ ID NO: 58 - protein, 15 aa, synthetic construct
  REGION 1..15: note = VL-FR2
WYQQKPGKAPKLWIY

SEQ ID NO: 59 - protein, 15 aa, synthetic construct
  REGION 1..15: note = VL-FR2
WYQQKPDQSPKLWIY

SEQ ID NO: 60 - protein, 15 aa, synthetic construct
  REGION 1..15: note = VL-FR2
WYQQKPGQAPRLWIY

SEQ ID NO: 61 - protein, 114 aa, synthetic construct
  REGION 1..114: note = Murine HCVR
QVQLQQSGAELVRPGTSVKMSCKAAGYTFTNYYIGWVKQRPGHGLEWIGDIFPGGDYANSNEKFKGKATLTADTSSSTAYMQLSSLTSEDSAIYYCVRRNFDYWGQGTTLTVSS

SEQ ID NO: 62 - protein, 114 aa, synthetic construct
  REGION 1..114: note = Humanized HCVR
QVQLVQSGAEVKKPGASVKVSCKASGYTFTNYYIGWVRQAPGQGLEWIGDIFPGGDYANSNEKFKGRVTLTADTSISTAYMELSRLRSDDTVVYYCVRRNFDYWGQGTLVTVSS

SEQ ID NO: 63 - protein, 114 aa, synthetic construct
  REGION 1..114: note = Humanized HCVR
EVQLVQSGAEVKKPGESLKISCKASGYTFTNYYIGWVRQMPGKGLEWIGDIFPGGDYANSNEKFKGQVTLSADKSISTAYLQLSSLKASDTAMYYCVRRNFDYWGQGTLVTVSS

SEQ ID NO: 64 - protein, 114 aa, synthetic construct
  REGION 1..114: note = Humanized HCVR
QVQLVESGGGLVKPGGSLRLSCAASGYTFTNYYIGWIRQAPGKGLEWIGDIFPGGDYANSNEKVKGRFTLSADTAKNSAYLQMNSLRAEDTAVYYCVRRNFDYWGQGTLVTVSS

SEQ ID NO: 65 - protein, 114 aa, synthetic construct
  REGION 1..114: note = Humanized HCVR
QVQLVQSGAEVKKPGASVKVSCKASGYTFTNYYIGWVRQAPGQGLEWIGDIFPGGGYTNYAEKFQGRVTLTADTSISTAYMELSRLRSDDTVVYYCVRRNFDYWGQGTLVTVSS

SEQ ID NO: 66 - protein, 114 aa, synthetic construct
  REGION 1..114: note = Humanized HCVR
EVQLVQSGAEVKKPGESLKISCKGSGYTFTNYYIGWVRQMPGKGLEWIGDIFPGGSYTNYSESFQGQVTLSADKSISTAYLQLSSLKASDTAMYYCVRRNFDYWGQGTLVTVSS

SEQ ID NO: 67 - protein, 114 aa, synthetic construct
  REGION 1..114: note = Humanized HCVR
QVQLVESGGGLVKPGGSLRLSCAASGFTFSNYYIGWIRQAPGKGLEWIGDIFPGGSYTNYADSVKGRFTLSADTAKNSLYLQMNSLRAEDTAVYYCVRRNFDYWGQGTLVTVSS

SEQ ID NO: 68 - protein, 114 aa, synthetic construct
  REGION 1..114: note = Humanized HCVR
QVQLVQSGAEVKKPGASVKVSCKASGYTFTNYYIGWVRQAPGQGLEWIGRIFPGGGYTNYAQKFQGRVTLTADTSISTAYMELSRLRSDDTVVYYCVRRNFDYWGQGTLVTVSS

SEQ ID NO: 69 - protein, 114 aa, synthetic construct
  REGION 1..114: note = Humanized HCVR
EVQLVQSGAEVKKPGESLKISCKGSGYSFTNYYIGWVRQMPGKGLEWIGIIFPGGSYTNYSPSFQGQVTLSADKSISTAYLQLSSLKASDTAMYYCVRRNFDYWGQGTLVTVSS

SEQ ID NO: 70 - protein, 114 aa, synthetic construct
  REGION 1..114: note = Humanized HCVR
QVQLVESGGGLVKPGGSLRLSCAASGFTFSNYYIGWIRQAPGKGLEWVGDIFSGGSYTNYADSVKGRFTLSADTAKNSLYLQMNSLRAEDTAVYYCVRRNFDYWGQGTLVTVSS

SEQ ID NO: 71 - protein, 107 aa, synthetic construct
  REGION 1..107: note = Murine LCVR
  VARIANT 31: note = X is selected from Asn (N), Ser (S) and Gly (G)
QIVLTQSPTIMSASPGEKVTITCSASSSVSXYMHWFQQKTGTSPRLWIYNTSNLPSGVPARFSGSGSGTSFSLTISRMEAEDAATYYCQQRSSYPLTFGAGTKLELK

SEQ ID NO: 72 - protein, 107 aa, synthetic construct
  REGION 1..107: note = Humanized LCVR
  VARIANT 31: note = X is selected from Asn (N), Ser (S) and Gly (G)
DIQLTQSPSFLSASVGDRVTITCSASSSVSXYMHWFQQKPGKAPKLWIYNTSNLPSGVPSRFSGSGSGTEFTLTISSLQPEDFATYYCQQRSSYPLTFGGGTKVEIK

SEQ ID NO: 73 - protein, 107 aa, synthetic construct
  REGION 1..107: note = Humanized LCVR
  VARIANT 31: note = X is selected from Asn (N), Ser (S) and Gly (G)
EIVLTQSPDFQSVTPKEKVTITCSASSSVSXYMHWFQQKPDQSPKLWIYNTSNLPSGVPSRFSGSGSGTDFTLTINSLEAEDAATYYCQQRSSYPLTFGGGTKVEIK

SEQ ID NO: 74 - protein, 107 aa, synthetic construct
  REGION 1..107: note = Humanized LCVR
  VARIANT 31: note = X is selected from Asn (N), Ser (S) and Gly (G)
EIVLTQSPATLSLSPGERATLSCSASSSVSXYMHWFQQKPGQAPRLWIYNTSNLPSGIPARFSGSGSGTDFTLTISSLEPEDFAVYYCQQRSSYPLTFGGGTKVEIK

SEQ ID NO: 75 - protein, 107 aa, synthetic construct
  REGION 1..107: note = Humanized LCVR
  VARIANT 31: note = X is selected from Asn (N), Ser (S) and Gly (G)
DIQLTQSPSFLSASVGDRVTITCRASSSVSXYMHWYQQKPGKAPKLWIYNTSNLPSGVPSRFSGSGSGTEFTLTISSLQPEDFATYYCQQRSSYPLTFGGGTKVEIK

SEQ ID NO: 76 - protein, 107 aa, synthetic construct
  REGION 1..107: note = Humanized LCVR
  VARIANT 31: note = X is selected from Asn (N), Ser (S) and Gly (G)
EIVLTQSPDFQSVTPKEKVTITCRASSSVSXYMHWYQQKPDQSPKLWIYNTSNSPSGVPSRFSGSGSGTDFTLTINSLEAEDAATYYCQQRSSYPLTFGGGTKVEIK

SEQ ID NO: 77 - protein, 107 aa, synthetic construct
  REGION 1..107: note = Humanized LCVR
  VARIANT 31: note = X is selected from Asn (N), Ser (S) and Gly (G)
EIVLTQSPATLSLSPGERATLSCRASSSVSXYMHWFQQKPGQAPRLWIYNTSNLPSGIPARFSGSGSGTDFTLTISSLEPEDFAVYYCQQRSSYPLTFGGGTKVEIK

SEQ ID NO: 78 - protein, 107 aa, synthetic construct
  REGION 1..107: note = Humanized LCVR
  VARIANT 31: note = X is selected from Asn (N), Ser (S) and Gly (G)
DIQLTQSPSFLSASVGDRVTITCRASSSVSXYMHWYQQKPGKAPKLWIYATSNLQSGVPSRFSGSGSGTEFTLTISSLQPEDFATYYCQQRSSYPLTFGGGTKVEIK

SEQ ID NO: 79 - protein, 107 aa, synthetic construct
  REGION 1..107: note = Humanized LCVR
  VARIANT 31: note = X is selected from Asn (N), Ser (S) and Gly (G)
EIVLTQSPDFQSVTPKEKVTITCRASSSVSXYMHWYQQKPDQSPKLWIYNTSNSPSGVPSRFSGSGSGTDFTLTINSLEAEDAATYYCHQRSSYPLTFGGGTKVEIK

SEQ ID NO: 80 - protein, 107 aa, synthetic construct
  REGION 1..107: note = Humanized LCVR
  VARIANT 31: note = X is selected from Asn (N), Ser (S) and Gly (G)
EIVLTQSPATLSLSPGERATLSCRASSSVSXYMHWYQQKPGQAPRLWIYNTSNRATGIPARFSGSGSGTDFTLTISSLEPEDFAVYYCQQRSSYPLTFGGGTKVEIK

SEQ ID NO: 81 - protein, 106 aa, synthetic construct
  REGION 1..106: note = Murine LCVR
QIVLTQSPTIMSASPGEKVTITCSASSSVSYMHWFQQKTGTSPRLWIYNTSNLPSGVPARFSGSGSGTSYSLTISRMEAEDAATYYCQQRSSYPLTFGAGTKLELK

SEQ ID NO: 82 - protein, 106 aa, synthetic construct
  REGION 1..106: note = Humanized LCVR
DIQLTQSPSFLSASVGDRVTITCSASSSVSYMHWFQQKPGKAPKLWIYNTSNLPSGVPSRFSGSGSGTEYTLTISSLQPEDFATYYCQQRSSYPLTFGGGTKVEIK

SEQ ID NO: 83 - protein, 106 aa, synthetic construct
  REGION 1..106: note = Humanized LCVR
EIVLTQSPDFQSVTPKEKVTITCSASSSVSYMHWFQQKPDQSPKLWIYNTSNLPSGVPSRFSGSGSGTDYTLTINSLEAEDAATYYCQQRSSYPLTFGGGTKVEIK

SEQ ID NO: 84 - protein, 106 aa, synthetic construct
  REGION 1..106: note = Humanized LCVR
EIVLTQSPATLSLSPGERATLSCSASSSVSYMHWFQQKPGQAPRLWIYNTSNLPSGIPARFSGSGSGTDYTLTISSLEPEDFAVYYCQQRSSYPLTFGGGTKVEIK

SEQ ID NO: 85 - protein, 106 aa, synthetic construct
  REGION 1..106: note = Humanized LCVR
DIQLTQSPSFLSASVGDRVTITCRASSSVSYMHWYQQKPGKAPKLWIYNTSNLPSGVPSRFSGSGSGTEYTLTISSLQPEDFATYYCQQRSSYPLTFGGGTKVEIK

SEQ ID NO: 86 - protein, 106 aa, synthetic construct
  REGION 1..106: note = Humanized LCVR
EIVLTQSPDFQSVTPKEKVTITCRASSSVSYMHWYQQKPDQSPKLWIYNTSNSPSGVPSRFSGSGSGTDFTLTINSLEAEDAATYYCQQRSSYPLTFGGGTKVEIK

SEQ ID NO: 87 - protein, 106 aa, synthetic construct
  REGION 1..106: note = Humanized LCVR
EIVLTQSPATLSLSPGERATLSCRASSSVSYMHWFQQKPGQAPRLWIYNTSNLPSGIPARFSGSGSGTDYTLTISSLEPEDFAVYYCQQRSSYPLTFGGGTKVEIK

SEQ ID NO: 88 - protein, 106 aa, synthetic construct
  REGION 1..106: note = Humanized LCVR
DIQLTQSPSFLSASVGDRVTITCRASSSVSYMHWYQQKPGKAPKLWIYATSNLQSGVPSRFSGSGSGTEFTLTISSLQPEDFATYYCQQRSSYPLTFGGGTKVEIK

SEQ ID NO: 89 - protein, 106 aa, synthetic construct
  REGION 1..106: note = Humanized LCVR
EIVLTQSPDFQSVTPKEKVTITCRASSSVSYMHWYQQKPDQSPKLWIYNTSNSPSGVPSRFSGSGSGTDFTLTINSLEAEDAATYYCHQRSSYPLTFGGGTKVEIK

SEQ ID NO: 90 - protein, 106 aa, synthetic construct
  REGION 1..106: note = Humanized LCVR
EIVLTQSPATLSLSPGERATLSCRASSSVSYMHWYQQKPGQAPRLWIYNTSNRATGIPARFSGSGSGTDFTLTISSLEPEDFAVYYCQQRSSYPLTFGGGTKVEIK

SEQ ID NO: 91 - protein, 330 aa, synthetic construct
  REGION 1..330: note = Human HCCR
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 107 aa, synthetic construct [sequence omitted]

SEQ ID NO: 93 - protein, 323 aa, synthetic construct
  REGION 1..323: note = Murine HCCR
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 94 - protein, 107 aa, synthetic construct
  REGION 1..107: note = Murine LCCR
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 95 - DNA, 342 bp, synthetic construct
  misc_feature 1..342: note = Murine HCVR
  source 1..342: mol_type = other DNA
caggtccagctgcaacagtctggcgctgagctggttaggcctgggacttcagtgaagatgtcctgcaaggccgctggatacaccttcactaactactacataggttgggtaaagcagaggcctggacatggccttgagtggatcggagatattttccctggaggtgactatgccaacagcaatgagaagttcaagggcaaagccacactgactgcagacacatcctccagcacagcctacatgcagctcagcagcctgacatctgaggactctgccatctattactgtgtgagaaggaactttgactactggggccaaggcaccactctcacagtgtcctca

SEQ ID NO: 96 - DNA, 318 bp, synthetic construct
  misc_feature 1..318: note = Murine LCVR
  source 1..318: mol_type = other DNA
caaattgttctcacccagtctccaacaatcatgtctgcatctccaggggagaaggtgaccataacctgcagtgccagctcaagtgtaagttacatgcactggttccagcagaagacaggcacttctcccagactctggatttataacacatccaacctgccttctggagtccccgctcgcttcagtggcagtggatctgggacctcttactctctcacaatcagccgaatggaggctgaagatgctgccacttattactgccagcaaaggagtagttacccactcacgttcggtgctgggaccaagctggagctgaaa

SEQ ID NO: 97 - DNA, 990 bp, synthetic construct
  misc_feature 1..990: note = Human HCCR
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 98 - DNA, 321 bp, synthetic construct
  misc_feature 1..321: note = Human LCCR
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 99 - protein, 1306 aa, Homo sapiens
  REGION 1..1306: note = hCD45
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 100 - protein, 17 aa, synthetic construct
  REGION 1..17: note = VH-CDR2
DIFPGGDYTNYAEKFQG

SEQ ID NO: 101 - protein, 114 aa, synthetic construct
  REGION 1..114: note = Humanized HCVR
QVQLVQSGAEVKKPGASVKVSCKASGYTFTNYYIGWVRQAPGQGLEWIGDIFPGGDYTNYAEKFQGRVTLTADTSISTAYMELSRLRSDDTVVYYCVRRNFDYWGQGTLVTVSS

SEQ ID NO: 102 - protein, 107 aa, synthetic construct
  REGION 1..107: note = Humanized LCVR
  VARIANT 31: note = X is selected from Asn (N), Ser (S) and Gly (G)
DIQLTQSPSFLSASVGDRVTITCRASSSVSXYMHWFQQKPGKAPKLWIYNTSNLPSGVPSRFSGSGSGTEFTLTISSLQPEDFATYYCQQRSSYPLTFGGGTKVEIK

SEQ ID NO: 103 - protein, 107 aa, synthetic construct
  REGION 1..107: note = Humanized LCVR
DIQLTQSPSFLSASVGDRVTITCRASSSVSSYMHWFQQKPGKAPKLWIYNTSNLPSGVPSRFSGSGSGTEFTLTISSLQPEDFATYYCQQRSSYPLTFGGGTKVEIK

SEQ ID NO: 104 - protein, 1193 aa, Homo sapiens
  REGION 1..1193: note = hCD45RC
MTMYLWLKLLAFGFAFLDTEVFVTGQSPTPSPTDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 105 - protein, 66 aa, Homo sapiens
  REGION 1..66: note = A determinant of hCD45
GLTTAKMPSVPLSSDPLPTHTTAFSPASTFERENDFSETTTSLSPDNTSTQVSPDSLDNASAFNTT

SEQ ID NO: 106 - protein, 47 aa, Homo sapiens
  REGION 1..47: note = B determinant of hCD45
GVSSVQTPHLPTHADSQTPSAGTDTQTFSGSAANAKLNPTPGSNAIS

SEQ ID NO: 107 - protein, 1211 aa, Homo sapiens
  REGION 1..1211: note = hCD45RA
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 108 - protein, 1192 aa, Homo sapiens
  REGION 1..1192: note = hCD45RB
MTMYLWLKLLAFGFAFLDTEVFVTGQSPTPSPTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 109 - protein, 1258 aa, Homo sapiens
  REGION 1..1258: note = hCD45RAB
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 110 - protein, 1145 aa, Homo sapiens
  REGION 1..1145: note = hCD45R0
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 111 - protein, 7 aa, synthetic construct
  REGION 1..7: note = VL-CDR2
ATSNLPS

SEQ ID NO: 112 - protein, 107 aa, synthetic construct
  REGION 1..107: note = Humanized LCVR
  VARIANT 31: note = X is selected from Asn (N), Ser (S) and Gly (G)
DIQLTQSPSFLSASVGDRVTITCRASSSVSXYMHWYQQKPGKAPKLWIYATSNLPSGVPSRFSGSGSGTEFTLTISSLQPEDFATYYCQQRSSYPLTFGGGTKVEIK

SEQ ID NO: 113 - protein, 106 aa, synthetic construct
  REGION 1..106: note = Humanized LCVR
DIQLTQSPSFLSASVGDRVTITCRASSSVSYMHWYQQKPGKAPKLWIYATSNLPSGVPSRFSGSGSGTEYTLTISSLQPEDFATYYCQQRSSYPLTFGGGTKVEIK

SEQ ID NO: 114 - protein, 330 aa, synthetic construct
  REGION 1..330: note = Human HCCR
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 113 aa, synthetic construct
  REGION 1..113: note = Human HCCR
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCVAAAHHHHHH

SEQ ID NO: 116 - protein, 17 aa, synthetic construct
  REGION 1..17: note = VH-CDR2
DIFPGGGYANYAEKFQG

SEQ ID NO: 117 - protein, 17 aa, synthetic construct
  REGION 1..17: note = VH-CDR2
DIFPGGGYTNYAEKFKG

SEQ ID NO: 118 - protein, 17 aa, synthetic construct
  REGION 1..17: note = VH-CDR2
DIFPGGGYTNYNEKFQG

SEQ ID NO: 119 - protein, 17 aa, synthetic construct
  REGION 1..17: note = VH-CDR2
DIFPGGGYTNSAEKFQG

SEQ ID NO: 120 - protein, 7 aa, synthetic construct
  REGION 1..7: note = VL-CDR2
NTANLPS

SEQ ID NO: 121 - protein, 114 aa, synthetic construct
  REGION 1..114: note = Humanized HCVR
QVQLVQSGAEVKKPGASVKVSCKASGYTFTNYYIGWVRQAPGQGLEWIGDIFPGGGYANYAEKFQGRVTLTADTSISTAYMELSRLRSDDTVVYYCVRRNFDYWGQGTLVTVSS

SEQ ID NO: 122 - protein, 114 aa, synthetic construct
  REGION 1..114: note = Humanized HCVR
QVQLVQSGAEVKKPGASVKVSCKASGYTFTNYYIGWVRQAPGQGLEWIGDIFPGGGYTNYAEKFKGRVTLTADTSISTAYMELSRLRSDDTVVYYCVRRNFDYWGQGTLVTVSS

SEQ ID NO: 123 - protein, 114 aa, synthetic construct
  REGION 1..114: note = Humanized HCVR
QVQLVQSGAEVKKPGASVKVSCKASGYTFTNYYIGWVRQAPGQGLEWIGDIFPGGGYTNYNEKFQGRVTLTADTSISTAYMELSRLRSDDTVVYYCVRRNFDYWGQGTLVTVSS

SEQ ID NO: 124 - protein, 114 aa, synthetic construct
  REGION 1..114: note = Humanized HCVR
QVQLVQSGAEVKKPGASVKVSCKASGYTFTNYYIGWVRQAPGQGLEWIGDIFPGGGYTNSAEKFQGRVTLTADTSISTAYMELSRLRSDDTVVYYCVRRNFDYWGQGTLVTVSS

SEQ ID NO: 125 - protein, 107 aa, synthetic construct
  REGION 1..107: note = Humanized LCVR
  VARIANT 31: note = X is selected from Asn (N), Ser (S) and Gly (G)
QIVLTQSPTIMSASPGEKVTITCSASSSVSXYMHWFQQKTGTSPRLWIYNTANLPSGVPARFSGSGSGTSFSLTISRMEAEDAATYYCQQRSSYPLTFGAGTKLELK

SEQ ID NO: 126 - protein, 106 aa, synthetic construct
  REGION 1..106: note = Humanized LCVR
QIVLTQSPTIMSASPGEKVTITCSASSSVSYMHWFQQKTGTSPRLWIYNTANLPSGVPARFSGSGSGTSYSLTISRMEAEDAATYYCQQRSSYPLTFGAGTKLELK

SEQ ID NO: 127 - protein, 7 aa, synthetic construct
  VARIANT 1: note = X is any amino acid but Ala (A) or Asn (N)
  REGION 1..7: note = VL-CDR2
XTSNLPS

SEQ ID NO: 128 - protein, 107 aa, synthetic construct
  REGION 1..107: note = Humanized LCVR
  VARIANT 31: note = X is selected from Asn (N), Ser (S) and Gly (G)
  VARIANT 50: note = X is any amino acid but Ala (A) or Asn (N)
DIQLTQSPSFLSASVGDRVTITCRASSSVSXYMHWYQQKPGKAPKLWIYXTSNLPSGVPSRFSGSGSGTEFTLTISSLQPEDFATYYCQQRSSYPLTFGGGTKVEIK

SEQ ID NO: 129 - protein, 106 aa, synthetic construct
  REGION 1..106: note = Humanized LCVR
  VARIANT 49: note = X is any amino acid but Ala (A) or Asn (N)
DIQLTQSPSFLSASVGDRVTITCRASSSVSYMHWYQQKPGKAPKLWIYXTSNLPSGVPSRFSGSGSGTEYTLTISSLQPEDFATYYCQQRSSYPLTFGGGTKVEIK

(19) United States
(12) Patent Application Publication
(10) Pub. No.: US 2025/0122300 A1
(43) Pub. Date: Apr. 17, 2025

(54) ANTI-HUMAN CD45RC ANTIBODIES AND USES THEREOF

(71) Applicants: INSERM (INSTITUT NATIONAL DE LA SANTÉ ET DE LA RECHERCHE MÉDICALE), Paris Cedex 13 (FR); UNIVERSITÉ DE NANTES, Nantes (FR); CENTRE HOSPITALIER UNIVERSITAIRE DE NANTES, Nantes Cedex 1 (FR)

(72) Inventors: Carole GUILLONNEAU, La Chevrolière (FR); Ignacio ANEGON, Nantes (FR)

(73) Assignees: INSERM (INSTITUT NATIONAL DE LA SANTÉ ET DE LA RECHERCHE MÉDICALE), Paris Cedex 13 (FR); UNIVERSITÉ DE NANTES, Nantes Cedex 1 (FR); CENTRE HOSPITALIER UNIVERSITAIRE DE NANTES, Nantes Cedex 1 (FR)

(21) Appl. No.: 18/934,376

(22) Filed: Nov. 1, 2024

Related U.S. Application Data
(63) Continuation of application No. 17/277,511, filed on Mar. 18, 2021, now Pat. No. 12,168,694.

(30) Foreign Application Priority Data
Sep. 21, 2018 (EP) 18306230.6

(51) Int. Cl. C07K 16/28; A61K 39/00; A61P 37/06

(52) U.S. Cl. CPC C07K 16/289; A61P 37/06; A61K 2039/505; C07K 2317/24; C07K 2317/565; C07K 2317/92

(57) ABSTRACT

Isolated anti-human CD45RC antibodies or binding fragments thereof, nucleic acids and expression vector encoding the same, compositions including the same, and uses thereof as medicaments, including for the prevention and/or treatment of CD45RC^{high}-related diseases (including autoimmune diseases, undesired immune responses, monogenic diseases, and lymphoma or cancer), in particular for use in preventing and/or treating graft-versus-host disease (GVHD).

### CROSS REFERENCE TO RELATED APPLICATIONS

This application is a Continuation of U.S. patent application Ser. No. 17/277,511, filed Mar. 18, 2021, which is a National Stage Application PCT/EP2019/075374, filed Sep. 20, 2019, which claims the benefit of priority from European Patent Application No. 18306230.6, filed Sep. 21, 2018, the contents of which are incorporated herein by reference in its entirety.

### REFERENCE TO A SEQUENCE LISTING

In accordance with 37 CFR § 1.831, the present specification makes reference to a Sequence Listing submitted electronically as a .xml file named “1093/US/2.xml”. The .xml file was generated on Oct. 31, 2024, and is 189,534 bytes in size. The entire contents of the Sequence Listing are hereby incorporated by reference.

### FIELD

The present invention relates to isolated anti-human CD45RC antibodies or binding fragments thereof, to nucleic acids and expression vector encoding the same, to compositions comprising the same, and to uses thereof as medicaments, including for the prevention and/or treatment of CD45RC^{high}-related diseases (including autoimmune diseases, undesired immune responses, monogenic diseases, and lymphoma or cancer), in particular for use in preventing and/or treating graft-versus-host disease (GVHD).

### BACKGROUND

CD45 (also known as leukocyte common antigen (LCA), EC3.1.3.48, T200, Ly5, and PTPRC) constitutes the first and prototypic receptor-like protein tyrosine phosphatase (RPTP). Its expression is restricted to all nucleated hematopoietic cells, where it is one of the most abundant cell surface glycoproteins, constituting almost 10 percent of the cell surface, and estimated to be present at approximately 25 μM in the plasma membrane (Trowbridge & Thomas, 1994. Annu Rev Immunol. 12:85-116; Hermiston et al., 2003. Annu Rev Immunol. 21:107-37; Holmes, 2006. Immunology. 117(2):145-55).

CD45 comprises an extracellular domain, a single transmembrane domain and a large cytoplasmic domain. The transmembrane and cytoplasmic domains are highly conserved amongst species. In particular, the cytoplasmic domain of CD45 comprises two tandemly duplicated phosphatase domains, of which only the membrane-proximal domain has enzymatic activity (Desai et al., 1994. EMBO J. 13(17):4002-10). The function of the more C-terminal second phosphatase domain in CD45 remains uncertain although it is suggested that it may contribute to CD45 activity indirectly by stabilizing the first domain. Through this cytoplasmic domain, CD45 functions as a central regulator of phosphotyrosine levels in hematopoietic cells, by modulating the activity of Src family of tyrosine-protein kinases (such as Lek in T cells; or Lyn, Fyn and Lek in B cells) (Palacios & Weiss, 2004. Oncogene. 23(48):7990-8000; Lowell, 2004. Mol Immunol. 41(6-7):631-43).

By contrast with the transmembrane and cytoplasmic domains, the extracellular domain of CD45 shows a higher polymorphism among different leukocyte lineages. Indeed, this extracellular domain is heavily glycosylated and contains three alternatively spliced exons (4, 5, and 6—which encode the A, B and C determinants, respectively) that are both O-linked glycosylated and sialylated (Hermiston et al., 2003. Annu Rev Immunol. 21:107-37; Holmes, 2006. Immunology. 117(2):145-55). CD45 isoforms differing in size, shape, and charge can therefore be generated by a dynamically-controlled alternative splicing in both leukocyte differentiation and cellular activation, leading to changes in the extracellular domain of the molecule (Hall et al., 1988. J Immunol. 141(8):2781-7; Lynch, 2004. Nat Rev Immunol. 4(12):931-40).

The largest CD45 isoform containing all three alternatively spliced exons, CD45RABC, is approximately 235 kDa, while the smallest isoform lacking all three exons, CD45RO, is approximately 180 kDa. In between, isoforms comprising only two (CD45RAB, CD45RAC, CD45RBC) or only one (CD45RA, CD45RB, CD45RC) of the three exons are possible.

While the function of the different CD45 isoforms is not clear, differential expression of these isoforms has been associated with the level of activation of T cells and allows dissociation of naive vs memory T cells (Birkeland et al., 1989. Proc Natl Acad Sci USA. 86(17):6734-8). For example, CD45RA is present on peripheral naive mature CD4⁺ T cells, while CD45RO is expressed on activated and memory CD4⁺ T cells. CD45RABC is expressed on B cells and their precursors, on a sub-group of dendritic cells and other antigen-presenting cells. Effector memory RA T cells (T_{EMRA}), a subtype of terminally differentiated memory T cells, also re-express the naive T cell marker CD45RA (Koch et al., 2008. Immun Ageing. 5:6). Importantly, this pattern of isoforms expression is highly conserved across species emphasizing its functional role and importance (Hermiston et al., 2003. Annu Rev Immunol. 21:107-37).

The expression pattern of the CD45RC isoform on CD4⁺ and CD8⁺ T cells allows to differentiate between functionally distinct alloreactive T cell subsets that behave differently in terms of proliferation and cytokine secretion. In rodents for example, it has been shown that both CD4⁺ and CD8⁺ T cells CD45RC^{high} are potent Th1 effector cells capable of promoting transplant rejection and organ inflammation (Spickett et al., 1983. J Exp Med. 158(3):795-810; Xystrakis et al., 2004. Eur J Immunol. 34(2):408-17), while T cells expressing undetectable or low levels of CD45RC^{low/-} are Th2 and regulatory T cells and inhibit allograft rejection, graft-versus-host disease (GVHD) and cell-mediated autoimmune diseases (Xystrakis et al., 2004. Blood. 104(10):3294-30; Guillonneau et al., 2007. J Clin Invest. 117(4):1096-106; Powrie & Mason, 1990. J Exp Med. 172(6):1701-8). In humans, a high proportion of CD45RC⁺ CD8⁺ T cells before transplantation has been correlated with decreased graft survival in kidney transplanted patients (Ordonez et al., 2013. PLoS One. 8(7):e69791).

The elimination of the CD45RC^{high} T cells population represents therefore a promising approach for inducing immune tolerance in human, thus for preventing, reducing and/or treating transplant rejection (in particular GVHD) and autoimmune diseases.

GVHD is a significant cause of morbidity and mortality in stem cell transplant patients. It is a T cell-mediated immunoreactive process in which donor cells react against recipient cells. Presently, immunosuppression with immunomodulating drugs such as corticosteroids are the mainstay of GVHD prevention. Whilst progresses have been made with improvements in survival outcomes over time, corticosteroids do not prevent GVHD in a high proportion of patients (less than 50% of patients with acute GVHD and 40-50% of patients with chronic GVHD depending on initial disease severity—Garnett et al., 2013. Ther Adv Hematol. 4(6):366-378), are associated with significant toxicities, and many of the currently available salvage therapies are associated with increased immunosuppression and infectious complications. Thus, there remains an unmet need for the development of new treatment strategies for GVHD to improve long-term post-transplant outcomes.

The Inventors have previously described that the depletion of CD45RC^{high} T cells may represent a potential new therapy in preventing or reducing transplant rejection by decreasing aggressive effector T cells and B cells, while increasing tolerogenic regulatory T cells. Indeed, transient anti-CD45RC mAb treatment triggers rapid CD45RC^{high} T cell apoptosis, while preserving memory immunity. Moreover, the Inventors showed that short term anti-CD45RC antibody treatment results in permanent allograft survival with no signs of chronic rejection (International patent WO2016016442; Picarda et al., 2017. JCI Insight. 2(3):e90088).

Here, the Inventors have developed a new monoclonal antibody directed against human CD45RC. This antibody competes with the anti-human CD45RC antibodies currently available on the market (such as the MT2 clone), exhibiting a comparable pattern of reactivity, however with a significantly better cytotoxic activity to T cells and at a lowest concentration. Indeed, the anti-hCD45RC antibody according to the present invention shows a better affinity than other antibodies currently available, and thereby have better therapeutic effects.

Interestingly, the antibody according to the invention may also be useful in the prevention or treatment of certain monogenic diseases in which immune responses are involved in the pathology. Monogenic diseases are caused by single-gene defects. Over 4000 human diseases are caused by these defects linked to one particular gene. Up to now, most treatment options revolve around treating the symptoms of the disorders, in an attempt to improve patient quality of life. Gene therapy is the main hope for durable treatments of this type of diseases. However, major obstacles have been encountered during the development of techniques for the delivery of genes to the appropriate cells affected by the disorder as well as the fact that immune responses against the transgene product or the vector limit the therapeutic efficacy.

Among monogenic diseases, some are linked to genes involved in the immune system (such as T and/or B cells primary immunodeficiencies and polyendocrinopathy candidiasis-ectodermal dystrophy [APECED]), or to genes not associated with immune functions but whose deficiency is associated with inflammation and/or immune reactions [such as Duchenne muscular dystrophy (DMD)]

APECED, also known as auto-immune polyglandular syndrome type I (APS 1) is a rare multi-organ autosomal recessive auto-immune disease caused by mutations in the AIRE gene, a transcription regulator that allows the expression of tissue-restricted antigens (TRA) in medullary epithelial thymic cells (mTECs) and auto-reactive T cells deletion. In human, more than 100 mutations have been described in the AIRE gene to cause APECED with a prevalence of 1-9:1000000 (Orphanet, http://www.orpha.net). The clinical phenotype of APECED is usually defined by the presence of 2 of the 3 major symptoms: hypoparathyroidism, adrenal insufficiency (Addison's disease) and chronical muco-cutaneous candidiasis (CMC). This disease is also associated with multiple autoimmune and ectodermal features such as type 1 diabetes, enamel hypoplasia, vitiligo, premature ovarian failure, keratitis, pernicious anemia, alopecia, exocrine pancreatitis, interstitial lung disease, nephritis and other disorders.

DMD is a monogenic disease wherein mutations of the DMD gene coding for the protein dystrophin lead to severe X-linked muscular dystrophy, which affects all voluntary muscles as well as the heart and breathing muscles in later stages. Immune responses are involved in the pathophysiology of disease in both DMD patients and mdx mice (for a review, see Rosenberg et al., 2015. Sci Transl Med. 7(299):299rv4). The standard treatments of DMD are corticoids, such as prednisolone. In mdx mice, treatments decreasing effector immune responses or inflammation have also been employed, such as intravenous immunoglobulins, tranilast, heme oxygenase-1 inducers, IL-1 receptor antagonist and IL-2, to amplify regulatory T cells (Tregs) (Villalta et al., 2014. Sci Transl Med. 6(258):258ra142; Rosenberg et al., 2015. Sci Transl Med. 7(299):299rv4). However, despite recent promising new treatments, the average life expectancy of DMD patients is still severely reduced.

Surprisingly, the Inventors have demonstrated that treatment of Dmd^{-/-} rats (Dmd^{mdx}) with an anti-CD45RC antibody specifically depleting CD45RC^{high} cells ameliorated muscle strength (Ouisse et al., 2019. Front Immunol. In press). They have also demonstrated that administration to Aire^{-/-} rats of an anti-CD45RC monoclonal antibody results in a strong depletion of CD45RC^{high} T cells, and to the removal of symptoms characteristics of APECED (manuscript in preparation; International patent application WO2019115791).

The antibody according to the present invention represents therefore a promising approach for preventing and/or treating monogenic diseases such as DMD and APECED.

### SUMMARY

The present invention relates to an isolated anti-human CD45RC antibody or binding fragment thereof, wherein said antibody or binding fragment thereof comprises:
(a) a HCVR which comprises the following three CDRs:
   (i) VH-CDR1 of sequence SEQ ID NO: 1;
   (ii) VH-CDR2 with a sequence selected from the group comprising sequences SEQ ID NOs: 4, 5, 6, 8, 100, 116, 117, 118 and 119; and
   (iii) VH-CDR3 of sequence SEQ ID NO: 3; and
(b) a LCVR which comprises the following three CDRs:
   (i) VL-CDR1 with a sequence selected from the group comprising sequences SEQ ID NO: 15 (SASSSVS-X₁₂-YMH) and 18 (RASSSVS-X₁₂-YMH), wherein X₁₂ is absent or is selected from Asn (N), Ser (S) and Gly (G);
   (ii) VL-CDR2 of sequence SEQ ID NO: 16; and
   (iii) VL-CDR3 of sequence SEQ ID NO: 17.

In one embodiment, said antibody or binding fragment thereof comprises:
(a) a HCVR which comprises the following three CDRs:
   (i) VH-CDR1 of sequence SEQ ID NO: 1;
   (ii) VH-CDR2 with a sequence selected from the group comprising sequences SEQ ID NOs: 4 and 5; and
   (iii) VH-CDR3 of sequence SEQ ID NO: 3; and
(b) a LCVR which comprises the following three CDRs:
   (i) VL-CDR1 of sequence SEQ ID NO: 15, wherein X₁₂ is absent;
   (ii) VL-CDR2 of sequence SEQ ID NO: 16; and
   (iii) VL-CDR3 of sequence SEQ ID NO: 17.

In one embodiment, said antibody or binding fragment thereof comprises:
(a) a HCVR which comprises the following three CDRs:
   (i) VH-CDR1 of sequence SEQ ID NO: 1;
   (ii) VH-CDR2 of sequence 4; and
   (iii) VH-CDR3 of sequence SEQ ID NO: 3; and
(b) a LCVR which comprises the following three CDRs:
   (i) VL-CDR1 of sequence SEQ ID NO: 15, wherein X₁₂ is absent;
   (ii) VL-CDR2 of sequence SEQ ID NO: 16; and
   (iii) VL-CDR3 of sequence SEQ ID NO: 17.

In one embodiment, said antibody or binding fragment thereof comprises:
(a) a HCVR which comprises the following three CDRs:
   (i) VH-CDR1 of sequence SEQ ID NO: 1;
   (ii) VH-CDR2 with a sequence selected from the group comprising sequences SEQ ID NOs: 4, 6, and 100; and
   (iii) VH-CDR3 of sequence SEQ ID NO: 3; and
(b) a LCVR which comprises the following three CDRs:
   (i) VL-CDR1 with a sequence selected from the group comprising sequences SEQ ID NOs: 15 and 18, wherein X₁₂ is absent;
   (ii) VL-CDR2 of sequence SEQ ID NO: 16; and
   (iii) VL-CDR3 of sequence SEQ ID NO: 17.

In one embodiment, said antibody or binding fragment thereof comprises:
1) a HCVR of sequence SEQ ID NO: 61 and a LCVR of sequence SEQ ID NO: 81;
2) a HCVR of sequence SEQ ID NO: 62 and a LCVR of sequence SEQ ID NO: 82;
3) a HCVR of sequence SEQ ID NO: 62 and a LCVR of sequence SEQ ID NO: 83;
4) a HCVR of sequence SEQ ID NO: 62 and a LCVR of sequence SEQ ID NO: 84;
5) a HCVR of sequence SEQ ID NO: 63 and a LCVR of sequence SEQ ID NO: 82;
6) a HCVR of sequence SEQ ID NO: 63 and a LCVR of sequence SEQ ID NO: 83;
7) a HCVR of sequence SEQ ID NO: 63 and a LCVR of sequence SEQ ID NO: 84;
8) a HCVR of sequence SEQ ID NO: 64 and a LCVR of sequence SEQ ID NO: 82;
9) a HCVR of sequence SEQ ID NO: 64 and a LCVR of sequence SEQ ID NO: 83;
10) a HCVR of sequence SEQ ID NO: 64 and a LCVR of sequence SEQ ID NO: 84;
11) a HCVR of sequence SEQ ID NO: 101 and a LCVR of sequence SEQ ID NO: 85;
12) a HCVR of sequence SEQ ID NO: 101 and a LCVR of sequence SEQ ID NO: 103;
13) a HCVR of sequence SEQ ID NO: 65 and a LCVR of sequence SEQ ID NO: 85;
14) a HCVR of sequence SEQ ID NO: 65 and a LCVR of sequence SEQ ID NO: 103;
15) a HCVR of sequence SEQ ID NO: 62 and a LCVR of sequence SEQ ID NO: 85;
16) a HCVR of sequence SEQ ID NO: 101 and a LCVR of sequence SEQ ID NO: 82;
17) a HCVR of sequence SEQ ID NO: 121 and a LCVR of sequence SEQ ID NO: 85;
18) a HCVR of sequence SEQ ID NO: 122 and a LCVR of sequence SEQ ID NO: 85;
19) a HCVR of sequence SEQ ID NO: 123 and a LCVR of sequence SEQ ID NO: 85;
20) a HCVR of sequence SEQ ID NO: 124 and a LCVR of sequence SEQ ID NO: 85;
21) a HCVR of sequence SEQ ID NO: 63 and a LCVR of sequence SEQ ID NO: 85;
22) a HCVR of sequence SEQ ID NO: 67 and a LCVR of sequence SEQ ID NO: 85;
23) a HCVR of sequence SEQ ID NO: 67 and a LCVR of sequence SEQ ID NO: 103; or
24) a HCVR and a LCVR comprising a sequence of the non-CDR regions sharing at least 70% of identity with the sequence of the non-CDR regions of the HCVR and LCVR according to 1) to 23).

In one embodiment, said antibody or binding fragment thereof comprises:
(a) a HCVR which comprises the following three CDRs:
   (i) VH-CDR1 of sequence SEQ ID NO: 1;
   (ii) VH-CDR2 with a sequence selected from the group comprising sequences SEQ ID NOs: 4, 5, 6, 8, 100, 116, 117, 118 and 119; and
   (iii) VH-CDR3 of sequence SEQ ID NO: 3; and
(b) a LCVR which comprises the following three CDRs:
   (i) VL-CDR1 with a sequence selected from the group comprising sequences SEQ ID NOs: 15 and 18, wherein X₁₂ in SEQ ID NOs: 15 and 18 is selected from Asn (N), Ser (S) and Gly (G);
   (ii) VL-CDR2 of sequence SEQ ID NO: 16; and
   (iii) VL-CDR3 of sequence SEQ ID NO: 17;
      preferably wherein the amino acid residue at Kabat position L71 of the LCVR is Phe (F).

The present invention further relates to a nucleic acid encoding the isolated antibody or binding fragment thereof according to the invention.

The present invention further relates to an expression vector comprising the nucleic acid according to claim 7.

The present invention further relates to a cell comprising the nucleic acid according to the invention or the expression vector according to the invention.

The present invention further relates to a pharmaceutical composition comprising the isolated antibody or binding fragment thereof according to the invention, the nucleic acid according to the invention, the expression vector according to the invention or the cell according to the invention, and at least one pharmaceutically acceptable excipient.

The present invention further relates to the isolated antibody or binding fragment thereof according to the invention, the nucleic acid according to the invention, the expression vector according to the invention, the cell according to the invention or the pharmaceutical composition according to the invention, for use as a medicament.

The present invention further relates to the isolated antibody or binding fragment thereof according to the invention, the nucleic acid according to the invention, the expression vector according to the invention, the cell according to the invention or the pharmaceutical composition according to the invention, for use in:
inducing immune tolerance in a subject in need thereof; and/or
preventing and/or reducing transplant rejections.

The present invention further relates to the isolated antibody or binding fragment thereof according to the invention, the nucleic acid according to the invention, the expression vector according to the invention, the cell according to the invention or the pharmaceutical composition according to the invention, for use in preventing, reducing and/or treating CD45RC^{high}-related conditions, preferably wherein the CD45RC^{high}-related condition is selected from the group comprising autoimmune diseases, undesired immune responses, monogenic diseases, and lymphoma or cancer.

The present invention further relates to the isolated antibody or binding fragment thereof according to the invention, the nucleic acid according to the invention, the expression vector according to the invention, the cell according to the invention or the pharmaceutical composition according to the invention, for use in preventing and/or treating graft-versus-host disease (GVHD).

The present invention further relates to an in vitro method for detecting or quantifying hCD45RC in a sample, cell, tissue or organ, comprising contacting said sample, cell, tissue or organ with the isolated antibody or binding fragment thereof according to the present invention, optionally wherein the isolated antibody or binding fragment thereof is labelled.

### “Antibody” or “Immunoglobulin”

As used herein, the term “immunoglobulin” refers to a protein having a combination of two heavy and two light chains whether or not it possesses any relevant specific immunoreactivity. “Antibodies” refers to such assemblies which have significant known specific immunoreactive activity to an antigen of interest (e.g., human CD45RC). The term “anti-hCD45RC antibodies” is used herein to refer to antibodies which exhibit immunological specificity for human CD45RC protein. As explained elsewhere herein, “specificity” for human CD45RC does not exclude cross-reaction with species homologues of hCD45RC.

Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (V_{L} domain) and heavy chain variable domain (V_{H} domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the V_{H} and V_{L} chains.

“Characterized as Having [ . . . ] Amino Acids being Substituted by a Different Amino Acid”

As used herein, the phrase “characterized as having [ . . . ]amino acids being substituted by a different amino acid” in reference to a given sequence, refers to the occurrence, in said sequence, of “conservative amino acid modifications”.

### “Conservative Amino Acid Modifications”

“Conservative amino acid modifications” refers to modifications that do not significantly affect or alter the binding characteristics of the antibody or binding fragment thereof containing the amino acid sequence. Such conservative modifications include amino acid substitutions, additions and deletions. Modifications can be introduced into an antibody or binding fragment thereof by standard techniques known in the art, such as site-directed mutagenesis and PCR-mediated mutagenesis.

Conservative amino acid substitutions are typically those in which an amino acid residue is replaced with an amino acid residue having a side chain with similar physicochemical properties. Specified variable region and CDR sequences may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or more amino acid insertions, deletions and/or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine) and aromatic side chains (e.g., tyrosine, phenylalanine, tryptophan, histidine). Thus, one or more amino acid residues within the CDRs and/or variable regions of the antibody or binding fragment thereof according to the present invention can be replaced with other amino acid residues from the same side chain family and the altered antibody can be tested for retained function (i.e., the properties set forth herein, such as, e.g., the binding to hCD45RC) using the assays described herein. In another embodiments, a string of amino acids within the CDRs and/or variable regions of the antibody or binding fragment thereof according to the present invention can be replaced with a structurally similar string that differs in order and/or composition of side chain family members.

### “CD45”

As used herein, the term “CD45” (also known as CD45R or PTPRC) refers to a transmembrane glycoprotein existing in different isoforms. These distinct isoforms of CD45 differ in their extracellular domain structures which arise from alternative splicing of 3 variable exons (exons 4, 5 and 6) coding for the A, B and C determinants, respectively, of the CD45 extracellular region. Antibodies reactive with restricted epitope are clustered as “CD45R”. Hence, anti-CD45RA, anti-CD45RB and anti-CD45RC antibodies recognize CD45 isoforms which include the expression of the A, B and C determinants, respectively. The various isoforms of CD45 have different extracellular domains, but have an identical extracellular sequence proximal to the membrane, as well as for the transmembrane domain and a large cytoplasmic tail segments containing two tandemly homologous highly conserved phosphatase domains of approximately 300 residues. CD45 and its isoforms non-covalently associate with lymphocyte phosphatase-associated phosphoprotein (LPAP) on T and B lymphocytes. CD45 has been reported to be associated with several other cell surface antigens, including CD1, CD2, CD3, and CD4. CD45 is involved in signaling lymphocytes activation. When preceded by the letter “h” (e.g., hCD45), it is implied that the CD45 is of human origin.

### “CD45RC”

As used herein, the term “CD45RC” refers to a 200-220 kDa single chain type I membrane glycoprotein well-known from the skilled artisan. CD45RC is an alternative splicing isoform of CD45 comprising exon 6 encoding the C determinant (hence the terminology CD45RC, i.e., CD45 Restricted to the C determinant), but lacking exons 4 and 5, respectively encoding the A and B determinants. An amino acid sequence of human CD45RC is given in SEQ ID NO: 104, corresponding to UniProt Accession P08575-10 (version 10, modified Mar. 28, 2018—Checksum: F92C874C9A114890). This CD45RC isoform is expressed on B cells, and a subset of CD8⁺ T cells and CD4⁺ T cells, but not on CD8⁺ or CD4⁺ Treg, CD14⁺ monocytes or PMN (Picarda et al., 2017. JCI Insight. 2(3):e90088). While some monoclonal antibodies can recognize an epitope in the portion of CD45 common to all the different isoforms (these are termed anti-CD45 antibodies), other monoclonal antibodies have restricted specificity to a given isoform, depending on which determinant they recognize (A, B or C). When preceded by the letter “h” (e.g., hCD45RC), it is implied that the CD45RC is of human origin.

“CD45RC^{high} Cell Antigen” or “CD45RC^{high} Cell Surface Marker”

As used herein, the terms “CD45RC^{high} cell antigen” or “CD45RC^{high} cell surface marker” refer to an antigen (or epitope) of sequence SEQ ID NO: 23, which is expressed or displayed at the surface of a CD45RC^{high} cells (including T cells, B cells and natural killer (NK) cells) which can be targeted with an anti-CD45RC agent which binds thereto (such as an antibody or an aptamer). Exemplary CD45RC^{high} T cell surface markers include but are not limited to the CD45RC as previously described or other antigens that characterize said population of T cells. The CD45RC^{high} T cells surface marker of particular interest is preferentially expressed on CD45RC^{high} T cells compared to other non-CD45RC^{high} T cells of a mammal.

Then, after raising antibodies directed against the CD45RC cell surface marker as above described, the skilled man in the art can easily select those that act on CD45RC^{high} cells, and that can be used to deplete CD45RC^{high} cells via antibody-dependent cell mediated cytotoxicity (ADCC), complement dependent cytotoxicity (CDC), or induction of CD45RC^{high} but not CD45RC^{low/-} cell death (e.g., via apoptosis) after direct binding of the antibody (Picarda et al., 2017. JCI Insight. 2(3):e90088).

### “CDR” or “Complementarity Determining Region”

As used herein, the term “CDR” or “complementarity determining region” means the non-contiguous antigen combining sites found within the variable region of both heavy and light chain polypeptides. CDRs were identified according to the rules of Table 1, as deduced from Kabat et al., 1991. Sequences of proteins of immunological interest (5^{th} ed.). Bethesda, MD: U.S. Dep. of Health and Human Services; and Chothia and Lesk, 1987. J Mol Biol. 196(4):901-17:

TABLE 1
Heavy chain variable region (HCVR or V_{H})

| | V_{H}-CDR1 | V_{H}-CDR2 | V_{H}-CDR3 |
|---|---|---|---|
| Start | Approx. at residue 26 | Always 15 residues after | Always 33 residues after |
| | (always 4 after a Cys) | the end of V_{H}-CDR1 | end of V_{H}-CDR2 |
| | according to | according to Kabat/AbM′s | Always 2 residues after a |
| | Chothia/AbM′s definition | definition | Cys |
| | Kabat′s definition starts 5 | | |
| | residues later | | |
| Residue before | Always Cys-Xaa-Xaa-Xaa, | Typically, Leu-Glu-Trp-Ile- | Always Cys-Xaa-Xaa, with |
| | with Xaa being any amino | Gly, but a number of | Xaa being any amino acid |
| | acid according to | variations | Typically, Cys-Ala-Arg |
| | Chothia/AbM′s definition | | |
| Residue after | Always Trp | Lys/Arg- | Always Trp-Gly-Xaa-Gly, |
| | Typically, Trp-Val, but | Leu/Ile/Val/Phe/Thr/Ala- | with Xaa being any amino |
| | also, Trp-Ile or Trp-Ala | Thr/Ser/Ile/Ala | acid |
| Length | 10 to 12 residues according | 16 to 19 residues according | 3 to 25 residues |
| | to AbM′s definition | to Kabat′s definition | |
| | Chothia′s definition | AbM′s definition ends 7 | |
| | excludes the last 4 residues | residues earlier | |
| | 5 to 7 residues according to | | |
| | Kabat′s definition | | |

Light chain variable region (LCVR or V_{L})

| | V_{L}-CDR1 | V_{L}-CDR2 | V_{L}-CDR3 |
|---|---|---|---|
| Start | Approx. at residue 24 | Always 16 residues after | Always 33 residues after |
| | | the end of V_{L}-CDR1 | end of V_{L}-CDR2 (except |
| | | | NEW (PDB ID: 7FAB) |
| | | | which has the deletion at |
| | | | the end of CDR-L2*) |
| Residue before | Always Cys | Generally, Ile-Tyr, but also, | Always Cys |
| | | Val-Tyr, Ile-Lys or Ile-Phe | |
| Residue after | Always Trp | | Always Phe-Gly-Xaa-Gly, |
| | Typically, Trp-Tyr-Gln, but | | with Xaa being any amino |
| | also, Trp-Leu-Gln, Trp- | | acid |
| | Phe-Gln or Trp-Tyr-Leu | | |
| Length | 10 to 17 residues | Always 7 residues (except | 7 to 11 residues |
| | | NEW (PDB ID: 7FAB) | |
| | | which has a deletion in this | |
| | | region*) | |

* Saul & Poljak, 1992. Proteins. 14(3):363-71

### “Epitope”

As used herein, the term “epitope” refers to a specific arrangement of amino acids located on a protein or proteins to which an antibody or binding fragment thereof binds. Epitopes often consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the antigen that may not necessarily be contiguous.

### “Framework Region” or “FR” or “Non-CDR Regions”

As used herein, the terms “framework region”, “FR” or “non-CDR regions” include the amino acid residues that are part of the variable region, but are not part of the CDRs (e.g., using the Kabat/Chothia definition of CDRs). Therefore, a variable region framework is between about 100-120 amino acids in length but includes only those amino acids outside of the CDRs.

For the specific example of a HCVR and for the CDRs as defined by Kabat/Chothia:
FR1 may correspond to the domain of the variable region encompassing amino acids 1-25 according to Chothia/AbM's definition, or 5 residues later according to Kabat's definition;
FR2 may correspond to the domain of the variable region encompassing amino acids 36-49;
FR3 may correspond to the domain of the variable region encompassing amino acids 67-98; and
FR4 may correspond to the domain of the variable region from amino acids 104-110 to the end of the variable region.

The framework regions for the light chain are similarly separated by each of the LCVR's CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions. The framework regions largely adopt a β-sheet conformation and the CDRs form loops which connect, and in some cases form part of, the β-sheet structure.

Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

### “Heavy Chain Region”

As used herein, the term “heavy chain region” includes amino acid sequences derived from the constant domains of an immunoglobulin heavy chain. A protein comprising a heavy chain region comprises at least one of a C_{H}1 domain, a hinge (e.g., upper, middle, and/or lower hinge region) domain, a C_{H}2 domain, a C_{H}3 domain, or a variant or fragment thereof. In an embodiment, the antibody or binding fragment thereof according to the present invention may comprise the Fc region of an immunoglobulin heavy chain (e.g., a hinge portion, a C_{H}2 domain, and a C_{H}3 domain). In another embodiment, the antibody or binding fragment thereof according to the present invention lacks at least a region of a constant domain (e.g., all or part of a C_{H}2 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a protein may comprise a C_{H}2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the antibody or binding fragment thereof according to the present invention may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

### “Hinge Region”

As used herein, the term “hinge region” includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J Immunol. 161(8):4083-90).

### “Hypervariable Loop”

The term “hypervariable loop” is not strictly synonymous to complementarity determining region (CDR), since the hypervariable loops (HVs) are defined on the basis of structure, whereas CDRs are defined based on sequence variability (Kabat et al., 1991. Sequences of proteins of immunological interest (5^{th} ed.). Bethesda, MD: U.S. Dep. of Health and Human Services) and the limits of the HVs and the CDRs may be different in some V_{H} and V_{L} domains. The CDRs of the V_{L} and V_{H} domains can typically be defined by the Kabat/Chothia definition as already explained hereinabove.

### “Identity” or “Identical”

As used herein, the term “identity” or “identical”, when used in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”).

Identity of related amino acid sequences or nucleic acid sequences can be readily calculated by known methods. Such methods include, but are not limited to, those described in Lesk A. M. (1988). Computational molecular biology: Sources and methods for sequence analysis. New York, NY: Oxford University Press; Smith D. W. (1993). Biocomputing: Informatics and genome projects. San Diego, CA: Academic Press; Griffin A. M. & Griffin H. G. (1994). Computer analysis of sequence data, Part 1. Totowa, NJ: Humana Press; von Heijne G. (1987). Sequence analysis in molecular biology: treasure trove or trivial pursuit. San Diego, CA: Academic press; Gribskov M. R. & Devereux J. (1991). Sequence analysis primer. New York, NY: Stockton Press; Carillo et al., 1988. SIAM J Appl Math. 48(5):1073-82.

Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, WI; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

### “Immunospecific”, “Specific For” or “Specifically Bind”

As used herein, an antibody or binding fragment thereof is said to be “immunospecific”, “specific for” or to “specifically bind” an antigen if it reacts at a detectable level with said antigen (e.g., hCD45RC), preferably with an affinity constant (K_{A}) of greater than or equal to about 10⁶ M⁻¹, preferably greater than or equal to about 10⁷ M⁻¹, 10⁸ M⁻¹, 5×10⁸ M⁻¹, 10⁹ M⁻¹, 5×10⁹ M⁻¹ or more.

Affinity of an antibody or binding fragment thereof for its cognate antigen is also commonly expressed as an equilibrium dissociation constant (K_{D}). an antibody or binding fragment thereof is said to be “immunospecific”, “specific for” or to “specifically bind” an antigen if it reacts at a detectable level with said antigen (e.g., hCD45RC), preferably with a K_{D} of less than or equal to 10⁻⁶ M, preferably less than or equal to 10⁻⁷ M, 5×10⁻⁸ M, 10⁻⁸ M, 5×10⁻⁹ M, 10⁻⁹ M or less.

Affinities of antibodies or binding fragment thereof can be readily determined using conventional techniques, for example, those described by Scatchard, 1949. Ann NY Acad Sci. 51:660-672. Binding properties of an antibody or binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®).

### “Monoclonal Antibody”

As used herein, the term “monoclonal antibody” refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies or binding fragment thereof according to the present invention may be prepared by the hybridoma methodology first described by Kohler et al., 1975. Nature. 256(5517):495-7, or may be made using recombinant DNA methods in bacterial, eukaryotic animal or plant cells (U.S. Pat. No. 4,816,567). The “monoclonal antibodies” may also be isolated from phage antibody libraries using the techniques described in Clackson et al., 1991. Nature. 352(6336):624-8 and Marks et al., 1991. J Mol Biol. 222(3):581-97, for example.

### “Prevent” or “Preventing” or “Prevention”

As used herein, the terms “prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

### “Subject”

As used herein, the term “subject” refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease.

The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human.

### “Variable Region” or “Variable Domain”

As used herein, the term “variable” refers to the fact that certain regions of the variable domains V_{H} and V_{L} differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the V_{L} domain and the V_{H} domain which form part of the antigen binding site.

The first, second and third hypervariable loops of the Vλ light chain domain are referred to herein as L1 (λ), L2 (λ) and L3 (λ) and may be defined as comprising residues 24-33 (L1(λ), consisting of 9, 10 or 11 amino acid residues), 49-53 L2 (λ), consisting of 3 residues) and 90-96 (L3(λ)), consisting of 6 residues) in the V_{L} domain (Morea et al., 2000. Methods. 20(3):267-79).

The first, second and third hypervariable loops of the Vκ light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the V_{L} domain (Morea et al., 2000. Methods. 20(3):267-79).

The first, second and third hypervariable loops of the V_{H} domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the V_{H} domain (Morea et al., 2000. Methods. 20(3):267-79).

Unless otherwise indicated, the terms L1, L2 and L3 respectively refer to the first, second and third hypervariable loops of a V_{L} domain, and encompass hypervariable loops obtained from both Vκ and Vλ isotypes. The terms H1, H2 and H3 respectively refer to the first, second and third hypervariable loops of the V_{H} domain, and encompass hypervariable loops obtained from any of the known heavy chain isotypes, including gamma (γ), mu (μ), alpha (α), delta (δ) or epsilon (ε). The hypervariable loops L1, L2, L3, H1, H2 and H3 may each comprise part of a “complementarity determining region” or “CDR”, as defined hereinabove.

### “Treating” or “Treatment” or “Alleviation”

As used herein, the terms “treating” or “treatment” or “alleviation” refer to therapeutic treatment, excluding prophylactic or preventative measures; wherein the object is to slow down (lessen) the targeted pathologic condition or disorder. Those in need of treatment include those already with the disorder as well those suspected to have the disorder. A subject is successfully “treated” for the targeted pathologic condition or disorder if, after receiving a therapeutic amount of the isolated antibody or binding fragment thereof, nucleic acid, expression vector, composition, pharmaceutical composition or medicament according to the present invention, said subject shows observable and/or measurable reduction in or absence of one or more of the following: reduction in the number of CD45RC^{high} cells; reduction in the percent of total cells that are CD45RC^{high}; relief to some extent, of one or more of the symptoms associated with the specific disease or condition; reduced morbidity and mortality; and/or improvement in quality of life issues. The above parameters for assessing successful treatment and improvement in the disease are readily measurable by routine procedures familiar to a physician.

### BRIEF DESCRIPTION OF THE DRAWINGS

FIGS. 1 A(1), 1A(2) 1A(3), 1B(1) and 1B(2) show the expression level of CD45RC (as detected by ABIS-45RC or by the commercially available MT2 antibody) on different leukocyte types in human blood. Staining with ABIS-45RC or MT2 was realized on different cell types in total blood (EDTA) from healthy volunteers. Red blood cells were then lysed (Versalyse, Beckman Coulter) before cytometer analysis (Navios, Beckman Coulter). Cells were first gated on morphology, doublet cells and lived cells. (A(1) through A(3)) Representative dot plot analysis of CD45RC expression detected by ABIS-45RC or MT2 from one out of three healthy volunteers analyzed on different leukocyte types. ABIS-45RC is shown on left panels and MT2 on right panels. x-axis shows the fluorescence intensity of cell lineage markers labelling for each type of leukocytes as indicated; y-axis represents the fluorescence intensity of anti-CD45RC antibody labelling. The horizontal lines define cells with high, intermediate/low and negative levels of CD45RC expression as indicated in the upper left dot plot and numbers represent the percentage of cells in each category. (B(1) and B(2)) Mean expression+/−SEM of CD45RC^{high}, CD45RC^{low} and CD45RC⁻ on different leukocyte types of three donors labelled with ABIS-45RC (B(1)) or MT2 (B(2)).

FIG. 2 shows that both ABIS-45RC and the commercial anti-CD45RC MT2 antibody compete for the same epitope. PBMCs were isolated from blood of healthy volunteers and T cells were labeled with an anti-CD3 labeled Mab, with chimeric ABIS-45RC (at the indicated concentrations) and anti-CD45RC (mouse clone MT2)-FITC labeled at 1.33 mg/mL. ABIS-45RC reactivity was revealed using a biotin donkey anti-human IgG+Strepta PercpCy 5.5 secondary antibody. Numbers in the windows of the dot plots of the upper row represent the percentage of cells that were co-labeled by both antibodies.

FIGS. 3A-E and 3F show that cytotoxicity induced by ABIS-45RC is higher compared to commercial anti-CD45RC MT2. PBMCs from healthy volunteers (n=3) were incubated at 37° C. with medium, isotype negative control (2.5 or 10 mg/mL), ABIS-CD45RC (2.5 or 10 mg/mL) or dexamethasone (10 mg/mL) as a positive control for the indicated time points and then cells were labeled with an anti-CD3-FITC mouse antibody and apoptotic cells by labeling with Annexin-V-PE. (A-E) Graphs indicate % of Annexin V⁺ cells in indicated cell populations. (F) Representative dot plots of Annexin V⁺ (early apoptotic) and DAPI+(late apoptotic) cells. Numbers indicate the percentage of cells in each category.

FIGS. 4A and 4B-C show the use of ABIS-45RC to treat GVHD in immune humanized NSG immunodeficient mice. (A) Experimental procedure showing that peripheral blood mononuclear cells (PBMCs) from healthy donor volunteers were infused intravenously (iv) (day 0) into previously (day −1) sublethaly (2 Gy) irradiated NSG immunodeficient mice. ABIS-45RC was administered intraperitoneally (ip) with the indicated protocol between day 0 to 20. Isotype control were human IgG (IVIg preparation) and was administered using the same protocol as ABIS-45RC. (B-C) Survival curves for NSG mice and statistics were analyzed using a Kaplan-Meier analysis (* p<0.01, ** p<0.001).

FIGS. 5A-D, 5E-G, and 5H-J are a combination of flow cytometry dot plots, showing the reactivity against human T cells of humanized ABIS-45RC antibodies variants and of the murine ABIS-45RC antibody, at two concentrations (2 μg/mL on the left panel, 1 μg/mL on the right panel). (A) humanized ABIS-45RC variant A; (B) humanized ABIS-45RC variant B; (C) humanized ABIS-45RC variant C; (D) humanized ABIS-45RC variant D; (E) humanized ABIS-45RC variant E; (F) humanized ABIS-45RC variant F; (G) humanized ABIS-45RC variant G; (H) humanized ABIS-45RC variant H; (I) humanized ABIS-45RC variant I; (J) murine ABIS-45RC.

FIG. 6 shows two dot plots of flow cytometry showing that both ABIS-45RC (left panel) and engineered Asn/Phe ABIS-45RC (right panel) have an equivalent pattern of reactivity against human T cells.

FIGS. 7A, 7B, and 7C show the expression level of CD45RC on CD3⁺ leukocyte in human blood from three healthy volunteers. Cells were first gated on morphology, doublet cells and lived cells. (A) representative dot plot analysis of CD45RC expression detected by murine ABIS-45RC from one out of three healthy volunteers analyzed; (B) representative dot plot analysis of CD45RC expression detected by humanized ABIS-45RC variant A1 from one out of three healthy volunteers analyzed; (C) representative dot plot analysis of CD45RC expression detected by humanized ABIS-45RC variant A3 from one out of three healthy volunteers analyzed. x-axis shows the FSC; y-axis represents the fluorescence intensity of anti-CD45RC antibody labelling. The squares define cells with high, intermediate/low and negative levels of CD45RC expression as indicated and numbers represent the percentage of cells in each category.

FIGS. 8A and 8B show that cytotoxicity induced by ABIS-45RC or by the humanized variants A1 and A3 is comparable. PBMCs from healthy volunteers were incubated at 37° C. with an isotype negative control (10 μg/mL), with murine ABIS-CD45RC (10 μg/mL), with the humanized variant A1 (10 μg/mL) or with the humanized variant A3 (10 μg/mL) for the indicated time points, and cells were then labeled with anti-CD3 and anti-CD45RA antibodies and apoptotic cells by labeling with Annexin-V-PE. The graphs indicate fold apoptosis in CD3⁺CD45RA^{hi} cells (A) and in CD3⁻ cells (B), compared to the isotype control condition.

FIGS. 9A and 9B show the expression level of CD45RC (as detected by ABIS-45RC or humanized variants A, A1 and A3) on CD3⁻ cells (A) and CD3⁻ cells (B) in macaque blood. Cells were first gated on morphology, doublet cells and lived cells. Representative dot plot analysis of CD45RC expression detected by ABIS-45RC or ABIS-45RC humanized variants A, A1 and A3. x-axis shows the FSC; y-axis represents the fluorescence intensity of anti-CD45RC antibody labelling. The squares define cells with positive levels of CD45RC expression as indicated and numbers represent the percentage of cells.

FIG. 10 shows the skin graft survival of treated humanized mice with anti-human CD45RC treatment. NSG mice transferred with total human PBMCs to induce human skin rejection were treated with murine ABIS-45RC or humanized variant A1, together with rapamycin (Rapa). Results are expressed in skin graft survival score.

### DETAILED DESCRIPTION

The present invention relates to an isolated antibody or a binding fragment thereof, binding to human CD45RC (hCD45RC).

An “isolated antibody”, as used herein, is intended to refer to an antibody that is substantially free of other antibodies having different antigenic specificities (e.g., an isolated antibody that specifically binds hCD45RC is substantially free of antibodies that specifically bind antigens other than hCD45RC). An isolated antibody that specifically binds hCD45RC may, however, have cross-reactivity to other antigens, such as CD45RC molecules from other species. Moreover, an isolated antibody may be substantially free of other cellular material and/or chemicals, in particular those that would interfere with diagnostic or therapeutic uses of the antibody, including without limitation, enzymes, hormones, and other proteinaceous or non-proteinaceous components.

In one embodiment, the isolated antibody or a binding fragment thereof is purified.

In one embodiment, the isolated antibody or a binding fragment thereof is purified to:
(1) greater than 80%, 85%, 90%, 91%, 92%, 93%, 94%, 95% or more by weight of antibody or binding fragment thereof as determined by the Lowry method, and most preferably more than 96%, 97%, 98% or 99% by weight;
(2) a degree sufficient to obtain at least 15 residues of N-terminal or internal amino acid sequence by use of a spinning cup sequenator; or
(3) homogeneity as shown by SDS-PAGE under reducing or non-reducing conditions and using Coomassie blue or, preferably, silver staining.

In one embodiment, the antibody or binding fragment thereof according to the present invention binds to the extracellular domain of hCD45RC. In one embodiment, the antibody or binding fragment thereof according to the present invention binds to at least one epitope present on the extracellular domain of hCD45RC.

In one embodiment, the antibody or binding fragment thereof according to the present invention binds to the C determinant encoded by exon 6 of hCD45. In one embodiment, the antibody or binding fragment thereof according to the present invention binds to at least one epitope on the C determinant encoded by exon 6 of hCD45.

In one embodiment, the amino acid sequence of the C determinant encoded by exon 6 of hCD45 comprises or consists of SEQ ID NO: 23. In one embodiment, the nucleic acid sequence of exon 6 encoding the C determinant of hCD45 comprises or consists of SEQ ID NO: 24.

SEQ ID NO: 23
DVPGERSTASTFPTDPVSPLTTTLSLAHHSSAALPARTSNTTITANTS
SEQ ID NO: 24
GATGTCCCAGGAGAGAGGAGTACAGCCAGCACCTTTCCTACAGACCCAGT
TTCCCCATTGACAACCACCCTCAGCCTTGCACACCACAGCTCTGCTGCCT
TACCTGCACGCACCTCCAACACCACCATCACAGCGAACACCTCA

In one embodiment, the antibody or binding fragment thereof according to the present invention binds to at least one epitope comprising or consisting of SEQ ID NO: 23 or a fragment thereof.

In one embodiment, the antibody or binding fragment thereof according to the present invention binds to at least one epitope comprising or consisting of a sequence sharing at least about 70%, preferably at least 75%, 80%, 85%, 90%, 91%, 92%, 93%, 94%, 95%, 96%, 97%, 98%, 99% or more identity with SEQ ID NO: 23 or a fragment thereof.

In one embodiment, the antibody or binding fragment thereof according to the present invention binds to at least one epitope encoded by a nucleic acid sequence comprising or consisting of SEQ ID NO: 24 or a fragment thereof.

In one embodiment, the antibody or binding fragment thereof according to the present invention binds to at least one epitope encoded by a nucleic acid sequence comprising or consisting of a sequence sharing at least about 70%, preferably at least 75%, 80%, 85%, 90%, 91%, 92%, 93%, 94%, 95%, 96%, 97%, 98%, 99% or more identity with SEQ ID NO: 24 or a fragment thereof.

In one embodiment, the antibody or binding fragment thereof according to the present invention binds to at least one epitope comprising or consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acids of SEQ ID NO: 23 or a fragment thereof; or of a sequence sharing at least about 70%, preferably at least 75%, 80%, 85%, 90%, 91%, 92%, 93%, 94%, 95%, 96%, 97%, 98%, 99% or more identity with SEQ ID NO: 23 or a fragment thereof.

In one embodiment, the antibody or binding fragment thereof according to the present invention binds to at least one epitope comprising or consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 contiguous amino acids of SEQ ID NO: 23 or a fragment thereof; or of a sequence sharing at least about 70%, preferably at least 75%, 80%, 85%, 90%, 91%, 92%, 93%, 94%, 95%, 96%, 97%, 98%, 99% or more identity with SEQ ID NO: 23 or a fragment thereof.

In one embodiment, a fragment of the at least one epitope comprising or consisting of SEQ ID NO: 23 comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acid residues.

In one embodiment, a fragment of the at least one epitope comprising or consisting of SEQ ID NO: 23 comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acid residues spread over a span of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450,460, 470, 480, 490, 500, 510, 520, 530, 540, 550, 560, 570, 580, 590, 600, 610, 620, 630, 640, 650, 660, 670, 680, 690, 700, 710, 720, 73, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100 or more contiguous amino acid residues of a sequence comprising or consisting of SEQ ID NO: 23.

In one embodiment, a sequence comprising SEQ ID NO: 23 is the sequence of hCD45 set forth in SEQ ID NO: 99, corresponding to UniProt Accession P08575-3 (version 3, modified Mar. 28, 2018—Checksum: 6E942E2BF6B17AC5).

SEQ ID NO: 99
(wherein SEQ ID NO: 23 is underlined)
MTMYLWLKLLAFGFAFLDTEVFVTGQSPTPSPTGLTTAKMPSVPLSSDPL
PTHTTAFSPASTFERENDFSETTTSLSPDNTSTQVSPDSLDNASAFNTTG
VSSVQTPHLPTHADSQTPSAGTDTQTFSGSAANAKLNPTPGSNAISDVPG
ERSTASTFPTDPVSPLTTTLSLAHHSSAALPARTSNTTITANTSDAYLNA
SETTTLSPSGSAVISTTTIATTPSKPTCDEKYANITVDYLYNKETKLFTA
KLNVNENVECGNNTCTNNEVHNLTECKNASVSISHNSCTAPDKTLILDVP
PGVEKFQLHDCTQVEKADTTICLKWKNIETFTCDTQNITYRFQCGNMIFD
NKEIKLENLEPEHEYKCDSEILYNNHKFTNASKIIKTDFGSPGEPQIIFC
RSEAAHQGVITWNPPQRSFHNFTLCYIKETEKDCLNLDKNLIKYDLQNLK
PYTKYVLSLHAYIIAKVQRNGSAAMCHFTTKSAPPSQVWNMTVSMTSDNS
MHVKCRPPRDRNGPHERYHLEVEAGNTLVRNESHKNCDFRVKDLQYSTDY
TFKAYFHNGDYPGEPFILHHSTSYNSKALIAFLAFLIIVTSIALLVVLYK
IYDLHKKRSCNLDEQQELVERDDEKQLMNVEPIHADILLETYKRKIADEG
RLFLAEFQSIPRVFSKFPIKEARKPFNQNKNRYVDILPYDYNRVELSEIN
GDAGSNYINASYIDGFKEPRKYIAAQGPRDETVDDFWRMIWEQKATVIVM
VTRCEEGNRNKCAEYWPSMEEGTRAFGDVVVKINQHKRCPDYIIQKLNIV
NKKEKATGREVTHIQFTSWPDHGVPEDPHLLLKLRRRVNAFSNFFSGPIV
VHCSAGVGRTGTYIGIDAMLEGLEAENKVDVYGYVVKLRRQRCLMVQVEA
QYILIHQALVEYNQFGETEVNLSELHPYLHNMKKRDPPSEPSPLEAEFQR
LPSYRSWRTQHIGNQEENKSKNRNSNVIPYDYNRVPLKHELEMSKESEHD
SDESSDDDSDSEEPSKYINASFIMSYWKPEVMIAAQGPLKETIGDFWQMI
FQRKVKVIVMLTELKHGDQEICAQYWGEGKQTYGDIEVDLKDTDKSSTYT
LRVFELRHSKRKDSRTVYQYQYTNWSVEQLPAEPKELISMIQVVKQKLPQ
KNSSEGNKHHKSTPLLIHCRDGSQQTGIFCALLNLLESAETEEVVDIFQV
VKALRKARPGMVSTFEQYQFLYDVIASTYPAQNGQVKKNNHQEDKIEFDN
EVDKVKQDANCVNPLGAPEKLPEAKEQAEGSEPTSGTEGPEHSVNGPASP
ALNQGS

In one embodiment, a sequence comprising SEQ ID NO: 23 is the sequence of hCD45RC set forth in SEQ ID NO: 104, corresponding to UniProt Accession P08575-10 (version 10, modified Mar. 28, 2018—Checksum: F92C874C9A114890).

SEQ ID NO: 104
(wherein SEQ ID NO: 23 is underlined)
MTMYLWLKLLAFGFAFLDTEVFVTGQSPTPSPTDVPGERSTASTFPTDPV
SPLTTTLSLAHHSSAALPARTSNTTITANTSDAYLNASETTTLSPSGSAV
ISTTTIATTPSKPTCDEKYANITVDYLYNKETKLFTAKLNVNENVECGNN
TCTNNEVHNLTECKNASVSISHNSCTAPDKTLILDVPPGVEKFQLHDCTQ
VEKADTTICLKWKNIETFTCDTQNITYRFQCGNMIFDNKEIKLENLEPEH
EYKCDSEILYNNHKFTNASKIIKTDFGSPGEPQIIFCRSEAAHQGVITWN
PPQRSFHNFTLCYIKETEKDCLNLDKNLIKYDLQNLKPYTKYVLSLHAYI
IAKVQRNGSAAMCHFTTKSAPPSQVWNMTVSMTSDNSMHVKCRPPRDRNG
PHERYHLEVEAGNTLVRNESHKNCDFRVKDLQYSTDYTFKAYFHNGDYPG
EPFILHHSTSYNSKALIAFLAFLIIVTSIALLVVLYKIYDLHKKRSCNLD
EQQELVERDDEKQLMNVEPIHADILLETYKRKIADEGRLFLAEFQSIPRV
FSKFPIKEARKPFNQNKNRYVDILPYDYNRVELSEINGDAGSNYINASYI
DGFKEPRKYIAAQGPRDETVDDFWRMIWEQKATVIVMVTRCEEGNRNKCA
EYWPSMEEGTRAFGDVVVKINQHKRCPDYIIQKLNIVNKKEKATGREVTH
IQFTSWPDHGVPEDPHLLLKLRRRVNAFSNFFSGPIVVHCSAGVGRTGTY
IGIDAMLEGLEAENKVDVYGYVVKLRRQRCLMVQVEAQYILIHQALVEYN
QFGETEVNLSELHPYLHNMKKRDPPSEPSPLEAEFQRLPSYRSWRTQHIG
NQEENKSKNRNSNVIPYDYNRVPLKHELEMSKESEHDSDESSDDDSDSEE
PSKYINASFIMSYWKPEVMIAAQGPLKETIGDFWQMIFQRKVKVIVMLTE
LKHGDQEICAQYWGEGKQTYGDIEVDLKDTDKSSTYTLRVFELRHSKRKD
SRTVYQYQYTNWSVEQLPAEPKELISMIQVVKQKLPQKNSSEGNKHHKST
PLLIHCRDGSQQTGIFCALLNLLESAETEEVVDIFQVVKALRKARPGMVS
TFEQYQFLYDVIASTYPAQNGQVKKNNHQEDKIEFDNEVDKVKQDANCVN
PLGAPEKLPEAKEQAEGSEPTSGTEGPEHSVNGPASPALNQGS

In one embodiment, the antibody or binding fragment thereof according to the present invention does not bind to the A determinant encoded by exon 4 of hCD45. In one embodiment, the antibody or binding fragment thereof according to the present invention does not bind to at least one epitope on the A determinant encoded by exon 4 of hCD45.

In one embodiment, the amino acid sequence of the A determinant encoded by exon 4 of hCD45 comprises or consists of SEQ ID NO: 105.

SEQ ID NO: 105
GLTTAKMPSVPLSSDPLPTHTTAFSPASTFERENDFSETTTSLSPDNTST
QVSPDSLDNASAFNTT

In one embodiment, the antibody or binding fragment thereof according to the present invention does not bind to the B determinant encoded by exon 5 of hCD45. In one embodiment, the antibody or binding fragment thereof according to the present invention does not bind to at least one epitope on the B determinant encoded by exon 5 of hCD45.

In one embodiment, the amino acid sequence of the B determinant encoded by exon 5 of hCD45 comprises or consists of SEQ ID NO: 106.

SEQ ID NO: 106
GVSSVQTPHLPTHADSQTPSAGTDTQTFSGSAANAKLNPTPGSNAIS

In one embodiment, the antibody or binding fragment thereof according to the present invention does not bind to hCD45RA. In one embodiment, the antibody or binding fragment thereof according to the present invention does not bind to at least one epitope of hCD45RA.

In one embodiment, the amino acid sequence of hCD45RA comprises or consists of SEQ ID NO: 107, corresponding to UniProt Accession P08575-8 (version 8, modified Mar. 28, 2018—Checksum: F42C1FEC9EDE4BC0).

SEQ ID NO: 107
MTMYLWLKLLAFGFAFLDTEVFVTGQSPTPSPTGLTTAKMPSVPLSSDPL
PTHTTAFSPASTFERENDFSETTTSLSPDNTSTQVSPDSLDNASAFNTTD
AYLNASETTTLSPSGSAVISTTTIATTPSKPTCDEKYANITVDYLYNKET
KLFTAKLNVNENVECGNNTCTNNEVHNLTECKNASVSISHNSCTAPDKTL
ILDVPPGVEKFQLHDCTQVEKADTTICLKWKNIETFTCDTQNITYRFQCG
NMIFDNKEIKLENLEPEHEYKCDSEILYNNHKFTNASKIIKTDFGSPGEP
QIIFCRSEAAHQGVITWNPPQRSFHNFTLCYIKETEKDCLNLDKNLIKYD
LQNLKPYTKYVLSLHAYIIAKVQRNGSAAMCHFTTKSAPPSQVWNMTVSM
TSDNSMHVKCRPPRDRNGPHERYHLEVEAGNTLVRNESHKNCDFRVKDLQ
YSTDYTFKAYFHNGDYPGEPFILHHSTSYNSKALIAFLAFLIIVTSIALL
VVLYKIYDLHKKRSCNLDEQQELVERDDEKQLMNVEPIHADILLETYKRK
IADEGRLFLAEFQSIPRVFSKFPIKEARKPFNQNKNRYVDILPYDYNRVE
LSEINGDAGSNYINASYIDGFKEPRKYIAAQGPRDETVDDFWRMIWEQKA
TVIVMVTRCEEGNRNKCAEYWPSMEEGTRAFGDVVVKINQHKRCPDYIIQ
KLNIVNKKEKATGREVTHIQFTSWPDHGVPEDPHLLLKLRRRVNAFSNFF
SGPIVVHCSAGVGRTGTYIGIDAMLEGLEAENKVDVYGYVVKLRRQRCLM
VQVEAQYILIHQALVEYNQFGETEVNLSELHPYLHNMKKRDPPSEPSPLE
AEFQRLPSYRSWRTQHIGNQEENKSKNRNSNVIPYDYNRVPLKHELEMSK
ESEHDSDESSDDDSDSEEPSKYINASFIMSYWKPEVMIAAQGPLKETIGD
FWQMIFQRKVKVIVMLTELKHGDQEICAQYWGEGKQTYGDIEVDLKDTDK
SSTYTLRVFELRHSKRKDSRTVYQYQYTNWSVEQLPAEPKELISMIQVVK
QKLPQKNSSEGNKHHKSTPLLIHCRDGSQQTGIFCALLNLLESAETEEVV
DIFQVVKALRKARPGMVSTFEQYQFLYDVIASTYPAQNGQVKKNNHQEDK
IEFDNEVDKVKQDANCVNPLGAPEKLPEAKEQAEGSEPTSGTEGPEHSVN
GPASPALNQGS

In one embodiment, the antibody or binding fragment thereof according to the present invention does not bind to hCD45RB. In one embodiment, the antibody or binding fragment thereof according to the present invention does not bind to at least one epitope of hCD45RB.

In one embodiment, the amino acid sequence of hCD45RB comprises or consists of SEQ ID NO: 108, corresponding to UniProt Accession P08575-9 (version 9, modified Mar. 28, 2018—Checksum: 745870037910C575).

SEQ ID NO: 108
MTMYLWLKLLAFGFAFLDTEVFVTGQSPTPSPTGVSSVQTPHLPTHADSQ
TPSAGTDTQTFSGSAANAKLNPTPGSNAISDAYLNASETTTLSPSGSAVI
STTTIATTPSKPTCDEKYANITVDYLYNKETKLFTAKLNVNENVECGNNT
CTNNEVHNLTECKNASVSISHNSCTAPDKTLILDVPPGVEKFQLHDCTQV
EKADTTICLKWKNIETFTCDTQNITYRFQCGNMIFDNKEIKLENLEPEHE
YKCDSEILYNNHKFTNASKIIKTDFGSPGEPQIIFCRSEAAHQGVITWNP
PQRSFHNFTLCYIKETEKDCLNLDKNLIKYDLQNLKPYTKYVLSLHAYII
AKVQRNGSAAMCHFTTKSAPPSQVWNMTVSMTSDNSMHVKCRPPRDRNGP
HERYHLEVEAGNTLVRNESHKNCDFRVKDLQYSTDYTFKAYFHNGDYPGE
PFILHHSTSYNSKALIAFLAFLIIVTSIALLVVLYKIYDLHKKRSCNLDE
QQELVERDDEKQLMNVEPIHADILLETYKRKIADEGRLFLAEFQSIPRVF
SKFPIKEARKPFNQNKNRYVDILPYDYNRVELSEINGDAGSNYINASYID
GFKEPRKYIAAQGPRDETVDDFWRMIWEQKATVIVMVTRCEEGNRNKCAE
YWPSMEEGTRAFGDVVVKINQHKRCPDYIIQKLNIVNKKEKATGREVTHI
QFTSWPDHGVPEDPHLLLKLRRRVNAFSNFFSGPIVVHCSAGVGRTGTYI
GIDAMLEGLEAENKVDVYGYVVKLRRQRCLMVQVEAQYILIHQALVEYNQ
FGETEVNLSELHPYLHNMKKRDPPSEPSPLEAEFQRLPSYRSWRTQHIGN
QEENKSKNRNSNVIPYDYNRVPLKHELEMSKESEHDSDESSDDDSDSEEP
SKYINASFIMSYWKPEVMIAAQGPLKETIGDFWQMIFQRKVKVIVMLTEL
KHGDQEICAQYWGEGKQTYGDIEVDLKDTDKSSTYTLRVFELRHSKRKDS
RTVYQYQYTNWSVEQLPAEPKELISMIQVVKQKLPQKNSSEGNKHHKSTP
LLIHCRDGSQQTGIFCALLNLLESAETEEVVDIFQVVKALRKARPGMVST
FEQYQFLYDVIASTYPAQNGQVKKNNHQEDKIEFDNEVDKVKQDANCVNP
LGAPEKLPEAKEQAEGSEPTSGTEGPEHSVNGPASPALNQGS

In one embodiment, the antibody or binding fragment thereof according to the present invention does not bind to hCD45RAB. In one embodiment, the antibody or binding fragment thereof according to the present invention does not bind to at least one epitope of hCD45RAB.

In one embodiment, the amino acid sequence of hCD45RAB comprises or consists of SEQ ID NO: 109, corresponding to UniProt Accession P08575-5 (version 5, modified Mar. 28, 2018—Checksum: EA40BE995CD98F7C).

SEQ ID NO: 109
MTMYLWLKLLAFGFAFLDTEVFVTGQSPTPSPTGLTTAKMPSVPLSSDPL
PTHTTAFSPASTFERENDFSETTTSLSPDNTSTQVSPDSLDNASAFNTTG
VSSVQTPHLPTHADSQTPSAGTDTQTFSGSAANAKLNPTPGSNAISDAYL
NASETTTLSPSGSAVISTTTIATTPSKPTCDEKYANITVDYLYNKETKLF
TAKLNVNENVECGNNTCTNNEVHNLTECKNASVSISHNSCTAPDKTLILD
VPPGVEKFQLHDCTQVEKADTTICLKWKNIETFTCDTQNITYRFQCGNMI
FDNKEIKLENLEPEHEYKCDSEILYNNHKFTNASKIIKTDFGSPGEPQII
FCRSEAAHQGVITWNPPQRSFHNFTLCYIKETEKDCLNLDKNLIKYDLQN
LKPYTKYVLSLHAYIIAKVQRNGSAAMCHFTTKSAPPSQVWNMTVSMTSD
NSMHVKCRPPRDRNGPHERYHLEVEAGNTLVRNESHKNCDFRVKDLQYST
DYTFKAYFHNGDYPGEPFILHHSTSYNSKALIAFLAFLIIVTSIALLVVL
YKIYDLHKKRSCNLDEQQELVERDDEKQLMNVEPIHADILLETYKRKIAD
EGRLFLAEFQSIPRVFSKFPIKEARKPFNQNKNRYVDILPYDYNRVELSE
INGDAGSNYINASYIDGFKEPRKYIAAQGPRDETVDDFWRMIWEQKATVI
VMVTRCEEGNRNKCAEYWPSMEEGTRAFGDVVVKINQHKRCPDYIIQKLN
IVNKKEKATGREVTHIQFTSWPDHGVPEDPHLLLKLRRRVNAFSNFFSGP
IVVHCSAGVGRTGTYIGIDAMLEGLEAENKVDVYGYVVKLRRQRCLMVQV
EAQYILIHQALVEYNQFGETEVNLSELHPYLHNMKKRDPPSEPSPLEAEF
QRLPSYRSWRTQHIGNQEENKSKNRNSNVIPYDYNRVPLKHELEMSKESE
HDSDESSDDDSDSEEPSKYINASFIMSYWKPEVMIAAQGPLKETIGDFWQ
MIFQRKVKVIVMLTELKHGDQEICAQYWGEGKQTYGDIEVDLKDTDKSST
YTLRVFELRHSKRKDSRTVYQYQYTNWSVEQLPAEPKELISMIQVVKQKL
PQKNSSEGNKHHKSTPLLIHCRDGSQQTGIFCALLNLLESAETEEVVDIF
QVVKALRKARPGMVSTFEQYQFLYDVIASTYPAQNGQVKKNNHQEDKIEF
DNEVDKVKQDANCVNPLGAPEKLPEAKEQAEGSEPTSGTEGPEHSVNGPA
SPALNQGS

In one embodiment, the antibody or binding fragment thereof according to the present invention does not bind to hCD45R0. In one embodiment, the antibody or binding fragment thereof according to the present invention does not bind to at least one epitope of hCD45R0.

In one embodiment, the amino acid sequence of hCD45R0 comprises or consists of SEQ ID NO: 110, corresponding to UniProt Accession P08575-4 (version 4, modified Mar. 28, 2018—Checksum: D3CB364EF4243384).

SEQ ID NO: 110
MTMYLWLKLLAFGFAFLDTEVFVTGQSPTPSPTDAYLNASETTTLSPSGS
AVISTTTIATTPSKPTCDEKYANITVDYLYNKETKLFTAKLNVNENVECG
NNTCTNNEVHNLTECKNASVSISHNSCTAPDKTLILDVPPGVEKFQLHDC
TQVEKADTTICLKWKNIETFTCDTQNITYRFQCGNMIFDNKEIKLENLEP
EHEYKCDSEILYNNHKFTNASKIIKTDFGSPGEPQIIFCRSEAAHQGVIT
WNPPQRSFHNFTLCYIKETEKDCLNLDKNLIKYDLQNLKPYTKYVLSLHA
YIIAKVQRNGSAAMCHFTTKSAPPSQVWNMTVSMTSDNSMHVKCRPPRDR
NGPHERYHLEVEAGNTLVRNESHKNCDFRVKDLQYSTDYTFKAYFHNGDY
PGEPFILHHSTSYNSKALIAFLAFLIIVTSIALLVVLYKIYDLHKKRSCN
LDEQQELVERDDEKQLMNVEPIHADILLETYKRKIADEGRLFLAEFQSIP
RVFSKFPIKEARKPFNQNKNRYVDILPYDYNRVELSEINGDAGSNYINAS
YIDGFKEPRKYIAAQGPRDETVDDFWRMIWEQKATVIVMVTRCEEGNRNK
CAEYWPSMEEGTRAFGDVVVKINQHKRCPDYIIQKLNIVNKKEKATGREV
THIQFTSWPDHGVPEDPHLLLKLRRRVNAFSNFFSGPIVVHCSAGVGRTG
TYIGIDAMLEGLEAENKVDVYGYVVKLRRQRCLMVQVEAQYILIHQALVE
YNQFGETEVNLSELHPYLHNMKKRDPPSEPSPLEAEFQRLPSYRSWRTQH
IGNQEENKSKNRNSNVIPYDYNRVPLKHELEMSKESEHDSDESSDDDSDS
EEPSKYINASFIMSYWKPEVMIAAQGPLKETIGDFWQMIFQRKVKVIVML
TELKHGDQEICAQYWGEGKQTYGDIEVDLKDTDKSSTYTLRVFELRHSKR
KDSRTVYQYQYTNWSVEQLPAEPKELISMIQVVKQKLPQKNSSEGNKHHK
STPLLIHCRDGSQQTGIFCALLNLLESAETEEVVDIFQVVKALRKARPGM
VSTFEQYQFLYDVIASTYPAQNGQVKKNNHQEDKIEFDNEVDKVKQDANC
VNPLGAPEKLPEAKEQAEGSEPTSGTEGPEHSVNGPASPALNQGS

In one embodiment, the at least one epitope is a conformational epitope. In another embodiment, the at least one epitope is a sequential epitope.

In one embodiment, the antibody or binding fragment thereof according to the present invention binds to hCD45RC with an equilibrium dissociation constant (K_{d}) of about 5×10⁻⁷ M or less, preferably of about 2.5×10⁻⁷ M or less, about 1×10⁻⁷ M or less, about 7.5×10⁻⁸ M or less, about 5×10⁻⁸ M or less, about 1×10⁻⁸ M or less.

In one embodiment, the antibody or binding fragment thereof according to the present invention binds to hCD45RC with an association rate (Kₒₙ) of about 1×10⁴ M⁻¹ sec⁻¹ or more, preferably of about 5×10⁴ M⁻¹ sec⁻¹ or more, about 1×10⁵ M⁻¹ sec⁻¹ or more, about 2.5×10⁵ M⁻¹ sec⁻¹ or more, about 5×10⁵ M⁻¹ sec⁻¹ or more.

In one embodiment, the antibody or binding fragment thereof according to the present invention binds to hCD45RC with a dissociation rate (K_{off}) of about 5×10⁻² sec⁻¹ or less, preferably of about 4×10⁻² sec⁻¹ or less, about 3×10⁻² sec⁻¹ or less, about 2×10⁻² sec⁻¹ or less, about 1.5×10⁻² sec⁻¹ or less.

In one embodiment, the antibody or binding fragment thereof according to the present invention binds to hCD45RC with at least one of, preferably at least two of, more preferably the three of:
an equilibrium dissociation constant (K_{d}) of about 5×10⁻⁷ M or less, preferably of about 2.5×10⁻⁷ M or less, about 1×10⁻⁷ M or less, about 7.5×10⁻⁸ M or less, about 5×10⁻⁸ M or less, about 1×10⁻⁸ M or less;
an association rate (Kₒₙ) of about 1×10⁴ M⁻¹ sec⁻¹ or more, preferably of about 5×10⁴ M⁻¹ sec⁻¹ or more, about 1×10⁵ M⁻¹ sec⁻¹ or more, about 2.5×10⁵ M⁻¹ sec⁻¹ or more, about 5×10⁵ M⁻¹ sec⁻¹ or more; and
a dissociation rate (K_{off}) of about 5×10⁻² sec⁻¹ or less, preferably of about 4×10⁻² sec⁻¹ or less, about 3×10⁻² sec⁻¹ or less, about 2×10⁻² sec⁻¹ or less, about 1.5×10⁻² sec⁻¹ or less.

Methods for determining the affinity (including, for example, determining the K_{d}, k_{off} and kₒₙ) of an antibody or binding fragment thereof for its ligand are well-known in the art, and include, without limitation, surface plasmon resonance (SPR), fluorescence-activated cell sorting (FACS), enzyme-linked immunosorbent assay (ELISA), AlphaLISA and KinExA.

A preferred method is BIAcore®, which relies on SPR using immobilized CD45RC to determine the affinity of an antibody or binding fragment thereof. A way of implementing this method will be further illustrated in the Examples section.

In one embodiment, the antibody or binding fragment thereof according to the present invention is a polyclonal antibody or binding fragment thereof.

In a preferred embodiment, the antibody or binding fragment thereof according to the present invention is a monoclonal antibody or binding fragment thereof.

In one embodiment, the antibody or binding fragment thereof according to the present invention is a molecule selected from the group comprising or consisting of a whole antibody, a single-chain antibody, a dimeric single chain antibody, a single-domain antibody, a Fv, a Fab, a Fab′, a Fab′-SH, a F(ab)′2, a Fd, a defucosylated antibody, a bispecific antibody, a diabody, a triabody and a tetrabody.

The term “binding fragment”, as used herein, refers to a part or region of the antibody according to the present invention, which comprises fewer amino acid residues than the whole antibody. A “binding fragment” binds antigen and/or competes with the whole antibody from which it was derived for antigen binding (e.g., specific binding to human CD45RC). Antibody binding fragments encompasses, without any limitation, single chain antibodies, Fv, Fab, Fab′, Fab′-SH, F(ab)′2, Fd, defucosylated antibodies, diabodies, triabodies and tetrabodies.

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding (Plückthun, 1994. Antibodies from Escherichia coli. In Rosenberg & Moore (Eds.), The pharmacology of monoclonal antibodies. Handbook of Experimental Pharmacology, 113:269-315. Springer: Berlin, Heidelberg).

“Fv”, as used herein, refers to the minimum antibody fragment that contains a complete antigen-recognition and -binding site. This fragment consists of a dimer of one HCVR and one LCVR in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Diabodies”, as used herein, refers to small antibody fragments prepared by constructing scFv fragments with short linkers (about 5-10 residues) between the HCVR and LCVR such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the HCVR and LCVR of the two antibodies are present on different polypeptide chains. Diabodies are described more fully in Patent EP0404097, Patent application WO1993011161; and Holliger et al., 1993. Proc Natl Acad Sci USA. 90(14):6444-8.

Antibody binding fragments can be obtained using standard methods. For instance, Fab or F(ab′)2 fragments may be produced by protease digestion of the isolated antibodies, according to conventional techniques.

It will also be appreciated that antibodies or binding fragments thereof according to the present invention can be modified using known methods. For example, to slow clearance in vivo and obtain a more desirable pharmacokinetic profile, the antibody or binding fragment thereof may be modified with polyethylene glycol (PEG). Methods for coupling and site-specifically conjugating PEG to an antibody or binding fragment thereof are described in, e.g., Leong et al., 2001. Cytokine. 16(3):106-19; Delgado et al., 1996. Br J Cancer. 73(2):175-82.

In one embodiment, the antibody or binding fragment thereof according to the present invention is a molecule selected from the group comprising or consisting of a unibody, a domain antibody, and a nanobody.

“Unibodies” are well-known in the art and refer to antibody fragments lacking the hinge region of IgG4 antibodies. The deletion of the hinge region results in a molecule that is essentially half the size of traditional IgG4 antibodies and has a univalent binding region rather than the bivalent biding region of IgG4 antibodies.

“Domain antibodies” are well-known in the art and refer to the smallest functional binding units of antibodies, corresponding to the variable regions of either the heavy or light chains of antibodies.

“Single-domain antibodies” are well-known in the art and refer to antibody-derived proteins that contain the unique structural and functional properties of naturally-occurring heavy chain antibodies (Muyldermans, 2013. Annu Rev Biochem. 82:775-97). These heavy chain antibodies may contain a single variable domain (V_{H}H)—one such example is Nanobodies®—, or a single variable domain (V_{H}H) and two constant domains (C_{H}2 and C_{H}3)—such as camelid antibodies-, or a single variable domain (V_{H}H) and five constant domains (C_{H}1, C_{H}2, C_{H}3, C_{H}4 and C_{H}5)—such as shark antibodies.

In one embodiment, the antibody or binding fragment thereof according to the present invention is a mimetic selected from the group comprising or consisting of an affibody, an affilin, an affitin, an adnectin, an atrimer, an evasin, a DARPin, an anticalin, an avimer, a fynomer, a versabody and a duocalin.

“Affibodies” are well-known in the art and refer to affinity proteins based on a 58 amino acid residue protein domain, derived from one of the IgG binding domain of staphylococcal protein A (Frejd & Kim, 2017. Exp Mol Med. 49(3):e306; U.S. Pat. No. 5,831,012).

“DARPins” (Designed Ankyrin Repeat Proteins) are well-known in the art and refer to an antibody mimetic DRP (designed repeat protein) technology developed to exploit the binding abilities of non-antibody proteins (Binz et al., 2003. J Mol Biol. 332(2):489-503; Plüchthun, 2015. Annu Rev Pharmacol Toxicol. 55:489-511).

“Anticalins” are well-known in the art and refer to another antibody mimetic technology, wherein the binding specificity is derived from lipocalins (Skerra, 2008. FEBS J. 275(11):2677-83). Anticalins may also be formatted as dual targeting protein, called “duocalins” (Schlehuber & Skerra, 2001. Biol Chem. 382(9):1335-42).

“Avimers” are well-known in the art and refer to another antibody mimetic technology (Silverman et al., 2005. Nat Biotechnol. 23(12):1556-61).

“Versabodies” are well-known in the art and refer to another antibody mimetic technology (Patent Application US20070191272). They are small proteins of 3-5 kDa with >15% cysteines, which form a high disulfide density scaffold, replacing the hydrophobic core the typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T cell epitopes, because the residues that contribute most to MHC presentation are hydrophobic. All four of these properties are well-known to affect immunogenicity, and together they are expected to cause a large decrease in immunogenicity.

In one embodiment, the antibody or binding fragment thereof according to the present invention also encompasses multispecific antibodies or binding fragments thereof, i.e., being immunospecific for more than one, such as at least two, different antigens, one of which being hCD45RC according to the present invention.

In one embodiment, the antibody or binding fragment thereof according to the present invention also encompasses polymers of antibodies or binding fragments thereof, i.e., more than one, such as at least two, antibodies or binding fragments thereof, whether identical or different, being covalently linked together, directly or indirectly.

In one embodiment, the antibody or binding fragment thereof according to the present invention is an immunoconjugate comprising said antibody or binding fragment thereof, further conjugated to a therapeutic agent.

In one embodiment, the antibody or binding fragment thereof according to the present invention is a conjugate comprising said antibody or binding fragment thereof further conjugated to an imaging agent. Said conjugate could be used, e.g., for imaging applications.

In the following, and unless explicitly mentioned otherwise, CDR numbering and definitions are according to the Kabat/Chothia definition.

In one embodiment, the antibody or binding fragment thereof according to the present invention comprises a heavy chain variable region (abbreviated herein as HCVR or V_{H}) which comprises at least one, preferably at least two, more preferably the following three complementary-determining regions (CDRs):

V_{H}-CDR1:
(SEQ ID NO: 1)
NYYIG;
V_{H}-CDR2:
(SEQ ID NO: 2)
X₁-IF-X₂-GG-X₃-Y-X₄-N-X₅-X₆-X₇-X₈-X₉-X₁₀-G;
and
V_{H}-CDR3:
(SEQ ID NO: 3)
RNFDY,

with:
X₁ being selected from Asp (D), Ile (I) and Arg (R);
X₂ being selected from Pro (P) and Ser (S);
X₃ being selected from Asp (D), Ser (S) and Gly (G);
X₄ being selected from Ala (A) and Thr (T);
X₅ being selected from Ser (S) and Tyr (Y);
X₆ being selected from Asn (N), Ala (A) and Ser (S);
X₇ being selected from Glu (E), Asp (D), Pro (P) and Gln (Q);
X₈ being selected from Lys (K) and Ser (S);
X₉ being selected from Phe (F) and Val (V); and
X₁₀ being selected from Lys (K) and Gln (Q).

In one embodiment, the antibody or binding fragment thereof according to the present invention comprises a HCVR which comprises the following three CDRs:

V_{H}-CDR1:
(SEQ ID NO: 1)
NYYIG;
V_{H}-CDR2:
(SEQ ID NO: 2)
X₁-IF-X₂-GG-X₃-Y-X₄-N-X₅-X₆-X₇-X₈-X₉-X₁₀-G;
and
V_{H}-CDR3:
(SEQ ID NO: 3)
RNFDY,

with: DIFPGGDYANSNEKFKG
X₁ being selected from Asp (D), Ile (I) and Arg (R);
X₂ being selected from Pro (P) and Ser (S);
X₃ being selected from Asp (D), Ser (S) and Gly (G);
X₄ being selected from Ala (A) and Thr (T);
X₅ being selected from Ser (S) and Tyr (Y);
X₆ being selected from Asn (N), Ala (A) and Ser (S);
X₇ being selected from Glu (E), Asp (D), Pro (P) and Gln (Q);
X₈ being selected from Lys (K) and Ser (S);
X₉ being selected from Phe (F) and Val (V); and
X₁₀ being selected from Lys (K) and Gln (Q).

In one embodiment, the antibody or binding fragment thereof according to the present invention comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 4)
DIFPGGDYANSNEKFKG;
and
V_{H}-CDR3:
(SEQ ID NO: 3)
RNFDY.

In one embodiment, the antibody or binding fragment thereof according to the present invention comprises a HCVR which comprises the following three CDRs:

V_{H}-CDR1:
(SEQ ID NO: 1)
NYYIG;
V_{H}-CDR2:
(SEQ ID NO: 4)
DIFPGGDYANSNEKFKG;
and
V_{H}-CDR3:
(SEQ ID NO: 3)
RNFDY.

In one embodiment, the antibody or binding fragment thereof according to the present invention comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 5)
DIFPGGDYANSNEKVKG;
and
V_{H}-CDR3:
(SEQ ID NO: 3)
RNFDY.

In one embodiment, the antibody or binding fragment thereof according to the present invention comprises a HCVR which comprises the following three CDRs:

V_{H}-CDR1:
(SEQ ID NO: 1)
NYYIG;
V_{H}-CDR2:
(SEQ ID NO: 5)
DIFPGGDYANSNEKVKG;
and
V_{H}-CDR3:
(SEQ ID NO: 3)
RNFDY.

In one embodiment, the antibody or binding fragment thereof according to the present invention comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 6)
DIFPGGGYTNYAEKFQG;
and
V_{H}-CDR3:
(SEQ ID NO: 3)
RNFDY.

In one embodiment, the antibody or binding fragment thereof according to the present invention comprises a HCVR which comprises the following three CDRs:

V_{H}-CDR1:
(SEQ ID NO: 1)
NYYIG;
V_{H}-CDR2:
(SEQ ID NO: 6)
DIFPGGGYTNYAEKFQG;
and
V_{H}-CDR3:
(SEQ ID NO: 3)
RNFDY.

In one embodiment, the antibody or binding fragment thereof according to the present invention comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 7)
DIFPGGSYTNYSESFQG;
and
V_{H}-CDR3:
(SEQ ID NO: 3)
RNFDY.

In one embodiment, the antibody or binding fragment thereof according to the present invention comprises a HCVR which comprises the following three CDRs:

V_{H}-CDR1:
(SEQ ID NO: 1)
NYYIG;
V_{H}-CDR2:
(SEQ ID NO: 7)
DIFPGGSYTNYSESFQG;
and
V_{H}-CDR3:
(SEQ ID NO: 3)
RNFDY.

In one embodiment, the antibody or binding fragment thereof according to the present invention comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 8)
DIFPGGSYTNYADSVKG;
and
V_{H}-CDR3:
(SEQ ID NO: 3)
RNFDY.

In one embodiment, the antibody or binding fragment thereof according to the present invention comprises a HCVR which comprises the following three CDRs:

(SEQ ID NO: 1)
V_{H}-CDR1: NYYIG;
(SEQ ID NO: 8)
V_{H}-CDR2: DIFPGGSYTNYADSVKG;
and
(SEQ ID NO: 3)
V_{H}-CDR3: RNFDY.

In one embodiment, the antibody or binding fragment thereof according to the present invention comprises a HCVR which comprises at least one, preferably at least two, more preferably the following three CDRs:

(SEQ ID NO: 1)
V_{H}-CDR1: NYYIG;
(SEQ ID NO: 9)
V_{H}-CDR2: RIFPGGGYTNYAQKFQG;
and
(SEQ ID NO: 3)
V_{H}-CDR3: RNFDY.

In one embodiment, the antibody or binding fragment thereof according to the present invention comprises a HCVR which comprises the following three CDRs:

(SEQ ID NO: 1)
V_{H}-CDR1: NYYIG;
(SEQ ID NO: 9)
V_{H}-CDR2: RIFPGGGYTNYAQKFQG;
and
(SEQ ID NO: 3)
V_{H}-CDR3: RNFDY.

In one embodiment, the antibody or binding fragment thereof according to the present invention comprises a HCVR which comprises at least one, preferably at least two, more preferably the following three CDRs:

(SEQ ID NO: 1)
V_{H}-CDR1: NYYIG;
(SEQ ID NO: 10)
V_{H}-CDR2: IIFPGGSYTNYSPSFQG;
and
(SEQ ID NO: 3)
V_{H}-CDR3: RNFDY.

In one embodiment, the antibody or binding fragment thereof according to the present invention comprises a HCVR which comprises the following three CDRs:

(SEQ ID NO: 1)
V_{H}-CDR1: NYYIG;
(SEQ ID NO: 10)
V_{H}-CDR2: IIFPGGSYTNYSPSFQG;
and
(SEQ ID NO: 3)
V_{H}-CDR3: RNFDY.

In one embodiment, the antibody or binding fragment thereof according to the present invention comprises a HCVR which comprises at least one, preferably at least two, more preferably the following three CDRs:

(SEQ ID NO: 1)
V_{H}-CDR1: NYYIG;
(SEQ ID NO: 11)
V_{H}-CDR2: DIFSGGSYTNYADSVKG;
and
(SEQ ID NO: 3)
V_{H}-CDR3: RNFDY.

In one embodiment, the antibody or binding fragment thereof according to the present invention comprises a HCVR which comprises the following three CDRs:

(SEQ ID NO: 1)
V_{H}-CDR1: NYYIG;
(SEQ ID NO: 11)
V_{H}-CDR2: DIFSGGSYTNYADSVKG;
and
(SEQ ID NO: 3)
V_{H}-CDR3: RNFDY.

In one embodiment, the antibody or binding fragment thereof according to the present invention comprises a HCVR which comprises at least one, preferably at least two, more preferably the following three CDRs:

(SEQ ID NO: 1)
V_{H}-CDR1: NYYIG;
(SEQ ID NO: 100)
V_{H}-CDR2: DIFPGGDYTNYAEKFQG;
and
(SEQ ID NO: 3)
V_{H}-CDR3: RNFDY.

In one embodiment, the antibody or binding fragment thereof according to the present invention comprises a HCVR which comprises the following three CDRs:

(SEQ ID NO: 1)
V_{H}-CDR1: NYYIG;
(SEQ ID NO: 100)
V_{H}-CDR2: DIFPGGDYTNYAEKFQG;
and
(SEQ ID NO: 3)
V_{H}-CDR3: RNFDY.

In one embodiment, the antibody or binding fragment thereof according to the present invention comprises a HCVR which comprises at least one, preferably at least two, more preferably the following three CDRs:

(SEQ ID NO: 1)
V_{H}-CDR1: NYYIG;
(SEQ ID NO: 116)
V_{H}-CDR2: DIFPGGGYANYAEKFQG;
and
(SEQ ID NO: 3)
V_{H}-CDR3: RNFDY.

In one embodiment, the antibody or binding fragment thereof according to the present invention comprises a HCVR which comprises the following three CDRs:

(SEQ ID NO: 1)
V_{H}-CDR1: NYYIG;
(SEQ ID NO: 116)
V_{H}-CDR2: DIFPGGGYANYAEKFQG;
and
(SEQ ID NO: 3)
V_{H}-CDR3: RNFDY.

In one embodiment, the antibody or binding fragment thereof according to the present invention comprises a HCVR which comprises at least one, preferably at least two, more preferably the following three CDRs:

(SEQ ID NO: 1)
V_{H}-CDR1: NYYIG;
(SEQ ID NO: 117)
V_{H}-CDR2: DIFPGGGYTNYAEKFKG;
and
(SEQ ID NO: 3)
V_{H}-CDR3: RNFDY.

In one embodiment, the antibody or binding fragment thereof according to the present invention comprises a HCVR which comprises the following three CDRs:

(SEQ ID NO: 1)
V_{H}-CDR1: NYYIG;
(SEQ ID NO: 117)
V_{H}-CDR2: DIFPGGGYTNYAEKFKG;
and
(SEQ ID NO: 3)
V_{H}-CDR3: RNFDY.

In one embodiment, the antibody or binding fragment thereof according to the present invention comprises a HCVR which comprises at least one, preferably at least two, more preferably the following three CDRs:

(SEQ ID NO: 1)
V_{H}-CDR1: NYYIG;
(SEQ ID NO: 118)
V_{H}-CDR2: DIFPGGGYTNYNEKFQG;
and
(SEQ ID NO: 3)
V_{H}-CDR3: RNFDY.

In one embodiment, the antibody or binding fragment thereof according to the present invention comprises a HCVR which comprises the following three CDRs:

(SEQ ID NO: 1)
V_{H}-CDR1: NYYIG;
(SEQ ID NO: 118)
V_{H}-CDR2: DIFPGGGYTNYNEKFQG;
and
(SEQ ID NO: 3)
V_{H}-CDR3: RNFDY.

In one embodiment, the antibody or binding fragment thereof according to the present invention comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 119)
DIFPGGGYTNSAEKFQG;
and
V_{H}-CDR3:
(SEQ ID NO: 3)
RNFDY.

In one embodiment, the antibody or binding fragment thereof according to the present invention comprises a HCVR which comprises the following three CDRs:

V_{H}-CDR1:
(SEQ ID NO: 1)
NYYIG;
V_{H}-CDR2:
(SEQ ID NO: 119)
DIFPGGGYTNSAEKFQG;
and
V_{H}-CDR3:
(SEQ ID NO: 3)
RNFDY.

In one embodiment, the antibody or binding fragment thereof according to the present invention comprises a light chain variable region (abbreviated herein as LCVR or V_{L}) which comprises at least one, preferably at least two, more preferably the following three complementary-determining regions (CDRs):

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:
X₁₁ being selected from Ser (S) and Arg (R);
X₁₂ being absent or being selected from Asn (N), Ser (S) and Gly (G);
X₁₃ being selected from Asn (N) and Ala (A); or X₁₃ being any amino acid but Ala (A) or Asn (N);
X₁₄ being selected from Len (L), Ser (S) and Arg (R);
X₁₅ being selected from Pro (P), Ala (A) and Gln (Q);
X₁₆ being selected from Ser (S) and Thr (T); and
X₁₇ being selected from Gln (Q) and His (H).

In one embodiment, the antibody or binding fragment thereof according to the present invention comprises a LCVR which comprises the following three CDRs:

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:
X₁₁ being selected from Ser (S) and Arg (R);
X₁₂ being absent or being selected from Asn (N), Ser (S) and Gly (G);
X₁₃ being selected from Asn (N) and Ala (A); or X₁₃ being any amino acid but Ala (A) or Asn (N);
X₁₄ being selected from Len (L), Ser (S) and Arg (R);
X₁₅ being selected from Pro (P), Ala (A) and Gln (Q);
X₁₆ being selected from Ser (S) and Thr (T); and
X₁₇ being selected from Gln (Q) and His (H).

In one embodiment, the antibody or binding fragment thereof according to the present invention comprises a LCVR which comprises at least one, preferably at least two, more preferably the following three complementary-determining regions (CDRs):

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:
X₁₁ being Ser (S);
X₁₂ being absent or being selected from Asn (N), Ser (S) and Gly (G);
X₁₃ being Asn (N); or X₁₃ being any amino acid but Ala (A) or Asn (N);
X₁₄ being Len (L);
X₁₅ being Pro (P);
X₁₆ being Ser (S); and
X₁₇ being Gln (Q).

In one embodiment, the antibody or binding fragment thereof according to the present invention comprises a LCVR which comprises the following three CDRs:

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:
X₁₁ being Ser (S);
X₁₂ being absent or being selected from Asn (N), Ser (S) and Gly (G);
X₁₃ being Asn (N); or X₁₃ being any amino acid but Ala (A) or Asn (N);
X₁₄ being Len (L);
X₁₅ being Pro (P);
X₁₆ being Ser (S); and
X₁₇ being Gln (Q).

In one embodiment, the antibody or binding fragment thereof according to the present invention comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 15)
SASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 15)
SASSSVSYMH;
V_{L}-CDR2:
(SEQ ID NO: 16)
NTSNLPS;
and
V_{L}-CDR3:
(SEQ ID NO: 17)
QQRSSYPLTF.

In one embodiment, the antibody or binding fragment thereof according to the present invention comprises a LCVR which comprises the following three CDRs:

V_{L}-CDR1:
(SEQ ID NO: 15)
SASSSVSYMH;
V_{L}-CDR2:
(SEQ ID NO: 16)
NTSNLPS;
and
V_{L}-CDR3:
(SEQ ID NO: 17)
QQRSSYPLTF.

In one embodiment, the antibody or binding fragment thereof according to the present invention comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 15)
SASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR1:
(SEQ ID NO: 16)
NTSNLPS;
and
V_{L}-CDR1:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 16)
NTSNLPS;
and
V_{L}-CDR3:
(SEQ ID NO: 17)
QQRSSYPLTF.

In one embodiment, the antibody or binding fragment thereof according to the present invention comprises a LCVR which comprises the following three CDRs:

(SEQ ID NO: 18)
V_{L}-CDR1: RASSSVSYMH;
(SEQ ID NO: 16)
V_{L}-CDR2: NTSNLPS;
and
(SEQ ID NO: 17)
V_{L}-CDR3: QQRSSYPLTF.

In one embodiment, the antibody or binding fragment thereof according to the present invention comprises a LCVR which comprises at least one, preferably at least two, more preferably the following three CDRs:

(SEQ ID NO: 18)
V_{L}-CDR1: RASSSVS-X₁₂-YMH;
(SEQ ID NO: 16)
V_{L}-CDR2: NTSNLPS;
and
(SEQ ID NO: 17)
V_{L}-CDR3: QQRSSYPLTF,

with:
X₁₂ being selected from Asn (N), Ser (S) and Gly (G).

In one embodiment, the antibody or binding fragment thereof according to the present invention comprises a LCVR which comprises the following three CDRs:

(SEQ ID NO: 18)
V_{L}-CDR1: RASSSVS-X₁₂-YMH;
(SEQ ID NO: 16)
V_{L}-CDR2: NTSNLPS;
and
(SEQ ID NO: 17)
V_{L}-CDR3: QQRSSYPLTF,

with:
X₁₂ being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 19)
NTSNSPS;
and
V_{L}-CDR3:
(SEQ ID NO: 17)
QQRSSYPLTF.

In one embodiment, the antibody or binding fragment thereof according to the present invention comprises a LCVR which comprises the following three CDRs:

V_{L}-CDR1:
(SEQ ID NO: 18)
RASSSVSYMH;
V_{L}-CDR2:
(SEQ ID NO: 19)
NTSNSPS;
and
V_{L}-CDR3:
(SEQ ID NO: 17)
QQRSSYPLTF.

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 20)
ATSNLQS;
and
V_{L}-CDR3:
(SEQ ID NO: 17)
QQRSSYPLTF.

In one embodiment, the antibody or binding fragment thereof according to the present invention comprises a LCVR which comprises the following three CDRs:

V_{L}-CDR1:
(SEQ ID NO: 18)
RASSSVSYMH;
V_{L}-CDR2:
(SEQ ID NO: 20)
ATSNLQS;
and
V_{L}-CDR3:
(SEQ ID NO: 17)
QQRSSYPLTF.

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 21)
HQRSSYPLTF,

with:
X₁₂ being absent or being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 21)
HQRSSYPLTF,

with:
X₁₂ being absent or being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 19)
NTSNSPS;
and
V_{L}-CDR3:
(SEQ ID NO: 21)
HQRSSYPLTF.

In one embodiment, the antibody or binding fragment thereof according to the present invention comprises a LCVR which comprises the following three CDRs:

V_{L}-CDR1:
(SEQ ID NO: 18)
RASSSVSYMH;
V_{L}-CDR2:
(SEQ ID NO: 19)
NTSNSPS;
and
V_{L}-CDR3:
(SEQ ID NO: 21)
HQRSSYPLTF.

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 21)
HQRSSYPLTF,

with:
X₁₂ being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 21)
HQRSSYPLTF,

with:
X₁₂ being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 22)
NTSNRAT;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 22)
NTSNRAT;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 22)
NTSNRAT;
and
V_{L}-CDR3:
(SEQ ID NO: 17)
QQRSSYPLTF.

In one embodiment, the antibody or binding fragment thereof according to the present invention comprises a LCVR which comprises the following three CDRs:

V_{L}-CDR1:
(SEQ ID NO: 18)
RASSSVSYMH;
V_{L}-CDR2:
(SEQ ID NO: 22)
NTSNRAT;
and
V_{L}-CDR3:
(SEQ ID NO: 17)
QQRSSYPLTF.

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 22)
NTSNRAT;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 22)
NTSNRAT;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 111)
ATSNLPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 111)
ATSNLPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 111)
ATSNLPS;
and
V_{L}-CDR3:
(SEQ ID NO: 17)
QQRSSYPLTF.

In one embodiment, the antibody or binding fragment thereof according to the present invention comprises a LCVR which comprises the following three CDRs:

V_{L}-CDR1:
(SEQ ID NO: 18)
RASSSVSYMH;
V_{L}-CDR2:
(SEQ ID NO: 111)
ATSNLPS;
and
V_{L}-CDR3:
(SEQ ID NO: 17)
QQRSSYPLTF.

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 111)
ATSNLPS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 111)
ATSNLPS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

with:
X₁₂ being absent or being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

In one embodiment, the antibody or binding fragment thereof according to the present invention comprises a LCVR which comprises the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

In one embodiment, the antibody or binding fragment thereof according to the present invention comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

with:
X₁₂ being selected from Asn (N), Ser (S) and Gly (G).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF,

with:
X₁₂ being absent or being selected from Asn (N), Ser (S) and Gly (G), and
X₁₃ being any amino acid but Ala (A) or Asn (N).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF,

with:
X₁₂ being absent or being selected from Asn (N), Ser (S) and Gly (G), and
X₁₃ being any amino acid but Ala (A) or Asn (N).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF.

with:
X₁₃ being any amino acid but Ala (A) or Asn (N).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF.

with:
X₁₃ being any amino acid but Ala (A) or Asn (N).

In one embodiment, the antibody or binding fragment thereof according to the present invention comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF,

with:
X₁₂ being absent or being selected from Asn (N), Ser (S) and Gly (G), and
X₁₃ being any amino acid but Ala (A) or Asn (N).

In one embodiment, the antibody or binding fragment thereof according to the present invention comprises a LCVR which comprises the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF,

with:
X₁₂ being absent or being selected from Asn (N), Ser (S) and Gly (G), and
X₁₃ being any amino acid but Ala (A) or Asn (N).

In one embodiment, the antibody or binding fragment thereof according to the present invention comprises:
a HCVR which comprises at least one, preferably at least two, more preferably the following three CDRs:
   V_{H}-CDR1: NYYIG (SEQ ID NO: 1);
   V_{H}-CDR2: X₁-IF-X₂-GG-X₃-Y-X₄-N-X₅-X₆-X₇-X₈-X₉-X₁₀-G (SEQ ID NO: 2); and
   V_{H}-CDR3: RNFDY (SEQ ID NO: 3); and
a LCVR which comprises at least one, preferably at least two, more preferably the following three CDRs:

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X17-QRSSYPLTF,

with:
X₁ being selected from Asp (D), Ile (I) and Arg (R);
X₂ being selected from Pro (P) and Ser (S);
X₃ being selected from Asp (D), Ser (S) and Gly (G);
X₄ being selected from Ala (A) and Thr (T);
X₅ being selected from Ser (S) and Tyr (Y);
X₆ being selected from Asn (N), Ala (A) and Ser (S);
X₇ being selected from Glu (E), Asp (D), Pro (P) and Gln (Q);
X₈ being selected from Lys (K) and Ser (S);
X₉ being selected from Phe (F) and Val (V);
X₁₀ being selected from Lys (K) and Gln (Q);
X₁₁ being selected from Ser (S) and Arg (R);
X₁₂ being absent or being selected from Asn (N), Ser (S) and Gly (G);
X₁₃ being selected from Asn (N) and Ala (A); or X₁₃ being any amino acid but Ala (A) or Asn (N);
X₁₄ being selected from Len (L), Ser (S) and Arg (R);
X₁₅ being selected from Pro (P), Ala (A) and Gln (Q);
X₁₆ being selected from Ser (S) and Thr (T); and
X₁₇ being selected from Gln (Q) and His (H).

In one embodiment, the antibody or binding fragment thereof according to the present invention comprises:
a HCVR which comprises the following three CDRs:
   V_{H}-CDR1: NYYIG (SEQ ID NO: 1);
   V_{H}-CDR2: X₁-IF-X₂-GG-X₃-Y-X₄-N-X₅-X₆-X₇-X₈-X₉-X₁₀-G (SEQ ID NO: 2); and
   V_{H}-CDR3: RNFDY (SEQ ID NO: 3); and
a LCVR which comprises the following three CDRs:

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:
X₁ being selected from Asp (D), Ile (I) and Arg (R);
X₂ being selected from Pro (P) and Ser (S);
X₃ being selected from Asp (D), Ser (S) and Gly (G);
X₄ being selected from Ala (A) and Thr (T);
X₅ being selected from Ser (S) and Tyr (Y);
X₆ being selected from Asn (N), Ala (A) and Ser (S);
X₇ being selected from Glu (E), Asp (D), Pro (P) and Gln (Q);
X₈ being selected from Lys (K) and Ser (S);
X₉ being selected from Phe (F) and Val (V);
X₁₀ being selected from Lys (K) and Gln (Q);
X₁₁ being selected from Ser (S) and Arg (R);
X₁₂ being absent or being selected from Asn (N), Ser (S) and Gly (G);
X₁₃ being selected from Asn (N) and Ala (A); or X₁₃ being any amino acid but Ala (A) or Asn (N);
X₁₄ being selected from Len (L), Ser (S) and Arg (R);
X₁₅ being selected from Pro (P), Ala (A) and Gln (Q);
X₁₆ being selected from Ser (S) and Thr (T); and
X₁₇ being selected from Gln (Q) and His (H).

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being as defined in Table 2.

In one embodiment, the antibody or binding fragment thereof according to the present invention comprises a combination of (i) three HCVR's CDRs and (ii) three LCVR's CDRs, said combination being as defined in Table 2.

TABLE 2
Preferred combinations of HCVR's and LCVR's CDRs. The CDRs are
defined by their SEQ ID NOs (with, wherever applicable, X₁₂ being absent or being
selected from Asn (N), Ser (S) and Gly (G); and X₁₃ being any amino acid but
Ala (A) or Asn (N)).

| CDRs′ | V_{H}- | V_{H}- | V_{H}- | V_{L}- | V_{L}- | V_{L}- |
|---|---|---|---|---|---|---|
| combination # | CDR1 | CDR2 | CDR3 | CDR1 | CDR2 | CDR3 |
| #1 | 1 | 4 | 3 | 15 | 16 | 17 |
| #2 | 1 | 4 | 3 | 18 | 16 | 17 |
| #3 | 1 | 4 | 3 | 18 | 19 | 17 |
| #4 | 1 | 4 | 3 | 18 | 20 | 17 |
| #5 | 1 | 4 | 3 | 18 | 19 | 21 |
| #6 | 1 | 4 | 3 | 18 | 22 | 17 |
| #7 | 1 | 5 | 3 | 15 | 16 | 17 |
| #8 | 1 | 5 | 3 | 18 | 16 | 17 |
| #9 | 1 | 5 | 3 | 18 | 19 | 17 |
| #10 | 1 | 5 | 3 | 18 | 20 | 17 |
| #11 | 1 | 5 | 3 | 18 | 19 | 21 |
| #12 | 1 | 5 | 3 | 18 | 22 | 17 |
| #13 | 1 | 6 | 3 | 15 | 16 | 17 |
| #14 | 1 | 6 | 3 | 18 | 16 | 17 |
| #15 | 1 | 6 | 3 | 18 | 19 | 17 |
| #16 | 1 | 6 | 3 | 18 | 20 | 17 |
| #17 | 1 | 6 | 3 | 18 | 19 | 21 |
| #18 | 1 | 6 | 3 | 18 | 22 | 17 |
| #19 | 1 | 7 | 3 | 15 | 16 | 17 |
| #20 | 1 | 7 | 3 | 18 | 16 | 17 |
| #21 | 1 | 7 | 3 | 18 | 19 | 17 |
| #22 | 1 | 7 | 3 | 18 | 20 | 17 |
| #23 | 1 | 7 | 3 | 18 | 19 | 21 |
| #24 | 1 | 7 | 3 | 18 | 22 | 17 |
| #25 | 1 | 8 | 3 | 15 | 16 | 17 |
| #26 | 1 | 8 | 3 | 18 | 16 | 17 |
| #27 | 1 | 8 | 3 | 18 | 19 | 17 |
| #28 | 1 | 8 | 3 | 18 | 20 | 17 |
| #29 | 1 | 8 | 3 | 18 | 19 | 21 |
| #30 | 1 | 8 | 3 | 18 | 22 | 17 |
| #31 | 1 | 9 | 3 | 15 | 16 | 17 |
| #32 | 1 | 9 | 3 | 18 | 16 | 17 |
| #33 | 1 | 9 | 3 | 18 | 19 | 17 |
| #34 | 1 | 9 | 3 | 18 | 20 | 17 |
| #35 | 1 | 9 | 3 | 18 | 19 | 21 |
| #36 | 1 | 9 | 3 | 18 | 22 | 17 |
| #37 | 1 | 10 | 3 | 15 | 16 | 17 |
| #38 | 1 | 10 | 3 | 18 | 16 | 17 |
| #39 | 1 | 10 | 3 | 18 | 19 | 17 |
| #40 | 1 | 10 | 3 | 18 | 20 | 17 |
| #41 | 1 | 10 | 3 | 18 | 19 | 21 |
| #42 | 1 | 10 | 3 | 18 | 22 | 17 |
| #43 | 1 | 11 | 3 | 15 | 16 | 17 |
| #44 | 1 | 11 | 3 | 18 | 16 | 17 |
| #45 | 1 | 11 | 3 | 18 | 19 | 17 |
| #46 | 1 | 11 | 3 | 18 | 20 | 17 |
| #47 | 1 | 11 | 3 | 18 | 19 | 21 |
| #48 | 1 | 11 | 3 | 18 | 22 | 17 |
| #49 | 1 | 100 | 3 | 15 | 16 | 17 |
| #50 | 1 | 100 | 3 | 18 | 16 | 17 |
| #51 | 1 | 100 | 3 | 18 | 19 | 17 |
| #52 | 1 | 100 | 3 | 18 | 20 | 17 |
| #53 | 1 | 100 | 3 | 18 | 19 | 21 |
| #54 | 1 | 100 | 3 | 18 | 22 | 17 |
| #55 | 1 | 4 | 3 | 18 | 111 | 17 |
| #56 | 1 | 5 | 3 | 18 | 111 | 17 |
| #57 | 1 | 6 | 3 | 18 | 111 | 17 |
| #58 | 1 | 7 | 3 | 18 | 111 | 17 |
| #59 | 1 | 8 | 3 | 18 | 111 | 17 |
| #60 | 1 | 9 | 3 | 18 | 111 | 17 |
| #61 | 1 | 10 | 3 | 18 | 111 | 17 |
| #62 | 1 | 11 | 3 | 18 | 111 | 17 |
| #63 | 1 | 100 | 3 | 18 | 111 | 17 |
| #64 | 1 | 116 | 3 | 15 | 16 | 17 |
| #65 | 1 | 116 | 3 | 18 | 16 | 17 |
| #66 | 1 | 116 | 3 | 18 | 19 | 17 |
| #67 | 1 | 116 | 3 | 18 | 20 | 17 |
| #68 | 1 | 116 | 3 | 18 | 19 | 21 |
| #69 | 1 | 116 | 3 | 18 | 22 | 17 |
| #70 | 1 | 116 | 3 | 18 | 111 | 17 |
| #71 | 1 | 117 | 3 | 15 | 16 | 17 |
| #72 | 1 | 117 | 3 | 18 | 16 | 17 |
| #73 | 1 | 117 | 3 | 18 | 19 | 17 |
| #74 | 1 | 117 | 3 | 18 | 20 | 17 |
| #75 | 1 | 117 | 3 | 18 | 19 | 21 |
| #76 | 1 | 117 | 3 | 18 | 22 | 17 |
| #77 | 1 | 117 | 3 | 18 | 111 | 17 |
| #78 | 1 | 118 | 3 | 15 | 16 | 17 |
| #79 | 1 | 118 | 3 | 18 | 16 | 17 |
| #80 | 1 | 118 | 3 | 18 | 19 | 17 |
| #81 | 1 | 118 | 3 | 18 | 20 | 17 |
| #82 | 1 | 118 | 3 | 18 | 19 | 21 |
| #83 | 1 | 118 | 3 | 18 | 22 | 17 |
| #84 | 1 | 118 | 3 | 18 | 111 | 17 |
| #85 | 1 | 119 | 3 | 15 | 16 | 17 |
| #86 | 1 | 119 | 3 | 18 | 16 | 17 |
| #87 | 1 | 119 | 3 | 18 | 19 | 17 |
| #88 | 1 | 119 | 3 | 18 | 20 | 17 |
| #89 | 1 | 119 | 3 | 18 | 19 | 21 |
| #90 | 1 | 119 | 3 | 18 | 22 | 17 |
| #91 | 1 | 119 | 3 | 18 | 111 | 17 |
| #92 | 1 | 4 | 3 | 15 | 120 | 17 |
| #93 | 1 | 5 | 3 | 15 | 120 | 17 |
| #94 | 1 | 6 | 3 | 15 | 120 | 17 |
| #95 | 1 | 7 | 3 | 15 | 120 | 17 |
| #96 | 1 | 8 | 3 | 15 | 120 | 17 |
| #97 | 1 | 9 | 3 | 15 | 120 | 17 |
| #98 | 1 | 10 | 3 | 15 | 120 | 17 |
| #99 | 1 | 11 | 3 | 15 | 120 | 17 |
| #100 | 1 | 100 | 3 | 15 | 120 | 17 |
| #101 | 1 | 116 | 3 | 15 | 120 | 17 |
| #102 | 1 | 117 | 3 | 15 | 120 | 17 |
| #103 | 1 | 118 | 3 | 15 | 120 | 17 |
| #104 | 1 | 119 | 3 | 15 | 120 | 17 |
| #105 | 1 | 4 | 3 | 15 | 127 | 17 |
| #106 | 1 | 5 | 3 | 15 | 127 | 17 |
| #107 | 1 | 6 | 3 | 15 | 127 | 17 |
| #108 | 1 | 7 | 3 | 15 | 127 | 17 |
| #109 | 1 | 8 | 3 | 15 | 127 | 17 |
| #110 | 1 | 9 | 3 | 15 | 127 | 17 |
| #111 | 1 | 10 | 3 | 15 | 127 | 17 |
| #112 | 1 | 11 | 3 | 15 | 127 | 17 |
| #113 | 1 | 100 | 3 | 15 | 127 | 17 |
| #114 | 1 | 116 | 3 | 15 | 127 | 17 |
| #115 | 1 | 117 | 3 | 15 | 127 | 17 |
| #116 | 1 | 118 | 3 | 15 | 127 | 17 |
| #117 | 1 | 119 | 3 | 15 | 127 | 17 |

In one embodiment, any of V_{H}-CDR1, V_{H}-CDR2, V_{H}-CDR3, V_{L}-CDR1, V_{L}-CDR2 and/or V_{L}-CDR3 as defined hereinabove can be characterized as having 1, 2, 3, 4, 5 or more amino acids being substituted by a different amino acid.

In one embodiment, any of V_{H}-CDR1, V_{H}-CDR2, V_{H}-CDR3, V_{L}-CDR1, V_{L}-CDR2 and/or V_{L}-CDR3 as defined hereinabove can be characterized as having an amino acid sequence that shares at least 70%, 75%, 80%, 85%, 90%, 95%, 96%, 97%, 98%, 99% or more of identity with the particular CDR or sets of CDRs as defined hereinabove.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being selected from combinations #1, #2, #7, #14, #20, #26, #49, #50, #63, #65, #72, #79, #86 and #92 as defined in Table 2.

In one embodiment, the antibody or binding fragment thereof according to the present invention comprises a combination of (i) three HCVR's CDRs and (ii) three LCVR's CDRs, said combination being selected from combinations #1, #2, #7, #14, #20, #26, #49, #50, #63, #65, #72, #79, #86 and #92 as defined in Table 2.

In one embodiment, the antibody or binding fragment thereof according to the present invention comprises:
a HCVR which comprises the following three CDRs:
   V_{H}-CDR1 of sequence SEQ ID NO: 1;
   V_{H}-CDR2 selected from the group comprising or consisting of sequences SEQ ID NOs: 4, 5, 6, 7, 8, 100, 116, 117, 118 and 119; and
   V_{H}-CDR3 of sequence SEQ ID NO: 3; and
a LCVR which comprises the following three CDRs:
   V_{L}-CDR1 selected from the group comprising or consisting of sequences SEQ ID NOs: 15 and 18, wherein X₁₂ is absent or is selected from Asn (N), Ser (S) and Gly (G), preferably X₁₂ is absent;
   V_{L}-CDR2 selected from the group comprising or consisting of sequences SEQ ID NOs: 16, 111 and 120; and
   V_{L}-CDR3 of sequence SEQ ID NO: 17.

In one embodiment, the antibody or binding fragment thereof according to the present invention comprises:
a HCVR which comprises the following three CDRs:
   V_{H}-CDR1 of sequence SEQ ID NO: 1;
   V_{H}-CDR2 selected from the group comprising or consisting of sequences SEQ ID NOs: 4 and 5; and
   V_{H}-CDR3 of sequence SEQ ID NO: 3; and
a LCVR which comprises the following three CDRs:
   V_{L}-CDR1 of sequence SEQ ID NO: 15, wherein X₁₂ is absent or is selected from Asn (N), Ser (S) and Gly (G), preferably X₁₂ is absent;
   V_{L}-CDR2 of sequence SEQ ID NO: 16; and
   V_{L}-CDR3 of sequence SEQ ID NO: 17.

In one embodiment, the antibody or binding fragment thereof according to the present invention comprises:
a HCVR which comprises the following three CDRs:
   V_{H}-CDR1 of sequence SEQ ID NO: 1;
   V_{H}-CDR2 selected from the group comprising or consisting of sequences SEQ ID NOs: 4, 5, 6 and 100; and
   V_{H}-CDR3 of sequence SEQ ID NO: 3; and
a LCVR which comprises the following three CDRs:
   V_{L}-CDR1 selected from the group comprising or consisting of sequences SEQ ID NOs: 15 and 18, wherein X₁₂ is absent or is selected from Asn (N), Ser (S) and Gly (G), preferably X₁₂ is absent;
   V_{L}-CDR2 of sequence SEQ ID NO: 16; and
   V_{L}-CDR3 of sequence SEQ ID NO: 17.

In one embodiment, the antibody or binding fragment thereof according to the present invention comprises:
a HCVR which comprises the following three CDRs:
   V_{H}-CDR1 of sequence SEQ ID NO: 1;
   V_{H}-CDR2 selected from the group comprising or consisting of sequences SEQ ID NOs: 4, 6 and 100; and
   V_{H}-CDR3 of sequence SEQ ID NO: 3; and
a LCVR which comprises the following three CDRs:
   V_{L}-CDR1 selected from the group comprising or consisting of sequences SEQ ID NOs: 15 and 18, wherein X₁₂ is absent or is selected from Asn (N), Ser (S) and Gly (G), preferably X₁₂ is absent;
   V_{L}-CDR2 of sequence SEQ ID NO: 16; and
   V_{L}-CDR3 of sequence SEQ ID NO: 17.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #1 as defined in Table 2.

In one embodiment, the antibody or binding fragment thereof according to the present invention comprises a combination of (i) three HCVR's CDRs and (ii) three LCVR's CDRs, said combination being combination #1 as defined in Table 2.

In one embodiment, the antibody or binding fragment thereof according to the present invention comprises a combination of (i) three HCVR's CDRs set forth as SEQ ID NOs: 1, 4 and 3; and (ii) three LCVR's CDRs set forth as SEQ ID NOs: 15, 16 and 17; wherein X₁₂ in SEQ ID NOs: 15 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #2 as defined in Table 2.

In one embodiment, the antibody or binding fragment thereof according to the present invention comprises a combination of (i) three HCVR's CDRs and (ii) three LCVR's CDRs, said combination being combination #2 as defined in Table 2.

In one embodiment, the antibody or binding fragment thereof according to the present invention comprises a combination of (i) three HCVR's CDRs set forth as SEQ ID NOs: 1, 4 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #7 as defined in Table 2.

In one embodiment, the antibody or binding fragment thereof according to the present invention comprises a combination of (i) three HCVR's CDRs and (ii) three LCVR's CDRs, said combination being combination #7 as defined in Table 2.

In one embodiment, the antibody or binding fragment thereof according to the present invention comprises a combination of (i) three HCVR's CDRs set forth as SEQ ID NOs: 1, 5 and 3; and (ii) three LCVR's CDRs set forth as SEQ ID NOs: 15, 16 and 17; wherein X₁₂ in SEQ ID NOs: 15 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #14 as defined in Table 2.

In one embodiment, the antibody or binding fragment thereof according to the present invention comprises a combination of (i) three HCVR's CDRs and (ii) three LCVR's CDRs, said combination being combination #14 as defined in Table 2.

In one embodiment, the antibody or binding fragment thereof according to the present invention comprises a combination of (i) three HCVR's CDRs set forth as SEQ ID NOs: 1, 6 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #20 as defined in Table 2.

In one embodiment, the antibody or binding fragment thereof according to the present invention comprises a combination of (i) three HCVR's CDRs and (ii) three LCVR's CDRs, said combination being combination #20 as defined in Table 2.

In one embodiment, the antibody or binding fragment thereof according to the present invention comprises a combination of (i) three HCVR's CDRs set forth as SEQ ID NOs: 1, 7 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #26 as defined in Table 2.

In one embodiment, the antibody or binding fragment thereof according to the present invention comprises a combination of (i) three HCVR's CDRs and (ii) three LCVR's CDRs, said combination being combination #26 as defined in Table 2.

In one embodiment, the antibody or binding fragment thereof according to the present invention comprises a combination of (i) three HCVR's CDRs set forth as SEQ ID NOs: 1, 8 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #49 as defined in Table 2.

In one embodiment, the antibody or binding fragment thereof according to the present invention comprises a combination of (i) three HCVR's CDRs and (ii) three LCVR's CDRs, said combination being combination #49 as defined in Table 2.

In one embodiment, the antibody or binding fragment thereof according to the present invention comprises a combination of (i) three HCVR's CDRs set forth as SEQ ID NOs: 1, 100 and 3; and (ii) three LCVR's CDRs set forth as SEQ ID NOs: 15, 16 and 17; wherein X₁₂ in SEQ ID NOs: 15 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #50 as defined in Table 2.

In one embodiment, the antibody or binding fragment thereof according to the present invention comprises a combination of (i) three HCVR's CDRs and (ii) three LCVR's CDRs, said combination being combination #50 as defined in Table 2.

In one embodiment, the antibody or binding fragment thereof according to the present invention comprises a combination of (i) three HCVR's CDRs set forth as SEQ ID NOs: 1, 100 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #63 as defined in Table 2.

In one embodiment, the antibody or binding fragment thereof according to the present invention comprises a combination of (i) three HCVR's CDRs and (ii) three LCVR's CDRs, said combination being combination #63 as defined in Table 2.

In one embodiment, the antibody or binding fragment thereof according to the present invention comprises a combination of (i) three HCVR's CDRs set forth as SEQ ID NOs: 1, 100 and 3; and (ii) three LCVR's CDRs set forth as SEQ ID NOs: 18, 111 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #65 as defined in Table 2.

In one embodiment, the antibody or binding fragment thereof according to the present invention comprises a combination of (i) three HCVR's CDRs and (ii) three LCVR's CDRs, said combination being combination #65 as defined in Table 2.

In one embodiment, the antibody or binding fragment thereof according to the present invention comprises a combination of (i) three HCVR's CDRs set forth as SEQ ID NOs: 1, 116 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #72 as defined in Table 2.

In one embodiment, the antibody or binding fragment thereof according to the present invention comprises a combination of (i) three HCVR's CDRs and (ii) three LCVR's CDRs, said combination being combination #72 as defined in Table 2.

In one embodiment, the antibody or binding fragment thereof according to the present invention comprises a combination of (i) three HCVR's CDRs set forth as SEQ ID NOs: 1, 117 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #79 as defined in Table 2.

In one embodiment, the antibody or binding fragment thereof according to the present invention comprises a combination of (i) three HCVR's CDRs and (ii) three LCVR's CDRs, said combination being combination #79 as defined in Table 2.

In one embodiment, the antibody or binding fragment thereof according to the present invention comprises a combination of (i) three HCVR's CDRs set forth as SEQ ID NOs: 1, 118 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #86 as defined in Table 2.

In one embodiment, the antibody or binding fragment thereof according to the present invention comprises a combination of (i) three HCVR's CDRs and (ii) three LCVR's CDRs, said combination being combination #86 as defined in Table 2.

In one embodiment, the antibody or binding fragment thereof according to the present invention comprises a combination of (i) three HCVR's CDRs set forth as SEQ ID NOs: 1, 119 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof according to the present invention comprises a combination of (i) at least one, preferably at least two, more preferably three HCVR's CDRs and (ii) at least one, preferably at least two, more preferably three LCVR's CDRs, said combination being combination #92 as defined in Table 2.

In one embodiment, the antibody or binding fragment thereof according to the present invention comprises a combination of (i) three HCVR's CDRs and (ii) three LCVR's CDRs, said combination being combination #92 as defined in Table 2.

In one embodiment, the antibody or binding fragment thereof according to the present invention comprises a combination of (i) three HCVR's CDRs set forth as SEQ ID NOs: 1, 4 and 3; and (ii) three LCVR's CDRs set forth as SEQ ID NOs: 15, 120 and 17; wherein X₁₂ in SEQ ID NOs: 15 is absent or is selected from Asn (N), Ser (S) and Gly (G), preferably X₁₂ is absent.

In one embodiment, the antibody or binding fragment thereof according to the present invention comprises a HCVR which comprises at least one, preferably at least two, more preferably at least three, even more preferably the four following framework regions (FRs):

V_{H}-FR1:
(SEQ ID NO: 25)
QVQLQQSGAELVRPGTSVKMSCKAAGYTFT;
V_{H}-FR2:
(SEQ ID NO: 26)
WVKQRPGHGLEWIG;
V_{H}-FR3:
(SEQ ID NO: 27)
KATLTADTSSSTAYMQLSSLTSEDSAIYYCVR;
V_{H}-FR4:
(SEQ ID NO: 28)
WGQGTTLTVSS.

In one embodiment, the antibody or binding fragment thereof according to the present invention comprises a HCVR which comprises the four following FRs:

V_{H}-FR1:
(SEQ ID NO: 25)
QVQLQQSGAELVRPGTSVKMSCKAAGYTFT;
V_{H}-FR2:
(SEQ ID NO: 26)
WVKQRPGHGLEWIG;
V_{H}-FR3:
(SEQ ID NO: 27)
KATLTADTSSSTAYMQLSSLTSEDSAIYYCVR;
V_{H}-FR4:
(SEQ ID NO: 28)
WGQGTTLTVSS.

In one embodiment, the antibody or binding fragment thereof according to the present invention comprises a HCVR which comprises at least one, preferably at least two, more preferably at least three, even more preferably the four following FRs:

V_{H}-FR1:
(SEQ ID NO: 29)
QVQLVQSGAEVKKPGASVKVSCKASGYTFT;
V_{H}-FR2:
(SEQ ID NO: 30)
WVRQAPGQGLEWIG;
V_{H}-FR3:
(SEQ ID NO: 31)
RVTLTADTSISTAYMELSRLRSDDTVVYYCVR;
V_{H}-FR4:
(SEQ ID NO: 32)
WGQGTLVTVSS.

In one embodiment, the antibody or binding fragment thereof according to the present invention comprises a HCVR which comprises the four following FRs:

V_{H}-FR1:
(SEQ ID NO: 29)
QVQLVQSGAEVKKPGASVKVSCKASGYTFT;
V_{H}-FR2:
(SEQ ID NO: 30)
WVRQAPGQGLEWIG;
V_{H}-FR3:
(SEQ ID NO: 31)
RVTLTADTSISTAYMELSRLRSDDTVVYYCVR;
V_{H}-FR4:
(SEQ ID NO: 32)
WGQGTLVTVSS.

In one embodiment, the antibody or binding fragment thereof according to the present invention comprises a HCVR which comprises at least one, preferably at least two, more preferably at least three, even more preferably the four following FRs:

V_{H}-FR1:
(SEQ ID NO: 33)
EVQLVQSGAEVKKPGESLKISCKASGYTFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof according to the present invention comprises a HCVR which comprises the four following FRs:

V_{H}-FR1:
(SEQ ID NO: 33)
EVQLVQSGAEVKKPGESLKISCKASGYTFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof according to the present invention comprises a HCVR which comprises at least one, preferably at least two, more preferably at least three, even more preferably the four following FRs:

V_{H}-FR1:
(SEQ ID NO: 36)
QVQLVESGGGLVKPGGSLRLSCAASGYTFT;
V_{H}-FR2:
(SEQ ID NO: 37)
WIRQAPGKGLEWIG;
V_{H}-FR3:
(SEQ ID NO: 38)
RFTLSADTAKNSAYLQMNSLRAEDTAVYYCVR;
V_{H}-FR4:
(SEQ ID NO: 32)
WGQGTLVTVSS.

In one embodiment, the antibody or binding fragment thereof according to the present invention comprises a HCVR which comprises the four following FRs:

V_{H}-FR1:
(SEQ ID NO: 36)
QVQLVESGGGLVKPGGSLRLSCAASGYTFT;
V_{H}-FR2:
(SEQ ID NO: 37)
WIRQAPGKGLEWIG;
V_{H}-FR3:
(SEQ ID NO: 38)
RFTLSADTAKNSAYLQMNSLRAEDTAVYYCVR;
V_{H}-FR4:
(SEQ ID NO: 32)
WGQGTLVTVSS.

In one embodiment, the antibody or binding fragment thereof according to the present invention comprises a HCVR which comprises at least one, preferably at least two, more preferably at least three, even more preferably the four following FRs:

V_{H}-FR1:
(SEQ ID NO: 39)
EVQLVQSGAEVKKPGESLKISCKGSGYTFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof according to the present invention comprises a HCVR which comprises the four following FRs:

V_{H}-FR1:
(SEQ ID NO: 39)
EVQLVQSGAEVKKPGESLKISCKGSGYTFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof according to the present invention comprises a HCVR which comprises at least one, preferably at least two, more preferably at least three, even more preferably the four following FRs:

V_{H}-FR1:
(SEQ ID NO: 40)
QVQLVESGGGLVKPGGSLRLSCAASGFTFS;
V_{H}-FR2:
(SEQ ID NO: 37)
WIRQAPGKGLEWIG;
V_{H}-FR3:
(SEQ ID NO: 41)
RFTLSADTAKNSLYLQMNSLRAEDTAVYYCVR;
V_{H}-FR4:
(SEQ ID NO: 32)
WGQGTLVTVSS.

In one embodiment, the antibody or binding fragment thereof according to the present invention comprises a HCVR which comprises the four following FRs:

V_{H}-FR1:
(SEQ ID NO: 40)
QVQLVESGGGLVKPGGSLRLSCAASGFTFS;
V_{H}-FR2:
(SEQ ID NO: 37)
WIRQAPGKGLEWIG;
V_{H}-FR3:
(SEQ ID NO: 41)
RFTLSADTAKNSLYLQMNSLRAEDTAVYYCVR;
V_{H}-FR4:
(SEQ ID NO: 32)
WGQGTLVTVSS.

In one embodiment, the antibody or binding fragment thereof according to the present invention comprises a HCVR which comprises at least one, preferably at least two, more preferably at least three, even more preferably the four following FRs:

V_{H}-FR1:
(SEQ ID NO: 42)
EVQLVQSGAEVKKPGESLKISCKGSGYSFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof according to the present invention comprises a HCVR which comprises the four following FRs:

V_{H}-FR1:
(SEQ ID NO: 42)
EVQLVQSGAEVKKPGESLKISCKGSGYSFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof according to the present invention comprises a HCVR which comprises at least one, preferably at least two, more preferably at least three, even more preferably the four following FRs:

| | V_{H}-FR1: |
|---|---|
| (SEQ ID NO: 40) | |
| | QVQLVESGGGLVKPGGSLRLSCAASGFTFS; |
| | V_{H}-FR2: |
| (SEQ ID NO: 43) | |
| | WIRQAPGKGLEWVG; |
| | V_{H}-FR3: |
| (SEQ ID NO: 41) | |
| | RFTLSADTAKNSLYLQMNSLRAEDTAVYYCVR; |
| | V_{H}-FR4: |
| (SEQ ID NO: 32) | |
| | WGQGTLVTVSS. |

In one embodiment, the antibody or binding fragment thereof according to the present invention comprises a HCVR which comprises the four following FRs:

| | V_{H}-FR1: |
|---|---|
| (SEQ ID NO: 40) | |
| | QVQLVESGGGLVKPGGSLRLSCAASGFTFS; |
| | V_{H}-FR2: |
| (SEQ ID NO: 43) | |
| | WIRQAPGKGLEWVG; |
| | V_{H}-FR3: |
| (SEQ ID NO: 41) | |
| | RFTLSADTAKNSLYLQMNSLRAEDTAVYYCVR; |
| | V_{H}-FR4: |
| (SEQ ID NO: 32) | |
| | WGQGTLVTVSS. |

In one embodiment, the antibody or binding fragment thereof according to the present invention comprises a LCVR which comprises at least one, preferably at least two, more preferably at least three, even more preferably the four following FRs:

| | V_{L}-FR1: |
|---|---|
| (SEQ ID NO: 44) | |
| | QIVLTQSPTIMSASPGEKVTITC; |
| | V_{L}-FR2: |
| (SEQ ID NO: 45) | |
| | WFQQKTGTSPRLWIY; |
| | V_{L}-FR3: |
| (SEQ ID NO: 46) | |
| | GVPARFSGSGSGTS-X₁₈-SLTISRMEAEDAATYYC; |
| | V_{L}-FR4: |
| (SEQ ID NO: 47) | |
| | GAGTKLELK, |

with:
X₁₈ being selected from Tyr (Y) and Phe (F), preferably X₁₈ being Tyr (Y).

In one embodiment, the antibody or binding fragment thereof according to the present invention comprises a LCVR which comprises the four following FRs:

| | V_{L}-FR1: |
|---|---|
| (SEQ ID NO: 44) | |
| | QIVLTQSPTIMSASPGEKVTITC; |
| | V_{L}-FR2: |
| (SEQ ID NO: 45) | |
| | WFQQKTGTSPRLWIY; |
| | V_{L}-FR3: |
| (SEQ ID NO: 46) | |
| | GVPARFSGSGSGTS-X₁₈-SLTISRMEAEDAATYYC; |
| | V_{L}-FR4: |
| (SEQ ID NO: 47) | |
| | GAGTKLELK |

with:
X₁₈ being selected from Tyr (Y) and Phe (F), preferably X₁₈ being Tyr (Y).

In one embodiment, the antibody or binding fragment thereof according to the present invention comprises a LCVR which comprises at least one, preferably at least two, more preferably at least three, even more preferably the four following FRs:

| | V_{L}-FR1: |
|---|---|
| (SEQ ID NO: 48) | |
| | DIQLTQSPSFLSASVGDRVTITC; |
| | V_{L}-FR2: |
| (SEQ ID NO: 49) | |
| | WFQQKPGKAPKLWIY; |
| | V_{L}-FR3: |
| (SEQ ID NO: 50) | |
| | GVPSRFSGSGSGTE-X₁₈-TLTISSLQPEDFATYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Tyr (Y).

In one embodiment, the antibody or binding fragment thereof according to the present invention comprises a LCVR which comprises the four following FRs:

| | V_{L}-FR1: |
|---|---|
| (SEQ ID NO: 48) | |
| | DIQLTQSPSFLSASVGDRVTITC; |
| | V_{L}-FR2: |
| (SEQ ID NO: 49) | |
| | WFQQKPGKAPKLWIY; |
| | V_{L}-FR3: |
| (SEQ ID NO: 50) | |
| | GVPSRFSGSGSGTE-X₁₈-TLTISSLQPEDFATYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Tyr (Y).

In one embodiment, the antibody or binding fragment thereof according to the present invention comprises a LCVR which comprises at least one, preferably at least two, more preferably at least three, even more preferably the four following FRs:

| | V_{L}-FR1: |
|---|---|
| (SEQ ID NO: 52) | |
| | EIVLTQSPDFQSVTPKEKVTITC; |
| | V_{L}-FR2: |
| (SEQ ID NO: 53) | |
| | WFQQKPDQSPKLWIY; |
| | V_{L}-FR3: |
| (SEQ ID NO: 54) | |
| | GVPSRFSGSGSGTD-X₁₈-TLTINSLEAEDAATYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Tyr (Y).
In one embodiment, the antibody or binding fragment thereof according to the present invention comprises a LCVR which comprises the four following FRs:

| | V_{L}-FR1: |
|---|---|
| (SEQ ID NO: 52) | |
| | EIVLTQSPDFQSVTPKEKVTITC; |
| | V_{L}-FR2: |
| (SEQ ID NO: 53) | |
| | WFQQKPDQSPKLWIY; |
| | V_{L}-FR3: |
| (SEQ ID NO: 54) | |
| | GVPSRFSGSGSGTD-X₁₈-TLTINSLEAEDAATYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Tyr (Y).

In one embodiment, the antibody or binding fragment thereof according to the present invention comprises a LCVR which comprises at least one, preferably at least two, more preferably at least three, even more preferably the four following FRs:

| | V_{L}-FR1: |
|---|---|
| (SEQ ID NO: 55) | |
| | EIVLTQSPATLSLSPGERATLSC; |
| | V_{L}-FR2: |
| (SEQ ID NO: 56) | |
| | WFQQKPGQAPRLWIY; |
| | V_{L}-FR3: |
| (SEQ ID NO: 57) | |
| | GIPARFSGSGSGTD-X₁₈-TLTISSLEPEDFAVYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Tyr (Y).

In one embodiment, the antibody or binding fragment thereof according to the present invention comprises a LCVR which comprises the four following FRs:

| | V_{L}-FR1: |
|---|---|
| (SEQ ID NO: 55) | |
| | EIVLTQSPATLSLSPGERATLSC; |
| | V_{L}-FR2: |
| (SEQ ID NO: 56) | |
| | WFQQKPGQAPRLWIY; |
| | V_{L}-FR3: |
| (SEQ ID NO: 57) | |
| | GIPARFSGSGSGTD-X₁₈-TLTISSLEPEDFAVYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Tyr (Y).

In one embodiment, the antibody or binding fragment thereof according to the present invention comprises a LCVR which comprises at least one, preferably at least two, more preferably at least three, even more preferably the four following FRs:

| | V_{L}-FR1: |
|---|---|
| (SEQ ID NO: 48) | |
| | DIQLTQSPSFLSASVGDRVTITC; |
| | V_{L}-FR2: |
| (SEQ ID NO: 58) | |
| | WYQQKPGKAPKLWIY; |
| | V_{L}-FR3: |
| (SEQ ID NO: 50) | |
| | GVPSRFSGSGSGTE-X₁₈-TLTISSLQPEDFATYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Tyr (Y).

In one embodiment, the antibody or binding fragment thereof according to the present invention comprises a LCVR which comprises the four following FRs:

| | V_{L}-FR1: |
|---|---|
| (SEQ ID NO: 48) | |
| | DIQLTQSPSFLSASVGDRVTITC; |
| | V_{L}-FR2: |
| (SEQ ID NO: 58) | |
| | WYQQKPGKAPKLWIY; |
| | V_{L}-FR3: |
| (SEQ ID NO: 50) | |
| | GVPSRFSGSGSGTE-X₁₈-TLTISSLQPEDFATYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Tyr (Y).

In one embodiment, the antibody or binding fragment thereof according to the present invention comprises a LCVR which comprises at least one, preferably at least two, more preferably at least three, even more preferably the four following FRs:

| | V_{L}-FR1: |
|---|---|
| (SEQ ID NO: 48) | |
| | DIQLTQSPSFLSASVGDRVTITC; |
| | V_{L}-FR2: |
| (SEQ ID NO: 58) | |
| | WYQQKPGKAPKLWIY; |
| | V_{L}-FR3: |
| (SEQ ID NO: 50) | |
| | GVPSRFSGSGSGTE-X₁₈-TLTISSLQPEDFATYYC; |
| | V_{L}-FR4: |
| (SEQ ID NO: 51) | |
| | GGGTKVEIK, |

with:
   X₁₈ being selected from Tyr (Y) and Phe (F), preferably X₁₈ being Phe (F).

In one embodiment, the antibody or binding fragment thereof according to the present invention comprises a LCVR which comprises the four following FRs:

| | V_{L}-FR1: |
|---|---|
| (SEQ ID NO: 48) | |
| | DIQLTQSPSFLSASVGDRVTITC; |
| | V_{L}-FR2: |
| (SEQ ID NO: 58) | |
| | WYQQKPGKAPKLWIY; |
| | V_{L}-FR3: |
| (SEQ ID NO: 50) | |
| | GVPSRFSGSGSGTE-X₁₈-TLTISSLQPEDFATYYC; |
| | V_{L}-FR4: |
| (SEQ ID NO: 51) | |
| | GGGTKVEIK, |

with:
X₁₈ being selected from Tyr (Y) and Phe (F), preferably X₁₈ being Phe (F).

In one embodiment, the antibody or binding fragment thereof according to the present invention comprises a LCVR which comprises at least one, preferably at least two, more preferably at least three, even more preferably the four following FRs:

V_{L}-FR1:
(SEQ ID NO: 52)
EIVLTQSPDFQSVTPKEKVTITC;
V_{L}-FR2:
(SEQ ID NO: 59)
WYQQKPDQSPKLWIY;
V_{L}-FR3:
(SEQ ID NO: 54)
GVPSRFSGSGSGTD-X₁₈-TLTINSLEAEDAATYYC;
V_{L}-FR4:
(SEQ ID NO: 51)
GGGTKVEIK,

with:
X₁₈ being selected from Tyr (Y) and Phe (F), preferably X₁₈ being Phe (F).

In one embodiment, the antibody or binding fragment thereof according to the present invention comprises a LCVR which comprises the four following FRs:

V_{L}-FR1:
(SEQ ID NO: 52)
EIVLTQSPDFQSVTPKEKVTITC;
V_{L}-FR2:
(SEQ ID NO: 59)
WYQQKPDQSPKLWIY;
V_{L}-FR3:
(SEQ ID NO: 54)
GVPSRFSGSGSGTD-X₁₈-TLTINSLEAEDAATYYC;
V_{L}-FR4:
(SEQ ID NO: 51)
GGGTKVEIK,

with:
X₁₈ being selected from Tyr (Y) and Phe (F), preferably X₁₈ being Phe (F).

In one embodiment, the antibody or binding fragment thereof according to the present invention comprises a LCVR which comprises at least one, preferably at least two, more preferably at least three, even more preferably the four following FRs:

V_{L}-FR1:
(SEQ ID NO: 55)
EIVLTQSPATLSLSPGERATLSC;
V_{L}-FR2:
(SEQ ID NO: 60)
WYQQKPGQAPRLWIY;
V_{L}-FR3:
(SEQ ID NO: 57)
GIPARFSGSGSGTD-X₁₈-TLTISSLEPEDFAVYYC;
V_{L}-FR4:
(SEQ ID NO: 51)
GGGTKVEIK,

with:
X₁₈ being selected from Tyr (Y) and Phe (F), preferably X₁₈ being Phe (F).

In one embodiment, the antibody or binding fragment thereof according to the present invention comprises a LCVR which comprises the four following FRs:

V_{L}-FR1:
(SEQ ID NO: 55)
EIVLTQSPATLSLSPGERATLSC;
V_{L}-FR2:
(SEQ ID NO: 60)
WYQQKPGQAPRLWIY;
V_{L}-FR3:
(SEQ ID NO: 57)
GIPARFSGSGSGTD-X₁₈-TLTISSLEPEDFAVYYC;
V_{L}-FR4:
(SEQ ID NO: 51)
GGGTKVEIK,

with:
X₁₈ being selected from Tyr (Y) and Phe (F), preferably X₁₈ being Phe (F).

In one embodiment, the antibody or binding fragment thereof according to the present invention comprises a LCVR which comprises at least one, preferably at least two, more preferably at least three, even more preferably the four following FRs:

V_{L}-FR1:
(SEQ ID NO: 48)
DIQLTQSPSFLSASVGDRVTITC;
V_{L}-FR2:
(SEQ ID NO: 49)
WFQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:
X₁₈ being selected from Tyr (Y) and Phe (F), preferably X₁₈ being Phe (F).

In one embodiment, the antibody or binding fragment thereof according to the present invention comprises a LCVR which comprises the four following FRs:

V_{L}-FR1:
(SEQ ID NO: 48)
DIQLTQSPSFLSASVGDRVTITC;
V_{L}-FR2:
(SEQ ID NO: 49)
WFQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:
X₁₈ being selected from Tyr (Y) and Phe (F), preferably X₁₈ being Phe (F).

In one embodiment, the antibody or binding fragment thereof according to the present invention comprises a combination of (i) at least one, preferably at least two, more preferably at least three, even more preferably four HCVR's FRs and (ii) at least one, preferably at least two, more preferably at least three, even more preferably four LCVR's FRs, said combination being as defined in Table 3.

In one embodiment, the antibody or binding fragment thereof according to the present invention comprises a combination of (i) four HCVR's FRs and (ii) four LCVR's FRs, said combination being as defined in Table 3.

TABLE 3
Preferred combinations of HCVR's and
LCVR's FRs. The FRs are defined by
their SEQ ID NOs (with, wherever applicable,
X₁₈ being selected from Tyr (Y) and Phe (F)).

| FRs′ | | | | |
|---|---|---|---|---|
| combination # | V_{H}-FR1 | V_{H}-FR2 | V_{H}-FR3 | V_{H}-FR4 |
| #1 | 25 | 26 | 27 | 28 |
| #2 | 25 | 26 | 27 | 28 |
| #3 | 25 | 26 | 27 | 28 |
| #4 | 25 | 26 | 27 | 28 |
| #5 | 25 | 26 | 27 | 28 |
| #6 | 25 | 26 | 27 | 28 |
| #7 | 25 | 26 | 27 | 28 |
| #8 | 29 | 30 | 31 | 32 |
| #9 | 29 | 30 | 31 | 32 |
| #10 | 29 | 30 | 31 | 32 |
| #11 | 29 | 30 | 31 | 32 |
| #12 | 29 | 30 | 31 | 32 |
| #13 | 29 | 30 | 31 | 32 |
| #14 | 29 | 30 | 31 | 32 |
| #15 | 33 | 34 | 35 | 32 |
| #16 | 33 | 34 | 35 | 32 |
| #17 | 33 | 34 | 35 | 32 |
| #18 | 33 | 34 | 35 | 32 |
| #19 | 33 | 34 | 35 | 32 |
| #20 | 33 | 34 | 35 | 32 |
| #21 | 33 | 34 | 35 | 32 |
| #22 | 36 | 37 | 38 | 32 |
| #23 | 36 | 37 | 38 | 32 |
| #24 | 36 | 37 | 38 | 32 |
| #25 | 36 | 37 | 38 | 32 |
| #26 | 36 | 37 | 38 | 32 |
| #27 | 36 | 37 | 38 | 32 |
| #28 | 36 | 37 | 38 | 32 |
| #29 | 39 | 34 | 35 | 32 |
| #30 | 39 | 34 | 35 | 32 |
| #31 | 39 | 34 | 35 | 32 |
| #32 | 39 | 34 | 35 | 32 |
| #33 | 39 | 34 | 35 | 32 |
| #34 | 39 | 34 | 35 | 32 |
| #35 | 39 | 34 | 35 | 32 |
| #36 | 40 | 37 | 41 | 32 |
| #37 | 40 | 37 | 41 | 32 |
| #38 | 40 | 37 | 41 | 32 |
| #39 | 40 | 37 | 41 | 32 |
| #40 | 40 | 37 | 41 | 32 |
| #41 | 40 | 37 | 41 | 32 |
| #42 | 40 | 37 | 41 | 32 |
| #43 | 42 | 34 | 35 | 32 |
| #44 | 42 | 34 | 35 | 32 |
| #45 | 42 | 34 | 35 | 32 |
| #46 | 42 | 34 | 35 | 32 |
| #47 | 42 | 34 | 35 | 32 |
| #48 | 42 | 34 | 35 | 32 |
| #49 | 42 | 34 | 35 | 32 |
| #50 | 40 | 43 | 41 | 32 |
| #51 | 40 | 43 | 41 | 32 |
| #52 | 40 | 43 | 41 | 32 |
| #53 | 40 | 43 | 41 | 32 |
| #54 | 40 | 43 | 41 | 32 |
| #55 | 40 | 43 | 41 | 32 |
| #56 | 40 | 43 | 41 | 32 |
| #1 | 44 | 45 | 46 | 47 |
| #2 | 48 | 49 | 50 | 51 |
| #3 | 52 | 53 | 54 | 51 |
| #4 | 55 | 56 | 57 | 51 |
| #5 | 48 | 58 | 50 | 51 |
| #6 | 52 | 59 | 54 | 51 |
| #7 | 55 | 60 | 57 | 51 |
| #8 | 44 | 45 | 46 | 47 |
| #9 | 48 | 49 | 50 | 51 |
| #10 | 52 | 53 | 54 | 51 |
| #11 | 55 | 56 | 57 | 51 |
| #12 | 48 | 58 | 50 | 51 |
| #13 | 52 | 59 | 54 | 51 |
| #14 | 55 | 60 | 57 | 51 |
| #15 | 44 | 45 | 46 | 47 |
| #16 | 48 | 49 | 50 | 51 |
| #17 | 52 | 53 | 54 | 51 |
| #18 | 55 | 56 | 57 | 51 |
| #19 | 48 | 58 | 50 | 51 |
| #20 | 52 | 59 | 54 | 51 |
| #21 | 55 | 60 | 57 | 51 |
| #22 | 44 | 45 | 46 | 47 |
| #23 | 48 | 49 | 50 | 51 |
| #24 | 52 | 53 | 54 | 51 |
| #25 | 55 | 56 | 57 | 51 |
| #26 | 48 | 58 | 50 | 51 |
| #27 | 52 | 59 | 54 | 51 |
| #28 | 55 | 60 | 57 | 51 |
| #29 | 44 | 45 | 46 | 47 |
| #30 | 48 | 49 | 50 | 51 |
| #31 | 52 | 53 | 54 | 51 |
| #32 | 55 | 56 | 57 | 51 |
| #33 | 48 | 58 | 50 | 51 |
| #34 | 52 | 59 | 54 | 51 |
| #35 | 55 | 60 | 57 | 51 |
| #36 | 44 | 45 | 46 | 47 |
| #37 | 48 | 49 | 50 | 51 |
| #38 | 52 | 53 | 54 | 51 |
| #39 | 55 | 56 | 57 | 51 |
| #40 | 48 | 58 | 50 | 51 |
| #41 | 52 | 59 | 54 | 51 |
| #42 | 55 | 60 | 57 | 51 |
| #43 | 44 | 45 | 46 | 47 |
| #44 | 48 | 49 | 50 | 51 |
| #45 | 52 | 53 | 54 | 51 |
| #46 | 55 | 56 | 57 | 51 |
| #47 | 48 | 58 | 50 | 51 |
| #48 | 52 | 59 | 54 | 51 |
| #49 | 55 | 60 | 57 | 51 |
| #50 | 44 | 45 | 46 | 47 |
| #51 | 48 | 49 | 50 | 51 |
| #52 | 52 | 53 | 54 | 51 |
| #53 | 55 | 56 | 57 | 51 |
| #54 | 48 | 58 | 50 | 51 |
| #55 | 52 | 59 | 54 | 51 |
| #56 | 55 | 60 | 57 | 51 |

In one embodiment, any of V_{H}-FR1, V_{H}-FR2, V_{H}-FR3, V_{H}-FR4, V_{L}-FR1, V_{L}-FR2, V_{L}-FR3 and/or V_{L}-FR4 as defined hereinabove can be characterized as having 1, 2, 3, 4, 5, 6, 7, 8, 9, 10 or more amino acids being substituted by a different amino acid.

In one embodiment, any of V_{H}-FR1, V_{H}-FR2, V_{H}-FR3, V_{H}-FR4, V_{L}-FR1, V_{L}-FR2, V_{L}-FR3 and/or V_{L}-FR4 as defined hereinabove can be characterized as having an amino acid sequence that shares at least 70%, 75%, 80%, 85%, 90%, 95%, 96%, 97%, 98%, 99% or more of identity with the particular FR or sets of FRs as defined hereinabove.

In one embodiment, the antibody or binding fragment thereof according to the present invention comprises a HCVR comprising or consisting of:
a V_{H}-FR1 as described hereinabove,
a V_{H}-CDR1 as described hereinabove,
a V_{H}-FR2 as described hereinabove,
a V_{H}-CDR2 as described hereinabove,
a V_{H}-FR3 as described hereinabove,
a V_{H}-CDR3 as described hereinabove, and
a V_{H}-FR4 as described hereinabove.

In one embodiment, the antibody or binding fragment thereof according to the present invention comprises a HCVR comprising or consisting of:
a V_{H}-FR1 selected from SEQ ID NOs 25, 29, 33, 36, 39, 40 and 42;
a V_{H}-CDR1 selected from SEQ ID NO 1;
a V_{H}-FR2 selected from SEQ ID NOs 26, 30, 34, 37 and 43;
a V_{H}-CDR2 selected from SEQ ID NO 2;
a V_{H}-FR3 selected from SEQ ID NOs 27, 31, 35, 38 and 41;
a V_{H}-CDR3 selected from SEQ ID NO 3; and
a V_{H}-FR4 selected from SEQ ID NOs 28 and 32.

In one embodiment, the antibody or binding fragment thereof according to the present invention comprises a HCVR comprising or consisting of:
a V_{H}-FR1 selected from SEQ ID NOs 25, 29, 33, 36, 39, 40 and 42;
a V_{H}-CDR1 selected from SEQ ID NO 1;
a V_{H}-FR2 selected from SEQ ID NOs 26, 30, 34, 37 and 43;
a V_{H}-CDR2 selected from SEQ ID NOs 4, 5, 6, 7, 8, 9, 10, 11, 100, 116, 117, 118 and 119;
a V_{H}-FR3 selected from SEQ ID NOs 27, 31, 35, 38 and 41;
a V_{H}-CDR3 selected from SEQ ID NO 3; and
a V_{H}-FR4 selected from SEQ ID NOs 28 and 32.

In a preferred embodiment, the antibody or binding fragment thereof according to the present invention comprises a HCVR comprising or consisting of a combination of a V_{H}-FR1, a V_{H}-CDR1, a V_{H}-FR2, a V_{H}-CDR2, a V_{H}-FR3, a V_{H}-CDR3 and a V_{H}-FR4, said combination being as defined in Table 4.

TABLE 4
Preferred HCVR. The CDRs and FRs are defined by their SEQ ID NOs. The
penultimate column refers to the SEQ ID NOs of the whole HCVR.

| | V_{H}- | V_{H}- | V_{H}- | V_{H}- | V_{H}- | V_{H}- | V_{H}- | HCVR | |
|---|---|---|---|---|---|---|---|---|---|
| HCVR # | FR1 | CDR1 | FR2 | CDR2 | FR3 | CDR3 | FR4 | sequence | Name |
| #1 | 25 | 1 | 26 | 4 | 27 | 3 | 28 | 61 | Mouse VH |
| #2 | 29 | 1 | 30 | 4 | 31 | 3 | 32 | 62 | 12VHA |
| #3 | 33 | 1 | 34 | 4 | 35 | 3 | 32 | 63 | 551VHA |
| #4 | 36 | 1 | 37 | 5 | 38 | 3 | 32 | 64 | 311VHA |
| #5 | 29 | 1 | 30 | 6 | 31 | 3 | 32 | 65 | 12VHB |
| #6 | 39 | 1 | 34 | 7 | 35 | 3 | 32 | 66 | 551VHB |
| #7 | 40 | 1 | 37 | 8 | 41 | 3 | 32 | 67 | 311VHB |
| #8 | 29 | 1 | 30 | 9 | 31 | 3 | 32 | 68 | 12VHC |
| #9 | 42 | 1 | 34 | 10 | 35 | 3 | 32 | 69 | 551VHC |
| #10 | 40 | 1 | 43 | 11 | 41 | 3 | 32 | 70 | 331VHC |
| #11 | 29 | 1 | 30 | 100 | 31 | 3 | 32 | 101 | 12VHD |
| #12 | 29 | 1 | 30 | 116 | 31 | 3 | 32 | 121 | 12VHE |
| #13 | 29 | 1 | 30 | 117 | 31 | 3 | 32 | 122 | 12VHF |
| #14 | 29 | 1 | 30 | 118 | 31 | 3 | 32 | 123 | 12VHG |
| #15 | 29 | 1 | 30 | 119 | 31 | 3 | 32 | 124 | 12VHH |

In a preferred embodiment, the antibody or binding fragment thereof according to the present invention comprises a HCVR comprising or consisting of the sequence SEQ ID NO: 61; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 61.

SEQ ID NO: 61
QVQLQQSGAELVRPGTSVKMSCKAAGYTFTNYYIGWVKQRPGHGLEWIGD
IFPGGDYANSNEKFKGKATLTADTSSSTAYMQLSSLTSEDSAIYYCVRRN
FDYWGQGTTLTVSS

In a preferred embodiment, the antibody or binding fragment thereof according to the present invention comprises a HCVR comprising or consisting of the sequence SEQ ID NO: 62; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 62.

SEQ ID NO: 62
QVQLVQSGAEVKKPGASVKVSCKASGYTFTNYYIGWVRQAPGQGLEWIGD
IFPGGDYANSNEKFKGRVTLTADTSISTAYMELSRLRSDDTVVYYCVRRN
FDYWGQGTLVTVSS

In a preferred embodiment, the antibody or binding fragment thereof according to the present invention comprises a HCVR comprising or consisting of the sequence SEQ ID NO: 63; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 63.

SEQ ID NO: 63
EVQLVQSGAEVKKPGESLKISCKASGYTFTNYYIGWVRQMPGKGLEWIGD
IFPGGDYANSNEKFKGQVTLSADKSISTAYLQLSSLKASDTAMYYCVRRN
FDYWGQGTLVTVSS

In a preferred embodiment, the antibody or binding fragment thereof according to the present invention comprises a HCVR comprising or consisting of the sequence SEQ ID NO: 64; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 64.

SEQ ID NO: 64
QVQLVESGGGLVKPGGSLRLSCAASGYTFTNYYIGWIRQAPGKGLEWIGD
IFPGGDYANSNEKVKGRFTLSADTAKNSAYLQMNSLRAEDTAVYYCVRRN
FDYWGQGTLVTVSS

In a preferred embodiment, the antibody or binding fragment thereof according to the present invention comprises a HCVR comprising or consisting of the sequence SEQ ID NO: 65; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 65.

SEQ ID NO: 65
QVQLVQSGAEVKKPGASVKVSCKASGYTFTNYYIGWVRQAPGQGLEWIGD
IFPGGGYTNYAEKFQGRVTLTADTSISTAYMELSRLRSDDTVVYYCVRRN
FDYWGQGTLVTVSS

In a preferred embodiment, the antibody or binding fragment thereof according to the present invention comprises a HCVR comprising or consisting of the sequence SEQ ID NO: 66; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 66.

SEQ ID NO: 66
EVQLVQSGAEVKKPGESLKISCKGSGYTFTNYYIGWVRQMPGKGLEWIGD
IFPGGSYTNYSESFQGQVTLSADKSISTAYLQLSSLKASDTAMYYCVRRN
FDYWGQGTLVTVSS

In a preferred embodiment, the antibody or binding fragment thereof according to the present invention comprises a HCVR comprising or consisting of the sequence SEQ ID NO: 67; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 67.

SEQ ID NO: 67
QVQLVESGGGLVKPGGSLRLSCAASGFTFSNYYIGWIRQAPGKGLEWIGD
IFPGGSYTNYADSVKGRFTLSADTAKNSLYLQMNSLRAEDTAVYYCVRRN
FDYWGQGTLVTVSS

In a preferred embodiment, the antibody or binding fragment thereof according to the present invention comprises a HCVR comprising or consisting of the sequence SEQ ID NO: 68; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 68.

SEQ ID NO: 68
QVQLVQSGAEVKKPGASVKVSCKASGYTFTNYYIGWVRQAPGQGLEWIGR
IFPGGGYTNYAQKFQGRVTLTADTSISTAYMELSRLRSDDTVVYYCVRRN
FDYWGQGTLVTVSS

In a preferred embodiment, the antibody or binding fragment thereof according to the present invention comprises a HCVR comprising or consisting of the sequence SEQ ID NO: 69; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 69.

SEQ ID NO: 69
EVQLVQSGAEVKKPGESLKISCKGSGYSFTNYYIGWVRQMPGKGLEWIGI
IFPGGSYTNYSPSFQGQVTLSADKSISTAYLQLSSLKASDTAMYYCVRRN
FDYWGQGTLVTVSS

In a preferred embodiment, the antibody or binding fragment thereof according to the present invention comprises a HCVR comprising or consisting of the sequence SEQ ID NO: 70; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 70.

SEQ ID NO: 70
QVQLVESGGGLVKPGGSLRLSCAASGFTFSNYYIGWIRQAPGKGLEWVGD
IFSGGSYTNYADSVKGRFTLSADTAKNSLYLQMNSLRAEDTAVYYCVRRN
FDYWGQGTLVTVSS

In a preferred embodiment, the antibody or binding fragment thereof according to the present invention comprises a HCVR comprising or consisting of the sequence SEQ ID NO: 101; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 101.

SEQ ID NO: 101
QVQLVQSGAEVKKPGASVKVSCKASGYTFTNYYIGWVRQAPGQGLEWIGD
IFPGGDYTNYAEKFQGRVTLTADTSISTAYMELSRLRSDDTVVYYCVRRN
FDYWGQGTLVTVSS

In a preferred embodiment, the antibody or binding fragment thereof according to the present invention comprises a HCVR comprising or consisting of the sequence SEQ ID NO: 121; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 121.

SEQ ID NO: 121
QVQLVQSGAEVKKPGASVKVSCKASGYTFTNYYIGWVRQAPGQGLEWIGD
IFPGGGYANYAEKFQGRVTLTADTSISTAYMELSRLRSDDTVVYYCVRRN
FDYWGQGTLVTVSS

In a preferred embodiment, the antibody or binding fragment thereof according to the present invention comprises a HCVR comprising or consisting of the sequence SEQ ID NO: 122; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 122.

SEQ ID NO: 122
QVQLVQSGAEVKKPGASVKVSCKASGYTFTNYYIGWVRQAPGQGLEWIGD
IFPGGGYTNYAEKFKGRVTLTADTSISTAYMELSRLRSDDTVVYYCVRRN
FDYWGQGTLVTVSS

In a preferred embodiment, the antibody or binding fragment thereof according to the present invention comprises a HCVR comprising or consisting of the sequence SEQ ID NO: 123; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 123.

SEQ ID NO: 123
QVQLVQSGAEVKKPGASVKVSCKASGYTFTNYYIGWVRQAPGQGLEWIGD
IFPGGGYTNYNEKFQGRVTLTADTSISTAYMELSRLRSDDTVVYYCVRRN
FDYWGQGTLVTVSS

In a preferred embodiment, the antibody or binding fragment thereof according to the present invention comprises a HCVR comprising or consisting of the sequence SEQ ID NO: 124; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 124.

SEQ ID NO: 124
QVQLVQSGAEVKKPGASVKVSCKASGYTFTNYYIGWVRQAPGQGLEWIGD
IFPGGGYTNSAEKFQGRVTLTADTSISTAYMELSRLRSDDTVVYYCVRRN
FDYWGQGTLVTVSS

In one embodiment, the antibody or binding fragment thereof according to the present invention comprises a LCVR comprising or consisting of:
a V_{L}-FR1 as described hereinabove,
a V_{L}-CDR1 as described hereinabove,
a V_{L}-FR2 as described hereinabove,
a V_{L}-CDR2 as described hereinabove,
a V_{L}-FR3 as described hereinabove,
a V_{L}-CDR3 as described hereinabove, and
a V_{L}-FR4 as described hereinabove.

In one embodiment, the antibody or binding fragment thereof according to the present invention comprises a HCVR comprising or consisting of:
a V_{L}-FR1 selected from SEQ ID NOs 44, 48, 52 and 55;
a V_{L}-CDR1 selected from SEQ ID NO 12;
a V_{L}-FR2 selected from SEQ ID NOs 45, 49, 53, 56, 58, 59 and 60;
a V_{L}-CDR2 selected from SEQ ID NO 13;
a V_{L}-FR3 selected from SEQ ID NOs 46, 50, 54 and 57;
a V_{L}-CDR3 selected from SEQ ID NO 14; and
a V_{L}-FR4 selected from SEQ ID NOs 47 and 51.

In one embodiment, the antibody or binding fragment thereof according to the present invention comprises a HCVR comprising or consisting of:
a V_{L}-FR1 selected from SEQ ID NOs 44, 48, 52 and 55;
a V_{L}-CDR1 selected from SEQ ID NOs 15 and 18;
a V_{L}-FR2 selected from SEQ ID NOs 45, 49, 53, 56, 58, 59 and 60;
a V_{L}-CDR2 selected from SEQ ID NOs 16, 19, 20, 22, 111 and 120;
a V_{L}-FR3 selected from SEQ ID NOs 46, 50, 54 and 57;
a V_{L}-CDR3 selected from SEQ ID NOs 17 and 21; and
a V_{L}-FR4 selected from SEQ ID NOs 47 and 51.

In a preferred embodiment, the antibody or binding fragment thereof according to the present invention comprises a LCVR comprising or consisting of a combination of a V_{L}-FR1, a V_{L}-CDR1, a V_{L}-FR2, a V_{L}-CDR2, a V_{L}-FR3, a V_{L}-CDR3 and a V_{L}-FR4, said combination being as defined in Table 5.

TABLE 5
Preferred LCVR. The CDRs and FRs are defined by their SEQ ID NOs. The
penultimate column refers to the SEQ ID NOs of the whole LCVR (with a first sequence
number with X₁₂ being absent or being selected from Asn (N), Ser (S) and Gly (G); and
X₁₈ being selected from Tyr (Y) and Phe (F); and a second sequence number where
preferred X₁₂ and X₁₈ are defined).

| LCVR | V_{L}- | V_{L}- | V_{L}- | V_{L}- | V_{L}- | V_{L}- | V_{L}- | LCVR | |
|---|---|---|---|---|---|---|---|---|---|
| # | FR1 | CDR1 | FR2 | CDR2 | FR3 | CDR3 | FR4 | sequences | Name |
| #1 | 44 | 15 | 45 | 16 | 46 | 17 | 47 | 71/81 | Mouse VL |
| #2 | 48 | 15 | 49 | 16 | 50 | 17 | 51 | 72/82 | 19VLA |
| #3 | 52 | 15 | 53 | 16 | 54 | 17 | 51 | 73/83 | 621VLA |
| #4 | 55 | 15 | 56 | 16 | 57 | 17 | 51 | 74/84 | 311VLA |
| #5 | 48 | 18 | 58 | 16 | 50 | 17 | 51 | 75/85 | 19VLB |
| #6 | 52 | 18 | 59 | 19 | 54 | 17 | 51 | 76/86 | 621VLB |
| #7 | 55 | 18 | 56 | 16 | 57 | 17 | 51 | 77/87 | 311VLB |
| #8 | 48 | 18 | 58 | 20 | 50 | 17 | 51 | 78/88 | 19VLC |
| #9 | 52 | 18 | 59 | 19 | 54 | 21 | 51 | 79/89 | 621VLC |
| #10 | 55 | 18 | 60 | 22 | 57 | 17 | 51 | 80/90 | 311VLC |
| #11 | 48 | 18 | 49 | 16 | 50 | 17 | 51 | 102/103 | 19VLD |
| #12 | 48 | 18 | 58 | 111 | 50 | 17 | 51 | 112/113 | 19VLB |
| | | | | | | | | | “N50A” |
| #13 | 44 | 15 | 45 | 120 | 46 | 17 | 47 | 125/126 | Mouse |
| | | | | | | | | | “S52A” |
| #14 | 44 | 15 | 45 | 127 | 46 | 17 | 47 | 128/129 | 19VLB |
| | | | | | | | | | “N50X” |

In a one embodiment, the antibody or binding fragment thereof according to the present invention comprises a LCVR comprising or consisting of a combination of a V_{L}-FR1, a V_{L}-CDR1, a V_{L}-FR2, a V_{L}-CDR2, a V_{L}-FR3, a V_{L}-CDR3 and a V_{L}-FR4 as defined hereinabove, wherein X₁₈ is Phe (F) if X₁₂ is not absent (i.e., if X₁₂ is any of Asn (N), Ser (S) or Gly (G)). In a one embodiment, the antibody or binding fragment thereof according to the present invention comprises a LCVR comprising or consisting of a combination of a V_{L}-FR1, a V_{L}-CDR1, a V_{L}-FR2, a V_{L}-CDR2, a V_{L}-FR3, a V_{L}-CDR3 and a V_{L}-FR4 as defined hereinabove, wherein X₁₈ is selected from Tyr (Y) and Phe (F) if X₁₂ is absent.

In a preferred embodiment, the antibody or binding fragment thereof according to the present invention comprises a LCVR comprising or consisting of the sequence SEQ ID NO: 71, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 71.

SEQ ID NO: 71
QIYLTQSPTIMSASPGEKVTITCSASSSYS-X₁₂-YMHWFQQKTGTSPRL
WIYNTSNLPSGVPARFSGSGSGTSFSLTISRMEAEDAATYYCQQRSSYPL
TFGAGTKLELK

In a preferred embodiment, the antibody or binding fragment thereof according to the present invention comprises a LCVR comprising or consisting of the sequence SEQ ID NO: 72, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 72.

SEQ ID NO: 72
DIQLTQSPSFLSASVGDRVTITCSASSSVS-X₁₂-YMHWFQQKPGKAPKL
WIYNTSNLPSGVPSRFSGSGSGTEFTLTISSLQPEDFATYYCQQRSSYPL
TFGGGTKVEIK

In a preferred embodiment, the antibody or binding fragment thereof according to the present invention comprises a LCVR comprising or consisting of the sequence SEQ ID NO: 73, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 73.

SEQ ID NO: 73
EIVLTQSPDFQSVTPKEKVTITCSASSSVS-X₁₂-YMHWFQQKPDQSPKL
WIYNTSNLPSGVPSRFSGSGSGTDFTLTINSLEAEDAATYYCQQRSSYPL
TFGGGTKVEIK

In a preferred embodiment, the antibody or binding fragment thereof according to the present invention comprises a LCVR comprising or consisting of the sequence SEQ ID NO: 74, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 74.

SEQ ID NO: 74
EIVLTQSPATLSLSPGERATLSCSASSSYS-X₁₂-YMHWFQQKPGQAPRL
WIYNTSNLPSGIPARFSGSGSGTDFTLTISSLEPEDFAVYYCQQRSSYPL
TFGGGTKVEIK

In a preferred embodiment, the antibody or binding fragment thereof according to the present invention comprises a LCVR comprising or consisting of the sequence SEQ ID NO: 75, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 75.

SEQ ID NO: 75
DIQLTQSPSFLSASVGDRVTITCRASSSVS-X₁₂-YMHWYQQKPGKAPKL
WIYNTSNLPSGVPSRFSGSGSGTEFTLTISSLQPEDFATYYCQQRSSYPL
TFGGGTKVEIK

In a preferred embodiment, the antibody or binding fragment thereof according to the present invention comprises a LCVR comprising or consisting of the sequence SEQ ID NO: 76, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 76.

SEQ ID NO: 76
EIVLTQSPDFQSVTPKEKVTITCRASSSVS-X₁₂-YMHWYQQKPDQSPKL
WIYNTSNSPSGVPSRFSGSGSGTDFTLTINSLEAEDAATYYCQQRSSYPL
TFGGGTKVEIK

In a preferred embodiment, the antibody or binding fragment thereof according to the present invention comprises a LCVR comprising or consisting of the sequence SEQ ID NO: 77, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 77.

SEQ ID NO: 77
EIVLTQSPATLSLSPGERATLSCRASSSVS-X₁₂-YMHWFQQKPGQAPRL
WIYNTSNLPSGIPARFSGSGSGTDFTLTISSLEPEDFAVYYCQQRSSYPL
TFGGGTKVEIK

In a preferred embodiment, the antibody or binding fragment thereof according to the present invention comprises a LCVR comprising or consisting of the sequence SEQ ID NO: 78, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 78.

SEQ ID NO: 78
DIQLTQSPSFLSASVGDRVTITCRASSSVS-X₁₂-YMHWYQQKPGKAPKL
WIYATSNLQSGVPSRFSGSGSGTEFTLTISSLQPEDFATYYCQQRSSYPL
TFGGGTKVEIK

In a preferred embodiment, the antibody or binding fragment thereof according to the present invention comprises a LCVR comprising or consisting of the sequence SEQ ID NO: 79, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 79.

SEQ ID NO: 79
EIVLTQSPDFQSVTPKEKVTITCRASSSVS-X₁₂-YMHWYQQKPDQSPKL
WIYNTSNSPSGVPSRFSGSGSGTDFTLTINSLEAEDAATYYCHQRSSYPL
TFGGGTKVEIK

In a preferred embodiment, the antibody or binding fragment thereof according to the present invention comprises a LCVR comprising or consisting of the sequence SEQ ID NO: 80, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 80.

SEQ ID NO: 80
EIVLTQSPATLSLSPGERATLSCRASSSVS-X₁₂-YMHWYQQKPGQAPRL
WIYNTSNRATGIPARFSGSGSGTDFTLTISSLEPEDFAVYYCQQRSSYPL
TFGGGTKVEIK

In a preferred embodiment, the antibody or binding fragment thereof according to the present invention comprises a LCVR comprising or consisting of the sequence SEQ ID NO: 102, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 102.

SEQ ID NO: 102
DIQLTQSPSFLSASVGDRVTITCRASSSVS-X₁₂-YMHWFQQKPGKAPK
LWIYNTSNLPSGVPSRFSGSGSGTEFTLTISSLQPEDFATYYCQQRSS
YPLTFGGGTKVEIK

In a preferred embodiment, the antibody or binding fragment thereof according to the present invention comprises a LCVR comprising or consisting of the sequence SEQ ID NO: 112, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 112.

SEQ ID NO: 112
DIQLTQSPSFLSASVGDRVTITCRASSSVS-X₁₂-YMHWYQQKPGKAPK
LWIYATSNLPSGVPSRFSGSGSGTEFTLTISSLQPEDFATYYCQQRSS
YPLTFGGGTKVEIK

In a preferred embodiment, the antibody or binding fragment thereof according to the present invention comprises a LCVR comprising or consisting of the sequence SEQ ID NO: 125, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 125.

SEQ ID NO: 125
QIVLTQSPTIMSASPGEKVTITCSASSSYS-X₁₂-YMHWFQQKTGTSPR
LWIYNTANLPSGVPARFSGSGSGTSFSLTISRMEAEDAATYYCQQRSS
YPLTFGAGTKLELK

In a preferred embodiment, the antibody or binding fragment thereof according to the present invention comprises a LCVR comprising or consisting of the sequence SEQ ID NO: 128, with X₁₂ being selected from Asn (N), Ser (S) and Gly (G); and X₁₃ being any amino acid but Ala (A) or Asn (N); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 128.

SEQ ID NO: 128
DIQLTQSPSFLSASVGDRVTITCRASSSVS-X₁₂-YMHWYQQKPGKAPK
LWIY-X₁₃-TSNLPSGVPSRFSGSGSGTEFTLTISSLQPEDFATYYCQQ
RSSYPLTFGGGTKVEIK

In one embodiment, the antibody or binding fragment thereof according to the present invention comprises a LCVR comprising or consisting of sequences SEQ ID NO: 71-80, 102, 112, 125 or 128, wherein X₁₂ is absent.

In a preferred embodiment, the antibody or binding fragment thereof according to the present invention comprises a LCVR comprising or consisting of the sequence SEQ ID NO: 81; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 81.

SEQ ID NO: 81
QIVLTQSPTIMSASPGEKVTITCSASSSVSYMHWFQQKTGTSPRLWIYN
TSNLPSGVPARFSGSGSGTSYSLTISRMEAEDAATYYCQQRSSYPLTFG
AGTKLELK

In a preferred embodiment, the antibody or binding fragment thereof according to the present invention comprises a LCVR comprising or consisting of the sequence SEQ ID NO: 82; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 82.

SEQ ID NO: 82
DIQLTQSPSFLSASVGDRVTITCSASSSVSYMHWFQQKPGKAPKLWIYN
TSNLPSGVPSRFSGSGSGTEYTLTISSLQPEDFATYYCQQRSSYPLTFG
GGTKVEIK

In a preferred embodiment, the antibody or binding fragment thereof according to the present invention comprises a LCVR comprising or consisting of the sequence SEQ ID NO: 83; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 83.

SEQ ID NO: 83
EIVLTQSPDFQSVTPKEKVTITCSASSSVSYMHWFQQKPDQSPKLWIYN
TSNLPSGVPSRFSGSGSGTDYTLTINSLEAEDAATYYCQQRSSYPLTFG
GGTKVEIK

In a preferred embodiment, the antibody or binding fragment thereof according to the present invention comprises a LCVR comprising or consisting of the sequence SEQ ID NO: 84; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 84.

SEQ ID NO: 84
EIVLTQSPATLSLSPGERATLSCSASSSVSYMHWFQQKPGQAPRLWIYN
TSNLPSGIPARFSGSGSGTDYTLTISSLEPEDFAVYYCQQRSSYPLTFG
GGTKVEIK

In a preferred embodiment, the antibody or binding fragment thereof according to the present invention comprises a LCVR comprising or consisting of the sequence SEQ ID NO: 85; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 85.

SEQ ID NO: 85
DIQLTQSPSFLSASVGDRVTITCRASSSVSYMHWYQQKPGKAPKLWIYN
TSNLPSGVPSRFSGSGSGTEYTLTISSLQPEDFATYYCQQRSSYPLTFG
GGTKVEIK

In a preferred embodiment, the antibody or binding fragment thereof according to the present invention comprises a LCVR comprising or consisting of the sequence SEQ ID NO: 86; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 86.

SEQ ID NO: 86
EIVLTQSPDFQSVTPKEKVTITCRASSSVSYMHWYQQKPDQSPKLWIYN
TSNSPSGVPSRFSGSGSGTDFTLTINSLEAEDAATYYCQQRSSYPLTFG
GGTKVEIK

In a preferred embodiment, the antibody or binding fragment thereof according to the present invention comprises a LCVR comprising or consisting of the sequence SEQ ID NO: 87; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 87.

SEQ ID NO: 87
EIVLTQSPATLSLSPGERATLSCRASSSVSYMHWFQQKPGQAPRLWIYN
TSNLPSGIPARFSGSGSGTDYTLTISSLEPEDFAVYYCQQRSSYPLTFG
GGTKVEIK

In a preferred embodiment, the antibody or binding fragment thereof according to the present invention comprises a LCVR comprising or consisting of the sequence SEQ ID NO: 88; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 88.

SEQ ID NO: 88
DIQLTQSPSFLSASVGDRVTITCRASSSVSYMHWYQQKPGKAPKLWIYA
TSNLQSGVPSRFSGSGSGTEFTLTISSLQPEDFATYYCQQRSSYPLTFG
GGTKVEIK

In a preferred embodiment, the antibody or binding fragment thereof according to the present invention comprises a LCVR comprising or consisting of the sequence SEQ ID NO: 89; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 89.

SEQ ID NO: 89
EIVLTQSPDFQSVTPKEKVTITCRASSSVSYMHWYQQKPDQSPKLWIYN
TSNSPSGVPSRFSGSGSGTDFTLTINSLEAEDAATYYCHQRSSYPLTFG
GGTKVEIK

In a preferred embodiment, the antibody or binding fragment thereof according to the present invention comprises a LCVR comprising or consisting of the sequence SEQ ID NO: 90; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 90.

SEQ ID NO: 90
EIVLTQSPATLSLSPGERATLSCRASSSVSYMHWYQQKPGQAPRLWIYN
TSNRATGIPARFSGSGSGTDFTLTISSLEPEDFAVYYCQQRSSYPLTFG
GGTKVEIK

In a preferred embodiment, the antibody or binding fragment thereof according to the present invention comprises a LCVR comprising or consisting of the sequence SEQ ID NO: 103; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 103.

SEQ ID NO: 103
DIQLTQSPSFLSASVGDRVTITCRASSSVSSYMHWFQQKPGKAPKLWIY
NTSNLPSGVPSRFSGSGSGTEFTLTISSLQPEDFATYYCQQRSSYPLTF
GGGTKVEIK

In a preferred embodiment, the antibody or binding fragment thereof according to the present invention comprises a LCVR comprising or consisting of the sequence SEQ ID NO: 113; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 113.

SEQ ID NO: 113
DIQLTQSPSFLSASVGDRVTITCRASSSVSYMHWYQQKPGKAPKLWIYAT
SNLPSGVPSRFSGSGSGTEYTLTISSLQPEDFATYYCQQRSSYPLTFGGG
TKVEIK

In a preferred embodiment, the antibody or binding fragment thereof according to the present invention comprises a LCVR comprising or consisting of the sequence SEQ ID NO: 126; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 126.

SEQ ID NO: 126
QIVLTQSPTIMSASPGEKVTITCSASSSVSYMHWFQQKTGTSPRLWIYNT
ANLPSGVPARFSGSGSGTSYSLTISRMEAEDAATYYCQQRSSYPLTFGAG
TKLELK

In a preferred embodiment, the antibody or binding fragment thereof according to the present invention comprises a LCVR comprising or consisting of the sequence SEQ ID NO: 129, with X₁₃ being any amino acid but Ala (A) or Asn (N); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 129.

SEQ ID NO: 129
DIQLTQSPSFLSASVGDRVTITCRASSSVSYMHWYQQKPGKAPKLWIY-
X₁₃-TSNLPSGVPSRFSGSGSGTEYTLTISSLQPEDFATYYCQQRSSYPL
TFGGGTKVEIK

In one embodiment, the antibody or binding fragment thereof according to the present invention comprises:
a HCVR as defined hereinabove; and
a LCVR as defined hereinabove.

In one embodiment, the antibody or binding fragment thereof according to the present invention comprises:
a HCVR selected from SEQ ID NOs: 61, 62, 63, 64, 65, 66, 67, 68, 69, 70, 101, 121, 122, 123 and 124; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NOs: 61, 62, 63, 64, 65, 66, 67, 68, 69, 70, 101, 121, 122, 123 or 124; and
a LCVR selected from SEQ ID NOs: 71, 72, 73, 74, 75, 76, 77, 78, 79, 80, 102, 112, 125 and 128, with X₁₂ being selected from Asn (N), Ser (S) and Gly (G), and X₁₃ being any amino acid but Ala (A) or Asn (N); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NOs: 71, 72, 73, 74, 75, 76, 77, 78, 79, 80, 102, 112, 125 or 128.

In one embodiment, the antibody or binding fragment thereof according to the present invention comprises:
a HCVR selected from SEQ ID NOs: 61, 62, 63, 64, 65, 66, 67, 68, 69, 70, 101, 121, 122, 123 and 124; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NOs: 61, 62, 63, 64, 65, 66, 67, 68, 69, 70, 101, 121, 122, 123 or 124; and
a LCVR selected from SEQ ID NOs: 81, 82, 83, 84, 85, 86, 87, 88, 89, 90, 103, 113, 126 and 129, with X₁₃ being any amino acid but Ala (A) or Asn (N); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NOs: 81, 82, 83, 84, 85, 86, 87, 88, 89, 90, 103, 113, 126 or 129.

In one embodiment, the antibody or binding fragment thereof according to the present invention comprises a HCVR of SEQ ID NO: 61 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 62 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 63 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 64 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 65 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 66 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 67 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 68 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 69 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 70 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 101 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 121 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 122 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 123 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 71, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 72, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 73, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 74, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 75, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 76, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 77, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 78, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 79, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 80, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 102, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 112, with X₁₂ being selected from Asn (N), Ser (S) and Gly (G).

In one embodiment, the antibody or binding fragment thereof according to the present invention comprises a HCVR of SEQ ID NO: 124 and a LCVR of SEQ ID NO: 125, with X₁₂ being selected from Asn (N), Ser (S) and Gly (G).

In one embodiment, the antibody or binding fragment thereof according to the present invention comprises a HCVR of SEQ ID NO: 61 and a LCVR of SEQ ID NO: 81.

In one embodiment, the antibody or binding fragment thereof according to the present invention comprises a HCVR of SEQ ID NO: 61 and a LCVR of SEQ ID NO: 82.

In one embodiment, the antibody or binding fragment thereof according to the present invention comprises a HCVR of SEQ ID NO: 61 and a LCVR of SEQ ID NO: 83.

In one embodiment, the antibody or binding fragment thereof according to the present invention comprises a HCVR of SEQ ID NO: 61 and a LCVR of SEQ ID NO: 84.

In one embodiment, the antibody or binding fragment thereof according to the present invention comprises a HCVR of SEQ ID NO: 61 and a LCVR of SEQ ID NO: 85.

In one embodiment, the antibody or binding fragment thereof according to the present invention comprises a HCVR of SEQ ID NO: 61 and a LCVR of SEQ ID NO: 86.

In one embodiment, the antibody or binding fragment thereof according to the present invention comprises a HCVR of SEQ ID NO: 61 and a LCVR of SEQ ID NO: 87.

In one embodiment, the antibody or binding fragment thereof according to the present invention comprises a HCVR of SEQ ID NO: 61 and a LCVR of SEQ ID NO: 88.

In one embodiment, the antibody or binding fragment thereof according to the present invention comprises a HCVR of SEQ ID NO: 61 and a LCVR of SEQ ID NO: 89.

In one embodiment, the antibody or binding fragment thereof according to the present invention comprises a HCVR of SEQ ID NO: 61 and a LCVR of SEQ ID NO: 90.

In one embodiment, the antibody or binding fragment thereof according to the present invention comprises a HCVR of SEQ ID NO: 61 and a LCVR of SEQ ID NO: 103.

In one embodiment, the antibody or binding fragment thereof according to the present invention comprises a HCVR of SEQ ID NO: 61 and a LCVR of SEQ ID NO: 113.

In one embodiment, the antibody or binding fragment thereof according to the present invention comprises a HCVR of SEQ ID NO: 61 and a LCVR of SEQ ID NO: 126.

In one embodiment, the antibody or binding fragment thereof according to the present invention comprises a HCVR of SEQ ID NO: 62 and a LCVR of SEQ ID NO: 81.

In one embodiment, the antibody or binding fragment thereof according to the present invention comprises a HCVR of SEQ ID NO: 62 and a LCVR of SEQ ID NO: 82.

In one embodiment, the antibody or binding fragment thereof according to the present invention comprises a HCVR of SEQ ID NO: 62 and a LCVR of SEQ ID NO: 83.

In one embodiment, the antibody or binding fragment thereof according to the present invention comprises a HCVR of SEQ ID NO: 62 and a LCVR of SEQ ID NO: 84.

In one embodiment, the antibody or binding fragment thereof according to the present invention comprises a HCVR of SEQ ID NO: 62 and a LCVR of SEQ ID NO: 85.

In one embodiment, the antibody or binding fragment thereof according to the present invention comprises a HCVR of SEQ ID NO: 62 and a LCVR of SEQ ID NO: 86.

In one embodiment, the antibody or binding fragment thereof according to the present invention comprises a HCVR of SEQ ID NO: 62 and a LCVR of SEQ ID NO: 87.

In one embodiment, the antibody or binding fragment thereof according to the present invention comprises a HCVR of SEQ ID NO: 62 and a LCVR of SEQ ID NO: 88.

In one embodiment, the antibody or binding fragment thereof according to the present invention comprises a HCVR of SEQ ID NO: 62 and a LCVR of SEQ ID NO: 89.

In one embodiment, the antibody or binding fragment thereof according to the present invention comprises a HCVR of SEQ ID NO: 62 and a LCVR of SEQ ID NO: 90.

In one embodiment, the antibody or binding fragment thereof according to the present invention comprises a HCVR of SEQ ID NO: 62 and a LCVR of SEQ ID NO: 103.

In one embodiment, the antibody or binding fragment thereof according to the present invention comprises a HCVR of SEQ ID NO: 62 and a LCVR of SEQ ID NO: 113.

In one embodiment, the antibody or binding fragment thereof according to the present invention comprises a HCVR of SEQ ID NO: 62 and a LCVR of SEQ ID NO: 126.

In one embodiment, the antibody or binding fragment thereof according to the present invention comprises a HCVR of SEQ ID NO: 63 and a LCVR of SEQ ID NO: 81.

In one embodiment, the antibody or binding fragment thereof according to the present invention comprises a HCVR of SEQ ID NO: 63 and a LCVR of SEQ ID NO: 82.

In one embodiment, the antibody or binding fragment thereof according to the present invention comprises a HCVR of SEQ ID NO: 63 and a LCVR of SEQ ID NO: 83.

In one embodiment, the antibody or binding fragment thereof according to the present invention comprises a HCVR of SEQ ID NO: 63 and a LCVR of SEQ ID NO: 84.

In one embodiment, the antibody or binding fragment thereof according to the present invention comprises a HCVR of SEQ ID NO: 63 and a LCVR of SEQ ID NO: 85.

In one embodiment, the antibody or binding fragment thereof according to the present invention comprises a HCVR of SEQ ID NO: 63 and a LCVR of SEQ ID NO: 86.

In one embodiment, the antibody or binding fragment thereof according to the present invention comprises a HCVR of SEQ ID NO: 63 and a LCVR of SEQ ID NO: 87.

In one embodiment, the antibody or binding fragment thereof according to the present invention comprises a HCVR of SEQ ID NO: 63 and a LCVR of SEQ ID NO: 88.

In one embodiment, the antibody or binding fragment thereof according to the present invention comprises a HCVR of SEQ ID NO: 63 and a LCVR of SEQ ID NO: 89.

In one embodiment, the antibody or binding fragment thereof according to the present invention comprises a HCVR of SEQ ID NO: 63 and a LCVR of SEQ ID NO: 90.

In one embodiment, the antibody or binding fragment thereof according to the present invention comprises a HCVR of SEQ ID NO: 63 and a LCVR of SEQ ID NO: 103.

In one embodiment, the antibody or binding fragment thereof according to the present invention comprises a HCVR of SEQ ID NO: 63 and a LCVR of SEQ ID NO: 113.

In one embodiment, the antibody or binding fragment thereof according to the present invention comprises a HCVR of SEQ ID NO: 63 and a LCVR of SEQ ID NO: 126.

In one embodiment, the antibody or binding fragment thereof according to the present invention comprises a HCVR of SEQ ID NO: 64 and a LCVR of SEQ ID NO: 81.

In one embodiment, the antibody or binding fragment thereof according to the present invention comprises a HCVR of SEQ ID NO: 64 and a LCVR of SEQ ID NO: 82.

In one embodiment, the antibody or binding fragment thereof according to the present invention comprises a HCVR of SEQ ID NO: 64 and a LCVR of SEQ ID NO: 83.

In one embodiment, the antibody or binding fragment thereof according to the present invention comprises a HCVR of SEQ ID NO: 64 and a LCVR of SEQ ID NO: 84.

In one embodiment, the antibody or binding fragment thereof according to the present invention comprises a HCVR of SEQ ID NO: 64 and a LCVR of SEQ ID NO: 85.

In one embodiment, the antibody or binding fragment thereof according to the present invention comprises a HCVR of SEQ ID NO: 64 and a LCVR of SEQ ID NO: 86.

In one embodiment, the antibody or binding fragment thereof according to the present invention comprises a HCVR of SEQ ID NO: 64 and a LCVR of SEQ ID NO: 87.

In one embodiment, the antibody or binding fragment thereof according to the present invention comprises a HCVR of SEQ ID NO: 64 and a LCVR of SEQ ID NO: 88.

In one embodiment, the antibody or binding fragment thereof according to the present invention comprises a HCVR of SEQ ID NO: 64 and a LCVR of SEQ ID NO: 89.

In one embodiment, the antibody or binding fragment thereof according to the present invention comprises a HCVR of SEQ ID NO: 64 and a LCVR of SEQ ID NO: 90.

In one embodiment, the antibody or binding fragment thereof according to the present invention comprises a HCVR of SEQ ID NO: 64 and a LCVR of SEQ ID NO: 103.

In one embodiment, the antibody or binding fragment thereof according to the present invention comprises a HCVR of SEQ ID NO: 64 and a LCVR of SEQ ID NO: 113.

In one embodiment, the antibody or binding fragment thereof according to the present invention comprises a HCVR of SEQ ID NO: 64 and a LCVR of SEQ ID NO: 126.

In one embodiment, the antibody or binding fragment thereof according to the present invention comprises a HCVR of SEQ ID NO: 65 and a LCVR of SEQ ID NO: 81.

In one embodiment, the antibody or binding fragment thereof according to the present invention comprises a HCVR of SEQ ID NO: 65 and a LCVR of SEQ ID NO: 82.

In one embodiment, the antibody or binding fragment thereof according to the present invention comprises a HCVR of SEQ ID NO: 65 and a LCVR of SEQ ID NO: 83.

In one embodiment, the antibody or binding fragment thereof according to the present invention comprises a HCVR of SEQ ID NO: 65 and a LCVR of SEQ ID NO: 84.

In one embodiment, the antibody or binding fragment thereof according to the present invention comprises a HCVR of SEQ ID NO: 65 and a LCVR of SEQ ID NO: 85.

In one embodiment, the antibody or binding fragment thereof according to the present invention comprises a HCVR of SEQ ID NO: 65 and a LCVR of SEQ ID NO: 86.

In one embodiment, the antibody or binding fragment thereof according to the present invention comprises a HCVR of SEQ ID NO: 65 and a LCVR of SEQ ID NO: 87.

In one embodiment, the antibody or binding fragment thereof according to the present invention comprises a HCVR of SEQ ID NO: 65 and a LCVR of SEQ ID NO: 88.

In one embodiment, the antibody or binding fragment thereof according to the present invention comprises a HCVR of SEQ ID NO: 65 and a LCVR of SEQ ID NO: 89.

In one embodiment, the antibody or binding fragment thereof according to the present invention comprises a HCVR of SEQ ID NO: 65 and a LCVR of SEQ ID NO: 90.

In one embodiment, the antibody or binding fragment thereof according to the present invention comprises a HCVR of SEQ ID NO: 65 and a LCVR of SEQ ID NO: 103.

In one embodiment, the antibody or binding fragment thereof according to the present invention comprises a HCVR of SEQ ID NO: 65 and a LCVR of SEQ ID NO: 113.

In one embodiment, the antibody or binding fragment thereof according to the present invention comprises a HCVR of SEQ ID NO: 65 and a LCVR of SEQ ID NO: 126.

In one embodiment, the antibody or binding fragment thereof according to the present invention comprises a HCVR of SEQ ID NO: 66 and a LCVR of SEQ ID NO: 81.

In one embodiment, the antibody or binding fragment thereof according to the present invention comprises a HCVR of SEQ ID NO: 66 and a LCVR of SEQ ID NO: 82.

In one embodiment, the antibody or binding fragment thereof according to the present invention comprises a HCVR of SEQ ID NO: 66 and a LCVR of SEQ ID NO: 83.

In one embodiment, the antibody or binding fragment thereof according to the present invention comprises a HCVR of SEQ ID NO: 66 and a LCVR of SEQ ID NO: 84.

In one embodiment, the antibody or binding fragment thereof according to the present invention comprises a HCVR of SEQ ID NO: 66 and a LCVR of SEQ ID NO: 85.

In one embodiment, the antibody or binding fragment thereof according to the present invention comprises a HCVR of SEQ ID NO: 66 and a LCVR of SEQ ID NO: 86.

In one embodiment, the antibody or binding fragment thereof according to the present invention comprises a HCVR of SEQ ID NO: 66 and a LCVR of SEQ ID NO: 87.

In one embodiment, the antibody or binding fragment thereof according to the present invention comprises a HCVR of SEQ ID NO: 66 and a LCVR of SEQ ID NO: 88.

In one embodiment, the antibody or binding fragment thereof according to the present invention comprises a HCVR of SEQ ID NO: 66 and a LCVR of SEQ ID NO: 89.

In one embodiment, the antibody or binding fragment thereof according to the present invention comprises a HCVR of SEQ ID NO: 66 and a LCVR of SEQ ID NO: 90.

In one embodiment, the antibody or binding fragment thereof according to the present invention comprises a HCVR of SEQ ID NO: 66 and a LCVR of SEQ ID NO: 103.

In one embodiment, the antibody or binding fragment thereof according to the present invention comprises a HCVR of SEQ ID NO: 66 and a LCVR of SEQ ID NO: 113.

In one embodiment, the antibody or binding fragment thereof according to the present invention comprises a HCVR of SEQ ID NO: 66 and a LCVR of SEQ ID NO: 126.

In one embodiment, the antibody or binding fragment thereof according to the present invention comprises a HCVR of SEQ ID NO: 67 and a LCVR of SEQ ID NO: 81.

In one embodiment, the antibody or binding fragment thereof according to the present invention comprises a HCVR of SEQ ID NO: 67 and a LCVR of SEQ ID NO: 82.

In one embodiment, the antibody or binding fragment thereof according to the present invention comprises a HCVR of SEQ ID NO: 67 and a LCVR of SEQ ID NO: 83.

In one embodiment, the antibody or binding fragment thereof according to the present invention comprises a HCVR of SEQ ID NO: 67 and a LCVR of SEQ ID NO: 84.

In one embodiment, the antibody or binding fragment thereof according to the present invention comprises a HCVR of SEQ ID NO: 67 and a LCVR of SEQ ID NO: 85.

In one embodiment, the antibody or binding fragment thereof according to the present invention comprises a HCVR of SEQ ID NO: 67 and a LCVR of SEQ ID NO: 86.

In one embodiment, the antibody or binding fragment thereof according to the present invention comprises a HCVR of SEQ ID NO: 67 and a LCVR of SEQ ID NO: 87.

In one embodiment, the antibody or binding fragment thereof according to the present invention comprises a HCVR of SEQ ID NO: 67 and a LCVR of SEQ ID NO: 88.

In one embodiment, the antibody or binding fragment thereof according to the present invention comprises a HCVR of SEQ ID NO: 67 and a LCVR of SEQ ID NO: 89.

In one embodiment, the antibody or binding fragment thereof according to the present invention comprises a HCVR of SEQ ID NO: 67 and a LCVR of SEQ ID NO: 90.

In one embodiment, the antibody or binding fragment thereof according to the present invention comprises a HCVR of SEQ ID NO: 67 and a LCVR of SEQ ID NO: 103.

In one embodiment, the antibody or binding fragment thereof according to the present invention comprises a HCVR of SEQ ID NO: 67 and a LCVR of SEQ ID NO: 113.

In one embodiment, the antibody or binding fragment thereof according to the present invention comprises a HCVR of SEQ ID NO: 67 and a LCVR of SEQ ID NO: 126.

In one embodiment, the antibody or binding fragment thereof according to the present invention comprises a HCVR of SEQ ID NO: 68 and a LCVR of SEQ ID NO: 81.

In one embodiment, the antibody or binding fragment thereof according to the present invention comprises a HCVR of SEQ ID NO: 68 and a LCVR of SEQ ID NO: 82.

In one embodiment, the antibody or binding fragment thereof according to the present invention comprises a HCVR of SEQ ID NO: 68 and a LCVR of SEQ ID NO: 83.

In one embodiment, the antibody or binding fragment thereof according to the present invention comprises a HCVR of SEQ ID NO: 68 and a LCVR of SEQ ID NO: 84.

In one embodiment, the antibody or binding fragment thereof according to the present invention comprises a HCVR of SEQ ID NO: 68 and a LCVR of SEQ ID NO: 85.

In one embodiment, the antibody or binding fragment thereof according to the present invention comprises a HCVR of SEQ ID NO: 68 and a LCVR of SEQ ID NO: 86.

In one embodiment, the antibody or binding fragment thereof according to the present invention comprises a HCVR of SEQ ID NO: 68 and a LCVR of SEQ ID NO: 87.

In one embodiment, the antibody or binding fragment thereof according to the present invention comprises a HCVR of SEQ ID NO: 68 and a LCVR of SEQ ID NO: 88.

In one embodiment, the antibody or binding fragment thereof according to the present invention comprises a HCVR of SEQ ID NO: 68 and a LCVR of SEQ ID NO: 89.

In one embodiment, the antibody or binding fragment thereof according to the present invention comprises a HCVR of SEQ ID NO: 68 and a LCVR of SEQ ID NO: 90.

In one embodiment, the antibody or binding fragment thereof according to the present invention comprises a HCVR of SEQ ID NO: 68 and a LCVR of SEQ ID NO: 103.

In one embodiment, the antibody or binding fragment thereof according to the present invention comprises a HCVR of SEQ ID NO: 68 and a LCVR of SEQ ID NO: 113.

In one embodiment, the antibody or binding fragment thereof according to the present invention comprises a HCVR of SEQ ID NO: 68 and a LCVR of SEQ ID NO: 126.

In one embodiment, the antibody or binding fragment thereof according to the present invention comprises a HCVR of SEQ ID NO: 69 and a LCVR of SEQ ID NO: 81.

In one embodiment, the antibody or binding fragment thereof according to the present invention comprises a HCVR of SEQ ID NO: 69 and a LCVR of SEQ ID NO: 82.

In one embodiment, the antibody or binding fragment thereof according to the present invention comprises a HCVR of SEQ ID NO: 69 and a LCVR of SEQ ID NO: 83.

In one embodiment, the antibody or binding fragment thereof according to the present invention comprises a HCVR of SEQ ID NO: 69 and a LCVR of SEQ ID NO: 84.

In one embodiment, the antibody or binding fragment thereof according to the present invention comprises a HCVR of SEQ ID NO: 69 and a LCVR of SEQ ID NO: 85.

In one embodiment, the antibody or binding fragment thereof according to the present invention comprises a HCVR of SEQ ID NO: 69 and a LCVR of SEQ ID NO: 86.

In one embodiment, the antibody or binding fragment thereof according to the present invention comprises a HCVR of SEQ ID NO: 69 and a LCVR of SEQ ID NO: 87.

In one embodiment, the antibody or binding fragment thereof according to the present invention comprises a HCVR of SEQ ID NO: 69 and a LCVR of SEQ ID NO: 88.

In one embodiment, the antibody or binding fragment thereof according to the present invention comprises a HCVR of SEQ ID NO: 69 and a LCVR of SEQ ID NO: 89.

In one embodiment, the antibody or binding fragment thereof according to the present invention comprises a HCVR of SEQ ID NO: 69 and a LCVR of SEQ ID NO: 90.

In one embodiment, the antibody or binding fragment thereof according to the present invention comprises a HCVR of SEQ ID NO: 69 and a LCVR of SEQ ID NO: 103.

In one embodiment, the antibody or binding fragment thereof according to the present invention comprises a HCVR of SEQ ID NO: 69 and a LCVR of SEQ ID NO: 113.

In one embodiment, the antibody or binding fragment thereof according to the present invention comprises a HCVR of SEQ ID NO: 69 and a LCVR of SEQ ID NO: 126.

In one embodiment, the antibody or binding fragment thereof according to the present invention comprises a HCVR of SEQ ID NO: 70 and a LCVR of SEQ ID NO: 81.

In one embodiment, the antibody or binding fragment thereof according to the present invention comprises a HCVR of SEQ ID NO: 70 and a LCVR of SEQ ID NO: 82.

In one embodiment, the antibody or binding fragment thereof according to the present invention comprises a HCVR of SEQ ID NO: 70 and a LCVR of SEQ ID NO: 83.

In one embodiment, the antibody or binding fragment thereof according to the present invention comprises a HCVR of SEQ ID NO: 70 and a LCVR of SEQ ID NO: 84.

In one embodiment, the antibody or binding fragment thereof according to the present invention comprises a HCVR of SEQ ID NO: 70 and a LCVR of SEQ ID NO: 85.

In one embodiment, the antibody or binding fragment thereof according to the present invention comprises a HCVR of SEQ ID NO: 70 and a LCVR of SEQ ID NO: 86.

In one embodiment, the antibody or binding fragment thereof according to the present invention comprises a HCVR of SEQ ID NO: 70 and a LCVR of SEQ ID NO: 87.

In one embodiment, the antibody or binding fragment thereof according to the present invention comprises a HCVR of SEQ ID NO: 70 and a LCVR of SEQ ID NO: 88.

In one embodiment, the antibody or binding fragment thereof according to the present invention comprises a HCVR of SEQ ID NO: 70 and a LCVR of SEQ ID NO: 89.

In one embodiment, the antibody or binding fragment thereof according to the present invention comprises a HCVR of SEQ ID NO: 70 and a LCVR of SEQ ID NO: 90.

In one embodiment, the antibody or binding fragment thereof according to the present invention comprises a HCVR of SEQ ID NO: 70 and a LCVR of SEQ ID NO: 103.

In one embodiment, the antibody or binding fragment thereof according to the present invention comprises a HCVR of SEQ ID NO: 70 and a LCVR of SEQ ID NO: 113.

In one embodiment, the antibody or binding fragment thereof according to the present invention comprises a HCVR of SEQ ID NO: 70 and a LCVR of SEQ ID NO: 126.

In one embodiment, the antibody or binding fragment thereof according to the present invention comprises a HCVR of SEQ ID NO: 101 and a LCVR of SEQ ID NO: 81.

In one embodiment, the antibody or binding fragment thereof according to the present invention comprises a HCVR of SEQ ID NO: 101 and a LCVR of SEQ ID NO: 82.

In one embodiment, the antibody or binding fragment thereof according to the present invention comprises a HCVR of SEQ ID NO: 101 and a LCVR of SEQ ID NO: 83.

In one embodiment, the antibody or binding fragment thereof according to the present invention comprises a HCVR of SEQ ID NO: 101 and a LCVR of SEQ ID NO: 84.

In one embodiment, the antibody or binding fragment thereof according to the present invention comprises a HCVR of SEQ ID NO: 101 and a LCVR of SEQ ID NO: 85.

In one embodiment, the antibody or binding fragment thereof according to the present invention comprises a HCVR of SEQ ID NO: 101 and a LCVR of SEQ ID NO: 86.

In one embodiment, the antibody or binding fragment thereof according to the present invention comprises a HCVR of SEQ ID NO: 101 and a LCVR of SEQ ID NO: 87.

In one embodiment, the antibody or binding fragment thereof according to the present invention comprises a HCVR of SEQ ID NO: 101 and a LCVR of SEQ ID NO: 88.

In one embodiment, the antibody or binding fragment thereof according to the present invention comprises a HCVR of SEQ ID NO: 101 and a LCVR of SEQ ID NO: 89.

In one embodiment, the antibody or binding fragment thereof according to the present invention comprises a HCVR of SEQ ID NO: 101 and a LCVR of SEQ ID NO: 90.

In one embodiment, the antibody or binding fragment thereof according to the present invention comprises a HCVR of SEQ ID NO: 101 and a LCVR of SEQ ID NO: 103.

In one embodiment, the antibody or binding fragment thereof according to the present invention comprises a HCVR of SEQ ID NO: 101 and a LCVR of SEQ ID NO: 113.

In one embodiment, the antibody or binding fragment thereof according to the present invention comprises a HCVR of SEQ ID NO: 101 and a LCVR of SEQ ID NO: 126.

In one embodiment, the antibody or binding fragment thereof according to the present invention comprises a HCVR of SEQ ID NO: 121 and a LCVR of SEQ ID NO: 81.

In one embodiment, the antibody or binding fragment thereof according to the present invention comprises a HCVR of SEQ ID NO: 121 and a LCVR of SEQ ID NO: 82.

In one embodiment, the antibody or binding fragment thereof according to the present invention comprises a HCVR of SEQ ID NO: 121 and a LCVR of SEQ ID NO: 83.

In one embodiment, the antibody or binding fragment thereof according to the present invention comprises a HCVR of SEQ ID NO: 121 and a LCVR of SEQ ID NO: 84.

In one embodiment, the antibody or binding fragment thereof according to the present invention comprises a HCVR of SEQ ID NO: 121 and a LCVR of SEQ ID NO: 85.

In one embodiment, the antibody or binding fragment thereof according to the present invention comprises a HCVR of SEQ ID NO: 121 and a LCVR of SEQ ID NO: 86.

In one embodiment, the antibody or binding fragment thereof according to the present invention comprises a HCVR of SEQ ID NO: 121 and a LCVR of SEQ ID NO: 87.

In one embodiment, the antibody or binding fragment thereof according to the present invention comprises a HCVR of SEQ ID NO: 121 and a LCVR of SEQ ID NO: 88.

In one embodiment, the antibody or binding fragment thereof according to the present invention comprises a HCVR of SEQ ID NO: 121 and a LCVR of SEQ ID NO: 89.

In one embodiment, the antibody or binding fragment thereof according to the present invention comprises a HCVR of SEQ ID NO: 121 and a LCVR of SEQ ID NO: 90.

In one embodiment, the antibody or binding fragment thereof according to the present invention comprises a HCVR of SEQ ID NO: 121 and a LCVR of SEQ ID NO: 103.

In one embodiment, the antibody or binding fragment thereof according to the present invention comprises a HCVR of SEQ ID NO: 121 and a LCVR of SEQ ID NO: 113.

In one embodiment, the antibody or binding fragment thereof according to the present invention comprises a HCVR of SEQ ID NO: 121 and a LCVR of SEQ ID NO: 126.

In one embodiment, the antibody or binding fragment thereof according to the present invention comprises a HCVR of SEQ ID NO: 122 and a LCVR of SEQ ID NO: 81.

In one embodiment, the antibody or binding fragment thereof according to the present invention comprises a HCVR of SEQ ID NO: 122 and a LCVR of SEQ ID NO: 82.

In one embodiment, the antibody or binding fragment thereof according to the present invention comprises a HCVR of SEQ ID NO: 122 and a LCVR of SEQ ID NO: 83.

In one embodiment, the antibody or binding fragment thereof according to the present invention comprises a HCVR of SEQ ID NO: 122 and a LCVR of SEQ ID NO: 84.

In one embodiment, the antibody or binding fragment thereof according to the present invention comprises a HCVR of SEQ ID NO: 122 and a LCVR of SEQ ID NO: 85.

In one embodiment, the antibody or binding fragment thereof according to the present invention comprises a HCVR of SEQ ID NO: 122 and a LCVR of SEQ ID NO: 86.

In one embodiment, the antibody or binding fragment thereof according to the present invention comprises a HCVR of SEQ ID NO: 122 and a LCVR of SEQ ID NO: 87.

In one embodiment, the antibody or binding fragment thereof according to the present invention comprises a HCVR of SEQ ID NO: 122 and a LCVR of SEQ ID NO: 88.

In one embodiment, the antibody or binding fragment thereof according to the present invention comprises a HCVR of SEQ ID NO: 122 and a LCVR of SEQ ID NO: 89.

In one embodiment, the antibody or binding fragment thereof according to the present invention comprises a HCVR of SEQ ID NO: 122 and a LCVR of SEQ ID NO: 90.

In one embodiment, the antibody or binding fragment thereof according to the present invention comprises a HCVR of SEQ ID NO: 122 and a LCVR of SEQ ID NO: 103.

In one embodiment, the antibody or binding fragment thereof according to the present invention comprises a HCVR of SEQ ID NO: 122 and a LCVR of SEQ ID NO: 113.

In one embodiment, the antibody or binding fragment thereof according to the present invention comprises a HCVR of SEQ ID NO: 122 and a LCVR of SEQ ID NO: 126.

In one embodiment, the antibody or binding fragment thereof according to the present invention comprises a HCVR of SEQ ID NO: 123 and a LCVR of SEQ ID NO: 81.

In one embodiment, the antibody or binding fragment thereof according to the present invention comprises a HCVR of SEQ ID NO: 123 and a LCVR of SEQ ID NO: 82.

In one embodiment, the antibody or binding fragment thereof according to the present invention comprises a HCVR of SEQ ID NO: 123 and a LCVR of SEQ ID NO: 83.

In one embodiment, the antibody or binding fragment thereof according to the present invention comprises a HCVR of SEQ ID NO: 123 and a LCVR of SEQ ID NO: 84.

In one embodiment, the antibody or binding fragment thereof according to the present invention comprises a HCVR of SEQ ID NO: 123 and a LCVR of SEQ ID NO: 85.

In one embodiment, the antibody or binding fragment thereof according to the present invention comprises a HCVR of SEQ ID NO: 123 and a LCVR of SEQ ID NO: 86.

In one embodiment, the antibody or binding fragment thereof according to the present invention comprises a HCVR of SEQ ID NO: 123 and a LCVR of SEQ ID NO: 87.

In one embodiment, the antibody or binding fragment thereof according to the present invention comprises a HCVR of SEQ ID NO: 123 and a LCVR of SEQ ID NO: 88.

In one embodiment, the antibody or binding fragment thereof according to the present invention comprises a HCVR of SEQ ID NO: 123 and a LCVR of SEQ ID NO: 89.

In one embodiment, the antibody or binding fragment thereof according to the present invention comprises a HCVR of SEQ ID NO: 123 and a LCVR of SEQ ID NO: 90.

In one embodiment, the antibody or binding fragment thereof according to the present invention comprises a HCVR of SEQ ID NO: 123 and a LCVR of SEQ ID NO: 103.

In one embodiment, the antibody or binding fragment thereof according to the present invention comprises a HCVR of SEQ ID NO: 123 and a LCVR of SEQ ID NO: 113.

In one embodiment, the antibody or binding fragment thereof according to the present invention comprises a HCVR of SEQ ID NO: 123 and a LCVR of SEQ ID NO: 126.

In one embodiment, the antibody or binding fragment thereof according to the present invention comprises a HCVR of SEQ ID NO: 124 and a LCVR of SEQ ID NO: 81.

In one embodiment, the antibody or binding fragment thereof according to the present invention comprises a HCVR of SEQ ID NO: 124 and a LCVR of SEQ ID NO: 82.

In one embodiment, the antibody or binding fragment thereof according to the present invention comprises a HCVR of SEQ ID NO: 124 and a LCVR of SEQ ID NO: 83.

In one embodiment, the antibody or binding fragment thereof according to the present invention comprises a HCVR of SEQ ID NO: 124 and a LCVR of SEQ ID NO: 84.

In one embodiment, the antibody or binding fragment thereof according to the present invention comprises a HCVR of SEQ ID NO: 124 and a LCVR of SEQ ID NO: 85.

In one embodiment, the antibody or binding fragment thereof according to the present invention comprises a HCVR of SEQ ID NO: 124 and a LCVR of SEQ ID NO: 86.

In one embodiment, the antibody or binding fragment thereof according to the present invention comprises a HCVR of SEQ ID NO: 124 and a LCVR of SEQ ID NO: 87.

In one embodiment, the antibody or binding fragment thereof according to the present invention comprises a HCVR of SEQ ID NO: 124 and a LCVR of SEQ ID NO: 88.

In one embodiment, the antibody or binding fragment thereof according to the present invention comprises a HCVR of SEQ ID NO: 124 and a LCVR of SEQ ID NO: 89.

In one embodiment, the antibody or binding fragment thereof according to the present invention comprises a HCVR of SEQ ID NO: 124 and a LCVR of SEQ ID NO: 90.

In one embodiment, the antibody or binding fragment thereof according to the present invention comprises a HCVR of SEQ ID NO: 124 and a LCVR of SEQ ID NO: 103.

In one embodiment, the antibody or binding fragment thereof according to the present invention comprises a HCVR of SEQ ID NO: 124 and a LCVR of SEQ ID NO: 113.

In one embodiment, the antibody or binding fragment thereof according to the present invention comprises a HCVR of SEQ ID NO: 124 and a LCVR of SEQ ID NO: 126.

In one embodiment, any of the HCVR and/or LCVR as defined hereinabove can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the sequence of the non-CDR regions of any of the HCVR and/or LCVR as defined hereinabove can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, any of the HCVR and/or LCVR as defined hereinabove can be characterized as having an amino acid sequence that shares at least 70%, 75%, 80%, 85%, 90%, 95%, 96%, 97%, 98%, 99% or more of identity with the particular HCVR and/or LCVR as defined hereinabove.

In one embodiment, the sequence of the non-CDR regions of any of the HCVR and/or LCVR as defined hereinabove can be characterized as having an amino acid sequence that shares at least 70%, 75%, 80%, 85%, 90%, 95%, 96%, 97%, 98%, 99% or more of identity with the sequence of the non-CDR regions of the particular HCVR and/or LCVR as defined hereinabove.

In one embodiment, the body or binding fragment thereof according to the present invention comprises a fully or substantially fully human heavy chain constant region (abbreviated herein as HCCR or C_{H}) and/or light chain constant region (abbreviated herein as LCCR or C_{L}).

In one embodiment, the antibody or binding fragment thereof according to the present invention comprises a HCCR comprising or consisting of the sequence SEQ ID NO: 91.

SEQ ID NO: 91
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In one embodiment, the antibody or binding fragment thereof according to the present invention comprises a HCCR comprising or consisting of the sequence SEQ ID NO: 114.

SEQ ID NO: 114
ASTKGPSVFPLAPSSKSTSGGTAALGCLVKDYFPEPVTVSWNSGALTSGV
TFPAVLQSSGLYSLSSVVTVPSSSLGTQTYICNVNHKPSNTKVDKRVEPK
SCDKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPSSIEKTISKAKGQPREPQVYTLPPSREEMTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

In one embodiment, the antibody or binding fragment thereof according to the present invention comprises a HCCR comprising or consisting of the sequence SEQ ID NO: 115.

SEQ ID NO: 115
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCVAAAHHHHHH

In one embodiment, the antibody or binding fragment thereof according to the present invention comprises a LCCR comprising or consisting of the sequence SEQ ID NO: 92.

SEQ ID NO: 92
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In one embodiment, the antibody or binding fragment thereof according to the present invention comprises:
a HCCR comprising or consisting of the sequence SEQ ID NO: 91, 114 or 115; and
a LCCR comprising or consisting of the sequence SEQ ID NO: 92.

In one embodiment, the antibody or binding fragment thereof according to the present invention comprises:
a HCCR comprising or consisting of the sequence SEQ ID NO: 91; and
a LCCR comprising or consisting of the sequence SEQ ID NO: 92.

In one embodiment, the antibody or binding fragment thereof according to the present invention comprises:
a HCCR comprising or consisting of the sequence SEQ ID NO: 114; and
a LCCR comprising or consisting of the sequence SEQ ID NO: 92.

In one embodiment, the antibody or binding fragment thereof according to the present invention comprises:
a HCCR comprising or consisting of the sequence SEQ ID NO: 115; and
a LCCR comprising or consisting of the sequence SEQ ID NO: 92.

In one embodiment, the HCCR with SEQ ID NO: 91, 114 or 115 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or more amino acids being substituted by a different amino acid.

In one embodiment, the LCCR with SEQ ID NO: 92 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more amino acids being substituted by a different amino acid.

In one embodiment, the HCCR with SEQ ID NO: 91, 114 or 115, and/or the LCCR with SEQ ID NO: 92, can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or more amino acids being substituted by a different amino acid.

In one embodiment, the HCCR with SEQ ID NO: 91, 114 or 115, can be characterized as having an amino acid sequence that shares at least 70%, 75%, 80%, 85%, 90%, 95%, 96%, 97%, 98%, 99% or more of identity with SEQ ID NO: 91, 114 or 115, respectively.

In one embodiment, the LCCR with SEQ ID NO: 92 can be characterized as having an amino acid sequence that shares at least 70%, 75%, 80%, 85%, 90%, 95%, 96%, 97%, 98%, 99% or more of identity with SEQ ID NO: 92.

In one embodiment, the HCCR with SEQ ID NO: 91, 114 or 115, and/or the LCCR with SEQ ID NO: 92 can be characterized as having an amino acid sequence that shares at least 70%, 75%, 80%, 85%, 90%, 95%, 96%, 97%, 98%, 99% or more of identity with SEQ ID NO: 91, 114 or 115, and/or SEQ ID NO: 92, respectively.

In one embodiment, the antibody or binding fragment thereof according to the present invention comprises a HCCR and a LCCR comprising amino acid sequences that are homologous to the amino acid sequences of SEQ ID NO: 91, 114 or 115, and SEQ ID NO: 92, respectively, and wherein said antibody or binding fragment thereof retains the desired functional properties.

In one embodiment, the body or binding fragment thereof according to the present invention comprises a fully or substantially fully murine HCCR and/or LCCR.

In one embodiment, the antibody or binding fragment thereof according to the present invention comprises a HCCR comprising or consisting of the sequence SEQ ID NO: 93.

SEQ ID NO: 93
AKTTPPSVYPLAPGSAAQTNSMVTLGCLVKGYFPEPVTVTWNSGSLSSGV
HTFPAVLQSDLYTLSSSVTVPSSTWPSETVTCNVAHPASSTKVDKKIVPR
DCGCKPCICTVPEVSSVFIFPPKPKDVLTITLTPKVTCVVVDISKDDPEV
QFSWFVDDVEVHTAQTQPREEQFNSTFRSVSELPIMHQDWLNGKEFKCRV
NSAAFPAPIEKTISKTKGRPKAPQVYTIPPPKEQMAKDKVSLTCMITDFF
PEDITVEWQWNGQPAENYKNTQPIMDTDGSYFVYSKLNVQKSNWEAGNTF
TCSVLHEGLHNHHTEKSLSHSPG

In one embodiment, the antibody or binding fragment thereof according to the present invention comprises a LCCR comprising or consisting of the sequence SEQ ID NO: 94.

SEQ ID NO: 94
RADAAPTVSIFPPSSEQLTSGGASVVCFLNNFYPKDINVKWKIDGSERQN
GVLNSWTDQDSKDSTYSMSSTLTLTKDEYERHNSYTCEATHKTSTSPIVK
SFNRNEC

In one embodiment, the antibody or binding fragment thereof according to the present invention comprises:
a HCCR comprising or consisting of the sequence SEQ ID NO: 93; and
a LCCR comprising or consisting of the sequence SEQ ID NO: 94.

In one embodiment, the HCCR with SEQ ID NO: 93 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or more amino acids being substituted by a different amino acid.

In one embodiment, the LCCR with SEQ ID NO: 94 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more amino acids being substituted by a different amino acid.

In one embodiment, the HCCR with SEQ ID NO: 93 and/or the LCCR with SEQ ID NO: 94 can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or more amino acids being substituted by a different amino acid.

In one embodiment, the HCCR with SEQ ID NO: 93 can be characterized as having an amino acid sequence that shares at least 70%, 75%, 80%, 85%, 90%, 95%, 96%, 97%, 98%, 99% or more of identity with SEQ ID NO: 93.

In one embodiment, the LCCR with SEQ ID NO: 94 can be characterized as having an amino acid sequence that shares at least 70%, 75%, 80%, 85%, 90%, 95%, 96%, 97%, 98%, 99% or more of identity with SEQ ID NO: 94.

In one embodiment, the HCCR with SEQ ID NO: 93 and/or the LCCR with SEQ ID NO: 94 can be characterized as having an amino acid sequence that shares at least 70%, 75%, 80%, 85%, 90%, 95%, 96%, 97%, 98%, 99% or more of identity with SEQ ID NO: 93 and/or SEQ ID NO: 94, respectively.

In one embodiment, the antibody or binding fragment thereof according to the present invention comprises a HCCR and a LCCR comprising amino acid sequences that are homologous to the amino acid sequences of SEQ ID NO: 93 and SEQ ID NO: 94, respectively, and wherein said antibody or binding fragment thereof retains the desired functional properties.

In one embodiment, the antibody or binding fragment thereof according to the present invention is a murine, a chimeric or a humanized antibody or fragment thereof.

In one embodiment, the antibody or binding fragment thereof according to the present invention is a murine antibody or fragment thereof.

A “murine antibody or binding fragment thereof”, as used herein, includes antibodies and binding fragment thereof in which the variable region (including the CDRs and FRs) and the constant region are derived from a mouse.

In one embodiment, the murine antibody or binding fragment thereof according to the present invention comprises at least one, preferably at least two, more preferably the three HCVR's CDRs set forth in SEQ ID NOs: 1, 2 and 3; and/or at least one, preferably at least two, more preferably the three LCVR's CDRs set forth in SEQ ID NOs: 12, 13 and 14, with, wherever applicable, X₁₂ being absent.

In a preferred embodiment, the murine antibody or binding fragment thereof according to the present invention comprises at least one, preferably at least two, more preferably the three HCVR's CDRs set forth in SEQ ID NOs: 1, 4 and 3; and/or at least one, preferably at least two, more preferably the three LCVR's CDRs set forth in SEQ ID NOs: 15, 16 and 17, with, wherever applicable, X₁₂ being absent.

In a preferred embodiment, the murine antibody or binding fragment thereof according to the present invention may further comprise at least one, preferably at least two, more preferably at least three, even more preferably the four HCVR's FRs set forth in SEQ ID NOs: 25, 26, 27 and 28; and/or at least one, preferably at least two, more preferably at least three, even more preferably the four LCVR's FRs set forth in SEQ ID NOs: 44, 45, 46 and 47, with, wherever applicable, X₁₈ being a Tyr (Y).

In a preferred embodiment, the murine antibody or binding fragment thereof according to the present invention may therefore comprise a HCVR as set forth in SEQ ID NO: 61; and/or a LCVR as set forth in SEQ ID NO: 71 or 81.

In a preferred embodiment, the murine antibody or binding fragment thereof according to the present invention may further comprise a HCCR as set forth in SEQ ID NO: 93; and/or a LCCR as set forth in SEQ ID NO: 94.

In a preferred embodiment, the murine antibody or binding fragment thereof according to the present invention may therefore comprise a heavy chain comprising a variable region as set forth in SEQ ID NO: 61; and/or constant region as set forth in SEQ ID NO: 93.

In a preferred embodiment, the murine antibody or binding fragment thereof according to the present invention may therefore comprise a light chain comprising a variable region as set forth in SEQ ID NO: 71 or 81; and/or constant region as set forth in SEQ ID NO: 94.

In one embodiment, the antibody or binding fragment thereof according to the present invention is a chimeric antibody or binding fragment thereof.

A “chimeric antibody or binding fragment thereof”, as used herein, broadly refers to an antibody or binding fragment thereof comprising a first amino acid sequence linked to a second amino acid sequence with which it is not naturally linked in nature. The amino acid sequences may normally exist in separate proteins that are brought together in the fusion protein or they may normally exist in the same protein but are placed in a new arrangement in the fusion protein. A chimeric protein may be created, for example, by chemical synthesis, or by creating and translating a polynucleotide in which the peptide regions are encoded in the desired relationship. The term “chimeric antibody or binding fragment thereof” encompasses herein antibodies and binding fragment thereof in which
(a) the constant region, or a portion thereof, is altered, replaced or exchanged so that the variable region is linked to a constant region of a different or altered class, effector function and/or species, or an entirely different molecule which confers new properties to the chimeric antibody, e.g., an enzyme, toxin, hormone, growth factor, drug, etc.; or
(b) the variable region, or a portion thereof, is altered, replaced or exchanged with a variable region, or portion thereof, having a different or altered antigen specificity; or with corresponding sequences from another species or from another antibody class or subclass.

In one embodiment, the chimeric antibody or binding fragment thereof according to the present invention comprises a heavy chain and/or a light chain, comprising a human constant region and a murine variable region (including murine CDRs and murine FRs).

In one embodiment, the chimeric antibody or binding fragment thereof according to the present invention comprises a human heavy chain and a murine light chain; or a murine heavy chain and a human light chain.

In one embodiment, the chimeric antibody or binding fragment thereof according to the present invention comprises at least one, preferably at least two, more preferably the three HCVR's CDRs set forth in SEQ ID NOs: 1, 2 and 3; and/or at least one, preferably at least two, more preferably the three LCVR's CDRs set forth in SEQ ID NOs: 12, 13 and 14.

In a preferred embodiment, the chimeric antibody or binding fragment thereof according to the present invention comprises at least one, preferably at least two, more preferably the three HCVR's CDRs set forth in SEQ ID NOs: 1, 4 and 3; and/or at least one, preferably at least two, more preferably the three LCVR's CDRs set forth in SEQ ID NOs: 15, 16 and 17.

In a preferred embodiment, the chimeric antibody or binding fragment thereof according to the present invention comprises at least one, preferably at least two, more preferably the three HCVR's CDRs set forth in SEQ ID NOs: 1, 4 and 3; and/or at least one, preferably at least two, more preferably the three LCVR's CDRs set forth in SEQ ID NOs: 18, 111 or 120, and 17, respectively.

In a preferred embodiment, the chimeric antibody or binding fragment thereof according to the present invention may further comprise at least one, preferably at least two, more preferably at least three, even more preferably the four HCVR's FRs set forth in SEQ ID NOs: 25, 26, 27 and 28; and/or at least one, preferably at least two, more preferably at least three, even more preferably the four LCVR's FRs set forth in SEQ ID NOs: 44, 45, 46 and 47.

In a preferred embodiment, the chimeric antibody or binding fragment thereof according to the present invention may further comprise at least one, preferably at least two, more preferably at least three, even more preferably the four HCVR's FRs set forth in SEQ ID NOs: 25, 26, 27 and 28; and/or at least one, preferably at least two, more preferably at least three, even more preferably the four LCVR's FRs set forth in SEQ ID NOs: 48, 58, 50 and 51.

In a preferred embodiment, the chimeric antibody or binding fragment thereof according to the present invention may therefore comprise a HCVR as set forth in SEQ ID NO: 61; and/or a LCVR as set forth in SEQ ID NO: 71 or 81.

In a preferred embodiment, the chimeric antibody or binding fragment thereof according to the present invention may therefore comprise a HCVR as set forth in SEQ ID NO: 61; and/or a LCVR as set forth in SEQ ID NO: 112, 113, 125, 126, 128 or 129.

In a preferred embodiment, the chimeric antibody or binding fragment thereof according to the present invention may further comprise a human HCCR as set forth in SEQ ID NO: 91, 114 or 115; and/or a human LCCR as set forth in SEQ ID NO: 92.

In a preferred embodiment, the chimeric antibody or binding fragment thereof according to the present invention may further comprise a human HCCR as set forth in SEQ ID NO: 91; and/or a human LCCR as set forth in SEQ ID NO: 92.

In a preferred embodiment, the chimeric antibody or binding fragment thereof according to the present invention may further comprise a human HCCR as set forth in SEQ ID NO: 114; and/or a human LCCR as set forth in SEQ ID NO: 92.

In a preferred embodiment, the chimeric antibody or binding fragment thereof according to the present invention may further comprise a human HCCR as set forth in SEQ ID NO: 115; and/or a human LCCR as set forth in SEQ ID NO: 92.

In a preferred embodiment, the chimeric antibody or binding fragment thereof according to the present invention may further comprise a human HCCR as set forth in SEQ ID NO: 91, 114 or 115; and/or a murine LCCR as set forth in SEQ ID NO: 94.

In a preferred embodiment, the chimeric antibody or binding fragment thereof according to the present invention may further comprise a human HCCR as set forth in SEQ ID NO: 91; and/or a murine LCCR as set forth in SEQ ID NO: 94.

In a preferred embodiment, the chimeric antibody or binding fragment thereof according to the present invention may further comprise a human HCCR as set forth in SEQ ID NO: 114; and/or a murine LCCR as set forth in SEQ ID NO: 94.

In a preferred embodiment, the chimeric antibody or binding fragment thereof according to the present invention may further comprise a human HCCR as set forth in SEQ ID NO: 115; and/or a murine LCCR as set forth in SEQ ID NO: 94.

In a preferred embodiment, the chimeric antibody or binding fragment thereof according to the present invention may further comprise a murine HCCR as set forth in SEQ ID NO: 93; and/or a human LCCR as set forth in SEQ ID NO: 92.

In a preferred embodiment, the chimeric antibody or binding fragment thereof according to the present invention may therefore comprise a heavy chain comprising a variable region as set forth in SEQ ID NO: 61; and/or constant region as set forth in SEQ ID NO: 91, 114 or 115.

In a preferred embodiment, the chimeric antibody or binding fragment thereof according to the present invention may therefore comprise a heavy chain comprising a variable region as set forth in SEQ ID NO: 61; and/or constant region as set forth in SEQ ID NO: 91.

In a preferred embodiment, the chimeric antibody or binding fragment thereof according to the present invention may therefore comprise a heavy chain comprising a variable region as set forth in SEQ ID NO: 61; and/or constant region as set forth in SEQ ID NO: 114.

In a preferred embodiment, the chimeric antibody or binding fragment thereof according to the present invention may therefore comprise a heavy chain comprising a variable region as set forth in SEQ ID NO: 61; and/or constant region as set forth in SEQ ID NO: 115.

In a preferred embodiment, the chimeric antibody or binding fragment thereof according to the present invention may therefore comprise a light chain comprising a variable region as set forth in SEQ ID NO: 71 or 81; and/or constant region as set forth in SEQ ID NO: 92.

In one embodiment, the antibody or binding fragment thereof according to the present invention is a humanized antibody or binding fragment thereof.

A “humanized antibody or binding fragment thereof”, as used herein, refers to a chimeric antibody or binding fragment thereof which contains minimal sequence derived from a non-human immunoglobulin. It includes antibodies made by a non-human cell having variable and constant regions which have been altered to more closely resemble antibodies that would be made by a human cell, e.g., by altering the non-human antibody amino acid sequence to incorporate amino acids found in human germline immunoglobulin sequences. Humanized antibodies or binding fragment thereof according to the present invention may include amino acid residues not encoded by human germline immunoglobulin sequences (e.g., mutations introduced by random or site-specific mutagenesis in vitro or by somatic mutation in vivo), for example in the CDRs. The term “humanized antibody or binding fragment thereof” also includes antibodies and binding fragment thereof in which CDR sequences derived from the germline of another mammalian species, such as a mouse, have been grafted onto human framework sequences. In other words, the term “humanized antibody or binding fragment thereof” refers to an antibody or binding fragment thereof in which the CDRs of a recipient human antibody are replaced by CDRs from a donor non-human antibody, e.g., a mouse antibody. Humanized antibodies or binding fragments thereof may also comprise residues of donor origin in the framework sequences. The humanized antibody or binding fragment thereof can also comprise at least a portion of a human immunoglobulin constant region. Humanized antibodies and binding fragments thereof may also comprise residues which are found neither in the recipient antibody, nor in the imported CDR or FR sequences. Humanization can be performed using methods known in the art (e.g., Jones et al., 1986. Nature. 321(6069):522-5; Riechmann et al., 1988. Nature. 332(6162):323-7; Verhoeyen et al., 1988. Science. 239(4847):1534-6; Presta, 1992. Curr Opin Biotechnol. 3(4):394-8; U.S. Pat. No. 4,816,567), including techniques such as “superhumanizing” antibodies (e.g., Tan et al., 2002. J Immunol. 169(2):1119-25) and “resurfacing” (e.g., Staelens et al., 2006. Mol Immunol. 43(8):1243-57; Roguska et al., 1994. Proc Natl Acad Sci USA. 91(3):969-73).

Methods for humanizing the antibody or binding fragment thereof according to the present invention are well-known in the art, and will be further detailed in the Example section below. The choice of human variable domains, both light and heavy, to be used in making the humanized antibody or binding fragment thereof is very important to reduce antigenicity. According to the so-called “best-fit” method, the sequence of the variable domain of an antibody or binding fragment thereof according to the present invention is screened against the entire library of known human variable-domain sequences. The human sequence that is closest to the mouse sequence is then accepted as the human framework (FR) for the humanized antibody (Sims et al., 1993. J Immunol. 151(4):2296-308; Chothia & Lesk, 1987. J Mol Biol. 196(4):901-17).

Another method for humanizing the antibody or binding fragment thereof according to the present invention uses a particular FR from the consensus sequence of all human antibodies of a particular subgroup of light or heavy chains. The same framework can be used for several different humanized antibodies (Carter et al., 1992. Proc Natl Acad Sci USA. 89(10):4285-9; Presta et al., 1993. J Immunol. 151(5):2623-32). It is further important that antibodies be humanized with retention of high affinity for hCD45RC and other favorable biological properties. To achieve this goal, according to a preferred method, humanized antibodies and binding fragments thereof are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its epitope. In this way, CDR residues can be selected and combined from the consensus and import sequences so that the desired antibody characteristic, such as an increased affinity for hCD45RC, is achieved.

In general, the CDR residues are directly and most substantially involved in influencing antigen binding.

Another method for humanizing the antibody or binding fragment thereof according to the present invention is to use a transgenic or transchromosomic animal carrying parts of the human immune system for immunization. As a host, these animals have had their immunoglobulin genes replaced by functional human immunoglobulin genes. Thus, antibodies produced by these animals or in hybridomas made from the B cells of these animals are already humanized. Examples of such transgenic or transchromosomic animal include, without limitation:
the XenoMouse (Abgenix, Fremont, CA), described in U.S. Pat. Nos. 5,939,598, 6,075,181, 6,114,598, 6,150,584 and 6,162,963;
the HuMAb Mouse® (Medarex, Inc.), described in Lonberg et al., 1994. Nature. 368(6474):856-859; Lonberg & Huszar, 1995. Int Rev Immunol. 13(1):65-93; Harding & Lonberg, 1995. Ann NY Acad Sci. 764:536-46; Taylor et al., 1992. Nucleic Acids Res. 20(23):6287-95; Chen et al., 1993. Int Immunol. 5(6):647-56; Tuaillon et al., 1993. Proc Natl Acad Sci USA. 90(8):3720-4; Choi et al., 1993. Nat Genet. 4(2):117-23; Chen et al., 1993. EMBO J. 12(3):821-30; Tuaillon et al., 1994. J Immunol. 152(6):2912-20; Taylor et al., 1994. Int Immunol. 6(4):579-91; Fishwild et al., 1996. Nat Biotechnol. 14(7):845-51;
the KM Mouse®, described in Patent application WO2002043478;
the TC mice, described in Tomizuka et al., 2000. Proc Natl Acad Sci USA. 97(2):722-7; and
the OmniRat™ (OMT, Inc.), described in Patent application WO2008151081; Geurts et al., 2009. Science. 325(5939):433; Menoret et al., 2010. Eur J Immunol. 40(10):2932-41; Osborn et al., 2013. J Immunol. 190(4):1481-90.

Humanized antibodies and binding fragments thereof may also be produced according to various other techniques, such as by using, for immunization, other transgenic animals that have been engineered to express a human antibody repertoire (Jakobovitz et al., 1993. Nature. 362(6417):255-8), or by selection of antibody repertoires using phage display methods. Such techniques are known to the skilled person and can be implemented starting from monoclonal antibodies or binding fragments thereof as disclosed in the present application.

In some embodiments, the antibody or binding fragment thereof according to the present invention comprising HCVR and LCVR (or CDRs thereof) may comprise a first constant domain (C_{H}1 and/or C_{L}), the amino acid sequence of which is fully or substantially human.

In some embodiment, especially when the antibody or binding fragment thereof according to the present invention is intended for human therapeutic uses, it is typical for the entire constant region, or at least a part thereof, to have a fully or substantially human amino acid sequence. Therefore, one or more of, or any combination of, the C_{H}1 domain, hinge region, C_{H}2 domain, C_{H}3 domain and C_{L} domain (and C_{H}4 domain if present) may be fully or substantially human with respect to its amino acid sequence. Advantageously, the C_{H}1 domain, hinge region, C_{H}2 domain, C_{H}3 domain and C_{L} domain (and C_{H}4 domain if present) may all have a fully or substantially human amino acid sequence.

The term “substantially human”, in the context of the constant region of a humanized or chimeric antibody or binding fragment thereof, refers to an amino acid sequence identity of at least 70%, preferably at least 75%, 80%, 85%, 90%, 95%, 96%, 97%, 98%, 99% or more with a human constant region.

The term “human amino acid sequence”, in this context, refers to an amino acid sequence which is encoded by a human immunoglobulin gene, which includes germline, rearranged and somatically mutated genes. The present invention also contemplates proteins comprising constant domains of “human” sequence which have been altered, by one or more amino acid additions, deletions or substitutions with respect to the human sequence, excepting those embodiments where the presence of a “fully human hinge region” is expressly required.

The presence of a “fully human hinge region” in the antibody or binding fragment thereof according to the present invention may be beneficial both to minimize immunogenicity and to optimize stability of the antibody. It is considered that one or more amino acid substitutions, insertions or deletions may be made within the constant region of the heavy and/or the light chain, particularly within the Fc region. Amino acid substitutions may result in replacement of the substituted amino acid with a different naturally occurring amino acid, or with a non-natural or modified amino acid. Other structural modifications are also permitted, such as for example changes in glycosylation pattern (e.g., by addition or deletion of N- or O-linked glycosylation sites). Depending on the intended use of the antibody or binding fragment thereof, it may be desirable to modify the antibody or binding fragment thereof according to the present invention with respect to its binding properties to Fc receptors, for example to modulate effector function. For example, cysteine residue(s) may be introduced in the Fc region, thereby allowing interchain disulfide bond formation in this region. The homodimeric antibody thus generated may have improved effector function (Caron et al., 1992. J Exp Med. 176(4):1191-5; Shopes, 1992. J Immunol. 148(9):2918-22).

In one embodiment, the humanized antibody or binding fragment thereof according to the present invention comprises at least one, preferably at least two, more preferably the three HCVR's CDRs set forth in SEQ ID NOs: 1, 2 and 3; and/or at least one, preferably at least two, more preferably the three LCVR's CDRs set forth in SEQ ID NOs: 12, 13 and 14, with, wherever applicable, X₁₂ being absent or being a Ser (S).

In one embodiment, the humanized antibody or binding fragment thereof according to the present invention comprises at least one, preferably at least two, more preferably the three HCVR's CDRs and/or at least one, preferably at least two, more preferably the three LCVR's CDRs, in a combination as set forth in Table 2 with, wherever applicable, X₁₂ being absent or being a Ser (S).

In one embodiment, the humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 selected from SEQ ID NOs: 4, 5, 6, 7, 8, 9, 10, 11, 100, 116, 117, 118, and 119; and
   a V_{H}-CDR3 with SEQ ID NO: 3; and
a LCVR which comprises at least one, preferably at least two, more preferably the following three CDRs:
   a V_{L}-CDR1 selected from SEQ ID NOs: 15 and 18, with X₁₂ being absent or being a Ser (S);
   a V_{L}-CDR2 selected from SEQ ID NOs: 16, 19, 20, 22, 111 and 120; and
   a V_{L}-CDR3 selected from SEQ ID NOs: 17 and 21.

In a preferred embodiment, the humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 selected from SEQ ID NOs: 4 and 5; and
   a V_{H}-CDR3 with SEQ ID NO: 3; and
a LCVR which comprises at least one, preferably at least two, more preferably the following three CDRs:
   a V_{L}-CDR1 with SEQ ID NO: 15, with X₁₂ being absent;
   a V_{L}-CDR2 with SEQ ID NO: 16; and
   a V_{L}-CDR3 with SEQ ID NO: 17.

In a preferred embodiment, the humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 selected from SEQ ID NOs: 6, 7 and 8; and
   a V_{H}-CDR3 with SEQ ID NO: 3; and
a LCVR which comprises at least one, preferably at least two, more preferably the following three CDRs:
   a V_{L}-CDR1 with SEQ ID NO: 18, with X₁₂ being absent;
   a V_{L}-CDR2 selected from SEQ ID NO: 16 and 19; and
   a V_{L}-CDR3 with SEQ ID NO: 17.

In a preferred embodiment, the humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 selected from SEQ ID NOs: 9, 10 and 11; and
   a V_{H}-CDR3 with SEQ ID NO: 3; and
a LCVR which comprises at least one, preferably at least two, more preferably the following three CDRs:
   a V_{L}-CDR1 with SEQ ID NO: 18, with X₁₂ being absent;
   a V_{L}-CDR2 selected from SEQ ID NO: 19, 20 and 22; and
   a V_{L}-CDR3 selected from SEQ ID NO: 17 and 21.

In a preferred embodiment, the humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 with SEQ ID NOs: 100; and
   a V_{H}-CDR3 with SEQ ID NO: 3; and
a LCVR which comprises at least one, preferably at least two, more preferably the following three CDRs:
   a V_{L}-CDR1 with SEQ ID NO: 18, with X₁₂ being a Ser (S);
   a V_{L}-CDR2 with SEQ ID NO: 16; and
   a V_{L}-CDR3 with SEQ ID NO: 17.

In a preferred embodiment, the humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 selected from SEQ ID NOs: 6, 7, 8 and 100; and
   a V_{H}-CDR3 with SEQ ID NO: 3; and
a LCVR which comprises at least one, preferably at least two, more preferably the following three CDRs:
   a V_{L}-CDR1 with SEQ ID NO: 18, with X₁₂ being absent or being a Ser (S);
   a V_{L}-CDR2 selected from SEQ ID NO: 16 and 19; and
   a V_{L}-CDR3 with SEQ ID NO: 17.

In a preferred embodiment, the humanized antibody or binding fragment thereof according to the present invention comprises a combination of (i) at least one, preferably at least two, more preferably three HCVR's CDRs and (ii) at least one, preferably at least two, more preferably three LCVR's CDRs, said combination being selected from combinations #1, #7, #14, #26 and #50 as set forth in Table 2 with, wherever applicable, X₁₂ being absent or being a Ser (S).

In one embodiment, the humanized antibody or binding fragment thereof according to the present invention may further comprise at least one, preferably at least two, more preferably at least three, even more preferably the four HCVR's FRs and/or at least one, preferably at least two, more preferably at least three, even more preferably the four LCVR's FRs, as set forth in Table 3.

In one embodiment, the humanized antibody or binding fragment thereof according to the present invention may further comprise:
a HCVR which comprises at least one, preferably at least two, more preferably at least three, even more preferably the four following FRs:
   a V_{H}-FR1 selected from SEQ ID NOs: 29, 33, 36, 39, 40 and 42;
   a V_{H}-FR2 selected from SEQ ID NOs: 30, 34, 37 and 43;
   a V_{H}-FR3 selected from SEQ ID NOs: 31, 35, 38 and 41; and
   a V_{H}-FR4 with SEQ ID NO: 32; and
a LCVR which comprises at least one, preferably at least two, more preferably at least three, even more preferably the four following FRs:
   a V_{L}-FR1 selected from SEQ ID NOs: 48, 52 and 55;
   a V_{L}-FR2 selected from SEQ ID NOs: 49, 53, 56, 58, 59 and 60;
   a V_{L}-FR3 selected from SEQ ID NOs: 50, 54 and 57; and
   a V_{L}-FR4 with SEQ ID NO: 51.

In one embodiment, the humanized antibody or binding fragment thereof according to the present invention may further comprise a combination of (i) at least one, preferably at least two, more preferably at least three, even more preferably the four HCVR's FRs and (ii) at least one, preferably at least two, more preferably at least three, even more preferably the four LCVR's FRs, said combination being selected from combinations #9, #10, #11, #12, #13, #14, #16, #17, #18, #19, #20, #21, #23, #24, #25, #26, #27, #28, #30, #31, #32, #33, #34, #35, #37, #38, #39, #40, #41, #42, #44, #45, #46, #47, #48, #49, #51, #52, #53, #54, #55 and #56 as set forth in Table 3.

In a preferred embodiment, the humanized antibody or binding fragment thereof according to the present invention may further comprise:
a HCVR which comprises at least one, preferably at least two, more preferably at least three, even more preferably the four following FRs:
   a V_{H}-FR1 selected from SEQ ID NOs: 29, 33 and 36;
   a V_{H}-FR2 selected from SEQ ID NOs: 30, 34 and 37;
   a V_{H}-FR3 selected from SEQ ID NOs: 31, 35 and 38; and
   a V_{H}-FR4 with SEQ ID NO: 32; and
a LCVR which comprises at least one, preferably at least two, more preferably at least three, even more preferably the four following FRs:
   a V_{L}-FR1 selected from SEQ ID NOs: 48, 52 and 55;
   a V_{L}-FR2 selected from SEQ ID NOs: 49, 53 and 56;
   a V_{L}-FR3 selected from SEQ ID NOs: 50, 54 and 57; and
   a V_{L}-FR4 with SEQ ID NO: 51.

In a preferred embodiment, the humanized antibody or binding fragment thereof according to the present invention may further comprise a combination of (i) at least one, preferably at least two, more preferably at least three, even more preferably the four HCVR's FRs and (ii) at least one, preferably at least two, more preferably at least three, even more preferably the four LCVR's FRs, said combination being selected from combinations #9, #10, #11, #16, #17, #18, #23, #24 and #25 as set forth in Table 3.

In one embodiment, the humanized antibody or binding fragment thereof according to the present invention may therefore comprise:
a HCVR selected from SEQ ID NOs: 62, 63, 64, 65, 66, 67, 68, 69, 70, 101, 121, 122, 123 and 124; and/or
a LCVR selected from SEQ ID NOs: 82, 83, 84, 85, 86, 87, 88, 89, 90, 103, 113, 126 and 129.

In one embodiment, the humanized antibody or binding fragment thereof according to the present invention may therefore comprise any combination of a HCVR selected from SEQ ID NOs: 62, 63, 64, 65, 66, 67, 68, 69, 70, 101, 121, 122, 123 and 124, and a LCVR selected from SEQ ID NOs: 82, 83, 84, 85, 86, 87, 88, 89, 90, 103, 113, 126 and 129, as detailed above.

In a preferred embodiment, the humanized antibody or binding fragment thereof according to the present invention may therefore comprise:
a HCVR selected from SEQ ID NOs: 62, 63 and 64; and/or
a LCVR selected from SEQ ID NOs: 82, 83 and 84.

In a preferred embodiment, the humanized antibody or binding fragment thereof according to the present invention may therefore comprise any combination of a HCVR selected from SEQ ID NOs: 62, 63 and 64 and a LCVR selected from SEQ ID NOs: 82, 83 and 84, as detailed above.

In a preferred embodiment, the humanized antibody or binding fragment thereof according to the present invention may therefore comprise:
a HCVR selected from SEQ ID NOs: 62, 63, 64, 65, 67 and 101; and/or
a LCVR selected from SEQ ID NOs: 82, 83, 84, 85 and 103.

In a preferred embodiment, the humanized antibody or binding fragment thereof according to the present invention may therefore comprise any combination of a HCVR selected from SEQ ID NOs: 62, 63, 64, 65, 67 and 101, and a LCVR selected from SEQ ID NOs: 82, 83, 84, 85 and 103, as detailed above.

In a preferred embodiment, the humanized antibody or binding fragment thereof according to the present invention may therefore comprise a HCVR as set forth in SEQ ID NO: 62 and a LCVR as set forth in SEQ ID NO: 82.

In a preferred embodiment, the humanized antibody or binding fragment thereof according to the present invention may therefore comprise a HCVR as set forth in SEQ ID NO: 62 and a LCVR as set forth in SEQ ID NO: 83.

In a preferred embodiment, the humanized antibody or binding fragment thereof according to the present invention may therefore comprise a HCVR as set forth in SEQ ID NO: 62 and a LCVR as set forth in SEQ ID NO: 84.

In a preferred embodiment, the humanized antibody or binding fragment thereof according to the present invention may therefore comprise a HCVR as set forth in SEQ ID NO: 63 and a LCVR as set forth in SEQ ID NO: 82.

In a preferred embodiment, the humanized antibody or binding fragment thereof according to the present invention may therefore comprise a HCVR as set forth in SEQ ID NO: 63 and a LCVR as set forth in SEQ ID NO: 83.

In a preferred embodiment, the humanized antibody or binding fragment thereof according to the present invention may therefore comprise a HCVR as set forth in SEQ ID NO: 63 and a LCVR as set forth in SEQ ID NO: 84.

In a preferred embodiment, the humanized antibody or binding fragment thereof according to the present invention may therefore comprise a HCVR as set forth in SEQ ID NO: 64 and a LCVR as set forth in SEQ ID NO: 82.

In a preferred embodiment, the humanized antibody or binding fragment thereof according to the present invention may therefore comprise a HCVR as set forth in SEQ ID NO: 64 and a LCVR as set forth in SEQ ID NO: 83.

In a preferred embodiment, the humanized antibody or binding fragment thereof according to the present invention may therefore comprise a HCVR as set forth in SEQ ID NO: 64 and a LCVR as set forth in SEQ ID NO: 84.

In a preferred embodiment, the humanized antibody or binding fragment thereof according to the present invention may therefore comprise a HCVR as set forth in SEQ ID NO: 101 and a LCVR as set forth in SEQ ID NO: 85.

In a preferred embodiment, the humanized antibody or binding fragment thereof according to the present invention may therefore comprise a HCVR as set forth in SEQ ID NO: 101 and a LCVR as set forth in SEQ ID NO: 103.

In a preferred embodiment, the humanized antibody or binding fragment thereof according to the present invention may therefore comprise a HCVR as set forth in SEQ ID NO: 65 and a LCVR as set forth in SEQ ID NO: 85.

In a preferred embodiment, the humanized antibody or binding fragment thereof according to the present invention may therefore comprise a HCVR as set forth in SEQ ID NO: 65 and a LCVR as set forth in SEQ ID NO: 103.

In a preferred embodiment, the humanized antibody or binding fragment thereof according to the present invention may therefore comprise a HCVR as set forth in SEQ ID NO: 62 and a LCVR as set forth in SEQ ID NO: 85.

In a preferred embodiment, the humanized antibody or binding fragment thereof according to the present invention may therefore comprise a HCVR as set forth in SEQ ID NO: 101 and a LCVR as set forth in SEQ ID NO: 82.

In a preferred embodiment, the humanized antibody or binding fragment thereof according to the present invention may therefore comprise a HCVR as set forth in SEQ ID NO: 121 and a LCVR as set forth in SEQ ID NO: 85.

In a preferred embodiment, the humanized antibody or binding fragment thereof according to the present invention may therefore comprise a HCVR as set forth in SEQ ID NO: 122 and a LCVR as set forth in SEQ ID NO: 85.

In a preferred embodiment, the humanized antibody or binding fragment thereof according to the present invention may therefore comprise a HCVR as set forth in SEQ ID NO: 123 and a LCVR as set forth in SEQ ID NO: 85.

In a preferred embodiment, the humanized antibody or binding fragment thereof according to the present invention may therefore comprise a HCVR as set forth in SEQ ID NO: 124 and a LCVR as set forth in SEQ ID NO: 85.

In a preferred embodiment, the humanized antibody or binding fragment thereof according to the present invention may therefore comprise a HCVR as set forth in SEQ ID NO: 63 and a LCVR as set forth in SEQ ID NO: 85.

In a preferred embodiment, the humanized antibody or binding fragment thereof according to the present invention may therefore comprise a HCVR as set forth in SEQ ID NO: 67 and a LCVR as set forth in SEQ ID NO: 85.

In a preferred embodiment, the humanized antibody or binding fragment thereof according to the present invention may therefore comprise a HCVR as set forth in SEQ ID NO: 67 and a LCVR as set forth in SEQ ID NO: 103.

In one embodiment, the humanized antibody or binding fragment thereof according to the present invention may further comprise a HCCR as set forth in SEQ ID NO: 91, 114 or 115; and/or a LCCR as set forth in SEQ ID NO: 92.

In one embodiment, the humanized antibody or binding fragment thereof according to the present invention may further comprise a HCCR as set forth in SEQ ID NO: 91; and/or a LCCR as set forth in SEQ ID NO: 92.

In one embodiment, the humanized antibody or binding fragment thereof according to the present invention may further comprise a HCCR as set forth in SEQ ID NO: 114; and/or a LCCR as set forth in SEQ ID NO: 92.

In one embodiment, the humanized antibody or binding fragment thereof according to the present invention may further comprise a HCCR as set forth in SEQ ID NO: 115; and/or a LCCR as set forth in SEQ ID NO: 92.

In one embodiment, the humanized antibody or binding fragment thereof according to the present invention may therefore comprise a heavy chain comprising a variable region as set forth in SEQ ID NOs: 62, 63, 64, 65, 66, 67, 68, 69, 70 or 101; and/or constant region as set forth in SEQ ID NO: 91, 114 or 115.

In one embodiment, the humanized antibody or binding fragment thereof according to the present invention may therefore comprise a heavy chain comprising a variable region as set forth in SEQ ID NOs: 62, 63, 64, 65, 66, 67, 68, 69, 70 or 101; and/or constant region as set forth in SEQ ID NO: 91.

In one embodiment, the humanized antibody or binding fragment thereof according to the present invention may therefore comprise a heavy chain comprising a variable region as set forth in SEQ ID NOs: 62, 63, 64, 65, 66, 67, 68, 69, 70 or 101; and/or constant region as set forth in SEQ ID NO: 114.

In one embodiment, the humanized antibody or binding fragment thereof according to the present invention may therefore comprise a heavy chain comprising a variable region as set forth in SEQ ID NOs: 62, 63, 64, 65, 66, 67, 68, 69, 70 or 101; and/or constant region as set forth in SEQ ID NO: 115.

In a preferred embodiment, the humanized antibody or binding fragment thereof according to the present invention may therefore comprise a heavy chain comprising a variable region as set forth in SEQ ID NOs: 62, 63 or 64; and/or constant region as set forth in SEQ ID NO: 91, 114 or 115.

In a preferred embodiment, the humanized antibody or binding fragment thereof according to the present invention may therefore comprise a heavy chain comprising a variable region as set forth in SEQ ID NOs: 62, 63 or 64; and/or constant region as set forth in SEQ ID NO: 91.

In a preferred embodiment, the humanized antibody or binding fragment thereof according to the present invention may therefore comprise a heavy chain comprising a variable region as set forth in SEQ ID NOs: 62, 63 or 64; and/or constant region as set forth in SEQ ID NO: 114.

In a preferred embodiment, the humanized antibody or binding fragment thereof according to the present invention may therefore comprise a heavy chain comprising a variable region as set forth in SEQ ID NOs: 62, 63 or 64; and/or constant region as set forth in SEQ ID NO: 115.

In a preferred embodiment, the humanized antibody or binding fragment thereof according to the present invention may therefore comprise a heavy chain comprising a variable region as set forth in SEQ ID NOs: 62, 63, 64, 65, 67 and 101; and/or constant region as set forth in SEQ ID NO: 91, 114 or 115.

In a preferred embodiment, the humanized antibody or binding fragment thereof according to the present invention may therefore comprise a heavy chain comprising a variable region as set forth in SEQ ID NOs: 62, 63, 64, 65, 67 and 101; and/or constant region as set forth in SEQ ID NO: 91.

In a preferred embodiment, the humanized antibody or binding fragment thereof according to the present invention may therefore comprise a heavy chain comprising a variable region as set forth in SEQ ID NOs: 62, 63, 64, 65, 67 and 101; and/or constant region as set forth in SEQ ID NO: 114.

In a preferred embodiment, the humanized antibody or binding fragment thereof according to the present invention may therefore comprise a heavy chain comprising a variable region as set forth in SEQ ID NOs: 62, 63, 64, 65, 67 and 101; and/or constant region as set forth in SEQ ID NO: 115.

In a preferred embodiment, the humanized antibody or binding fragment thereof according to the present invention may therefore comprise a heavy chain comprising a variable region as set forth in SEQ ID NO: 101; and/or constant region as set forth in SEQ ID NO: 114.

In a preferred embodiment, the humanized antibody or binding fragment thereof according to the present invention may therefore comprise a heavy chain comprising a variable region as set forth in SEQ ID NO: 101; and/or constant region as set forth in SEQ ID NO: 115.

In one embodiment, the humanized antibody or binding fragment thereof according to the present invention may therefore comprise a light chain comprising a variable region as set forth in SEQ ID NOs: 82, 83, 84, 85, 86, 87, 88, 89, 90 or 103; and/or constant region as set forth in SEQ ID NO: 92.

In a preferred embodiment, the humanized antibody or binding fragment thereof according to the present invention may therefore comprise a light chain comprising a variable region as set forth in SEQ ID NOs: 82, 83 or 84; and/or constant region as set forth in SEQ ID NO: 92.

In a preferred embodiment, the humanized antibody or binding fragment thereof according to the present invention may therefore comprise a light chain comprising a variable region as set forth in SEQ ID NOs: 82, 83, 84, 85 or 103; and/or constant region as set forth in SEQ ID NO: 92.

According to a specific embodiment, the murine, chimeric or humanized antibody or fragment thereof as previously defined hereinabove comprises a LCVR's CDR1 set forth in SEQ ID NO: 12 (preferably set forth in SEQ ID NO: 15 or 18, more preferably set forth in SEQ ID NO: 15), with X₁₂ being selected from Asn (N), Ser (S) and Gly (G). In this embodiment, where X₁₂ is not absent, the amino acid residue at Kabat position L71 of the LCVR is preferably Phe (F); in other words, the murine, chimeric or humanized antibody or fragment thereof according to the present invention preferably comprises a LCVR's FR3 set forth in SEQ ID NOs: 46, 50, 54 or 57, with X₁₈ being Phe (F). Upon satisfaction of these features (i.e., X₁₂ not being absent and preferably Kabat position L71 (i.e., X₁₈) further being a Phe (F)), any of the above embodiments relating to the murine, chimeric or humanized antibody or binding fragment thereof according to the present invention apply.

Therefore, according to this specific embodiment, the murine, chimeric or humanized antibody or binding fragment thereof according to the present invention comprises at least one, preferably at least two, more preferably the three HCVR's CDRs set forth in SEQ ID NOs: 1, 2 and 3; and/or at least one, preferably at least two, more preferably the three LCVR's CDRs set forth in SEQ ID NOs: 12, 13 and 14, with, wherever applicable, X₁₂ being selected from Asn (N), Ser (S) and Gly (G).

According to this specific embodiment, the murine, chimeric or humanized antibody or binding fragment thereof according to the present invention comprises at least one, preferably at least two, more preferably the three HCVR's CDRs and/or at least one, preferably at least two, more preferably the three LCVR's CDRs, in a combination as set forth in Table 2 with, wherever applicable, X₁₂ being selected from Asn (N), Ser (S) and Gly (G).

According to this specific embodiment, the murine, chimeric or humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 selected from SEQ ID NOs: 4, 5, 6, 7, 8, 9, 10, 11 and 100; and
   a V_{H}-CDR3 with SEQ ID NO: 3; and
a LCVR which comprises at least one, preferably at least two, more preferably the following three CDRs:
   a V_{L}-CDR1 selected from SEQ ID NOs: 15 and 18, with X₁₂ being selected from Asn (N), Ser (S) and Gly (G);
   a V_{L}-CDR2 selected from SEQ ID NOs: 16, 19, 20, 22, 111 and 120; and
   a V_{L}-CDR3 selected from SEQ ID NOs: 17 and 21.

According to this specific embodiment, the murine, chimeric or humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 selected from SEQ ID NOs: 4, 5, 6, 8, 100, 116, 117, 118 and 119; and
   a V_{H}-CDR3 with SEQ ID NO: 3; and
a LCVR which comprises at least one, preferably at least two, more preferably the following three CDRs:
   a V_{L}-CDR1 selected from SEQ ID NOs: 15 and 18, with X₁₂ being selected from Asn (N), Ser (S) and Gly (G);
   a V_{L}-CDR2 selected from SEQ ID NOs: 16, 111 and 120; and
   a V_{L}-CDR3 with SEQ ID NO: 17.

According to this specific embodiment, the murine, chimeric or humanized antibody or binding fragment thereof according to the present invention comprises:
a HCVR which comprises at least one, preferably at least two, more preferably the following three CDRs:
   a V_{H}-CDR1 with SEQ ID NO: 1;
   a V_{H}-CDR2 selected from SEQ ID NOs: 4 and 5; and
   a V_{H}-CDR3 with SEQ ID NO: 3; and
a LCVR which comprises at least one, preferably at least two, more preferably the following three CDRs:
   a V_{L}-CDR1 with SEQ ID NO: 15, with X₁₂ being selected from Asn (N), Ser (S) and Gly (G);
   a V_{L}-CDR2 with SEQ ID NO: 16; and
   a V_{L}-CDR3 with SEQ ID NO: 17.

According to this specific embodiment, the murine, chimeric or humanized antibody or binding fragment thereof according to the present invention comprises a combination of (i) at least one, preferably at least two, more preferably three HCVR's CDRs and (ii) at least one, preferably at least two, more preferably three LCVR's CDRs, said combination being selected from combinations #1, #7, #14, #26 and #50 as set forth in Table 2 with, wherever applicable, X₁₂ being selected from Asn (N), Ser (S) and Gly (G).

According to this specific embodiment, the humanized antibody or binding fragment thereof according to the present invention may further comprise at least one, preferably at least two, more preferably at least three, even more preferably the four HCVR's FRs and/or at least one, preferably at least two, more preferably at least three, even more preferably the four LCVR's FRs, as set forth in Table 3 with, wherever applicable, X₁₈ being Phe (F).

According to this specific embodiment, the murine, chimeric or humanized antibody or binding fragment thereof according to the present invention may further comprise:
a HCVR which comprises at least one, preferably at least two, more preferably at least three, even more preferably the four following FRs:
   a V_{H}-FR1 selected from SEQ ID NOs: 25, 29, 33, 36, 39, 40 and 42;
   a V_{H}-FR2 selected from SEQ ID NOs: 26, 30, 34, 37 and 43;
   a V_{H}-FR3 selected from SEQ ID NOs: 27, 31, 35, 38 and 41; and
   a V_{H}-FR4 selected from SEQ ID NO: 28 and 32; and
a LCVR which comprises at least one, preferably at least two, more preferably at least three, even more preferably the four following FRs:
   a V_{L}-FR1 selected from SEQ ID NOs: 44, 48, 52 and 55;
   a V_{L}-FR2 selected from SEQ ID NOs: 45, 49, 53, 56, 58, 59 and 60;
   a V_{L}-FR3 selected from SEQ ID NOs: 46, 50, 54 and 57 with X₁₈ being Phe (F); and
   a V_{L}-FR4 selected from SEQ ID NO: 47 and 51.

According to this specific embodiment, the murine, chimeric or humanized antibody or binding fragment thereof according to the present invention may further comprise a combination of (i) at least one, preferably at least two, more preferably at least three, even more preferably the four HCVR's FRs and (ii) at least one, preferably at least two, more preferably at least three, even more preferably the four LCVR's FRs, said combination being selected from any of the combinations set forth in Table 3 with, wherever applicable, X₁₈ being Phe (F).

According to this specific embodiment, the murine, chimeric or humanized antibody or binding fragment thereof according to the present invention may further comprise:
a HCVR which comprises at least one, preferably at least two, more preferably at least three, even more preferably the four following FRs:
   a V_{H}-FR1 selected from SEQ ID NOs: 29, 33, 36, 39, 40 and 42;
   a V_{H}-FR2 selected from SEQ ID NOs: 30, 34, 37 and 43;
   a V_{H}-FR3 selected from SEQ ID NOs: 31, 35, 38 and 41; and
   a V_{H}-FR4 with SEQ ID NO: 32; and
a LCVR which comprises at least one, preferably at least two, more preferably at least three, even more preferably the four following FRs:
   a V_{L}-FR1 selected from SEQ ID NOs: 48, 52 and 55;
   a V_{L}-FR2 selected from SEQ ID NOs: 49, 53, 56, 58, 59 and 60;
   a V_{L}-FR3 selected from SEQ ID NOs: 50, 54 and 57 with X₁₈ being Phe (F); and
   a V_{L}-FR4 with SEQ ID NO: 51.

According to this specific embodiment, the murine, chimeric or humanized antibody or binding fragment thereof according to the present invention may further comprise a combination of (i) at least one, preferably at least two, more preferably at least three, even more preferably the four HCVR's FRs and (ii) at least one, preferably at least two, more preferably at least three, even more preferably the four LCVR's FRs, said combination being selected from combinations #9, #10, #11, #12, #13, #14, #16, #17, #18, #19, #20, #21, #23, #24, #25, #26, #27, #28, #30, #31, #32, #33, #34, #35, #37, #38, #39, #40, #41, #42, #44, #45, #46, #47, #48, #49, #51, #52, #53, #54, #55 and #56 as set forth in Table 3 with, wherever applicable, X₁₈ being Phe (F).

According to this specific embodiment, the murine, chimeric or humanized antibody or binding fragment thereof according to the present invention may further comprise:
a HCVR which comprises at least one, preferably at least two, more preferably at least three, even more preferably the four following FRs:
   a V_{H}-FR1 selected from SEQ ID NOs: 29, 33 and 36;
   a V_{H}-FR2 selected from SEQ ID NOs: 30, 34 and 37;
   a V_{H}-FR3 selected from SEQ ID NOs: 31, 35 and 38; and
   a V_{H}-FR4 with SEQ ID NO: 32; and
a LCVR which comprises at least one, preferably at least two, more preferably at least three, even more preferably the four following FRs:
   a V_{L}-FR1 selected from SEQ ID NOs: 48, 52 and 55;
   a V_{L}-FR2 selected from SEQ ID NOs: 49, 53 and 56;
   a V_{L}-FR3 selected from SEQ ID NOs: 50, 54 and 57 with X₁₈ being Phe (F); and
   a V_{L}-FR4 with SEQ ID NO: 51.

According to this specific embodiment, the murine, chimeric or humanized antibody or binding fragment thereof according to the present invention may further comprise a combination of (i) at least one, preferably at least two, more preferably at least three, even more preferably the four HCVR's FRs and (ii) at least one, preferably at least two, more preferably at least three, even more preferably the four LCVR's FRs, said combination being selected from combinations #9, #10, #11, #16, #17, #18, #23, #24 and #25 as set forth in Table 3 with, wherever applicable, X₁₈ being Phe (F).

According to this specific embodiment, the murine, chimeric or humanized antibody or binding fragment thereof according to the present invention may therefore comprise:
a HCVR selected from SEQ ID NOs: 61, 62, 63, 64, 65, 66, 67, 68, 69, 70, 101, 121, 122, 123 and 124; and/or
a LCVR selected from SEQ ID NOs: 71, 72, 73, 74, 75, 76, 77, 78, 79, 80, 102, 112, 125 and 128, with X₁₂ being selected from Asn (N), Ser (S) and Gly (G), and X₁₃ being any amino acid but Ala (A) or Asn (N).

According to this specific embodiment, the murine, chimeric or humanized antibody or binding fragment thereof according to the present invention may therefore comprise any combination of a HCVR selected from SEQ ID NOs: 61, 62, 63, 64, 65, 66, 67, 68, 69, 70, 101, 121, 122, 123 and 124, and a LCVR selected from SEQ ID NOs: 71, 72, 73, 74, 75, 76, 77, 78, 79, 80, 102, 112, 125 and 128, as detailed above.

According to this specific embodiment, the murine, chimeric or humanized antibody or binding fragment thereof according to the present invention may therefore comprise:
a HCVR selected from SEQ ID NOs: 61; and/or
a LCVR selected from SEQ ID NOs: 71 with X₁₂ being selected from Asn (N), Ser (S) and Gly (G).

According to this specific embodiment, the murine, chimeric or humanized antibody or binding fragment thereof according to the present invention may therefore comprise any combination of a HCVR selected from SEQ ID NOs: 61 and a LCVR selected from SEQ ID NOs: 71, as detailed above.

According to this specific embodiment, the murine, chimeric or humanized antibody or binding fragment thereof according to the present invention may therefore comprise:
a HCVR selected from SEQ ID NOs: 62, 63 and 64; and/or
a LCVR selected from SEQ ID NOs: 72, 73 and 74, with X₁₂ being selected from Asn (N), Ser (S) and Gly (G).

According to this specific embodiment, the murine, chimeric or humanized antibody or binding fragment thereof according to the present invention may therefore comprise any combination of a HCVR selected from SEQ ID NOs: 62, 63 and 64 and a LCVR selected from SEQ ID NOs: 72, 73 and 74, as detailed above.

According to this specific embodiment, the murine, chimeric or humanized antibody or binding fragment thereof according to the present invention may therefore comprise:
a HCVR selected from SEQ ID NOs: 62, 63, 64, 65, 67, 101, 121, 122, 123 and 124; and/or
a LCVR selected from SEQ ID NOs: 72, 73, 74, 75 and 102.

According to this specific embodiment, the murine, chimeric or humanized antibody or binding fragment thereof according to the present invention may therefore comprise any combination of a HCVR selected from SEQ ID NOs: 62, 63, 64, 65, 67 and 101, and a LCVR selected from SEQ ID NOs: SEQ ID NOs: 72, 73, 74, 75 and 102, as detailed above.

According to this specific embodiment, the murine, chimeric or humanized antibody or binding fragment thereof according to the present invention may further comprise a HCCR as set forth in SEQ ID NO: 91, 93, 114 or 115; and/or a LCCR as set forth in SEQ ID NO: 92 or 94.

According to this specific embodiment, the murine, chimeric or humanized antibody or binding fragment thereof according to the present invention may further comprise a HCCR as set forth in SEQ ID NO: 91 or 93; and/or a LCCR as set forth in SEQ ID NO: 92 or 94.

According to this specific embodiment, the murine, chimeric or humanized antibody or binding fragment thereof according to the present invention may therefore comprise a heavy chain comprising a variable region as set forth in SEQ ID NOs: 61, 62, 63, 64, 65, 66, 67, 68, 69, 70 or 101; and/or constant region as set forth in SEQ ID NO: 91, 93, 114 or 115.

According to this specific embodiment, the murine, chimeric or humanized antibody or binding fragment thereof according to the present invention may therefore comprise a heavy chain comprising a variable region as set forth in SEQ ID NOs: 61, 62, 63, 64, 65, 66, 67, 68, 69, 70 or 101; and/or constant region as set forth in SEQ ID NO: 91 or 93.

According to this specific embodiment, the murine, chimeric or humanized antibody or binding fragment thereof according to the present invention may therefore comprise a heavy chain comprising a variable region as set forth in SEQ ID NOs: 62, 63 or 64; and/or constant region as set forth in SEQ ID NO: 91, 93, 114 or 115.

According to this specific embodiment, the murine, chimeric or humanized antibody or binding fragment thereof according to the present invention may therefore comprise a heavy chain comprising a variable region as set forth in SEQ ID NOs: 62, 63 or 64; and/or constant region as set forth in SEQ ID NO: 91 or 93.

According to this specific embodiment, the murine, chimeric or humanized antibody or binding fragment thereof according to the present invention may therefore comprise a heavy chain comprising a variable region as set forth in SEQ ID NOs: 62, 63, 64, 65, 67 and 101; and/or constant region as set forth in SEQ ID NO: 91, 93, 114 or 115.

According to this specific embodiment, the murine, chimeric or humanized antibody or binding fragment thereof according to the present invention may therefore comprise a heavy chain comprising a variable region as set forth in SEQ ID NOs: 62, 63, 64, 65, 67 and 101; and/or constant region as set forth in SEQ ID NO: 91 or 93.

According to this specific embodiment, the murine, chimeric or humanized antibody or binding fragment thereof according to the present invention may therefore comprise a light chain comprising a variable region as set forth in SEQ ID NOs: 81, 82, 83, 84, 85, 86, 87, 88, 89, 90, 103 or 113; and/or a constant region as set forth in SEQ ID NO: 92 or 94.

According to this specific embodiment, the murine, chimeric or humanized antibody or binding fragment thereof according to the present invention may therefore comprise a light chain comprising a variable region as set forth in SEQ ID NOs: 82, 83 or 84; and/or a constant region as set forth in SEQ ID NO: 92.

According to this specific embodiment, the murine, chimeric or humanized antibody or binding fragment thereof according to the present invention may therefore comprise a light chain comprising a variable region as set forth in SEQ ID NOs: 82, 83, 84, 85 or 103; and/or a constant region as set forth in SEQ ID NO: 92.

Another object of the invention is an isolated nucleic acid encoding the antibody or binding fragment thereof binding to hCD45RC according to the present invention.

An “isolated nucleic acid”, as used herein, is intended to refer to a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

In one embodiment, the isolated nucleic acid is purified.

In one embodiment, the isolated nucleic acid is purified to:
(1) greater than 80%, 85%, 90%, 91%, 92%, 93%, 94%, 95% or more by weight of nucleic acid as determined by absorbance methods or fluorescence methods (such as, e.g., by measuring the ratio of absorbance at 260 and 280 nm (A_{260/280})), and most preferably more than 96%, 97%, 98% or 99% by weight; or
(2) homogeneity as shown by agarose gel electrophoresis and using an intercalating agent such as ethidium bromide, SYBR Green, GelGreen or the like.

In one embodiment, the nucleic acid according to the present invention comprises or consists of a sequence encoding the HCVR of the antibody or binding fragment thereof according to the invention.

In one embodiment, the nucleic acid according to the present invention comprises or consists of a sequence SEQ ID NO: 95 or any sequence sharing at least 70%, 75%, 80%, 85%, 90%, 95%, 96%, 97%, 98%, 99% or more identity with SEQ ID NO: 95, encoding the HCVR of the murine or chimeric antibody or binding fragment thereof with SEQ ID NO: 61.

SEQ ID NO: 95
CAGGTCCAGCTGCAACAGTCTGGCGCTGAGCTGGTTAGGCCTGGGACTTC
AGTGAAGATGTCCTGCAAGGCCGCTGGATACACCTTCACTAACTACTACA
TAGGTTGGGTAAAGCAGAGGCCTGGACATGGCCTTGAGTGGATCGGAGAT
ATTTTCCCTGGAGGTGACTATGCCAACAGCAATGAGAAGTTCAAGGGCAA
AGCCiACACTGACTGCAGACACATCCTCCAGCACAGCCTACATGCAGCTC
AGCAGCCTGACATCTGAGGACTCTGCCATCTATTACTGTGTGAGAAGGAA
CTTTGACTACTGGGGCCAAGGCACCACTCTCACAGTGTCCTCA

In one embodiment, the nucleic acid according to the present invention comprises or consists of a sequence encoding the LCVR of the antibody or binding fragment thereof according to the invention.

In one embodiment, the nucleic acid according to the present invention comprises or consists of a sequence SEQ ID NO: 96 or any sequence sharing at least 70%, 75%, 80%, 85%, 90%, 95%, 96%, 97%, 98%, 99% or more identity with SEQ ID NO: 96, encoding the LCVR of the murine or chimeric antibody or binding fragment thereof with SEQ ID NO: 81.

SEQ ID NO: 96
CAAATTGTTCTCACCCAGTCTCCAACAATCATGTCTGCATCTCCAGGGGA
GAAGGTGACCATAACCTGCAGTGCCAGCTCAAGTGTAAGTTACATGCACT
GGTTCCAGCAGAAGACAGGCACTTCTCCCAGACTCTGGATTTATAACACA
TCCA1ACCTGCCTTCTGGAGTCCCCGCTCGCTTCAGTGGCAGTGGATCTG
GGACCTCTTACTCTCTCACAATCAGCCGAATGGAGGCTGAAGATGCTGCC
ACTTATTACTGCCAGCAAAGGAGTAGTTACCCACTCACGTTCGGTGCTGG
GACCAAGCTGGAGCTGAAA

In one embodiment, the nucleic acid according to the present invention comprises or consists of:
a sequence encoding the HCVR of the antibody or binding fragment thereof according to the invention, and
a sequence encoding the LCVR of the antibody or binding fragment thereof according to the invention.

In one embodiment, the nucleic acid according to the present invention comprises or consists of:
a sequence SEQ ID NO: 95 or any sequence sharing at least 70%, 75%, 80%, 85%, 90%, 95%, 96%, 97%, 98%, 99% or more identity with SEQ ID NO: 995 and
a sequence SEQ ID NO: 96 or any sequence sharing at least 70%, 75%, 80%, 85%, 90%, 95%, 96%, 97%, 98%, 99% or more identity with SEQ ID NO: 96.

It will be readily understood that the one skilled in the art can design nucleic acid sequences encoding all other HCVRs and LCVRs herein disclosed, in particular for the humanized antibodies or binding fragment thereof according to the invention.

It is further understood that the one skilled in the art is familiar with molecular biology methods aiming at modifying a nucleic acid sequence in order to improve, e.g., recombinant production rates, such as by codon optimization. Ultimately, the present application encompasses any nucleic acid encoding any HCVRs and/or LCVRs as herein disclosed.

In one embodiment, the nucleic acid according to the present invention comprises or consists of a sequence encoding a fully or substantially fully human HCCR of the antibody or binding fragment thereof according to the invention.

In one embodiment, the nucleic acid according to the present invention comprises or consists of a sequence SEQ ID NO: 97 or any sequence sharing at least 70%, 75%, 80%, 85%, 90%, 95%, 96%, 97%, 98%, 99% or more identity with SEQ ID NO: 97, encoding the HCCR of the chimeric or humanized antibody or binding fragment thereof with SEQ ID NO: 91.

SEQ ID NO: 97
GCCTCCACCAAGGGCCCATCGGTCTTCCCCCTGGCACCCTCCTCCAAGAG
CACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACCCAGACCTACATCTGCA
ACGTGAATCACAAGCCCAGCAACACCAAGGTGGACAAGAGAGTTGAGCCC
AAATCTTGTGACAAAACTCACACATGCCCACCGTGCCCAGCACCTGAACT
CCTGGGGGGACCGTCAGTCTTCCTCTTCCCCCCAAAACCCAAGGACACCC
TCATGATCTCCCGGACCCCTGAGGTCACATGCGTGGTGGTGGACGTGAGC
CACGAAGACCCTGAGGTCAAGTTCAACTGGTACGTGGACGGCGTGGAGGT
GCATAATGCCAAGACAAAGCCGCGGGAGGAGCAGTACAACAGCACGTACC
GTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCC
TGCCCCCATCCCGGGAGGAGATGACCAAGAACCAGGTCAGCCTGACCTGC
CTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCG
ACGGCTCCTTCTTCCTCTATAGCAAGCTCACCGTGGACAAGAGCAGGTGG
CAGCAGGGGAACGTCTTCTCATGCTCCGTGATGCATGAGGCTCTGCACAA
CCACTACACGCAGAAGAGCCTCTCCCTGTCCCCGGGTAAA

In one embodiment, the nucleic acid according to the present invention comprises or consists of a sequence encoding a fully or substantially fully human LCCR of the antibody or binding fragment thereof according to the invention.

In one embodiment, the nucleic acid according to the present invention comprises or consists of a sequence SEQ ID NO: 98 or any sequence sharing at least 70%, 75%, 80%, 85%, 90%, 95%, 96%, 97%, 98%, 99% or more identity with SEQ ID NO: 98, encoding the HCCR of the chimeric or humanized antibody or binding fragment thereof with SEQ ID NO: 92.

SEQ ID NO: 98
CGAACTGTGGCTGCACCATCTGTCTTCATCTTCCCGCCATCTGATGAGC
AGTTGAAATCTGGAACTGCCTCTGTTGTGTGCCTGCTGAATAACTTCTA
TCCCAGAGAGGCCAAAGTACAGTGGAAGGTGGATAACGCCCTCCAATCG
GGTAACTCCCAGGAGAGTGTCACAGAGCAGGACAGCAAGGACAGCACCT
ACAGCCTCAGCAGCACCCTGACGCTGAGCAAAGCAGACTACGAGAAACA
CAAAGTCTACGCCTGCGAAGTCACCCATCAGGGCCTGAGCTCGCCCGTC
ACAAAGAGCTTCAACAGGGGAGAGTGT

In one embodiment, the nucleic acid according to the present invention comprises or consists of:
a sequence encoding a fully or substantially fully human HCCR of the antibody or binding fragment thereof according to the invention, and
a sequence encoding a fully or substantially fully human LCCR of the antibody or binding fragment thereof according to the invention.

In one embodiment, the nucleic acid according to the present invention comprises or consists of:
a sequence SEQ ID NO: 97 or any sequence sharing at least 70%, 75%, 80%, 85%, 90%, 95%, 96%, 97%, 98%, 99% or more identity with SEQ ID NO: 97, and
a sequence SEQ ID NO: 98 or any sequence sharing at least 70%, 75%, 80%, 85%, 90%, 95%, 96%, 97%, 98%, 99% or more identity with SEQ ID NO: 98.

Another object of the present invention is an expression vector comprising the nucleic acid encoding the antibody or binding fragment thereof binding to hCD45RC according to the present invention.

In one embodiment, the expression vector according to the present invention comprises a sequence encoding the HCVR of the antibody or binding fragment thereof according to the invention, preferably operably linked to regulatory elements.

In one embodiment, the expression vector according to the present invention comprises a sequence SEQ ID NO: 95 or any sequence sharing at least 70%, 75%, 80%, 85%, 90%, 95%, 96%, 97%, 98%, 99% or more identity with SEQ ID NO: 95, preferably operably linked to regulatory elements.

In one embodiment, the expression vector according to the present invention comprises a sequence encoding the LCVR of the antibody or binding fragment thereof according to the invention, preferably operably linked to regulatory elements.

In one embodiment, the expression vector according to the present invention comprises a sequence SEQ ID NO: 96 or any sequence sharing at least 70%, 75%, 80%, 85%, 90%, 95%, 96%, 97%, 98%, 99% or more identity with SEQ ID NO: 96, preferably operably linked to regulatory elements.

In one embodiment, the expression vector according to the present invention comprises:
a sequence encoding the HCVR of the antibody or binding fragment thereof according to the invention, preferably operably linked to regulatory elements, and
a sequence encoding the LCVR of the antibody or binding fragment thereof according to the invention, preferably operably linked to regulatory elements.

In one embodiment, the expression vector according to the present invention comprises:
a sequence SEQ ID NO: 95 or any sequence sharing at least 70%, 75%, 80%, 85%, 90%, 95%, 96%, 97%, 98%, 99% or more identity with SEQ ID NO: 95, preferably operably linked to regulatory elements, and
a sequence SEQ ID NO: 96 or any sequence sharing at least 70%, 75%, 80%, 85%, 90%, 95%, 96%, 97%, 98%, 99% or more identity with SEQ ID NO: 96, preferably operably linked to regulatory elements.

In one embodiment, the expression vector according to the present invention comprises a sequence encoding the HCCR of the antibody or binding fragment thereof according to the invention, preferably operably linked to regulatory elements.

In one embodiment, the expression vector according to the present invention comprises a sequence SEQ ID NO: 97 or any sequence sharing at least 70%, 75%, 80%, 85%, 90%, 95%, 96%, 97%, 98%, 99% or more identity with SEQ ID NO: 97, preferably operably linked to regulatory elements.

In one embodiment, the expression vector according to the present invention comprises a sequence encoding the LCCR of the antibody or binding fragment thereof according to the invention, preferably operably linked to regulatory elements.

In one embodiment, the expression vector according to the present invention comprises a sequence SEQ ID NO: 98 or any sequence sharing at least 70%, 75%, 80%, 85%, 90%, 95%, 96%, 97%, 98%, 99% or more identity with SEQ ID NO: 98, preferably operably linked to regulatory elements.

In one embodiment, the expression vector according to the present invention comprises:
a sequence encoding the HCCR of the antibody or binding fragment thereof according to the invention, preferably operably linked to regulatory elements, and
a sequence encoding the LCCR of the antibody or binding fragment thereof according to the invention, preferably operably linked to regulatory elements.

In one embodiment, the expression vector according to the present invention comprises:
a sequence SEQ ID NO: 97 or any sequence sharing at least 70%, 75%, 80%, 85%, 90%, 95%, 96%, 97%, 98%, 99% or more identity with SEQ ID NO: 97, preferably operably linked to regulatory elements, and
a sequence SEQ ID NO: 98 or any sequence sharing at least 70%, 75%, 80%, 85%, 90%, 95%, 96%, 97%, 98%, 99% or more identity with SEQ ID NO: 98, preferably operably linked to regulatory elements.

In one embodiment, the expression vector according to the present invention comprises a sequence encoding the heavy chain of the antibody or binding fragment thereof according to the invention, preferably operably linked to regulatory elements.

In one embodiment, the expression vector according to the present invention comprises a sequence encoding the light chain of the antibody or binding fragment thereof according to the invention, preferably operably linked to regulatory elements.

In one embodiment, the expression vector according to the present invention comprises:
a sequence encoding the heavy chain of the antibody or binding fragment thereof according to the invention, preferably operably linked to regulatory elements, and
a sequence encoding the light chain of the antibody or binding fragment thereof according to the invention, preferably operably linked to regulatory elements.

In one embodiment, the expression vector according to the present invention is monocistronic.

By “monocistronic”, it is meant that a single nucleic acid encoding a single protein is expressed in a single expression vector.

In one embodiment, the expression vector according to the present invention is polycistronic.

By “polycistronic”, it is meant that at least two or more nucleic acids, each encoding a single protein, are expressed in a single expression vector.

Another object of the present invention is a cell comprising the nucleic acid encoding the antibody or binding fragment thereof binding to hCD45RC according to the present invention, or the expression vector comprising the nucleic acid encoding the antibody or binding fragment thereof binding to hCD45RC according to the present invention.

Another object of the present invention is a method of producing and purifying the isolated antibody or a binding fragment thereof, binding to hCD45RC according to the present invention.

In one embodiment, the method comprises:
culturing host cells transformed with the nucleic acid or expression vector according to the present invention, under conditions suitable for expression of the
antibody or binding fragment thereof, and
recovering the expressed antibody or binding fragment thereof.

This recombinant process can be used for large scale production of antibodies or binding fragments thereof, including monoclonal antibodies intended for in vitro, ex vivo and/or in vivo therapeutic and/or diagnostic uses.

These processes are well-known in the art (Subramanian (Ed.), 2004. Antibodies (1st ed., Vol. 1: Production and Purification). New York, NY: Springer US).

In an embodiment, the expressed antibody or binding fragment thereof is further purified.

Methods to purify the antibody or binding fragment thereof according to the present invention are well-known in the art (Subramanian (Ed.), 2004. Antibodies (1st ed., Vol. 1: Production and Purification). New York, NY: Springer US), and include, without limitation, chromatography, preferably by affinity chromatography, more preferably by affinity chromatography on protein L agarose.

In one embodiment, the antibody according to the present invention can be modified to enhance antibody-dependent cell-mediated cytotoxicity (ADCC), complement dependent cytotoxicity (CDC) and/or antibody-dependent phagocytosis. Such modifications are well-known in the art.

For example, antibodies comprising a low fucose content are known to enhance ADCC response via the FcγRIII receptor (International patent publication WO2014140322). Thus, the antibody according to the present invention may comprise a low fucose content.

The term “fucose content”, as used herein, refers to the percentage of fucosylated forms within the N-glycans attached to the N297 residue of the Fc fragment of each heavy chain of each antibody (corresponding to N180 of SEQ ID NO: 91 or SEQ ID NO: 114; or N174 of SEQ ID NO: 93).

The term “low fucose content”, as used herein, refers to a fucose content of less than, or equal to, 65%. Advantageously, the fucose content is less than or equal to 65%, preferably less than or equal to 60%, 55% or 50%, or even less than or equal to 45%, 40%, 35%, 30%, 25% or 20%. However, it is not necessary that the fucose content be zero, and it may for example be greater than or equal to 5%, 10%, 15% or 20%.

In one embodiment, the antibody according to the present invention may further comprises different types of glycosylation (N-glycans of the oligomannose or biantennary complex type, with a variable proportion of bisecting N-acetylglucosamine (GlcNAc) residues or galactose residues in the case of N-glycans of the biantennary complex type), provided that they have a low fucose content (International patent publication WO2007048077). For example, antibodies having slightly fucosylated N-glycans can be obtained as described in European patent publication EP1176195 or in International patent publications WO2001077181 or WO2012041768.

The N-glycans of the oligomannose type have reduced half-life in vivo as compared to N-glycans of the biantennary complex type. Consequently, advantageously, the antibodies according to the present invention have on their N-glycosylation sites of the Fc fragment glycan structures of the biantennary complex type, with a low fucose content, as defined above.

In some embodiments, the antibody according to the present invention is conjugated to a therapeutic moiety, i.e., a drug. In one embodiment, the therapeutic moiety is selected from a cytotoxin, a chemotherapeutic agent, a cytokine, an immunosuppressant, an immune stimulator, a lytic peptide and a radioisotope. Such conjugates are referred to herein as an “antibody drug conjugates” or “ADCs”.

In some embodiments, the antibody of the present invention is conjugated to a cytotoxic moiety. In one embodiment, the cytotoxic moiety is selected from taxol; cytochalasin B; gramicidin D; ethidium bromide; emetine; mitomycin; etoposide; tenoposide; vincristine; vinblastine; colchicin; doxorubicin; daunorubicin; dihydroxyanthracin dione; tubulin-inhibitors (such as, e.g., maytansine or an analog or derivative thereof); antimitotic agents (such as, e.g., monomethyl auristatin E or F or an analog or derivative thereof); dolastatin or an analogue thereof; irinotecan or an analogue thereof; mitoxantrone; mithramycin; actinomycin D; 1-dehydrotestosterone; glucocorticoids; procaine; tetracaine; lidocaine; propranolol; puromycin; calicheamicin or an analog or derivative thereof; antimetabolites (such as, e.g., methotrexate, mercaptopurine, 6-thioguanine, cytarabine, fludarabin, 5-fluorouracil, decarbazine, hydroxyurea, asparaginase, gemcitabine, or cladribine); an alkylating agent (such as, e.g., mechlorethamine, thioepa, chlorambucil, melphalan, carmustine, lomustine, cyclophosphamide, busulfan, dibromomannitol, streptozotocin, dacarbazine, procarbazine or mitomycin C); platinum derivatives (such as, e.g., cisplatin or carboplatin); duocarmycin A, duocarmycin SA, rachelmycin, or an analog or derivative thereof; antibiotics (such as, e.g., dactinomycin, bleomycin, daunorubicin, doxorubicin, idarubicin, mithramycin, mitomycin, mitoxantrone, plicamycin or anthramycin); pyrrolo[2,1-c][1,4]-benzodiazepines (such as, e.g., anthramycin); diphtheria toxin and related molecules (such as, e.g., diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alphasarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins); ribonucleases; DNase I; Staphylococcal enterotoxin A; pokeweed antiviral protein; diphtherin toxin; and Pseudomonas endotoxin.

In some embodiments, the antibody according to the present invention is conjugated to an auristatin or a peptide analog, derivative or prodrug thereof. Auristatins have been shown to interfere with microtubule dynamics, GTP hydrolysis and nuclear and cellular division (Woyke et al., 2001. Antimicrob Agents Chemother. 46(12):3802-8) and have anti-cancer (U.S. Pat. No. 5,663,149) and antifungal activity (Pettit et al., 1998. Antimicrob Agents Chemother. 42(11):2961-5). For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to antibodies, are described in, e.g., U.S. Pat. Nos. 5,635,483, 5,780,588 and 6,214,345 and in International patent publications WO2002088172, WO2004010957, WO2005081711, WO2005084390, WO2006132670, WO2003026577, WO200700860, WO2007011968 and WO2005082023.

In some embodiments, the antibody according to the present invention is conjugated to pyrrolo[2,1-c][1,4]-benzodiazepine (PDB) (such as, e.g., anthramycin) or an analog, derivative or prodrug thereof. Suitable PDBs and PDB derivatives, and related technologies are described in the art.

In some embodiments, the antibody according to the present invention is conjugated to a cytotoxic moiety selected from anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody according to the present invention is conjugated to an anthracycline or an analog, derivative or prodrug thereof.

In some embodiments, the antibody according to the present invention is conjugated to maytansine or an analog, derivative or prodrug thereof.

In some embodiments, the antibody according to the present invention is conjugated to calicheamicin or an analog, derivative or prodrug thereof.

In some embodiments, the antibody according to the present invention is conjugated to duocarmycin or an analog, derivative or prodrug thereof.

In some embodiments, the antibody according to the present invention is conjugated to rachelmycin (CC-1065) or an analog, derivative or prodrug thereof.

In some embodiments, the antibody according to the present invention is conjugated to dolastatin or an analog, derivative or prodrug thereof.

In some embodiments, the antibody according to the present invention is conjugated to monomethyl auristatin E or an analog, derivative or prodrug thereof.

In some embodiments, the antibody according to the present invention is conjugated to monomethyl auristatin F or an analog, derivative or prodrug thereof.

In some embodiments, the antibody according to the present invention is conjugated to irinotecan or an analog, derivative or prodrug thereof.

In some embodiments, the antibody according to the present invention is labelled.

By “labeled”, it is meant that the isolated antibody or binding fragment thereof has at least one element, isotope or chemical compound conjugated or attached to it, enabling for example the detection of said isolated antibody or binding fragment thereof.

Examples of labels include, but are not limited to, luminescent dyes (also termed fluorophores or photodetectable labels), isotopic labels (also termed radioactive labels, radiolabels or heavy isotopes), contrast agents, magnetic labels, electric labels, thermal labels, and colored labels.

Examples of luminescent dyes include, but are not limited to, Alexa Fluor® dyes, BODIPY© dyes, fluorescein, 5-carboxyfluorescein, 5-(4,6-dichlorotriazin-2-yl) aminofluorescein, 2′7′-dimethoxy-4′5′-dichloro-6-carboxyfluorescein, fluorescein isothiocyanate (FITC), QFITC, Oregon Green® 488, Oregon Green® 514, rhodamine and derivatives thereof (such as, e.g., rhodamine green, rhodamine green-X, rhodamine red-X. X-rhodamine, 6-carboxy-X-rhodamine (ROX), 6-carboxyrhodamine (R6G), N,N,N′,N′-tetraethyl-6-carboxyrhodamine (TAMRA), lissamine rhodamine B, rhodamine 123, rhodamine X isothiocyanate, sulforhodamine B, sulforhodamine 101 (Texas Red), tetramethyl rhodamine, tetramethyl rhodamine isothiocyanate (TRITC)), eosin, cosin isothiocyanate, erythrosine, erythrosine B, erythrosin isothiocyanate, Texas Red®, Texas Red®-X, naphthofluorescein, malachite green, malachite green isothiocyanate, coumarin derivatives, Pacific Orange, cascade blue, cascade yellow, dansyl chloride, dapoxyl dye, 1-dimethylamine-N(2-azido-ethyl)naphthalene-5-sulfonamide, 6-(6-amino-2-(2-azidoethyl)-1,3-dioxo-1H-benzo(de)-2(3H)isoquinoline, 6-(6-amino-2-(2-propinyl)-1,3-dioxo-1H-benzo(de)-2(3H)isoquinoline, 8-(4-azidoethyloxyphenyl)-2,6-diethyl-1,3,5,7-tetramethyl-4,4-difluoro-4-bora-3a,4a-diaza-s-indacene, 8-(4-propionyloxyphenyl)-2,6-diethyl-1,3,5,7-tetramethyl-4,4-difluoro-4-bora-3a,4a-diaza-s-indacene, 1-(3-azido-propoxy)-7-methylamino-phenoxazin-3-one, 1-(2-propynyl)-7-methylamino-phenoxazin-3-one, N-(5-(3-azidopropylamino)-9H-benzo(a)-phenoxa-2-in-9-ylidene)-N-methyl-methanaminium chloride, N-(5-(3-propynyl-1-amino)-9H-benzo(a)-phenoxazin-9-ylene)-N-n ethyl-methanaminium chloride, (9-(3-azido-propoxy)-7-piperidin-1-yl-phenoxazin-3-ylidene)-dimethyl-ammonium perchlorate, 4-acetamido-4′-isothiocyanatostilbene-2,2′-disulfonic acid, acridine, acridine isothiocyanate, 5-(2′-aminoethyl)aminonaphthalene-1-sulfonic acid, 4-amino-N-[3-vinylsulfonyl phenyl]naphthalimide-3,5 disulfonate, N(4-anilino-1-naphthyl)maleimide, anthranilamide, Brilliant Yellow, coumarin, coumarin derivatives, 7-amino-4-methylcoumarin 7-amino-trifluoromethylcouluarin, cyanosine, 4%6-diamidino-2-phenylindole, 5′,5″-dibromopyrogallol-sulfonephthalein, 7-diethylamino-3-(4′-isothiocyanatophenyl)-4-methylcoumarin-4,4′-diisothiocyaniatodihydro-stilbene-2,2-disulfonic acid, 4,4′-diisothiocyanatostilbene-2,2′-disulfonic acid, ethidium, IR144, IR1446, 4-methylumbelliferone, o-cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4, riboflavin, rosolic acid, lanthanide chelates, quantum dots, cyanines, pyrelium dyes, and squarainies.

Examples of isotopic labels include, but are not limited to, ^{99m}Tc compounds (such as, e.g., exametazime, medronic acid, macroaggregated albumin, sestamibi, tetrofosmin, exametazime, sulesomab, tilmanocept, arcitumomab, votumumab, hynic-octreotide, and the like); ¹²³I, ¹²⁵I or ¹³¹I compounds (such as, e.g., ioflupane, iofetamine, iomazenil, sodium iodohippurate, iobenguane, iodocholesterol, minretumomab, tositumomab, and the like); ¹⁸F compounds (such as, e.g., florbetapir, flutemetamol, fluciclovine, fludeoxyghcose, fluoroethyltyrosine, sodium fluoride, and the like); ⁶⁴Cu compounds (such as, e.g., Cu-ETS2, and the like); ⁷⁵Se compounds (such as, e.g. SeHCAT); ¹¹¹In compounds (such as, e.g., imciromab, capromab pendetide, satumomab pendetide, aid the like); ⁸²Rb compounds (such as, e.g., rubidium chloride); ¹⁵³Sm compounds (such as, e.g., lexidronam, and the like); ⁸⁹Sr compounds (such as, e.g., strontium-89 chloride, and the like): ⁹⁰Y compounds (such as, e.g., ibritumomab tiuxetan, and the like); ²²³Ra compounds (such as, e.g., radium-223 chloride, and the like): ¹⁷⁷Lu compounds (such as, e.g., oxodotreotide, and the like); and any compounds comprising at least one ²H, ³H, ¹¹C, ¹³N, ¹⁴C, ¹⁵O, ¹⁸F, ²²Na, ²⁴Na, ³²P, ⁴⁷Ca, ⁵¹Cr, ⁵⁷Co, ⁵⁸Co, ⁵⁹Fe, ⁶⁴Cu, ⁶⁷Ga, ⁶⁸Ga, ⁷⁵Se, ⁸¹mKr, ⁸²Rb, ⁸⁹Sr, ⁹⁰Y, ^{99m}Tc, ¹¹¹In, ¹²³I, ¹²⁵I, ¹³¹I , ¹²²Xe, ¹⁵³Sm, ¹⁶⁵Dv, ¹⁶⁹Er, ¹⁷⁷Lu, ¹⁵Re, ¹⁹⁸Au, ²⁰¹Tl and/or ²²³Ra atom.

Examples of contrast agents include, but are not limited to, diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, calcium iopodate, iopydol, propyliodone, iofendylate, lipiodol, barium sulfate, gadobenic acid, gadobutrol, gadodiamide, gadofosveset, gadolinium, gadopentetic acid, gadoteric acid, gadoteridol, gadoversetamide, gadoxetic acid, ferric ammonium citrate, mangafodipir, ferumoxsil, ferristene, perflubron, microspheres of human albumin, microparticles of galactose, perflenapent, microspheres of phospholipids, sulfur hexafluoride, and the like.

A further object of the present invention is a composition comprising the antibody or a binding fragment thereof, binding to hCD45RC according to the present invention.

A further object of the present invention is a composition comprising at least one nucleic acid encoding the antibody or binding fragment thereof binding to hCD45RC according to the present invention.

A further object of the present invention is a composition comprising at least one expression vector comprising at least one nucleic acid encoding the antibody or binding fragment thereof binding to hCD45RC according to the present invention.

A further object of the present invention is a pharmaceutical composition comprising the antibody or a binding fragment thereof, binding to hCD45RC according to the present invention; and at least one pharmaceutically acceptable excipient.

A further object of the present invention is a pharmaceutical composition comprising at least one nucleic acid encoding the antibody or binding fragment thereof binding to hCD45RC according to the present invention; and at least one pharmaceutically acceptable excipient.

A further object of the present invention is a pharmaceutical composition comprising at least one expression vector comprising at least one nucleic acid encoding the antibody or binding fragment thereof binding to hCD45RC according to the present invention; and at least one pharmaceutically acceptable excipient or vehicle.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

A further object of the present invention is a medicament comprising the antibody or a binding fragment thereof, binding to hCD45RC according to the present invention.

A further object of the present invention is a medicament comprising at least one nucleic acid encoding the antibody or binding fragment thereof binding to hCD45RC according to the present invention.

A further object of the present invention is a medicament comprising at least one expression vector comprising at least one nucleic acid encoding the antibody or binding fragment thereof binding to hCD45RC according to the present invention.

The present invention further relates to methods of inducing immune tolerance in a subject in need thereof, by administering the isolated antibody or binding fragment thereof, nucleic acid, expression vector, composition, pharmaceutical composition or medicament according to the present invention. It also relates to the isolated antibody or binding fragment thereof, nucleic acid, expression vector, composition, pharmaceutical composition or medicament according to the present invention, for use in inducing immune tolerance in a subject in need thereof.

The term “immune tolerance”, as used herein, relates to a state of unresponsiveness of the immune system to specific substances or tissues that have the capacity to elicit an immune response while preserving immune response against other substances or tissues.

The term “immune response”, as used herein, includes T cell-mediated and/or B cell-mediated immune responses. Exemplary immune responses include, but are not limited to, T cell responses (e.g., cytokine production and cellular cytotoxicity), but also immune responses that are indirectly effected by T cell activation (e.g., macrophages). Immune cells involved in the immune response include lymphocytes (such as B cells and T cells, including CD4⁺, CD8⁺, Tₕ1 and Tₕ2 cells), antigen presenting cells (e.g., professional antigen presenting cells such as dendritic cells), natural killer cells, myeloid cells (such as macrophages, eosinophils, mast cells, basophils, and granulocytes).

The present invention further relates to methods of depleting CD45RC^{high} cells in a subject in need thereof, by administering the isolated antibody or binding fragment thereof, nucleic acid, expression vector, composition, pharmaceutical composition or medicament according to the present invention. It also relates to the isolated antibody or binding fragment thereof, nucleic acid, expression vector, composition, pharmaceutical composition or medicament according to the present invention, for use in depleting CD45RC^{high} cells in a subject in need thereof.

The relative level of expression of hCD45RC is measured using cytometry. Three types of cells can be distinguished: cells presenting a high, intermediary or negative level of hCD45RC expression.

In one embodiment, the isolated antibody or binding fragment thereof, nucleic acid, expression vector, composition, pharmaceutical composition or medicament according to the present invention depletes CD45RC^{high} T cells. “CD45RC^{high} T cells” are T cells that express the CD45RC marker at a high level, as defined above. It is readily understood by the one skilled in the art that the isolated antibody or binding fragment thereof, nucleic acid, expression vector, composition, pharmaceutical composition or medicament according to the present invention that depletes CD45RC^{high} T cells may also be able to deplete other types of CD45RC^{high} cells, such as CD45RC^{high} NK cells or CD45RC^{high} B cells.

As used herein, the terms “deplete” or “depleting”, with respect to cells expressing CD45RC, refer to a measurable decrease in the number of cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the terms refer to a decrease in the number of CD45RC^{high} cells in a subject or in a sample to an amount below detectable limits. According to the present invention, the isolated antibody or binding fragment thereof, nucleic acid, expression vector, composition, pharmaceutical composition or medicament specifically mediates depletion of the effector cells strongly expressing CD45RC, in particular those designed as CD45RC^{high} T_{eff}.

In particular, said isolated antibody or binding fragment thereof, nucleic acid, expression vector, composition, pharmaceutical composition or medicament according to the present invention depletes CD45RC^{high} T cells by binding to hCD45RC and transducing pro-apoptotic signals and/or by activating antibody-dependent cell mediated cytotoxicity (ADCC), complement dependent cytotoxicity (CDC) or antibody dependent phagocytosis.

As used herein, the term “antibody-dependent cell-mediated cytotoxicity” or “ADCC” refers to a form of cytotoxicity in which secreted antibodies bound onto Fc receptors (FcRs) present on certain cytotoxic cells (e.g., NK cells, neutrophils, monocytes and macrophages) enable these cytotoxic effector cells to bind specifically to an antigen-bearing target cell and subsequently to kill the target cell. To assess ADCC activity of a molecule of interest, an in vitro ADCC assay, such as that described in U.S. Pat. No. 5,500,362 or U.S. Pat. No. 5,821,337, may be performed.

In some embodiments, the isolated antibody or binding fragment thereof, nucleic acid, expression vector, composition, pharmaceutical composition or medicament according to the present invention mediates complement dependent cytotoxicity.

The term “complement dependent cytotoxicity” or “CDC” refers to the lysis of a target cell in the presence of complement. Activation of the classical complement pathway is initiated by the binding of the first component of the complement system to antibodies which are bound to their cognate antigen. To assess complement activation, a CDC assay such as, e.g., the one described in Gazzano-Santoro et al., 1997. J Immunol Methods. 202(2):163-71, may be performed.

In a particular embodiment, the isolated antibody or binding fragment thereof according to the present invention may be conjugated to a cytotoxic agent or a growth inhibitory agent.

The present invention further relates to methods of expanding and/or potentiating regulatory T cells in a subject in need thereof, by administering the isolated antibody or binding fragment thereof, nucleic acid, expression vector, composition, pharmaceutical composition or medicament according to the present invention. It also relates to the isolated antibody or binding fragment thereof, nucleic acid, expression vector, composition, pharmaceutical composition or medicament according to the present invention, for use in expanding and/or potentiating regulatory T cells in a subject in need thereof.

As used herein, the term “expand” refers to the process of converting and/or amplifying a given population of cells (e.g., immune cells such as Tregs). Expansion of a population of cells can occur in vivo, in vitro or ex vivo.

As used herein, the term “potentiate” refers to the process of increasing the function of a given population of cells (e.g., increasing the suppressive capacity of Tregs cells). Potentiation of a population of cells can occur in vivo, in vitro or ex vivo.

“Regulatory T cells” or “Tregs” are T cells that suppress an abnormal or excessive immune response and play a role in immune tolerance. Tregs are typically “forkhead box P3 (Foxp3⁺) regulatory T cells” and/or “CD45RC^{low/-} cells”.

As used herein, the terms “forkhead box P3 (Foxp3⁺) regulatory T cells” and “CD45RC^{low/-} cells” refer to 0.1-10% of CD4⁺ and/or CD8⁺ T cells in humans and rodents whose characteristic marker is the transcription factor Foxp3.

In one embodiment, the methods and uses are for expanding and/or potentiating Foxp3⁺ and/or CD45RC^{low/-} Tregs.

In one embodiment, CD45RC^{low/-} Tregs are expanded by stimulation. In one embodiment, CD45^{low/-} Tregs are expanded by stimulation in the presence of IL-2 and IL-15. In one embodiment, CD45RC^{low/-} Tregs are expanded by stimulation with anti-CD3/anti-CD28 antibodies and/or allogeneic antigen-presenting cells (APCs) and/or specific antigens.

Additionally or alternatively, the invention relates to in vitro or ex vivo methods of purifying CD45RC^{low/-} Tregs.

In one embodiment, CD45RC^{low/-} Tregs are CD8⁺/CD4⁺ T cells. In one embodiment, CD45RC^{low/-} Tregs are CD8⁺/CD4⁻ T cells. In one embodiment, CD45RC^{low/-} Tregs are CD8⁻/CD4⁺ T cells.

In one embodiment, purified CD45RC^{low/-} Tregs can be further expanded and/or potentiated prior to, concomitantly with or after administration to a subject in need thereof.

The present invention further relates to methods of preventing and/or reducing transplant rejections, by administering to a subject in need thereof the isolated antibody or binding fragment thereof, nucleic acid, expression vector, composition, pharmaceutical composition or medicament according to the present invention. It also relates to the isolated antibody or binding fragment thereof, nucleic acid, expression vector, composition, pharmaceutical composition or medicament according to the present invention, for use in preventing and/or reducing transplant rejections in a subject in need thereof.

The terms “preventing transplant rejections” and “reducing transplant rejections” are meant to encompass prevention or inhibition of immune transplant rejection, as well as delaying the onset or the progression of immune transplant rejection. The terms are also meant to encompass prolonging survival of a transplant in a subject, or reversing failure of a transplant in a subject. Further, the terms are meant to encompass ameliorating a symptom of an immune transplant rejection, including, for example, ameliorating an immunological complication associated with immune rejection, such as, e.g., interstitial fibrosis, chronic graft arteriosclerosis, or vasculitis.

The term “transplantation” and variations thereof refer to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an isograft, derived from a human of the same or different genetic origins.

In another scenario, the graft is derived from a species different from that into which it is transplanted, including animals from phylogenically widely separated species, for example, a baboon heart being transplanted into a human host.

The term “transplant rejection”, as used herein, encompasses both acute and chronic transplant rejection.

“Acute rejection” is the rejection by the immune system of a tissue transplant-recipient when the transplanted tissue is immunologically foreign. Acute rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute rejection can be inhibited or suppressed with immunosuppressive drugs such as rapamycin, cyclosporin, anti-CD40L monoclonal antibody and the like.

“Chronic rejection” generally occurs in humans within several months to years after engraftment, even in the presence of successful immunosuppression of acute rejection. Fibrosis is a common factor in chronic rejection of all types of organ transplants.

In one embodiment, the transplant rejection is an allogeneic transplant rejection. Accordingly, in one embodiment, the donor of the transplant is a human. The donor of the transplant can be a living donor or a deceased donor, namely a cadaveric donor.

In one embodiment, the transplant is an organ, a tissue or cells.

As used herein, the term “organ” refers to a solid vascularized organ that performs a specific function or group of functions within an organism. The term organ includes, but is not limited to, heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus.

As used herein, the term “tissue” refers to any type of tissue in human or animals, and includes, but is not limited to, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, ophthalmic tissue.

As used herein, the term “cells” refers to a composition enriched for cells of interest, preferably a composition comprising at least 30%, preferably at least 50%, even more preferably at least 65% of said cells.

In one embodiment, the “cells” are selected from the group comprising or consisting of: multipotent hematopoietic stem cells derived from bone marrow, peripheral blood, or umbilical cord blood; or pluripotent (i.e., embryonic stem cells [ES] or induced pluripotent stem cells [iPS]) or multipotent stem cell-derived differentiated cells of different cell lineages, including, but not limited to, cardiomyocytes, β-pancreatic cells, hepatocytes, neurons and the like.

In one embodiment where the transplantation is an allogeneic hematopoietic stem cell transplantation (HSCT), the “cells” are selected from the group comprising or consisting of: multipotent hematopoietic stem cells, usually derived from bone marrow, peripheral blood, or umbilical cord blood.

“HSCT” or “hematopoietic stem cell transplantation” is a transplantation therapy which can be curative for patients affected with leukemia and lymphomas (including, without limitation, acute myeloid leukemia (AML), acute lymphoid leukemia (ALL), chronic myeloid leukemia (CML), myelodysplasia syndrome (MDS), myeloproliferative syndrome, Hodgkin lymphomas, non-Hodgkin lymphomas, chronic lymphatic leukemia (CLL) and multiple myeloma). However, an important limitation of allogeneic HSCT is the development of graft-versus-host-disease (GVHD), which occurs in a severe form in about 30-50% of humans who receive this therapy.

Therefore, in one embodiment, the isolated antibody or binding fragment thereof, nucleic acid, expression vector, composition, pharmaceutical composition or medicament according to the present invention is used to prevent and/or reduce GVHD.

In a further embodiment, the isolated antibody or binding fragment thereof, nucleic acid, expression vector, composition, pharmaceutical composition or medicament according to the present invention may be used in combination with multipotent hematopoietic stem cells to prevent and/or treat leukemia and/or lymphomas (including, without limitation, acute myeloid leukemia (AML), acute lymphoid leukemia (ALL), chronic myeloid leukemia (CML), myelodysplasia syndrome (MDS), myeloproliferative syndrome, Hodgkin lymphomas, non-Hodgkin lymphomas, chronic lymphatic leukemia (CLL) and multiple myeloma).

Additionally or alternatively, the isolated antibody or binding fragment thereof, nucleic acid, expression vector, composition, pharmaceutical composition or medicament according to the present invention may be used may be used for graft engineering.

In one embodiment, the transplant to be grafted is treated with the isolated antibody or binding fragment thereof, nucleic acid, expression vector, composition, pharmaceutical composition or medicament according to the present invention prior to transplantation, to deplete CD45RC^{high} cells.

In a preferred embodiment, the transplant is bone marrow, and is treated with the isolated antibody or binding fragment thereof, nucleic acid, expression vector, composition, pharmaceutical composition or medicament according to the present invention prior to transplantation to deplete CD45RC^{high} T cells. In one embodiment, the bone marrow comprises CD34⁺ cells containing CD45RC^{high} T cells and CD45RC^{low/-} T cells.

The present invention further relates to methods of preventing, reducing and/or treating hCD45RC^{high}-related diseases, disorders or conditions, by administering to a subject in need thereof the isolated antibody or binding fragment thereof, nucleic acid, expression vector, composition, pharmaceutical composition or medicament according to the present invention. It also relates to the isolated antibody or binding fragment thereof, nucleic acid, expression vector, composition, pharmaceutical composition or medicament according to the present invention, for use in preventing and/or treating hCD45RC^{high}-related diseases, disorders or conditions.

As used herein, the term “hCD45RC^{high}-related diseases, disorders or conditions” refers to diseases, disorders or conditions caused by or potentialized by an increased proportion of cells expressing hCD45RC cells in a subject and/or by an increased level of expression of hCD45RC in cells of the subject.

By “increased proportion of CD45RC^{high} cells in a subject” is meant an increase of about 5%, preferably about 10%, 15%, 20%, 25%, 30%, 35%, 40%, 45%, 50%, 60%, 70%, 80%, 90%, 100% or more of the number of cells expressing CD45RC (i.e., CD45RC^{high} cells) in a given subject as compared to a reference, such as, e.g., the number of CD45RC^{high} cells in a substantially healthy subject.

By “increased level of expression of hCD45RC in cells of the subject” is meant an increase of about 5%, preferably about 10%, 15%, 20%, 25%, 30%, 35%, 40%, 45%, 50%, 60%, 70%, 80%, 90%, 100% or more of the expression level of hCD45RC, whether at the mRNA level or at the protein level, in cells of a given subject as compared to a reference, such as, e.g., the expression level of hCD45RC in cells of a substantially healthy subject.

In one embodiment, hCD45RC^{high}-related diseases, disorders or conditions are selected from the group comprising or consisting of autoimmune diseases, undesired immune responses, monogenic diseases and lymphoma or cancer.

In one embodiment, hCD45RC^{high}-related diseases, disorders or conditions are selected from the group comprising or consisting of autoimmune diseases, undesired immune responses and monogenic diseases.

As used herein, the term “autoimmune disease” refers to a disease in which the immune system produces an immune response (e.g., a B cell or a T cell response) against an antigen that is part of the normal host (that is an auto-antigen), with consequent injury to tissues. In an autoimmune disease, the immune system of the host fails to recognize a particular antigen as “self” and an immune reaction is mounted against the host's tissues expressing the antigen.

Exemplary autoimmune diseases contemplated in the present invention include, but are not limited to, rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (including Crohn's disease and ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, acquired hemophilia, thrombotic thrombocytopenic purpura, uveitis, IgG4-associated autoimmune diseases (such as, e.g., diseases listed in Table 1 of Kleger et al., 2015. Dtsch Arztebl Int. 112(8):128-135, which Table is incorporated by reference) and the like.

In a preferred embodiment, the autoimmune disease is systemic lupus erythematosis.

In a preferred embodiment, the autoimmune disease is inflammatory bowel disease, including Crohn's disease and ulcerative colitis. In a preferred embodiment, the autoimmune disease is Crohn's disease. In a preferred embodiment, the autoimmune disease is ulcerative colitis.

As used herein, the term “undesired immune response” refers to any unwanted immune reaction, preferably any unwanted immune reaction directed to (i) proteins expressed in the course of gene therapy, (ii) vectors (such as, e.g., viral vectors) used in the course of gene therapy and/or (iii) therapeutic proteins. Such proteins include for example factor VIII (hemophilia A) and other coagulation factors, enzyme replacement therapies, monoclonal antibodies (e.g., natalizumab, rituximab, infliximab), polyclonal antibodies, enzymes and cytokines (e.g., IFNβ). The term “undesired immune response” also refers to allergies and allergic reactions.

In one embodiment, the isolated antibody or binding fragment thereof, nucleic acid, expression vector, composition, pharmaceutical composition or medicament according to the present invention may be administered to a subject in order to suppress an immune response, especially to prevent immune reactions to specific proteins when their expression is restored by gene therapy in those subjects with corresponding genetic deficiencies. Thus, the isolated antibody or binding fragment thereof, nucleic acid, expression vector, composition, pharmaceutical composition or medicament according to the present invention may be used to prevent immune reactivity towards proteins normally absent in the subject due to mutations, while their reconstitution is achieved by gene therapy. Moreover, protein therapy is an area of medical innovation that is becoming more widespread, and involves the application of proteins, such as enzymes, antibodies or cytokines, directly to subjects as therapeutic products. One of the major hurdles in delivery of such medicaments involves the immune responses directed against the therapeutic protein themselves. Administration of protein-based therapeutics is often accompanied by administration of immune suppressants, which are used in order to facilitate a longer lifetime of the protein and therefore increased uptake of the protein into the cells and tissues of the organism. General immune suppressants can however be disadvantageous due to the unspecific nature of the immune suppression that is carried out, resulting in unwanted side effects in the patient. Therefore, this approach can be applied to suppress an immune response against therapeutic proteins and peptides, such as therapeutic antibodies, cytokines, enzymes or any other protein administered to a patient.

In one embodiment, the isolated antibody or binding fragment thereof, nucleic acid, expression vector, composition, pharmaceutical composition or medicament according to the present invention may be administered to a subject in order to suppress an immune response, especially to prevent immune reactions to vectors used in gene therapy, in particular viral vectors used in gene therapy. Such viral vectors include, e.g., adeno-associated virus (AAV) vectors, adenoviral (Ad) vectors, lentiviral vectors and the like. For a review, see Nayak & Herzog, 2010. Gene Ther. 17(3):295-304.

As used herein, the term “allergy” or “allergies” refers to an improper reaction of the immune system. Allergic reactions occur to normally harmless environmental substances known as allergens; these reactions are acquired, predictable and rapid. Strictly, allergy is one of four forms of hypersensitivity and is called type I (or immediate) hypersensitivity. It is characterized by excessive activation of certain white blood cells called mast cells and basophils by a type of antibody known as IgE, resulting in an extreme inflammatory response. Common allergic reactions include eczema, hives, hay fever, asthma, food allergies, and reactions to the venom of stinging insects such as wasps and bees.

The term “monogenic diseases”, as used herein, refers to diseases resulting from a mutation in a single gene selected among the following genes:
(i) genes which are not associated with immune function but whose deficiency is associated with inflammation and/or immune reactions, such as genes deficient in the following diseases: Duchenne muscular dystrophy (DMD), cystic fibrosis, lysosomal diseases and al-anti-trypsin deficiency; and
(ii) genes involved in the immune system and whose deficiency generates inflammation and/or autoimmune reactions, such as genes deficient in the following diseases: T cell primary immunodeficiency such as IPEX (immunodysregulation polyendocrinopathy enteropathy X-linked syndrome), APECED (autoimmune polyendocrinopathy-candidiasis-ectodermal dystrophy), B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome.

In a preferred embodiment, the autoimmune disease is APECED (autoimmune polyendocrinopathy-candidiasis-ectodermal dystrophy).

In a preferred embodiment, the autoimmune disease is Duchenne muscular dystrophy (DMD).

The term “lymphoma or cancer”, as used herein, encompass lymphoma or cancer which are associated with CD45RC^{high} cells. Exemplary lymphoma or cancer associated with CD45RC^{high} cells include, but are not limited to, acute myeloid leukemia (AML), acute lymphoid leukemia (ALL), chronic myeloid leukemia (CML), myelodysplasia syndrome (MDS)/myeloproliferative syndrome, lymphomas (such as, e.g., Hodgkin and non-Hodgkin lymphomas), chronic lymphatic leukemia (CLL) and multiple myeloma.

The present invention relates therefore to a method of depleting CD45RC^{high} cells in a subject in need thereof, thereby expending and/or potentiating regulatory T cells, preferably Foxp3⁺ and/or CD45RC^{ow} Tregs, thereby preventing and/or reducing transplant rejections; or preventing, reducing and/or treating hCD45RC^{high}-related diseases, disorders or conditions, by administering the isolated antibody or binding fragment thereof, nucleic acid, expression vector, composition, pharmaceutical composition or medicament according to the present invention.

In a preferred embodiment, the hCD45RC^{high}-related disease, disorder or condition is systemic lupus erythematosis, inflammatory bowel disease (including Crohn's disease and ulcerative colitis), APECED (autoimmune polyendocrinopathy-candidiasis-ectodermal dystrophy), or Duchenne muscular dystrophy (DMD).

In a preferred embodiment, the hCD45RC^{high}-related disease, disorder or condition is systemic lupus erythematosis, inflammatory bowel disease (including Crohn's disease and ulcerative colitis), or APECED (autoimmune polyendocrinopathy-candidiasis-ectodermal dystrophy).

It also relates to the isolated antibody or binding fragment thereof, nucleic acid, expression vector, composition, pharmaceutical composition or medicament according to the present invention for use in depleting CD45RC^{high} cells in a subject in need thereof, thereby expending and/or potentiating regulatory T cells, preferably Foxp3⁺ and/or CD45RC^{OW} Tregs, thereby preventing and/or reducing transplant rejections; or preventing, reducing and/or treating hCD45RC^{high}-related diseases, disorders or conditions.

In one embodiment, the isolated antibody or binding fragment thereof, nucleic acid, expression vector, composition, pharmaceutical composition or medicament according to the present invention will be formulated for administration to the subject.

In one embodiment, the isolated antibody or binding fragment thereof, nucleic acid, expression vector, composition, pharmaceutical composition or medicament according to the present invention is to be administered systemically or locally.

In one embodiment, the isolated antibody or binding fragment thereof, nucleic acid, expression vector, composition, pharmaceutical composition or medicament according to the present invention is to be administered by injection, orally, topically, nasally, buccally, rectally, vaginaly, intratracheally, by endoscopy, transmucosally, or by percutaneous administration.

In one embodiment, the isolated antibody or binding fragment thereof, nucleic acid, expression vector, composition, pharmaceutical composition or medicament according to the present invention is to be injected, preferably systemically injected.

Examples of formulations adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of systemic injections include, but are not limited to, intravenous (iv), subcutaneous, intramuscular (im), intradermal (id), intraperitoneal (ip) injection and perfusion.

In one embodiment, when injected, the isolated antibody or binding fragment thereof, nucleic acid, expression vector, composition, pharmaceutical composition or medicament according to the present invention is sterile. Methods for obtaining a sterile composition include, but are not limited to, GMP synthesis (where GMP stands for “Good manufacturing practice”).

Sterile injectable forms of a composition may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

It will be understood that other suitable routes of administration are also contemplated in the present invention, and the administration mode will ultimately be decided by the attending physician within the scope of sound medical judgment. Apart from administration by injection (iv, ip, im and the like), other routes are available, such as nebulization (Respaud et al., 2014. MAbs. 6(5):1347-55; Guilleminault et al., 2014. J Control Release. 196:344-54; Respaud et al., 2015. Expert Opin Drug Deliv. 12(6):1027-39) or subcutaneous administration (Jackisch et al., 2014. Geburtshilfe Frauenheilkd. 74(4):343-349; Solal-Celigny, 2015. Expert Rev Hematol. 8(2):147-53).

In one embodiment, the isolated antibody or binding fragment thereof, nucleic acid, expression vector, composition, pharmaceutical composition or medicament according to the present invention is to be administered to the subject in need thereof in a therapeutically effective amount.

The term “therapeutically effective amount”, as used herein, refers to an amount effective, at dosages and for periods of time necessary, to achieve a desired preventive and/or therapeutic result.

It will be however understood that the total daily usage of the isolated antibody or binding fragment thereof, nucleic acid, expression vector, composition, pharmaceutical composition or medicament according to the present invention will be decided by the attending physician within the scope of sound medical judgment. The specific therapeutically effective dose level for any particular patient will depend upon a variety of factors including the disease being treated and the severity of the disease; activity of the isolated antibody or binding fragment thereof, nucleic acid, expression vector, composition, pharmaceutical composition or medicament employed; the age, body weight, general health, sex and diet of the subject; the time of administration, route of administration, and rate of excretion of the specific isolated antibody or binding fragment thereof, nucleic acid, expression vector, composition, pharmaceutical composition or medicament employed; the duration of the treatment; drugs used in combination or coincidental with the specific isolated antibody or binding fragment thereof, nucleic acid, expression vector, composition, pharmaceutical composition or medicament employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, a therapeutically effective amount of the isolated antibody or binding fragment thereof, nucleic acid, expression vector, composition, pharmaceutical composition or medicament according to the present invention ranges from about 0.1 mg/kg to about 5 mg/kg, from about 0.2 mg/kg to about 4 mg/kg, from about 0.3 mg/kg to about 3 mg/kg, from about 0.4 mg/kg to about 2.5 mg/kg, from about 0.5 mg/kg to about 2 mg/kg.

In one embodiment, a therapeutically effective amount of the isolated antibody or binding fragment thereof, nucleic acid, expression vector, composition, pharmaceutical composition or medicament according to the present invention ranges from about 10 μg/kg to about 400 μg/kg, from about 20 μg/kg to about 300 μg/kg, from about 30 μg/kg to about 250 μg/kg, from about 35 μg/kg to about 200 μg/kg, from about 40 μg/kg to about 160 μg/kg.

In one embodiment, a therapeutically effective amount of the isolated antibody or binding fragment thereof, nucleic acid, expression vector, composition, pharmaceutical composition or medicament according to the present invention is to be administered once a day, twice a day, three times a day or more.

In one embodiment, a therapeutically effective amount of the isolated antibody or binding fragment thereof, nucleic acid, expression vector, composition, pharmaceutical composition or medicament according to the present invention is to be administered every day, every two days, every three days, every four days, every five days, every six days.

In one embodiment, a therapeutically effective amount of the isolated antibody or binding fragment thereof, nucleic acid, expression vector, composition, pharmaceutical composition or medicament according to the present invention is to be administered every week, every two weeks, every three weeks.

In one embodiment, a therapeutically effective amount of the isolated antibody or binding fragment thereof, nucleic acid, expression vector, composition, pharmaceutical composition or medicament according to the present invention is to be administered every month, every two months, every three months, every four months, every five months, every six months.

In a preferred embodiment, a therapeutically effective amount of the isolated antibody or binding fragment thereof, nucleic acid, expression vector, composition, pharmaceutical composition or medicament according to the present invention is to be administered every 12 hours, every 24 hours, every 36 hours, every 48 hours, every 60 hours, every 72 hours, every 96 hours.

In a preferred embodiment, a therapeutically effective amount of the isolated antibody or binding fragment thereof, nucleic acid, expression vector, composition, pharmaceutical composition or medicament according to the present invention is to be administered every 60 hours.

In one embodiment, the isolated antibody or binding fragment thereof, nucleic acid, expression vector, composition, pharmaceutical composition or medicament according to the present invention is for acute administration. In one embodiment, the isolated antibody or binding fragment thereof, nucleic acid, expression vector, composition, pharmaceutical composition or medicament according to the present invention is for chronic administration.

In one embodiment, a therapeutically effective amount of the isolated antibody or binding fragment thereof, nucleic acid, expression vector, composition, pharmaceutical composition or medicament according to the present invention is to be administered for about 5 days, 7 days, 10 days, 14 days, 21 days, 28 days, 1 month, 2 months, 3 months, 6 months, 1 year or more.

In one embodiment, a therapeutically effective amount of the isolated antibody or binding fragment thereof, nucleic acid, expression vector, composition, pharmaceutical composition or medicament according to the present invention is to be administered for a period of time ranging from about one week to about eight weeks, from about two weeks to about seven weeks, from about two weeks to about six weeks, from about two weeks to about five weeks.

In a preferred embodiment, a therapeutically effective amount of the isolated antibody or binding fragment thereof, nucleic acid, expression vector, composition, pharmaceutical composition or medicament according to the present invention is to be administered for a period of time ranging from about 10 days to about 40 days, from about 15 days to about 35 days, from about 20 days to about 30 days.

In one embodiment, the isolated antibody or binding fragment thereof, nucleic acid, expression vector, composition, pharmaceutical composition or medicament according to the present invention is to be administered before, concomitantly with or after a therapeutic drug.

Some examples of therapeutic drugs suitable for co-administration with the isolated antibody or binding fragment thereof, nucleic acid, expression vector, composition, pharmaceutical composition or medicament according to the present invention include, without limitation, immunosuppressants, cytotoxins, chemotherapeutic agents, cytokines, immune stimulators, lytic peptides and radioisotopes.

It will be understood by the one skilled in the art that the co-administration of the isolated antibody or binding fragment thereof, nucleic acid, expression vector, composition, pharmaceutical composition or medicament according to the present invention with a particular therapeutic drug, which may be chosen among those recited herein but without being limited thereto, will depend on the disease or condition to be prevented and/or treated.

Examples of immunosuppressants include, without limitation, mTOR inhibitors such as, e.g., sirolimus, everolimus, ridaforolimus, temsirolimus, umirolimus and zotarolimus; IL-1 receptor antagonists such as, e.g., anakinra; antimetabolites such as, e.g., azathioprine, leflunomide, methotrexate, mycophenolic acid and teriflunomide; IMiDs such as, e.g., apremilast, lenalidomide, pomalidomide and thalidomide; and antibodies such as, e.g., eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, zolimomab aritox, atorolimumab, cedelizumab, fontolizumab, maslimomab, morolimumab, pexelizumab, reslizumab, rovelizumab, siplizumab, talizumab, telimomab aritox, vapaliximab, vepalimomab, abatacept, belatacept, etanercept, pegsunercept, aflibercept, alefacept and rilonacept.

Examples of cytotoxins include, without limitation, radionuclides (e.g., ³⁵S, ¹⁴C, ³²P, ³⁵P, ¹²⁵I, ¹³¹I, ⁹⁰Y, ⁸⁸Zr, ²⁰¹Tl, ¹⁸⁶Re, ¹⁸⁸Re, ⁵⁷Cu, ²¹³Bi, and ²¹¹At, conjugated radionuclides, and chemotherapeutic agents. Further examples of cytotoxins include, but are not limited to, antimetabolites (e.g., 5-fluorouricil (5-FU), methotrexate (MTX), fludarabine, etc.), anti-microtubule agents (e.g., vincristine, vinblastine, colchicine, taxanes (such as paclitaxel and doctaxel), etc.), alkylating agents (e.g. cyclophosphamide, melphalan, bischloroethylnitrosurea (BCNU), etc.), platinum agents (e.g., cisplatin (also termed cDDP), carboplatin, oxaliplatin, JM-216, C-973, etc.), anthracyclines (e.g., doxorubicin, daunorubicin, etc.), antibiotic agents (e.g., mitomycin-C), topoisomerase inhibitors (e.g., etoposide, tenoposide, and camptothecins), or other cytotoxic agents such as ricin, diptheria toxin (DT), Pseudomonas exotoxin (PE) A, PE40, abrin, saporin, pokeweed viral protein, ethidium bromide, glucocorticoid, anthrax toxin and others.

Examples of chemotherapeutic agents include, without limitation, platinum coordination compounds (such as, e.g., cisplatin, carboplatin or oxalyplatin); taxane compounds (such as, e.g., paclitaxel or docetaxel); topoisomerase I inhibitors (such as, e.g., irinotecan or topotecan); topoisomerase II inhibitors (such as, e.g., etoposide or teniposide); vinca alkaloids (such as, e.g., vinblastine, vincristine or vinorelbine); anti-tumor nucleoside derivatives (such as, e.g., 5-fluorouracil, gemcitabine or capecitabine); alkylating agents (such as, e.g., nitrogen mustard or nitrosourea, cyclophosphamide, chlorambucil, carmustine or lomustine; anti-tumor anthracycline derivatives (suc has, e.g., daunorubicin, doxorubicin, idarubicin or mitoxantrone); anti-HER2 antibodies (such as, e.g., trastuzumab); estrogen receptor antagonists or selective estrogen receptor modulators (such as, e.g., tamoxifen, toremifene, droloxifene, faslodex or raloxifene); aromatase inhibitors (such as, e.g., exemestane, anastrozole, letrazole or vorozole); differentiating agents (such as, e.g., retinoids, vitamin D and retinoic acid metabolism blocking agents [RAMBA] such as accutane); DNA methyl transferase inhibitors (such as, e.g., azacytidine); kinase inhibitors (such as, e.g., flavopiridol, imatinib mesylate or gefitinib); farnesyltransferase inhibitors; and HDAC inhibitors.

Examples of cytokines include, without limitation, chemokines (such as, e.g., CCL1, CCL2/MCP1, CCL3/MIP1α, CCL4/MIP13, CCL5/RANTES, CCL6, CCL7, CCL8, CCL9, CCL11, CCL12, CCL13, CCL14, CCL15, CCL16, CCL17, CCL18/PARC/DCCK1/AMAC1/MIP4, CCL19, CCL20, CCL21, CCL22, CCL23, CCL24, CCL25, CCL26, CCL27, CCL28, CXCL1/KC, CXCL2, CXCL3, CXCL4, CXCL5, CXCL6, CXCL7, CXCL8/IL8, CXCL9, CXCL10, CXCL11, CXCL12, CXCL13, CXCL14, CXCL15, CXCL16, CXCL17, CX3CL1, XCL1 and XCL2), tumor necrosis factors (such as, e.g., TNFA, Lymphotoxin, TNFSF4, TNFSF5/CD40LG, TNFSF6, TNFSF7, TNFSF8, TNFSF9, TNFSF10, TNFSF11, TNFSF13, TNFSF13B and EDA) and interleukins (such as, e.g., IL-la, IL-10, IL-1Ra, IL-2, IL-3, IL-4, IL-5, IL-6, IL-7, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36α, IL-36β, IL-36γ, IL-36Ra, IL-37, IL-38, IFNα, IFNβ, IFNκ, IFNω and GM-CSF).

Examples of immune stimulators include, without limitation, filgrastim, pegfilgrastim, lenograstim, molgramostim, sargramostim, ancestim, albinterferon, interferon alfa, peginterferon alfa, interferon beta, peginterferon beta, interferon gamma, aldesleukin, oprelvekin, growth hormone, immunocyanin, pegademase, prolactin, tasonermin, histamine dihydrochloride, poly ICLC, vitamin D, lentinan, plerixafor, roquinimex, mifamurtide, glatiramer acetate, thymopentin, thymosin al, thymulin, polyinosinic:polycytidylic acid, pidotimod, Bacillus Calmette-Guérin vaccine, melanoma vaccine and sipuleucel-T vaccine.

Examples of lytic peptides include, without limitation, toxins (such as, e.g., Diptheria toxin or Pseudomonas exotoxin).

Examples of radioisotopes include, without limitation, the radionuclides of technetium (e.g., Tc-99 and Tc-97), potassium (e.g., K-40), rubidium (e.g., Rb-82), iodine (e.g., I-123, I-124, I-125, I-129, I-131), cesium (e.g., Cs-135, Cs-137), cobalt (e.g., Co-60), palladium (e.g., Pd-103, Pd-107), cadmium (e.g., Cd-113), strontium (e.g., Sr-89, Sr-90), europium (e.g., Eu-55), tin (e.g., Sn-121, Sn-126), phosphorus (e.g., P-32, P-33), thallium (e.g., T1-201), indium (e.g., In-111), gallium (e.g., Ga-67, Ga-68), yttrium (e.g., Y-90), iridium (e.g., Ir-192), bismuth (e.g., Bi-213), radium (e.g., Ra-223, Ra-225), and ruthenium (e.g., Ru-106).

The invention further relates to the use of the isolated antibody or binding fragment thereof according to the present invention, for detecting or quantifying hCD45RC in a sample, cell, tissue or organ; and to methods for detecting or quantifying hCD45RC in a sample, cell, tissue or organ, comprising contacting said sample, cell, tissue or organ with the isolated antibody or binding fragment thereof according to the present invention.

In one embodiment, the uses and methods for detecting or quantifying hCD45RC may be in vitro or in vivo.

In one embodiment, the isolated antibody or binding fragment thereof according to the present invention is labeled, as described hereinabove, for detection or diagnostic purposes.

Assays suitable for detecting or quantifying hCD45RC using the isolated antibody or binding fragment thereof according to the present invention are well-known in the art, and include, without limitation, ELISA, sandwich ELISA, RIA, FACS, tissue immunohistochemistry, Western-blot and immunoprecipitation.

In one embodiment, the sample is a biological sample. Examples of biological samples include, but are not limited to, bodily fluids (preferably blood, more preferably blood serum), plasma, synovial fluid, bronchoalveolar lavage fluid, sputum, lymph, ascitic fluids, urine, amniotic fluid, peritoneal fluid, cerebrospinal fluid, pleural fluid, pericardial fluid, and alveolar macrophages, tissue lysates, biopsies and extracts prepared from diseased tissues.

In one embodiment, the sample is taken or retrieved from a subject prior to any analysis. Accordingly, in this embodiment, the uses and methods for detecting or quantifying hCD45RC are in vitro uses and methods.

The invention further relates to the use of the isolated antibody or binding fragment thereof according to the present invention, for diagnosing hCD45RC-related diseases in a subject; and to methods for diagnosing hCD45RC-related diseases in a subject comprising contacting a sample from said subject with the isolated antibody or binding fragment thereof according to the present invention.

Assays suitable for detecting or quantifying hCD45RC using the isolated antibody or binding fragment thereof according to the present invention are well-known in the art, and include, without limitation, ELISA, sandwich ELISA, RIA, FACS, tissue immunohistochemistry, Western-blot and immunoprecipitation.

In one embodiment, the sample is a biological sample. Examples of biological samples include, but are not limited to, bodily fluids (preferably blood, more preferably blood serum), plasma, synovial fluid, bronchoalveolar lavage fluid, sputum, lymph, ascitic fluids, urine, amniotic fluid, peritoneal fluid, cerebrospinal fluid, pleural fluid, pericardial fluid, and alveolar macrophages, tissue lysates, biopsies and extracts prepared from diseased tissues.

In one embodiment, the sample is taken or retrieved from a subject prior to any analysis. Accordingly, in this embodiment, the uses and methods for diagnosing hCD45RC-related diseases are in vitro uses and methods.

In one embodiment, the hCD45RC-related diseases are hCD45RC^{high}-related diseases, as defined hereinabove. According to this embodiment, the subject may be diagnosed as affected with or suffering from a hCD45RC-related disease if hCD45RC is detected in a sample from said subject at a level, amount or concentration higher than in a reference subject (e.g., in a substantially healthy subject, or in a subject who is known not to be affected with or suffering from a hCD45RC-related disease).

In one embodiment, the uses and methods for diagnosing hCD45RC-related diseases further comprise a step of treating the subject if said subject is diagnosed as being affected with or suffering from a hCD45RC-related disease.

The present application discloses an isolated anti-human CD45RC antibody or binding fragment thereof, wherein said antibody or binding fragment thereof binds to human CD45RC with:
(i) an equilibrium dissociation constant (K_{D}) of 5×10⁻⁷ M or less;
(ii) an association rate (Kₒₙ) of 1×10⁴ M⁻¹ sec⁻¹ or more; and/or
(iii) a dissociation rate (K_{off}) of 5×10⁻² sec⁻¹ or less;
preferably wherein the K_{D}, Kₒₙ and/or K_{off} are determined by surface plasmon resonance.

The antibody or binding fragment thereof binds to at least one epitope displayed in the C determinant of hCD45RC.

The antibody or binding fragment thereof binds to at least one epitope comprising at least 5 amino acids of SEQ ID NO: 23 or a fragment thereof; or of a sequence sharing at least about 70% identity with SEQ ID NO: 23 or a fragment thereof.

The antibody is monoclonal.

The antibody is murine, chimeric or humanized.
a) The variable region of the heavy chain (HCVR) of the antibody or binding fragment thereof comprises the following three complementary-determining regions (CDRs):
   VH-CDR1: NYYIG (SEQ ID NO: 1);
   VH-CDR2: X₁-IF-X₂-GG-X₃-Y-X₄-N-X₅-X₆-X₇-X₈-X₉-X₁₀-G (SEQ ID NO: 2); and
   VH-CDR3: RNFDY (SEQ ID NO: 3), and
b) the variable region of the light chain (LCVR) of said antibody or binding fragment thereof comprises the following three CDRs:

VL-CDR1:
(SEQ ID NO: 12)
X₁₁-ASSSVS-X₁₂-YMH;
VL-CDR2:
(SEQ ID NO: 13)
X₁₃-TSN-X₁₄-X₁₅-X₁₆;
and
VL-CDR3:
(SEQ ID NO: 14)
X₁₇-QRSSYPLTF;

wherein:
X₁ is selected from Asp (D), Ile (I) and Arg (R);
X₂ is selected from Pro (P) and Ser (S);
X₃ is selected from Asp (D), Ser (S) and Gly (G);
X₄ is selected from Thr (T) and Ala (A);
X₅ is selected from Tyr (Y) and Ser (S);
X₆ is selected from Ala (A), Asn (N) and Ser (S);
X₇ is selected from Glu (E), Asp (D), Pro (P) and Gln (Q);
X₈ is selected from Lys (K) and Ser (S);
X₉ is selected from Phe (F) and Val (V);
X₁₀ is selected from Lys (K) and Gln (Q);
X₁₁ is selected from Arg (R) and Ser (S);
X₁₂ is selected from Asn (N), Ser (S), Gly (G) and an empty position;
X₁₃ is selected from Asn (N) and Ala (A); or X₁₃ being any amino acid but Ala (A) or Asn (N);
X₁₄ is selected from Len (L), Ser (S) and Arg (R);
X₁₅ is selected from Pro (P), Ala (A) and Gln (Q);
X₁₆ is selected from Ser (S) and Thr (T); and
X₁₇ is selected from Gln (Q) and His (H).

The antibody or binding fragment thereof comprises a combination of CDRs as set forth in Table 2.

The antibody or binding fragment thereof comprises:
a) a HCVR selected from SEQ ID NOs: 61-70; and
b) a LCVR selected from SEQ ID NOs: 71-90.

The antibody or binding fragment thereof comprises a combination of HCVR and LCVR selected from:
SEQ ID NOs: 61 and 71; SEQ ID NOs: 61 and 81; SEQ ID NOs: 62 and 72;
SEQ ID NOs: 62 and 73; SEQ ID NOs: 62 and 74; SEQ ID NOs: 62 and 82;
SEQ ID NOs: 62 and 83; SEQ ID NOs: 62 and 84; SEQ ID NOs: 63 and 72;
SEQ ID NOs: 63 and 73; SEQ ID NOs: 63 and 74; SEQ ID NOs: 63 and 82;
SEQ ID NOs: 63 and 83; SEQ ID NOs: 63 and 84; SEQ ID NOs: 64 and 72;
SEQ ID NOs: 64 and 73; SEQ ID NOs: 64 and 74; SEQ ID NOs: 64 and 82;
SEQ ID NOs: 64 and 83; and SEQ ID NOs: 64 and 84.
wherein X₁₂ in SEQ ID NOs 71-74 is selected from Asn (N), Ser (S) and Gly (G).

The present application also discloses nucleic acid encoding the isolated antibody or binding fragment thereof described above.

The present application also discloses an expression vector comprising the nucleic acid described above.

The present application also discloses a composition comprising the isolated antibody or binding fragment thereof described above, the nucleic acid described above, or the expression vector described above.

The present application also discloses a pharmaceutical composition comprising the isolated antibody or binding fragment thereof described above, the nucleic acid described above, or the expression vector described above, and at least one pharmaceutically acceptable excipient.

The present application also discloses the isolated antibody or binding fragment thereof described above, the nucleic acid described above, the expression vector described above, the composition described above or the pharmaceutical composition described above, for use as a medicament.

The isolated antibody or binding fragment thereof described above, the nucleic acid described above, the expression vector described above, the composition described above or the pharmaceutical composition described above is/are for use in:
inducing immune tolerance in a subject in need thereof;
preventing and/or reducing transplant rejections; and/or
preventing, reducing and/or treating CD45RC^{high}-related conditions, preferably selected from autoimmune diseases, undesired immune responses, monogenic diseases and lymphoma or cancer.

The isolated antibody or binding fragment thereof described above, the nucleic acid described above, the expression vector described above, the composition described above or the pharmaceutical composition described above is/are for use in preventing and/or treating graft-versus-host disease (GVHD).

### EXAMPLES

The present invention is further illustrated by the following examples.

Throughout the examples, the following nomenclature applies:

“ABIS-45RC”: the murine anti-hCD45RC antibody of the invention, comprising:
a heavy chain variable region with SEQ ID NO: 61;
a heavy chain constant region with SEQ ID NO: 93;
a light chain variable region with SEQ ID NO: 81; and
a light chain constant region with SEQ ID NO: 94.

“Anti-45RC Variant A”: a humanized variant of ABIS-45RC, comprising:
a heavy chain variable region with SEQ ID NO: 62;
a heavy chain constant region with SEQ ID NO: 91;
a light chain variable region with SEQ ID NO: 82; and
a light chain constant region with SEQ ID NO: 92.

“Anti-45RC Variant B”: a humanized variant of ABIS-45RC, comprising:
a heavy chain variable region with SEQ ID NO: 62;
a heavy chain constant region with SEQ ID NO: 91;
a light chain variable region with SEQ ID NO: 83; and
a light chain constant region with SEQ ID NO: 92.

“Anti-45RC Variant C”: a humanized variant of ABIS-45RC, comprising:
a heavy chain variable region with SEQ ID NO: 62;
a heavy chain constant region with SEQ ID NO: 91;
a light chain variable region with SEQ ID NO: 84; and
a light chain constant region with SEQ ID NO: 92.

“Anti-45RC Variant D”: a humanized variant of ABIS-45RC, comprising:
a heavy chain variable region with SEQ ID NO: 63;
a heavy chain constant region with SEQ ID NO: 91;
a light chain variable region with SEQ ID NO: 82; and
a light chain constant region with SEQ ID NO: 92.

“Anti-45RC Variant E”: a humanized variant of ABIS-45RC, comprising:
a heavy chain variable region with SEQ ID NO: 63;
a heavy chain constant region with SEQ ID NO: 91;
a light chain variable region with SEQ ID NO: 83; and
a light chain constant region with SEQ ID NO: 92.

“Anti-45RC Variant F”: a humanized variant of ABIS-45RC, comprising:
a heavy chain variable region with SEQ ID NO: 63;
a heavy chain constant region with SEQ ID NO: 91;
a light chain variable region with SEQ ID NO: 84; and
a light chain constant region with SEQ ID NO: 92.

“Anti-45RC Variant G”: a humanized variant of ABIS-45RC, comprising:
a heavy chain variable region with SEQ ID NO: 64;
a heavy chain constant region with SEQ ID NO: 91;
a light chain variable region with SEQ ID NO: 83; and
a light chain constant region with SEQ ID NO: 92.

“Anti-45RC Variant H”: a humanized variant of ABIS-45RC, comprising:
a heavy chain variable region with SEQ ID NO: 64;
a heavy chain constant region with SEQ ID NO: 91;
a light chain variable region with SEQ ID NO: 84; and
a light chain constant region with SEQ ID NO: 92.

“Anti-45RC Variant I”: a humanized variant of ABIS-45RC, comprising:
a heavy chain variable region with SEQ ID NO: 64;
a heavy chain constant region with SEQ ID NO: 91;
a light chain variable region with SEQ ID NO: 82; and
a light chain constant region with SEQ ID NO: 92.

“Anti-45RC Variant A1”: a humanized variant of ABIS-45RC, comprising:
a heavy chain variable region with SEQ ID NO: 101;
a heavy chain constant region with SEQ ID NO: 91;
a light chain variable region with SEQ ID NO: 85; and
a light chain constant region with SEQ ID NO: 92.

“Anti-45RC Variant A2”: a humanized variant of ABIS-45RC, comprising:
a heavy chain variable region with SEQ ID NO: 101;
a heavy chain constant region with SEQ ID NO: 91;
a light chain variable region with SEQ ID NO: 103; and
a light chain constant region with SEQ ID NO: 92.

“Anti-45RC Variant A3”: a humanized variant of ABIS-45RC, comprising:
a heavy chain variable region with SEQ ID NO: 65;
a heavy chain constant region with SEQ ID NO: 91;
a light chain variable region with SEQ ID NO: 85; and
a light chain constant region with SEQ ID NO: 92.

“Anti-45RC Variant A4”: a humanized variant of ABIS-45RC, comprising:
a heavy chain variable region with SEQ ID NO: 65;
a heavy chain constant region with SEQ ID NO: 91;
a light chain variable region with SEQ ID NO: 103; and
a light chain constant region with SEQ ID NO: 92.

“Anti-45RC Variant A5”: a humanized variant of ABIS-45RC, comprising:
a heavy chain variable region with SEQ ID NO: 62;
a heavy chain constant region with SEQ ID NO: 91;
a light chain variable region with SEQ ID NO: 85; and
a light chain constant region with SEQ ID NO: 92.

“Anti-45RC Variant A6”: a humanized variant of ABIS-45RC, comprising:
a heavy chain variable region with SEQ ID NO: 101;
a heavy chain constant region with SEQ ID NO: 91;
a light chain variable region with SEQ ID NO: 82; and
a light chain constant region with SEQ ID NO: 92.

“Anti-45RC Variant A7”: a humanized variant of ABIS-45RC, comprising:
a heavy chain variable region with SEQ ID NO: 121;
a heavy chain constant region with SEQ ID NO: 91;
a light chain variable region with SEQ ID NO: 85; and
a light chain constant region with SEQ ID NO: 92.

“Anti-45RC Variant A8”: a humanized variant of ABIS-45RC, comprising:
a heavy chain variable region with SEQ ID NO: 122;
a heavy chain constant region with SEQ ID NO: 91;
a light chain variable region with SEQ ID NO: 85; and
a light chain constant region with SEQ ID NO: 92.

“Anti-45RC Variant A9”: a humanized variant of ABIS-45RC, comprising:
a heavy chain variable region with SEQ ID NO: 123;
a heavy chain constant region with SEQ ID NO: 91;
a light chain variable region with SEQ ID NO: 85; and
a light chain constant region with SEQ ID NO: 92.

“Anti-45RC Variant A10”: a humanized variant of ABIS-45RC, comprising:
a heavy chain variable region with SEQ ID NO: 124;
a heavy chain constant region with SEQ ID NO: 91;
a light chain variable region with SEQ ID NO: 85; and
a light chain constant region with SEQ ID NO: 92.

“Anti-45RC Variant D1”: a humanized variant of ABIS-45RC, comprising:
a heavy chain variable region with SEQ ID NO: 63;
a heavy chain constant region with SEQ ID NO: 91;
a light chain variable region with SEQ ID NO: 85; and
a light chain constant region with SEQ ID NO: 92.

“Anti-45RC Variant I1”: a humanized variant of ABIS-45RC, comprising:
a heavy chain variable region with SEQ ID NO: 67;
a heavy chain constant region with SEQ ID NO: 91;
a light chain variable region with SEQ ID NO: 85; and
a light chain constant region with SEQ ID NO: 92.

“Anti-45RC Variant I2”: a humanized variant of ABIS-45RC, comprising:
a heavy chain variable region with SEQ ID NO: 67;
a heavy chain constant region with SEQ ID NO: 91;
a light chain variable region with SEQ ID NO: 103; and
a light chain constant region with SEQ ID NO: 92.

“MT2”: a murine anti-hCD45RC antibody commercially available at OriGene, under Ref. AM39022PU-N.

“Engineered Asn/Phe ABIS-45RC”: the murine ABIS-45RC, chimerized by engineering its LCVR by insertion of one residue in the CDR1 and substitution of one residue in the FR3 (as described in Example 8). Engineered Asn/Phe ABIS-45RC comprises:
a heavy chain variable region with SEQ ID NO: 61;
a heavy chain constant region with SEQ ID NO: 93;
a light chain variable region with SEQ ID NO: 71 with X₁₂ being Asn (N); and
a light chain constant region with SEQ ID NO: 94.

“Engineered Ser/Phe ABIS-45RC”: the murine ABIS-45RC, chimerized by engineering its LCVR by insertion of one residue in the CDR1 and substitution of one residue in the FR3 (as described in Example 8). Engineered Ser/Phe ABIS-45RC comprises:
a heavy chain variable region with SEQ ID NO: 61;
a heavy chain constant region with SEQ ID NO: 93;
a light chain variable region with SEQ ID NO: 71 with X₁₂ being Ser (S); and
a light chain constant region with SEQ ID NO: 94.

“Engineered Gly/Phe ABIS-45RC”: the murine ABIS-45RC, chimerized by engineering its LCVR by insertion of one residue in the CDR1 and substitution of one residue in the FR3 (as described in Example 8). Engineered Gly/Phe ABIS-45RC comprises:
a heavy chain variable region with SEQ ID NO: 61;
a heavy chain constant region with SEQ ID NO: 93;
a light chain variable region with SEQ ID NO: 71 with X₁₂ being Gly (G); and
a light chain constant region with SEQ ID NO: 94.

“Chimeric N50A ABIS-45RC”: a chimeric variant of the murine ABIS-45RC, comprising the murine ABIS-45RC light chain and a humanized heavy chain. Chimeric N50A ABIS-45RC comprises:
a heavy chain variable region with SEQ ID NO: 61;
a heavy chain constant region with SEQ ID NO: 93;
a light chain variable region with SEQ ID NO: 113; and
a light chain constant region with SEQ ID NO: 92.

“Chimeric S52A ABIS-45RC”: a chimeric variant of the murine ABIS-45RC, comprising the murine ABIS-45RC light chain and a humanized heavy chain. Chimeric S52A ABIS-45RC comprises:
a heavy chain variable region with SEQ ID NO: 61;
a heavy chain constant region with SEQ ID NO: 93;
a light chain variable region with SEQ ID NO: 126; and
a light chain constant region with SEQ ID NO: 92.

“Chimeric N50X ABIS-45RC”: a chimeric variant of the murine ABIS-45RC, comprising the murine ABIS-45RC light chain and a humanized heavy chain. Chimeric N50X ABIS-45RC comprises:
a heavy chain variable region with SEQ ID NO: 61;
a heavy chain constant region with SEQ ID NO: 93;
a light chain variable region with SEQ ID NO: 129, with X₁₃ being any amino acid but Ala (A) or Asn (N); and
a light chain constant region with SEQ ID NO: 92.

### Example 1

### Reactivity of ABIS-45RC

### Material and Methods

### PBMC Staining and Data Acquisition

50 μL or 100 μL of fresh EDTA whole blood were stained with combinations of appropriate monoclonal antibodies (Abs) followed by erythrocyte lysis (versalyse, Beckman Coulter). After washing, cells were analyzed on a Navios flow cytometer and data analyzed using Kaluza software (Beckman Coulter, Marseille, France) and FlowJo Software (Tree Star Inc).

### Antibodies and Flow Cytometry

TABLE 6
Antibodies used for flow cytometry analysis. For each
antibody in the left column, the clone used is given in the right column.

| Antibody (specificity) | Clone |
|---|---|
| CD117 | 104D2D1 |
| CD11c | BUI5 |
| CD123 | 9F5 |
| CD127 | R34.34 |
| CD14 | RMO52 |
| CD16 | 3G8 |
| CD161 | 191B8 |
| CD19 | SJ25C1 |
| CD25 | 2A3 |
| CD3 | SK7 |
| CD3 | UCHT1 |
| CD336 | Z231 |
| CD4 | 13B8.2 |
| CD45 | J.33 |
| CD56 | N901 |
| CD56 | NCAM162 |
| CD8 | B9.11 |
| CD8 | SK1 |
| Cocktail ILCs | — |
| CRTH2 | BM16 |
| HLADR | L243 |
| Lineagel DCs | — |
| TCRab | IP26A |
| TCRgd | IMMU510 |
| Va24 | 6B11 |
| Va7,2 | REA179 |
| CD45RC | MT2 |
| CD45RC | ABIS |
| Streptavidine | Alexa fluor 405 |

### Results

As a first screening, ABIS-45RC did not react against CD45RC− cells sorted using the commercial anti-CD45RC mAb MT2 clone suggesting that ABIS45RC could recognize CD45RC (data not shown).

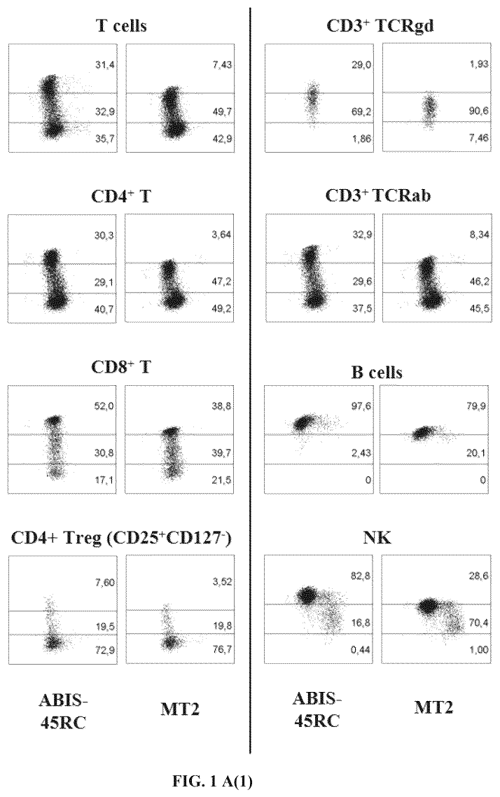

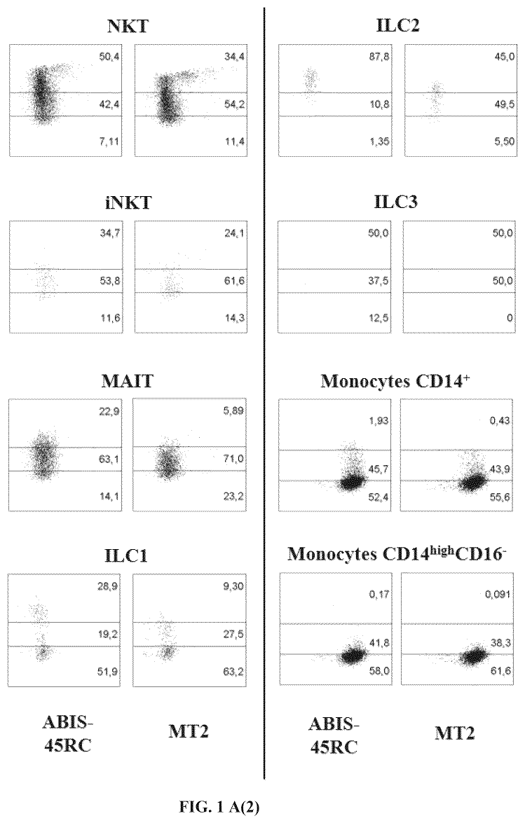

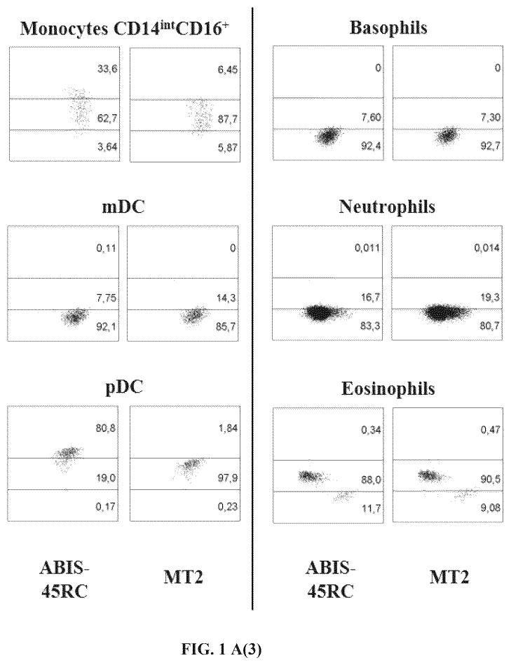

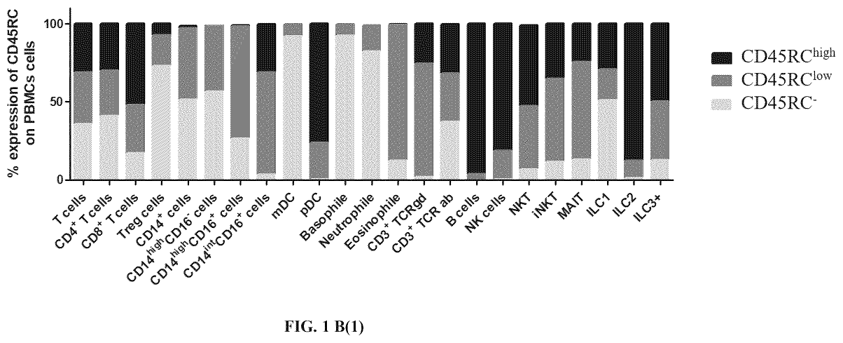

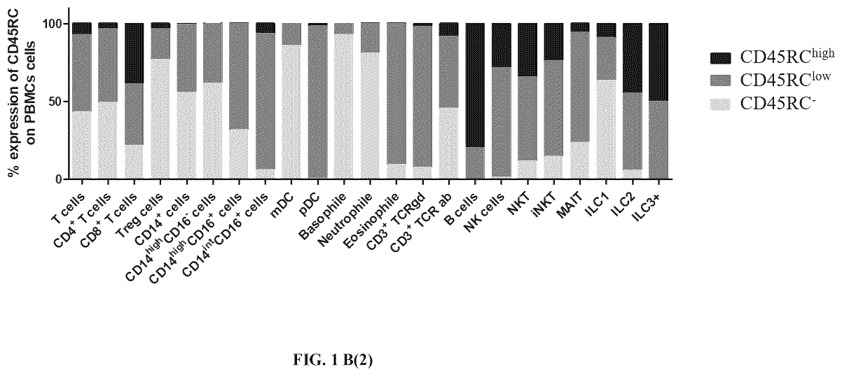

To further characterize ABIS-45RC, we analyzed its reactivity with human PBMCs. As shown in FIG. 1 A and B, a fraction of T CD4⁺ and T CD8⁺ cells is ABIS-45RC^{high} whereas the rest is ABIS-45RC^{low} or ABIS-45RC⁻. Most B and NK cells as well as pDCs were ABIS-45RC^{high} Most of NKT, iNKT MAIT, ILC2, ILC3 and of CD14^{int}CD16⁺ monocytes were ABIS-45RC^{high} or ABIS-45RC^{low}. CD14^{high}CD16⁻ monocytes, mDC, basophils and neutrophils were predominantly ABIS-45RC⁻. CD4⁺ Treg and CD8⁺Foxp3⁺ Treg were largely ABIS-45RC^{low/-}.

The analysis of the major PBMCs populations showed that ABIS-45RC had a pattern of reactivity comparable to the commercial anti-CD45RC mouse MT2 antibody (FIG. 1 and Picarda et al., 2017. JCI Insight. 2(3):e90088).

### Example 2

### Comparison of ABIS-45RC and the Commercial Anti-CD45RC Antibody “MT2”

### Material and Methods

### PBMC Isolation

Blood healthy volunteers is collected and peripheral blood mononuclear cells (PBMC) were isolated by Ficoll gradient centrifugation, which enables removal of unwanted fractions of blood products such as granulocytes, platelets and reaming red blood cell contaminants.

### Antibodies and Flow Cytometry

Human PBMC were labeled with the ABIS-45RC antibody (at the indicated concentrations), an anti-CD3 antibody and an anti-CD45RC (mouse clone MT2, Biolegend)-FITC labeled at 1.33 mg/mL. The ABIS-45RC reactivity was revealed using a biotin donkey anti-human IgG⁺ Streptavidin PercpCy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

### Cytotoxicity Analysis

Human PBMCs were incubated with medium at 37° C., isotype control antibody (Ms IgG1, clone 107.3, 10 g/ml), ABIS-45RC or anti-CD45RC (mouse clone MT2) at 2.5 or 10 μg/ml for 10 minutes to 18 hours. Then, cells were stained with anti-CD3 (clone SK7, BD Biosciences), Annexin-V, and DAPI. Percentage of apoptosis was obtained by gating on Annexin V⁺ and DAPI⁺ cells among T or non-T cells by flow cytometry.

### Results

### Both ABIS-45RC and Commercial Anti-CD45RC MT2 Antibodies Compete for the Same Epitope

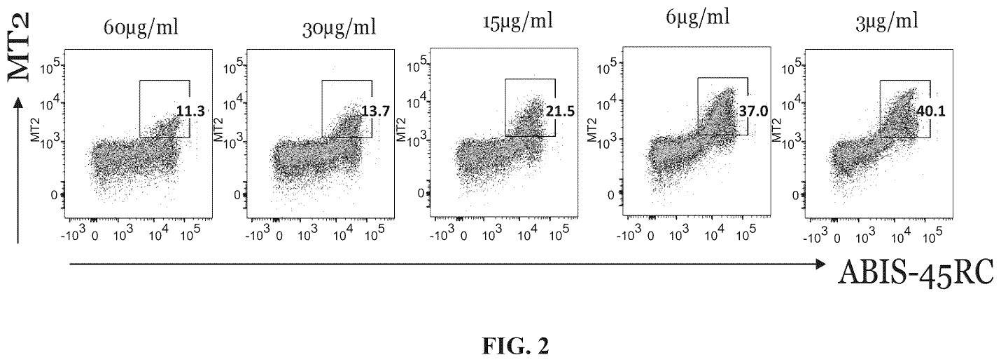

A shown in FIG. 2, co-labelling with the commercial anti-CD45RC MT2 clone showed that both antibodies competed and thus recognized the same or close epitope of human CD45RC.

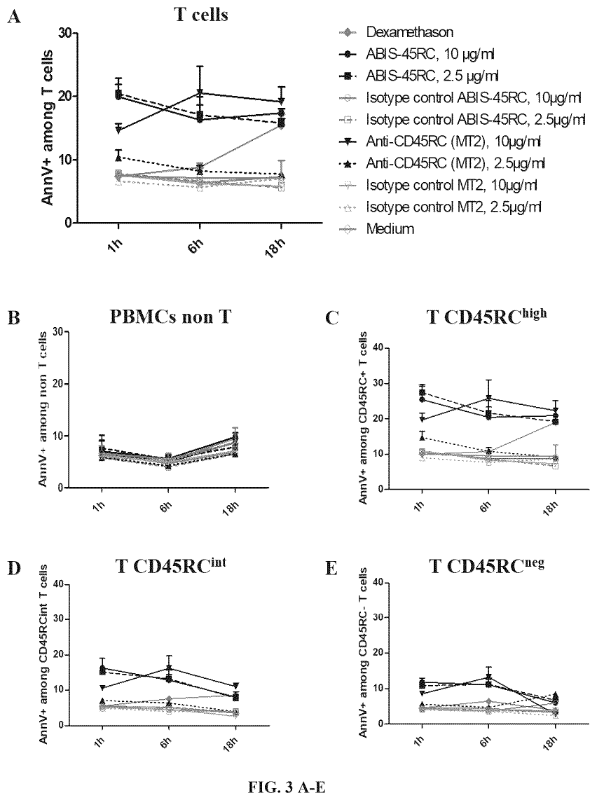

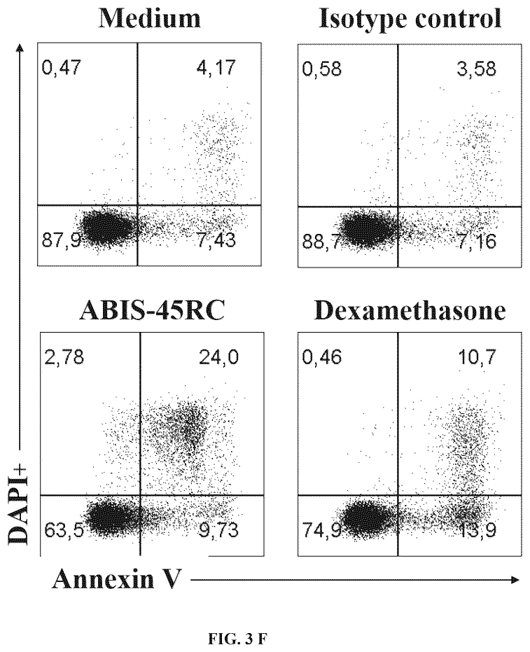

Cytotoxicity Induced by ABIS-45RC is Higher Compared to Commercial Anti-CD45RC As shown in FIG. 3, ABIS-45RC was cytotoxic to T cells but not non-T cells.

Moreover, the T cells cytotoxicity was directly correlated to the level of CD45RC expression and importantly, ABIS-45RC performed better at 2.5 μg/mL as compared to the MT2 clone at 10 μg/mL.

### Example 3

### Affinity of ABIS-45RC

### Material and Methods

Briefly, 1×10⁷ CD45RC^{high} PBMCs or CHO cells expressing CD45RC after plasmid transfection were solubilized using the Mem-PER membrane isolation kit (Thermo-fisher). ABIS-45RC was immobilized on a biochip CM5 and cell membranes were incubated at 25° C. to measure affinity constants using single cycle kinetics and calibration free concentration analysis on a BIAcore 3000 and a BIAcore T200.

### Results

Measurement of the affinity of CD45RC antibody was assessed by surface Plasmon Resonance (SPR), a technology for characterizing antibody-antigen interactions, and revealed an affinity (K_{D}) of 5×10⁻⁸ M, with a Kₒₙ of 2.91×10⁵ M⁻¹·sec⁻¹ and a K_{off} of 1.44×10⁻² sec⁻¹.

### Example 4

Treatment of Graft-Versus-Host-Disease (GVHD) with ABIS-45RC

### Material and Methods

### PBMC Isolation

Blood was collected at the Établissement Français du Sang (Nantes, France) from healthy individuals. Written informed consent was provided according to institutional guidelines. PBMC were isolated by Ficoll-Paque density-gradient centrifugation (Eurobio, Courtaboeuf, France). Remaining red cells and platelets were eliminated with a hypotonic solution and centrifugation.

### Animals

8- to 12-week-old NOD/SCID/IL2Rγ^{-/-} (NSG) mice were bred in our own animal facilities in SPF conditions (accreditation number C44-278).

### GVHD Model

Adult NSG immunodeficient mice were whole-body sublethaly irradiated (irradiation dose of 2 Gy at day −1) to induce lesions in tissues that will favor the development of GVHD. The following day (day 0), 1.5×10⁷ PBMCs (including CD45RC^{high} and CD45RC^{low/-} T cells) from healthy volunteers were injected intravenously in these mice. Human PBMCs, and in particular T cells, react against and attack mouse tissues inducing lesions. These T cells and the lesions observed in liver, intestine, lungs and skin mimic the GVHD observed following bone marrow transplantation in humans or other GVHD experimental systems using rodents as donors and recipients. In particular, these tissue lesions typically induce a body weight loss that begins—depending on the number of PBMCs injected and in our experimental system—around day 13 after injection of the PBMCs. Body weight loss is monitored daily and animals are sacrificed when it drops to 20% of the original body weight to avoid unnecessary suffering.

### Treatment

NSG mice were treated intraperitoneally with purified ABIS-45RC, with MT2 anti-CD45RC antibody or with an irrelevant control (an IVIg preparation used clinically comprising human purified IgG, and containing predominantly IgG1 antibodies) at 0.8 mg/kg from day 0 and every 2.5 days during 20 days.

NSG mice treated with ABIS-45RC or control antibodies also received intraperitoneally rapamycin from day 0 to day 10 at a suboptimal dose of 0.4 mg/day.

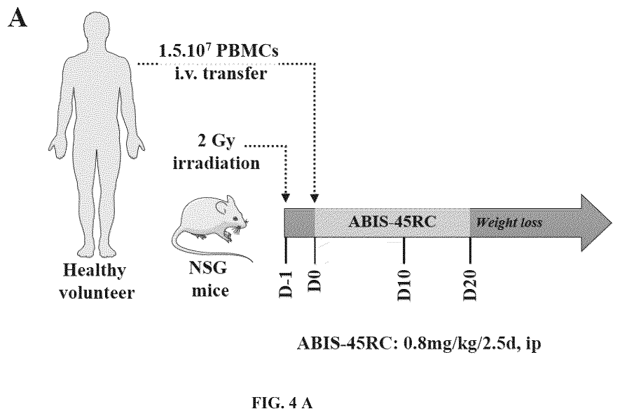

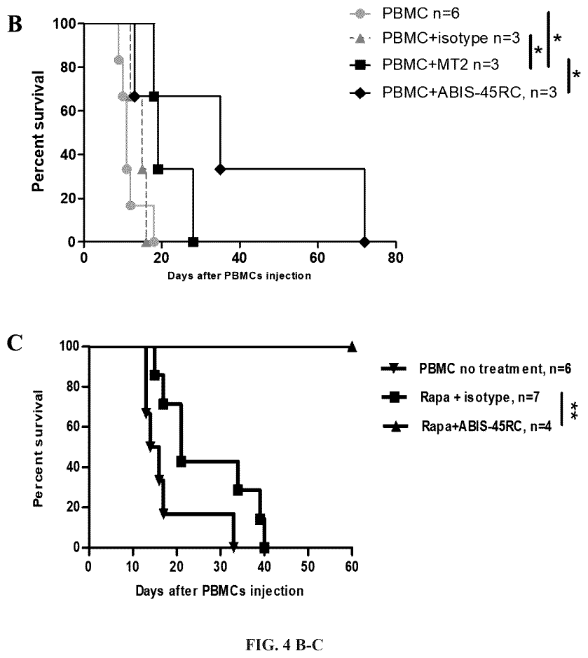

The experimental procedure is summarized in FIG. 4A.

### Results

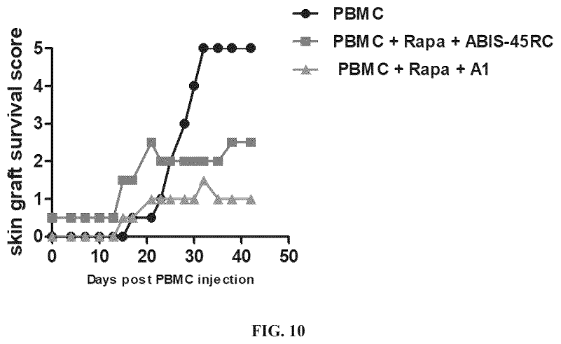

Treatment with PBMCs only induced weight loss, initiated around day 14, and, as shown in FIG. 4, death of all mice by day 33 (median survival: 11 days (FIG. 4B) to 15 days (FIG. 4C) days).

Treatment with control antibody and rapamycin only prolonged survival without reaching statistical significance (median survival: 21 days (FIG. 4C)).

While treatment with MT2 significantly prolonged survival of the mice (median survival: 19 days (FIG. 4B)), treatment with ABIS-45RC significantly increased this prolongation of survival of the mice up to 72 days (median survival: 35 days (FIG. 4B)).

Finally, combinatorial administration of ABIS-45RC with rapamycin completely prevented death as a consequence of GVHD (100% survival, FIG. 4C).

### Example 5

### Humanization of ABIS-45RC

The design for the humanization of ABIS-45RC by grafting of the CDRs into human germline antibody sequences was undertaken. ABIS-45RC was humanized by grafting the three CDRs from the LCVR (with SEQ ID NOs: 15, 16 and 17) into a human germline LCVR that was as homologous as possible to ABIS-45RC's LCVR. Similarly, the three CDRs from the HCVR (with SEQ ID NOs: 1, 4 and 3) were grafted into a human germline HCVR that was as homologous as possible to ABIS-45RC's HCVR.

In addition, a few amino acid residues in the framework regions (FR) of the selected human germline variable regions were changed to the amino acid residues that were present in the murine variable regions (so called back-mutations). Based upon information on the structure of immunoglobulin variable regions, and with the guidance of an homology molecular model of the Fv of ABIS-45RC, these few residues in the FRs were identified as having key roles in either maintaining the CDRs in the right conformation or in HCVR/LCVR packing, and thus they were retained in a first humanized version (version A) or substituted with their human germline counterparts, if possible, in subsequent humanized version (versions B and C). Under guidance of the homology molecular model, in versions B and C, when judged possible, the CDR residues were also substituted for their human germline counterparts.

### Homology Model Building

A model of ABIS-45RC was constructed according to established protocols (Ramos, 2012. Methods Mol Biol. 907:39-55).

### Light Chain

In this Section, Unless Specified Otherwise, Amino Acid Numbering is Based on SEQ ID NO: 81.

The LCVR's framework residues were used to search the sequences of solved antibody structures via protein BLAST. The top hits were Protein Data Bank (PDB) ID: 4NCC (2.50 Å resolution) having 83 of 89 FR residues identical, and 85 out of 89 FR residues similar, to those of ABIS-45RC's LCVR, and PDB ID: 1QOK (2.40 Å resolution) having 83 of 87 framework residues identical, and 84 out of 87 similar, to those of ABIS-45RC's LCVR.

The sequences of these two structures both differed from that of ABIS-45RC's LCVR (with SEQ ID NO: 81) with the substitutions T9A, T39P, R44K, N49S and P54A. In addition, PDB ID: 4NCC differed in sequence from ABIS-45RC's LCVR with the substitutions L95F, A99G and L105I.

A comparison of the two structures showed high homology. However, the carbon chains adopted slightly different conformations in the regions A13-E17 and E104-K106.

Based upon the results of the subsequent CDR searches, and the presence of the two N-terminal residues, the LCVR of the structure of PDB ID: 4NCC was selected as the LCVR framework template, and the rotameric conformation of L105I was selected (in PyMol) with reference to PDB ID: 1QOK.

Subsequently, the sequences of ABIS-45RC LCVR's CDR1, CDR2 and CDR3, with the addition of two residues on each end, were used to search the sequences of solved antibody structures via protein BLAST.

For CDR1, the top hits consisted of a cluster of sequences having 9 out of 9 identical residues. Amongst these were PDB ID: 4NCC and PDB ID: 1QOK. Thus, the PDB ID: 4NCC structure was adopted as the template for CDR1.

For CDR2, the top hits consisted of a cluster of sequences having 6 out of 7 identical residues. Amongst these were again PDB ID: 4NCC and PDB ID: 1QOK. The PDB ID: 4NCC structure was therefore also adopted as the template for CDR2.

For CDR3, the top hit, containing no gaps, was PDB ID: 1QOK, having 13 out of 13 identical residues. PDB ID: 4NCC was however a close second, having 12 out of 13 identical residues. A comparison of the two structures showed essentially identical conformations, excepting for the L95F substitution. Thus, the PDB ID: 4NCC structure was adopted as the template for CDR3, and the rotameric conformation of L95F was selected (in PyMol) with reference to PDB ID: 1QOK.

It was thus not necessary to fit any CDR templates to the LCVR framework template because PDB ID: 4NCC was selected as the primary template for all of the LCVR's CDRs.

Finally, the LCVR partial model was manually subjected to mutagenesis at 8 positions (in PyMol), with selection of optimal rotamers, in order to match the ABIS-45RC LCVR sequence.

### Heavy Chain

In this Section, Unless Specified Otherwise, Amino Acid Numbering is Based on SEQ ID NO: 61.

Next, the HCVR's framework residues were used to search the sequences of solved antibody structures via protein BLAST. The top hit was PDB ID: 30PZ (3.40 Å resolution), having 84 out of 90 framework residues identical, and 85 out of 90 similar, to those of ABIS-45RC's HCVR.

Since PDB ID: 30PZ was missing the N terminal residue, and was resolved with fairly poor resolution, additional hits with the highest identity/similarity scores were also surveyed. The top amongst these were PDB ID: 4CAD (2.50 Å resolution), having 78 out of 91 framework residues identical, and 87 out of 91 similar, to those of ABIS-45RC's HCVR; and PDB ID: 1RUR (1.50 Å resolution), having 75 out of 91 framework residues identical, and 87 out of 91 similar, to those of ABIS-45RC's HCVR.

A comparison of the PDB ID: 30PZ and PDB ID: 4CAD structures showed high homology with alternative residue rotamers being the principal differences.

A comparison of the PDB ID: 30PZ and PDB ID: 1RUR structures similarly showed high homology; however, there was a significant conformational change in the V_{H}-FR2 loop L45-G49 relative to the PDB ID: 30PZ and PDB ID: 4CAD structures.

Further, based upon sequence, ABIS-45RC's HCVR and PDB ID: 4CAD were predicted to exhibit the Honegger Type III (Honegger & Plückthun, 2001. J Mol Biol. 309(3):687-99) conformation of the N-terminal strand 5-12 because of the presence of a glutamine in position 6. However, PDB ID: 1RUR was predicted to exhibit the Honegger Type I conformation, due to the presence of a glutamic acid in position 6. Nevertheless, the three structures exhibited the identical conformation of the 5-12 strand. Also, the sequences of ABIS-45RC's HCVR, PDB ID: 4CAD and PDB ID: 1RUR were predicted to adopt the K-form (kinked base conformation) defined by the revised Shirai's rules for HCVR's CDR3 (Kuroda et al., 2008. Proteins. 73(3):608-20).

Based upon the results of the subsequent CDR searches, higher overall sequence similarity, structural concordance with PDB ID: 30PZ, and higher experimental resolution, the HCVR of the structure PDB ID: 1RUR was selected as the HCVR framework template; however, the 45-49 loop of PDB ID: 4CAD (having the same conformation as that of PDB ID: 30PZ) was substituted for that of PDB ID: 1RUR in the HCVR template using two residues N and C-terminal overhangs on the 45-49 ends to anchor the loop template fragment to the framework template.

Subsequently, the sequences of HCVR's CDR1, CDR2 and CDR3, with the addition of two residues on each end, were used to search the sequences of solved antibody structures via protein BLAST.

For CDR1, there was a cluster of sequence hits having 9 out of 12 identical residues. Amongst these was PDB ID: 1RUR. Thus, the PDB ID: 1RUR structure was selected as the template for CDR1.

For CDR2, the top hit was PDB ID: 3NTC (1.55 Å resolution) having 8 out of 12 residues identical, and 9 out of 12 similar, to those of ABIS-45RC's HCVR. However, PDB ID: 1RUR was a close second having 7 of 12 residues identical, and 9 out of 12 similar, to those of ABIS-45RC's HCVR. Comparison of the two structures showed essentially identical conformations, and the higher identity of PDB ID: 3NTC was due to 2 C terminal residues added to the CDR2 sequence for purposes of BLAST search. Thus, the PDB ID: 1RUR structure was preferred as the template for CDR2.

For CDR3, the top two hits, containing no gaps, were PDB ID: 1NGY (2.20 Å resolution) and PDB ID: 1NGZ (1.60 Å resolution), both having 8 out of 11 residues identical, and 9 out of 11 similar, to those of ABIS-45RC's HCVR. A comparison of the two structures showed a significantly different mainchain conformation. Without willing to be bound to a theory, the Inventors hypothesized that this difference might be due to the residue in position 101. In PDB ID: 1NGY, a larger methionine cannot adopt the orientation of the smaller serine of PDB ID: 1NGZ, which directs its sidechain into the core of the protein. Since the desired substitution to match the ABIS-45RC's HCVR sequence is F101, the PDB ID: 1NGY structure was adopted as the template for CDR3. Next, in order to complete the HCVR partial model, the CDR3 template was grafted onto the modified PDB ID: 1RUR HCVR template using the two residue overhang on its ends to anchor the CDR template fragment to the framework template (in PyMol).

Finally, the HCVR partial model was manually subjected to mutagenesis at 23 positions (in PyMol), with selection of optimal rotamers, in order to match the ABIS-45RC's HCVR sequence.

### Final Model Assembly

Subsequently, the best tertiary arrangement of the HCVR and LCVR partial models were selected to assemble the final model. The HCVR and LCVR template sequences were submitted to the Packing Angle Prediction Server (PAPS) (Abhinandan & Martin, 2010. Protein Eng Des Sel. 23(9):689-97) to find a predicted best-fit tertiary arrangement. The PAPS server predicted that the solved antibody structure PDB ID: 1MNU, with a relative packing angle of −45.6°, would provide the best tertiary arrangement of HCVR and LCVR. Thus, the final model was assembled by fitting the backbone coordinates of the conserved anchor segments of the HCVR and LCVR partial models to PDB ID: 1MNU (in PyMol).

Lastly, the coordinates of this final model were subjected to a round of energy minimization employing GROMACS (Van der Spoel et al., 2005. J Comput Chem. 26(16):1701-18) with the GROMOS96 force-field (Scott et al., 1999. J Phys Chem A. 103(19):3596-3607).

### Human Germlines

For the design of CDR-grafted versions of ABIS-45RC's HCVR and LCVR, two times three human germlines were selected:
IGHV1-2*01, IGHV5-51*01 and IGHV3-11*05 for the HCVR; and
IGKV1-9*01, IGKV6-21*02 and IGKV3-11*01 for the LCVR.

### Design of Humanized HCVR and LCVR

The humanized versions A for both HCVR and LCVR are conservative versions that explicitly minimize and/or avoid alteration of CDR residues. These versions are thus expected to give a similar or better binding and/or potency activity as a chimeric antibody (ABIS-45RC's HCVR [SEQ ID NO: 61] and LCVR [SEQ ID NO: 81] fused to human constant regions [SEQ ID NOs: 91 and 92]).

The humanized versions B for both HCVR and LCVR are designed to reach a percentage of sequence identity with the closest human germline of at least 85%. This can be achieved by germlining (i.e., substituting the mouse residue with the corresponding human germline residue) FR and/or CDR amino acid residues. 85% is the cut-off percentage identity necessary to get the substem -zu- for “humanized”, denomination according to the 2014 World Health Organization (WHO) guidance on antibody International Nonproprietary Names (INN).

The humanized versions C for both HCVR and LCVR are designed to reach the highest degree of humanness (i.e., the highest degree of sequence identity with the corresponding human germline). Following inspection of the homology molecular model, a number of residues have been identified as candidates for germlining. Therefore, all the residues that could reasonably be germlined have been taken into consideration.

HCVR, using IGHV1-2*01

To design humanized HCVR version A from IGHV1-2*01, the murine CDRs (with SEQ ID NOs: 1, 4 and 3) were grafted into IGHV1-2*01 and 4 residues in FR2 and FR3 were back-mutated to the parental murine residues, to maintain the full activity of the antibody. These residues are 148, L70, A72 and V97 in SEQ ID NO: 61. The resulting HCVR is as set forth in SEQ ID NO: 62 and shares 81.6% sequence identity with IGHV1-2*01 human germline.

To design humanized HCVR version B from IGHV1-2*01, in addition to version A, 5 amino acid residues in the CDR2 were further germlined (i.e., substituted by the corresponding IGHV1-2*01 human germline residues). These residues are D56G, A58T, S60Y, N61A and K65Q in SEQ ID NO: 61. The resulting HCVR is as set forth in SEQ ID NO: 65 and shares 86.7% sequence identity with IGHV1-2*01 human germline.

To design humanized HCVR version C from IGHV1-2*01, in addition to version B, 2 amino acid residues in the CDR2 were further germlined. These residues are D50R and E62Q in SEQ ID NO: 61. The resulting HCVR is as set forth in SEQ ID NO: 68 and shares 88.8% sequence identity with IGHV1-2*01 human germline.

Various other humanized versions of the HCVR from IGHV1-2*01 were further designed, starting from version B. Indeed, to get a well humanized monoclonal antibody, 85% is supposed to be sufficient (version B from IGHV1-2*01 shares 86.7% sequence identity with IGHV1-2*01 human germline). In order to reduce the risk of introducing mutations, versions D, E, F, G and H were thus designed to reach 85% and no more.

The resulting HCVR versions D, E, F, G and H from IGHV1-2*01 are as set forth in SEQ ID NOs: 101, 121, 122, 123 and 124, respectively, and all share 85.7% sequence identity with IGHV1-2*01 human germline.

### HCVR, Using IGHV5-51*01

To design humanized HCVR version A from IGHV5-51*01, the murine CDRs (with SEQ ID NOs: 1, 4 and 3) were grafted into IGHV5-51*01 and 6 residues in FR1, FR2 and FR3 were back-mutated to the parental murine residues, to maintain the full activity of the antibody. These residues are A24, T28, 148, L70, L83 and V97 in SEQ ID NO: 61. The resulting HCVR is as set forth in SEQ ID NO: 63 and shares 79.6% sequence identity with IGHV5-51*01 human germline.

To design humanized HCVR version B from IGHV5-51*01, in addition to version A, 1 amino acid residue in the FR1 and 6 amino acid residues in the CDR2 were further germlined. These residues are A24G, D56S, A58T, S60Y, N61S, K63S and K65Q in SEQ ID NO: 61. The resulting HCVR is as set forth in SEQ ID NO: 66 and shares 86.7% sequence identity with IGHV5-51*01 human germline.

To design humanized HCVR version C from IGHV5-51*01, in addition to version B, 1 amino acid residue in the FR1 and 2 amino acid residues in the CDR2 were further germlined. These residues are T28S, D50I and E62P in SEQ ID NO: 61. The resulting HCVR is as set forth in SEQ ID NO: 69 and shares 89.8% sequence identity with IGHV5-51*01 human germline.

### HCVR, Using IGHV3-11*05

To design humanized HCVR version A from IGHV3-11*05, the murine CDRs (with SEQ ID NOs: 1, 4 and 3) were grafted into IGHV3-11*05 and 9 residues in FR1, FR2 and FR3 were back-mutated to the parental murine residues, to maintain the full activity of the antibody. These residues are Y27, T30, 148, G49, L70, A72, T74, A79 and V97 in SEQ ID NO: 61. The resulting HCVR is as set forth in SEQ ID NO: 64 and shares 76.5% sequence identity with IGHV3-11*05 human germline.

To design humanized HCVR version B from IGHV3-11*05, in addition to version A, 2 amino acid residues in the FR1, 6 amino acid residues in the CDR2 and 1 amino acid residue in the FR3 were further germlined. These residues are Y27F, T30S, D56S, A58T, S60Y, N61A, E62D, K63S and A79L in SEQ ID NO: 61. The resulting HCVR is as set forth in SEQ ID NO: 67 and shares 89.8% sequence identity with IGHV3-11*05 human germline.

To design humanized HCVR version C from IGHV3-11*05, in addition to version B, 1 amino acid residue in the FR2 and 1 amino acid residue in the CDR2 were further germlined. These residues are I48V and P53S in SEQ ID NO: 61. The resulting HCVR is as set forth in SEQ ID NO: 70 and shares 87.8% sequence identity with IGHV3-11*05 human germline.

### LCVR, Using IGKV1-9*01

To design humanized LCVR version A from IGKV1-9*01, the murine CDRs (with SEQ ID NOs: 15, 16 and 17 with X₁₂ being absent) were grafted into IGKV1-9*01 and 3 residues in FR2 and FR3 were back-mutated to the parental murine residues, to maintain the full activity of the antibody. These residues are F35, W46 and Y70 in SEQ ID NO: 81. The resulting LCVR is as set forth in SEQ ID NO: 82 and shares 83.2% sequence identity with IGKV1-9*01 human germline.

To design humanized LCVR version B from IGKV1-9*01, in addition to version A, 1 amino acid residue in the CDR1 and 1 amino acid residue in the FR2 were further germlined. These residues are S24R and F35Y in SEQ ID NO: 81. The resulting LCVR is as set forth in SEQ ID NO: 85 and shares 85.3% sequence identity with IGKV1-9*01 human germline.

To design humanized LCVR version C from IGKV1-9*01, in addition to version B, 2 amino acid residues in the CDR2 and 1 amino acid residue in the FR3 were further germlined. These residues are N49A, P54Q and Y70F in SEQ ID NO: 81. The resulting LCVR is as set forth in SEQ ID NO: 88 and shares 88.4% sequence identity with IGKV1-9*01 human germline.

A LCVR version D from IGKV1-9*01 was further designed, in order to introduce an extra residue (Ser; S) in the CDR1 as found in the human germline IGKV1-9*01. The introduction of the extra residue in the CDR1 loop has shown that the binding activity was conserved (data not shown). The introduction of a serine residue in the CDR1 of version B brings the sequence identity to 86.3%, so in order to reduce the risk of introducing mutations, version D was designed where Kabat residue L36 was reverted to the original mouse residue Phe (F). The resulting LCVR is as set forth in SEQ ID NO: 103.

### LCVR, Using IGKV6-21*02

To design humanized LCVR version A from IGKV6-21*02, the murine CDRs (with SEQ ID NOs: 15, 16 and 17 with X₁₂ being absent) were grafted into IGKV6-21*02 and 4 residues in FR2 and FR3 were back-mutated to the parental murine residues, to maintain the full activity of the antibody. These residues are F35, W46, Y48 and Y70 in SEQ ID NO: 81. The resulting LCVR is as set forth in SEQ ID NO: 83 and shares 81.1% sequence identity with IGKV6-21*02 human germline.

To design humanized LCVR version B from IGKV6-21*02, in addition to version A, 1 amino acid residue in the CDR1, 1 amino acid residue in the FR2, 1 amino acid residue in the CDR2 and 1 amino acid residue in the FR3 were further germlined. These residues are S24R, F35Y, L53S and Y70F in SEQ ID NO: 81. The resulting LCVR is as set forth in SEQ ID NO: 86 and shares 85.3% sequence identity with IGKV6-21*02 human germline.

To design humanized LCVR version C from IGKV6-21*02, in addition to version B, 1 amino acid residue in the CDR3 was further germlined. This residue is Q88H in SEQ ID NO: 81. The resulting LCVR is as set forth in SEQ ID NO: 89 and shares 86.3% sequence identity with IGKV6-21*02 human germline.

### LCVR, Using IGKV3-11*01

To design humanized LCVR version A from IGKV3-11*01, the murine CDRs (with SEQ ID NOs: 15, 16 and 17 with X₁₂ being absent) were grafted into IGKV3-11*01 and 3 residues in FR2 and FR3 were back-mutated to the parental murine residues, to maintain the full activity of the antibody. These residues are F35, W46 and Y70 in SEQ ID NO: 81. The resulting LCVR is as set forth in SEQ ID NO: 84 and shares 84.2% sequence identity with IGKV3-11*01 human germline.

To design humanized LCVR version B from IGKV3-11*01, in addition to version A, 1 amino acid residue in the CDR1 was further germlined. This residue is S24R in SEQ ID NO: 81. The resulting LCVR is as set forth in SEQ ID NO: 87 and shares 85.3% sequence identity with IGKV3-11*01 human germline.

To design humanized LCVR version C from IGKV3-11*01, in addition to version B, 1 amino acid residue in the FR2, 3 amino acid residues in the CDR2 and 1 amino acid residue in the FR3 were further germlined. These residues are F35Y, L53R, P54A, S55 and Y70F in SEQ ID NO: 81. The resulting LCVR is as set forth in SEQ ID NO: 90 and shares 90.5% sequence identity with IGKV3-11*01 human germline.

### Example 6

### Production, Purification and Characterization of Humanized Anti-45RC Antibodies

Analytical size exclusion chromatography (SEC-HPLC) and differential scanning calorimetry (DSC) were used to compare the profile and the thermal stability, respectively, of 9 humanized anti-45RC variants A to I. These variants correspond to antibody comprising the “versions A” HCVR and LCVR described in Example 5.

Analytical size exclusion chromatography (SEC-HPLC) and differential scanning calorimetry (DSC) were also used to compare the profile and the thermal stability, respectively, of 4 other humanized anti-45RC variants A1, A2, I1 and I2.

### Material and Methods

### SEC-HPLC

A Shimadzu Prominence HPLC system was used, with a Superdex 200 Increase 5/150 GL column (GE Healthcare). The column was previously calibrated in the same buffer and conditions used during sample analysis (using the Molecular Weight SEC Calibration kits from GE Healthcare, in PBS 1×, at 0.25 mL/min, with the column oven set to 30° C.).

All samples were centrifuged (20.000 g, 5 minutes, 4° C.) and had their protein content quantitated by Nanodrop ND-1000 spectrophotometer with IgG analysis program, prior to SEC analysis.

The isocratic program was set to inject about 15 g of each sample, at 0.25 mL/min during 18 minutes. After SEC analysis, 280 nm chromatogram was extracted from the raw data, and analyzed by peak integration.

### DSC

A Microcal™ VP-Capillary DSC system was used to perform differential scanning calorimetry experiments.

Samples in 1×PBS buffer were centrifuged (20.000 g, 5 minutes, 4° C.), and had their protein content quantitated Nanodrop ND-1000 spectrophotometer with TgG analysis program, prior to DSC analysis. Samples were then diluted in PBS to a final concentration of 1 mg/mL.

The pre-equilibration time was 3 minutes and the thermograms that followed were acquired between 2° and 110° C. with a scanning rate of 60° C./hour, a filtering period of 25 seconds and medium feedback.

Prior to sample analysis, 5 buffer/buffer scans were measured to stabilize the instrument, and a buffer/buffer scan was performed between each protein/buffer scan.

The data was fitted to a non-2-state unfolding model, with the pre- and post-transition adjusted baseline subtracted. The calorimetric enthalpy (ΔH) is determined as the area under the peak of the transition, whereas the van't Hoff enthalpy (ΔHv) is determined from the model used.

### Results

### SEC-HPLC

A summary of the SEC parameters is given in Table 7 below.

TABLE 7
SEC parameters of the humanized
ABIS-45RC variants A-I, A1, A2; I1 and I2.

| ABIS-45RC | | | | Area | Calculated |
|---|---|---|---|---|---|
| variant | Peak # | RT | Area | % | MW |
| A | 1 | 4.939 | 373336 | 10.62 | 510 |
| | 2 | 5.212 | 790998 | 22.50 | 404 |
| | 3 | 6.099 | 2351531 | 66.88 | 190 |
| B | 1 | 4.671 | 1305266 | 26.47 | 640 |
| | 2 | 5.013 | 1451918 | 29.44 | 478 |
| | 3 | 5.899 | 2174527 | 44.10 | 225 |
| C | 1 | 4.896 | 405089 | 14.04 | 529 |
| | 2 | 5.148 | 527782 | 18.29 | 426 |
| | 3 | 5.980 | 1788411 | 61.97 | 210 |
| | 4 | 7.708 | 164621 | 5.70 | 48 |
| D | 1 | 4.939 | 126757 | 5.38 | 510 |
| | 2 | 5.268 | 430061 | 18.25 | 385 |
| | 3 | 6.137 | 1799214 | 76.37 | 184 |
| E | 1 | 4.738 | 607732 | 18.87 | 605 |
| | 2 | 5.039 | 686150 | 21.31 | 468 |
| | 3 | 5.925 | 1925975 | 59.82 | 220 |
| F | 1 | 4.811 | 791680 | 13.93 | 568 |
| | 2 | 5.124 | 1306598 | 22.99 | 435 |
| | 3 | 5.977 | 3585434 | 63.08 | 211 |
| G | 1 | 4.768 | 185266 | 3.54 | 589 |
| | 2 | 5.092 | 889883 | 16.99 | 447 |
| | 3 | 5.893 | 4159958 | 79.46 | 226 |
| H | 1 | 4.896 | 105313 | 1.90 | 529 |
| | 2 | 5.158 | 506367 | 9.11 | 423 |
| | 3 | 5.962 | 4941812 | 88.99 | 213 |
| I | 1 | 5.142 | 123472 | 3.16 | 429 |
| | 2 | 6.081 | 3783288 | 96.84 | 193 |
| A1 | 1 | 5.074 | 129770 | 2.65 | 478 |
| | 2 | 6.098 | 4775239 | 97.35 | 189 |
| A2 | 1 | 5.074 | 129770 | 2.65 | 478 |
| | 2 | 6.098 | 4775239 | 97.35 | 189 |
| I1 | 1 | 4.885 | 131671 | 2.5 | 567 |
| | 2 | 5.146 | 355754 | 6.78 | 447 |
| | 3 | 6.142 | 4760685 | 90.7 | 181 |
| I2 | 1 | 4.864 | 32276 | 0.73 | 578 |
| | 2 | 5.150 | 168687 | 3.79 | 446 |
| | 3 | 6.181 | 4246352 | 95.48 | 175 |

RT: retention time (in minutes)
MW: molecular weight (in kDa)

Table 7 above shows in bold the peaks corresponding to the anti-CD45RC antibodies (peak 3 for each of variants A to H, I1 and I2; and peak 2 for each of variants I, A1 and A2), with RT and calculated MW expected for a monomeric, non-precipitated and non-dissociated antibody.

### DSC

A summary of the DSC parameters is given in Table 8 below.

TABLE 8
DSC parameters of the humanized ABIS-45RC variants A-I, A1, A2; I1 and
I2. Denaturation of the antibody happens in two steps, hence two melting temperatures
are given, one of each step.

| ABIS-45RC variant | Conc. | T_{1/2} | ΔH | Tₒₙₛₑₜ | Tₘ₁ | Tₘ₂ |
|---|---|---|---|---|---|---|
| A | 0.0035 | 7.09 | 763 | 55.59 | 66.44 | 80.64 |
| B | 0.0056 | 7.09 | 776 | 55.96 | 64.73 | 81.01 |
| C | 0.0031 | 7.93 | 596 | 55.95 | 63.05 | 79.74 |
| D | 0.0023 | 5.83 | 863 | 60.09 | 70.91 | 80.10 |
| E | 0.0042 | 5.83 | 762 | 57.09 | 71.28 | 80.05 |
| F | 0.0099 | 5.41 | 867 | 58.63 | 71.15 | 80.34 |
| G | 0.0087 | 5.01 | 824 | 60.76 | 69.93 | 80.79 |
| H | 0.0093 | 4.59 | 877 | 59.79 | 68.14 | 80.24 |
| I | 0.0055 | 5.42 | 809 | 62.07 | 70.83 | 81.27 |
| A1 | 0.0049 | 3.32 | 969 | 61.09 | 68.17 | 81.91 |
| A2 | 0.0070 | 5.83 | 1150 | 60.21 | 66.92 | 81.94 |
| I1 | 0.0067 | 3.75 | 1080 | 63.08 | 73.08 | 81.84 |
| I2 | 0.0055 | 4.16 | 1030 | 63.68 | 72.43 | 82.02 |

Conc.: concentration, in mM.
T_{1/2}: width of transition at half height of the peak, in ° C.
ΔH: calorimetric enthalpy of unfolding, in cal/M.
Tₒₙₛₑₜ: temperature at which the unfolding transition begins, in ° C.
Tₘ₁: denaturing/melting temperature of the first step, in ° C.
Tₘ₂: denaturing/melting temperature of the second step, in ° C.

### Example 7

### Reactivity of Humanized ABIS-45RC Variants A-I

### Material and Methods

### PBMC Isolation

Blood healthy volunteers is collected and peripheral blood mononuclear cells (PBMC) were isolated by Ficoll gradient centrifugation, which enables removal of unwanted fractions of blood products such as granulocytes, platelets and reaming red blood cell contaminants.

### Antibodies and Flow Cytometry

Human PBMC were labeled with the murine ABIS-45RC antibody or each of the humanized ABIS-45RC antibodies variants A-I (at 2 μg/mL and 1 μg/mL); and an anti-CD3 antibody. The murine and humanized ABIS-45RC antibodies reactivity was revealed using a biotin donkey anti-human IgG⁺ Streptavidin PercpCy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

### Results

### Both ABIS-45RC and Commercial Anti-CD45RC MT2 Antibodies Compete for the Same Epitope

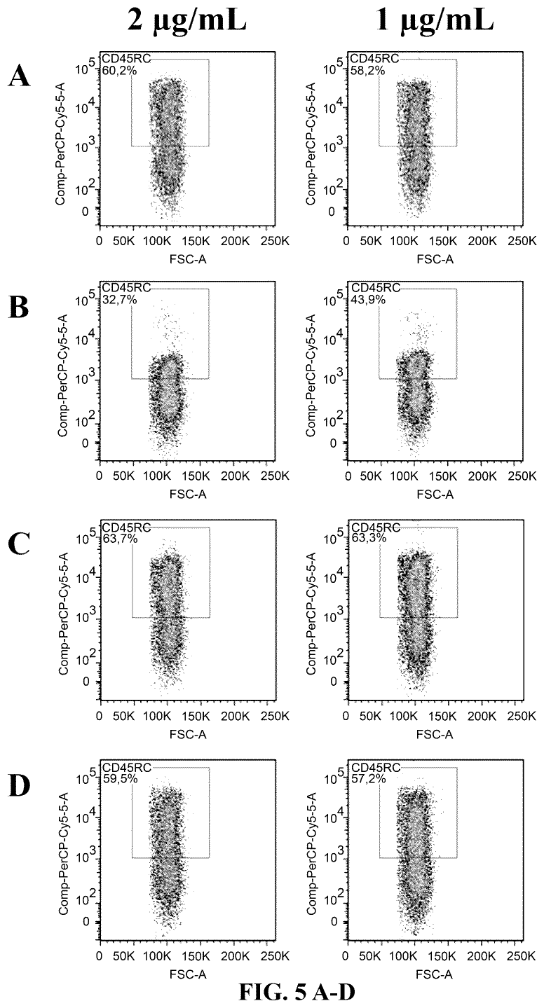

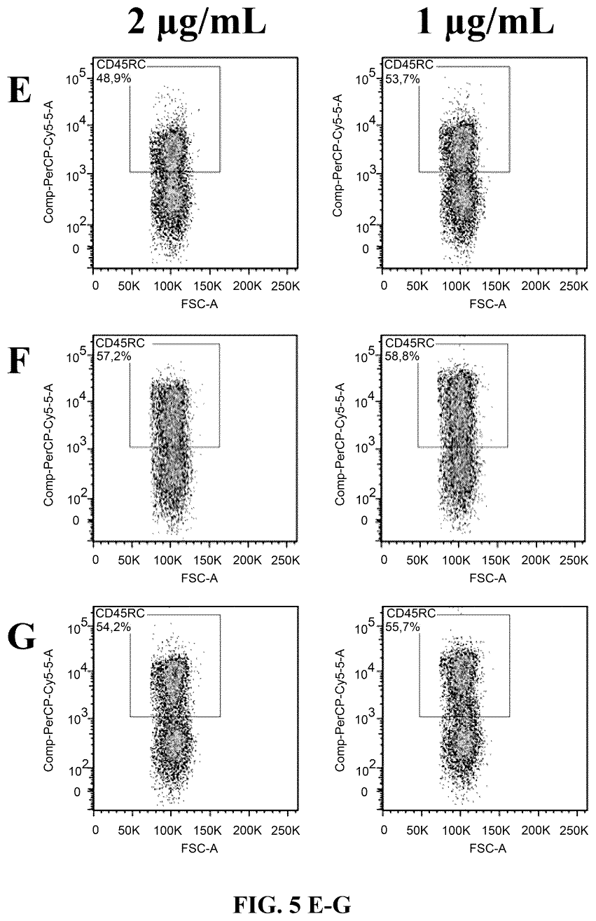

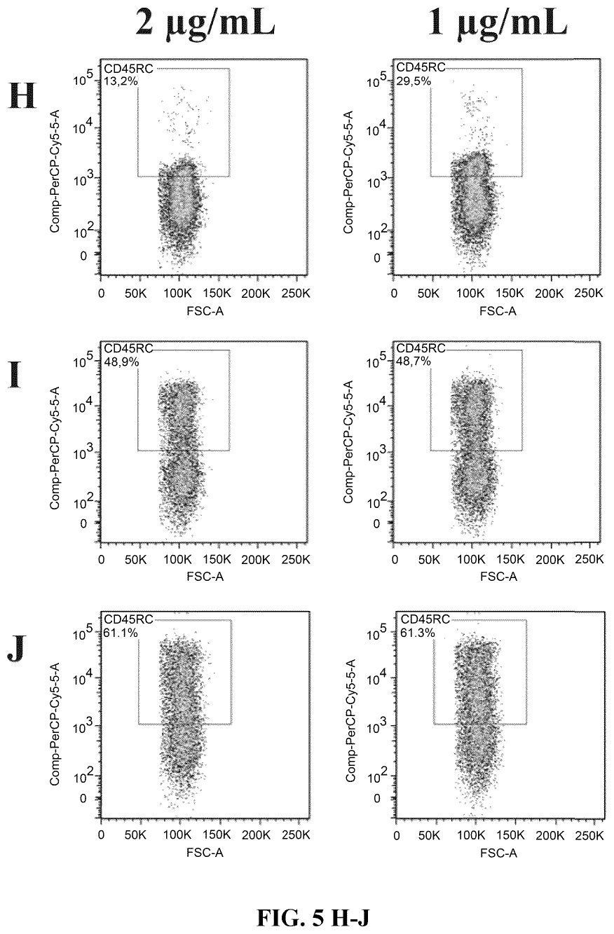

Labelling with either of the humanized ABIS-45RC antibodies (variant A, FIG. 5A; variant B, FIG. 5B; variant C, FIG. 5C; variant D, FIG. 5D; variant E, FIG. 5E; variant F, FIG. 5F; variant G, FIG. 5G; variant H, FIG. 5H; variant I, FIG. 5I) or the murine ABIS-45RC (FIG. 5J) shows that the antibodies recognized human CD45RC in a similar manner.

### Example 8

### Engineered Antibodies

The CDR1 of ABIS-45RC's LCVR has a canonical structure unique to mouse antibodies, with a length of 10 amino acid residues (SEQ ID NO: 15 with X₁₂ being absent, i.e., SASSSVSYMH).

For the design of humanized versions A, B and C of the LCVR described in Example 5, this 10-amino-acid-residue CDR1 was grafted into the human germlines, with backmutation and/or germlining but no addition or deletion of any residue. However, in human germlines, the LCVR's CDR1 has a minimum length of 11 amino acid residues.

Therefore, to increase the humanness of the humanized antibodies, the Inventors have sought to engineer ABIS-45RC VL-CDR1 “SASSSVSYMH” to extend it by one extra residue. One candidate position is position 8 in SEQ ID NO: 15 (designated as X₁₂), i.e., between S30 and Y31 in SEQ ID NO: 81. In all of the candidate germlines for the humanization design, this position is occupied with Asn (N), Ser (S) or Gly (G), while in the murine germline, this position is empty.

In order to investigate the structural relevance and stability of such insertion, ABIS-45RC VL-CDR1 was expanded by insertion of an asparagine, i.e., SEQ ID NO: 15 with X₁₂ being Asn (N), i.e., SASSSVSNYMH. A search of the sequences of solved antibody structures via protein BLAST was then conducted. The top hit was the LCVR CDR1 of the structure PDB ID: 5CMA. Subsequently, this structural segment was grafted onto the ABIS-45RC model using the two-residue overhang on its ends to anchor the CDR template fragment to the model. It was observed that, in order to accommodate the additional residue, there was a conformational change that shifted the neighboring residue, thereby presenting a slight steric clash with Y70. However, in all of the human germlines, this residue is a more accommodating phenylalanine.

### Engineered Mouse Antibody

Based on the above, the Inventors have engineered the ABIS-RC45 antibody, by inserting an asparagine residue (Asn, N) in the VL-CDR1, and further mutating Y70 of SEQ ID NO: 81 into a phenylalanine (Phe, F). The resulting “engineered Asn/Phe ABIS-RC45” LCVR is set forth in SEQ ID NO: 71, with X₁₂ being Asn (N).

Two other mouse antibodies have also been produced on the same basis, by inserting a serine residue (Ser, S) or a glycine residue (Gly, G) in the VL-CDR1, and further mutating Y70 of SEQ ID NO: 81 into a phenylalanine (Phe, F). The two resulting “engineered Ser/Phe ABIS-RC45” and “engineered Gly/Phe ABIS-RC45” LCVR are set forth in SEQ ID NO: 71, with X₁₂ being Ser (S) or Gly (G), respectively.

### Engineered Humanized Antibodies

Based on the above, engineered humanized LCVR versions A, B and C (as described in Example 5) can be further designed, as set forth in SEQ ID NOs: 72-80 where X₁₂ is Asn (N), Ser (S) or Gly (G) and the residue in position 70 is a Phe (F).

### Example 9

### Reactivity of Engineered Asn/Phe ABIS-45RC

### Material and Methods

Blood from healthy volunteers was collected and peripheral blood mononuclear cells (PBMC) were isolated by Ficoll gradient centrifugation, which enables removal of unwanted fractions of blood products such as granulocytes, platelets and reaming red blood cell contaminants.

Human PBMCs were labeled with ABIS-45RC or with engineered Asn/Phe ABIS-45RC and an anti-CD3 antibody. The reactivity was revealed using a biotin donkey anti-human IgG⁺ Streptavidin PercpCy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

### Results

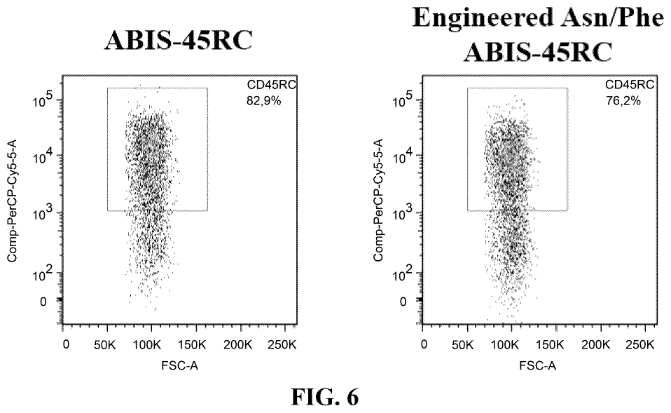

As shown in FIG. 6, labelling with either ABIS-45RC (left panel) or the engineered Asn/Phe ABIS-45RC (right panel) shows that both antibodies recognized human CD45RC in a similar manner.

### Example 10

### Reactivity of Humanized ABIS-45RC

### Material and Methods

Blood from healthy volunteers was collected and peripheral blood mononuclear cells (PBMC) were isolated by Ficoll gradient centrifugation, which enables removal of unwanted fractions of blood products such as granulocytes, platelets and reaming red blood cell contaminants.

Human PBMCs were labeled with ABIS-45RC or with humanized ABIS-45RC at 20, 5, 1.25 or 0.3 μg/mL and an anti-CD3 antibody. The reactivity was revealed using a biotin donkey anti-human IgG⁺ Streptavidin PercpCy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

### Results

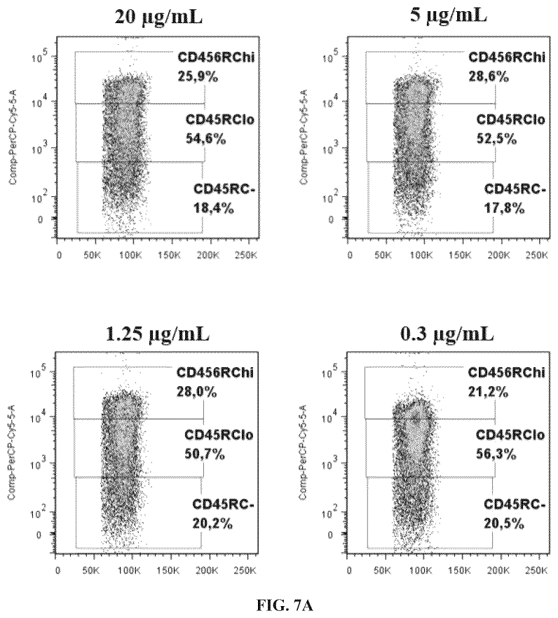

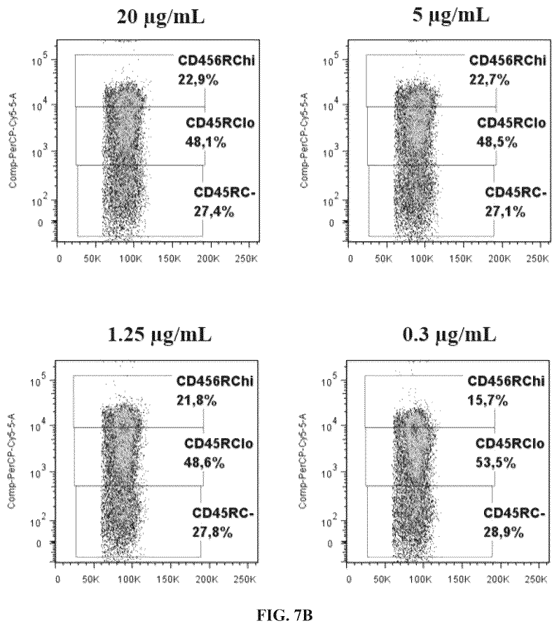

Labelling with either murine ABIS-45RC (FIG. 7A) or the humanized ABIS-45RC variant A1 (FIG. 7B) or variant A3 (FIG. 7C), at various concentrations, shows that both antibodies recognized human CD45RC in a similar manner.

### Example 11

### Cell Death Induction by Humanized ABIS-45RC Variants

### Material and Methods

Human PBMCs were incubated with medium, isotype control Ab or anti-CD45RC variants (10 μg/mL) for 6 hours. Then, cells were stained with anti-CD3 and anti-CD45RA, annexin V and DAPI. Percentage of total apoptosis was obtained by gating on DAPI⁺ Annexin V⁺+DAPI⁻ Annexin V⁺cells among T or non-T cells by flow cytometry.

### Results

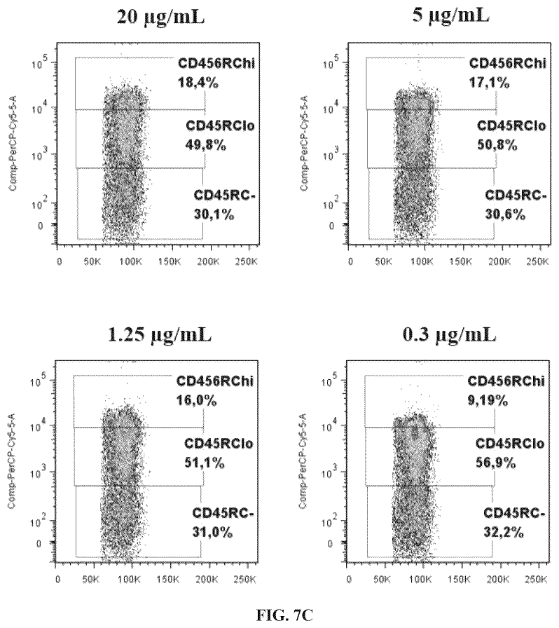

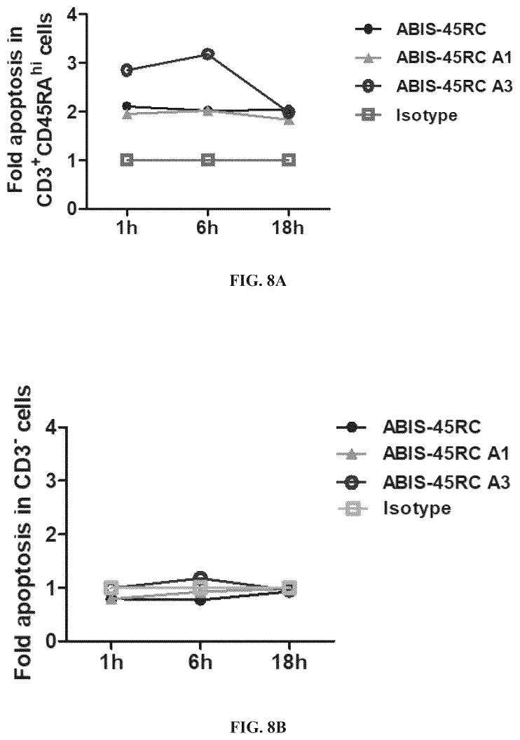

ABIS-45RC or the humanized variants A1 or A3 efficiently induced cell death of CD3⁺ cells (FIG. 8A) but not CD3⁻ cells (FIG. 8B).

### Example 12

Cross-Reactivity of Humanized ABIS-45RC with Primate

### Material and Methods

Blood from cynomolgus macaque was collected and peripheral blood mononuclear cells (PBMC) were isolated by Ficoll gradient centrifugation. PBMCs were labeled with ABIS-45RC or with humanized ABIS-45RC at 10 μg/ml and an anti-CD3 antibody. The reactivity was revealed using a biotin donkey anti-human IgG⁺ Streptavidin PercpCy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

### Results

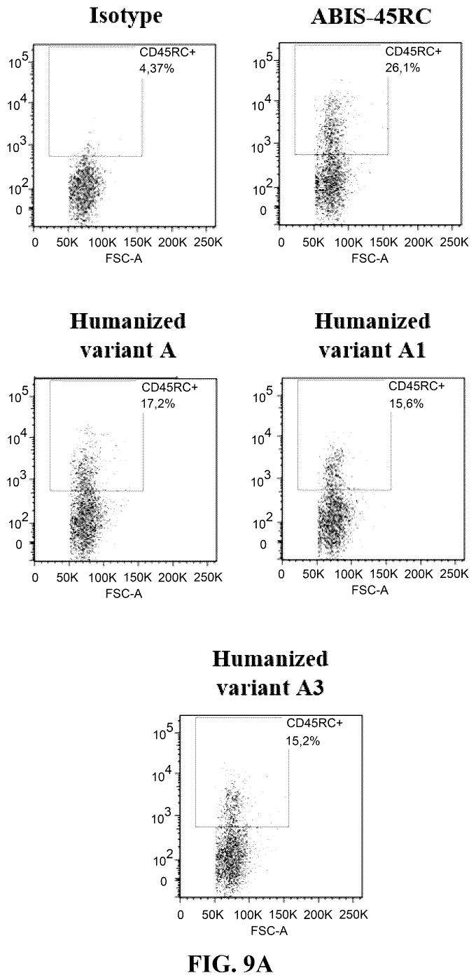

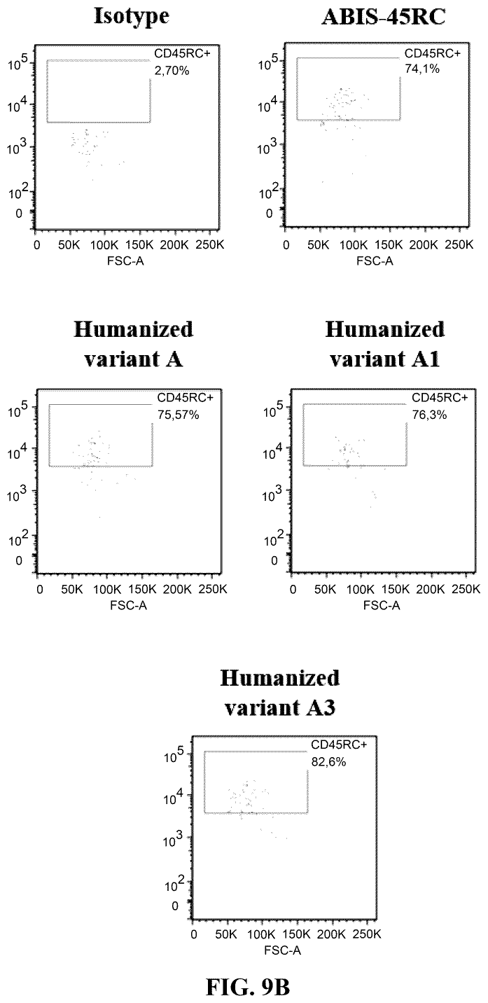

Labelling with either ABIS-45RC or the humanized variants A, A1 or A3 showed that all antibodies recognized macaque CD45RC in a similar manner in CD3+ cells (FIG. 9A) and CD3− cells (FIG. 9B).

### Example 13

Treatment of Human Skin Rejection with ABIS-45RC and Humanized Variant A1

### Material and Methods

### PBMC Isolation

Blood was collected at the Établissement Français du Sang (Nantes, France) from healthy individuals. Written informed consent was provided according to institutional guidelines. PBMC were isolated by Ficoll-Paque density-gradient centrifugation (Eurobio, Courtaboeuf, France). Remaining red cells and platelets were eliminated with a hypotonic solution and centrifugation.

### Animals

8- to 12-week-old NOD/SCID/IL2Rγ^{-/-} (NSG) mice were bred in our own animal facilities in SPF conditions (accreditation number C44-278).

### Human Skin Transplantation Model

Human skins were obtained from healthy volunteers from abdominoplasty surgery and transplantation was performed as previously described (Bézie et al., 2018. Front Immunol. 8:2014). One month later, 5×10⁶ PBMCs from allogeneic healthy volunteers were intravenously injected with or without antibodies.

Graft rejection was scored from 0 to 5 based on dryness (score 1), rigidity (score 2), scab (score 3), partial loss (score 4) and complete loss of the skin (score 5) by macroscopic observation.

Human PBMCs engraftment was monitored in blood by flow cytometry.

### Treatment

NSG mice were treated intraperitoneally with purified ABIS-45RC or humanized variant A1 antibodies at 0.8 mg/kg from day 0 and every 2.5 days during 20 days, together with intraperitoneal administration of rapamycin from day 0 to day 10 at a suboptimal dose of 0.4 mg/day.

### Results

Treatment with PBMCs only induced weight loss, initiated around day 14, and, as shown in Sure 10, death of all mice by day 33.

We previously showed that treatment with rapamycin only did not prolonged survival (median survival: 21 days (Bézie et al., 2018. Front Immunol. 8:2014)). Here, treatment with ABIS-45RC or the humanized variant A1 completely abrogated the skin graft rejection.

1. An isolated anti-human CD45RC antibody or binding fragment thereof, wherein said antibody or binding fragment thereof comprises:
(a) a heavy chain variable region (HCVR) which comprises the following three complementary-determining regions (CDRs):
(i) V_{H}-CDR1 of sequence SEQ ID NO: 1;
(ii) V_{H}-CDR2 with a sequence selected from the group consisting of sequences SEQ ID NOs: 4, 5, 6, 7, 8, 9, 10, 11, 100, 116, 117, 118 and 119; and
(iii) V_{H}-CDR3 of sequence SEQ ID NO: 3; and
(b) a light chain variable region (LCVR) which comprises the following three CDRs:
(i) V_{L}-CDR1 with a sequence selected from the group consisting of sequences SEQ ID NO: 15 (SASSSVS-X₁₂-YMH) and SEQ ID NO: 18 (RASSSVS-X₁₂-YMH), wherein X₁₂ is absent or is selected from the group consisting of Asn (N), Ser (S) and Gly (G);
(ii) V_{L}-CDR2 with a sequence selected from the group consisting of sequences SEQ ID NOs: 16, 19, 20, 22, 111, 120 and 127; and
(iii) V_{L}-CDR3 with a sequence selected from the group consisting of sequences SEQ ID NOs: 17 and 21.

2. The isolated antibody or binding fragment thereof according to claim 1, wherein said antibody or binding fragment thereof comprises a combination of 3 HCVR's CDRs and 3 LCVR's CDRs as set forth in Table 2.

3. The isolated antibody or binding fragment thereof according to claim 1, wherein said antibody or binding fragment thereof comprises:
(a) a HCVR which comprises the following three CDRs:
(i) V_{H}-CDR1 of sequence SEQ ID NO: 1;
(ii) V_{H}-CDR2 with a sequence selected from the group consisting of sequences SEQ ID NOs: 4 and 5; and
(iii) V_{H}-CDR3 of sequence SEQ ID NO: 3; and
(b) a LCVR which comprises the following three CDRs:
(i) V_{L}-CDR1 of sequence SEQ ID NO: 15, wherein X₁₂ is absent;
(ii) V_{L}-CDR2 of sequence SEQ ID NO: 16; and
(iii) V_{L}-CDR3 of sequence SEQ ID NO: 17.

4. The isolated antibody or binding fragment thereof according to claim 1, wherein said antibody or binding fragment thereof comprises:
(a) a HCVR which comprises the following three CDRs:
(i) V_{H}-CDR1 of sequence SEQ ID NO: 1;
(ii) V_{H}-CDR2 of sequence SEQ ID NO: 4; and
(iii) V_{H}-CDR3 of sequence SEQ ID NO: 3; and
(b) a LCVR which comprises the following three CDRs:
(i) V_{L}-CDR1 of sequence SEQ ID NO: 15, wherein X₁₂ is absent;
(ii) V_{L}-CDR2 of sequence SEQ ID NO: 16; and
(iii) V_{L}-CDR3 of sequence SEQ ID NO: 17.

5. The isolated antibody or binding fragment thereof according to claim 1, wherein said antibody or binding fragment thereof comprises:
(a) a HCVR which comprises the following three CDRs:
(i) V_{H}-CDR1 of sequence SEQ ID NO: 1;
(ii) V_{H}-CDR2 with a sequence selected from the group consisting of sequences SEQ ID NOs: 4, 6, and 100; and
(iii) V_{H}-CDR3 of sequence SEQ ID NO: 3; and
(b) a LCVR which comprises the following three CDRs:
(i) V_{L}-CDR1 with a sequence selected from the group consisting of sequences SEQ ID NOs: 15 and 18, wherein X₁₂ is absent;
(ii) V_{L}-CDR2 of sequence SEQ ID NO: 16; and
(iii) V_{L}-CDR3 of sequence SEQ ID NO: 17.

6. The isolated antibody or binding fragment thereof according to claim 1, wherein said antibody or binding fragment thereof comprises:
1) a HCVR of sequence SEQ ID NO: 61 and a LCVR of sequence SEQ ID NO: 81;
2) a HCVR of sequence SEQ ID NO: 62 and a LCVR of sequence SEQ ID NO: 82;
3) a HCVR of sequence SEQ ID NO: 62 and a LCVR of sequence SEQ ID NO: 83;
4) a HCVR of sequence SEQ ID NO: 62 and a LCVR of sequence SEQ ID NO: 84;
5) a HCVR of sequence SEQ ID NO: 63 and a LCVR of sequence SEQ ID NO: 82;
6) a HCVR of sequence SEQ ID NO: 63 and a LCVR of sequence SEQ ID NO: 83;
7) a HCVR of sequence SEQ ID NO: 63 and a LCVR of sequence SEQ ID NO: 84;
8) a HCVR of sequence SEQ ID NO: 64 and a LCVR of sequence SEQ ID NO: 82;
9) a HCVR of sequence SEQ ID NO: 64 and a LCVR of sequence SEQ ID NO: 83;
10) a HCVR of sequence SEQ ID NO: 64 and a LCVR of sequence SEQ ID NO: 84;
11) a HCVR of sequence SEQ ID NO: 101 and a LCVR of sequence SEQ ID NO: 85;
12) a HCVR of sequence SEQ ID NO: 101 and a LCVR of sequence SEQ ID NO: 103;
13) a HCVR of sequence SEQ ID NO: 65 and a LCVR of sequence SEQ ID NO: 85;
14) a HCVR of sequence SEQ ID NO: 65 and a LCVR of sequence SEQ ID NO: 103;
15) a HCVR of sequence SEQ ID NO: 62 and a LCVR of sequence SEQ ID NO: 85;
16) a HCVR of sequence SEQ ID NO: 101 and a LCVR of sequence SEQ ID NO: 82;
17) a HCVR of sequence SEQ ID NO: 121 and a LCVR of sequence SEQ ID NO: 85;
18) a HCVR of sequence SEQ ID NO: 122 and a LCVR of sequence SEQ ID NO: 85;
19) a HCVR of sequence SEQ ID NO: 123 and a LCVR of sequence SEQ ID NO: 85;
20) a HCVR of sequence SEQ ID NO: 124 and a LCVR of sequence SEQ ID NO: 85;
21) a HCVR of sequence SEQ ID NO: 63 and a LCVR of sequence SEQ ID NO: 85;
22) a HCVR of sequence SEQ ID NO: 67 and a LCVR of sequence SEQ ID NO: 85;
23) a HCVR of sequence SEQ ID NO: 67 and a LCVR of sequence SEQ ID NO: 103; or
24) a HCVR of sequence SEQ ID NO: 61 and a LCVR of sequence SEQ ID NO: 113; or
25) a HCVR of sequence SEQ ID NO: 61 and a LCVR of sequence SEQ ID NO: 126; or
26) a HCVR and a LCVR comprising a sequence of the non-CDR regions sharing at least 70% of identity with the sequence of the non-CDR regions of the HCVR and LCVR according to 1) to 25).

7. The isolated antibody or binding fragment thereof according to claim 1, wherein said antibody or binding fragment thereof comprises:
(a) a HCVR which comprises the following three CDRs:
(i) V_{H}-CDR1 of sequence SEQ ID NO: 1;
(ii) V_{H}-CDR2 with a sequence selected from the group consisting of sequences SEQ ID NOs: 4, 5, 6, 7, 8, 9, 10, 11, 100, 116, 117, 118 and 119; and
(iii) V_{H}-CDR3 of sequence SEQ ID NO: 3; and
(b) a LCVR which comprises the following three CDRs:
(i) V_{L}-CDR1 with a sequence selected from the group consisting of sequences SEQ ID NOs: 15 and 18, wherein X₁₂ in SEQ ID NOs: 15 and 18 is selected from the group consisting of Asn (N), Ser (S) and Gly (G);
(ii) V_{L}-CDR2 with a sequence selected from the group consisting of sequences SEQ ID NOs: 16, 19, 20, 22, 111, 120 and 127; and
(iii) V_{L}-CDR3 with a sequence selected from the group consisting of sequences SEQ ID NOs: 17 and 21.

8. The isolated antibody or binding fragment thereof according to claim 7, wherein the amino acid residue at Kabat position L71 of the LCVR is Phe (F).

9. A nucleic acid encoding the isolated antibody or binding fragment thereof according to claim 1.

10. An expression vector comprising the nucleic acid according to claim 9.

11. A cell comprising the nucleic acid according to claim 9 or an expression vector comprising the nucleic acid.

12. A pharmaceutical composition comprising the isolated antibody or binding fragment thereof according to claim 1, a nucleic acid encoding the isolated antibody or binding fragment thereof, an expression vector comprising the nucleic acid, or a cell comprising the nucleic acid or the expression vector, and at least one pharmaceutically acceptable excipient.

13. A method of inducing immune tolerance in a subject in need thereof, comprising administering to said subject the isolated antibody or binding fragment thereof according to claim 1, a nucleic acid encoding the isolated antibody or binding fragment thereof, an expression vector comprising the nucleic acid, or a cell comprising the nucleic acid or the expression vector.

14. A method of preventing and/or reducing transplant rejections in a subject in need thereof, comprising administering to said subject the isolated antibody or binding fragment thereof according to claim 1, a nucleic acid encoding the isolated antibody or binding fragment thereof, an expression vector comprising the nucleic acid, or a cell comprising the nucleic acid or the expression vector.

15. A method of preventing, reducing and/or treating CD45RC^{high}-related conditions in a subject in need thereof, comprising administering to said subject the isolated antibody or binding fragment thereof according to claim 1, a nucleic acid encoding the isolated antibody or binding fragment thereof, an expression vector comprising the nucleic acid, or a cell comprising the nucleic acid or the expression vector.

16. The method of preventing, reducing and/or treating CD45RC^{high}-related conditions according to claim 15, wherein the CD45RC^{high}-related condition is selected from the group consisting of autoimmune diseases, undesired immune responses, monogenic diseases, lymphoma and cancer.

17. A method of preventing and/or treating graft-versus-host disease (GVHD) in a subject in need thereof, comprising administering to said subject the isolated antibody or binding fragment thereof according to claim 1, a nucleic acid encoding the isolated antibody or binding fragment thereof, an expression vector comprising the nucleic acid, or a cell comprising the nucleic acid or the expression vector.

18. A method for detecting or quantifying hCD45RC in a sample, cell, tissue or organ, comprising contacting said sample, cell, tissue or organ with the isolated antibody or binding fragment thereof according to claim 1.

19. The method for detecting or quantifying hCD45RC according to claim 18, wherein the isolated antibody or binding fragment thereof is labelled.

20. The isolated antibody or binding fragment thereof according to claim 1, wherein said antibody or binding fragment thereof comprises:
(a) a HCVR which comprises the following three CDRs:
(i) V_{H}-CDR1 of sequence SEQ ID NO: 1;
(ii) V_{H}-CDR2 with a sequence selected from the group consisting of sequences SEQ ID NOs: 4 and 118; and
(iii) V_{H}-CDR3 of sequence SEQ ID NO: 3; and
(b) a LCVR which comprises the following three CDRs:
(i) V_{L}-CDR1 of sequence SEQ ID NO: 15, wherein X₁₂ is absent;
(ii) V_{L}-CDR2 of sequence SEQ ID NO: 120; and
(iii) V_{L}-CDR3 of sequence SEQ ID NO: 17.
